SEQUENCE LISTING (102 sequences)

SEQ ID NO: 1 - protein, 14 aa, Artificial
  Mutated sequence from lama glama.
RAPDTRLXPYXYDX

SEQ ID NO: 2 - protein, 18 aa, Artificial
  Mutated sequence from lama glama.
DRYIWARQGEYWGAYXDY

SEQ ID NO: 3 - protein, 17 aa, Lama glama
  MISC_FEATURE (16)..(16): Xaa may be Asp or Glu
SRAYGSSRLRLADTYXY

SEQ ID NO: 4 - protein, 124 aa, Lama glama
EVQLVESGGGLVQPGGSLRLSCAISGFTLDLHVIGWLRQAPGKEREWVSCISSSDGSTYYADSVKGRFTISRDNAKNTVYLQMNSLKPEDTAVYYCAAPWDSWYCGIGNDYDYWGQGTQVTVSS

SEQ ID NO: 5 - protein, 123 aa, Lama glama
EVQLVESEGGLVQAGGSLRLSCAASGSTFSSYAMGWYRQAPGKQRELVAVISNGGITNYPNSVKGRFTISRDNAKNTVYLQMNSLKPEDTAVYYCFYSGSYYYPTDVHEYDYWGQGTQVTVSS

SEQ ID NO: 6 - protein, 123 aa, Lama glama
EVQLVESGGGLVQAGGSLRLSCAASGSTFNSYAMGWYRQAPGKQREWVAAFSTGGSTNYADSVKGRFTISRDNAKNTVYLQMNSLKPEDTAVYYCFYSGSYYYPTDVFEYDYWGQGTQVTVSS

SEQ ID NO: 7 - protein, 130 aa, Lama glama
EVQLVESGGGLVQAGGSLRLSCAASGFALDYYAVGWFRQAPGKEREGVSCISSRGGSTFYADSVKGRFTTSRNNAKNTVYLQMNSLKPEDTAVYYCAAHPLQNCCGGSAYASPEAVYEYWGQGTQVTVSS

SEQ ID NO: 8 - protein, 125 aa, Lama glama
EVQLVESGGGLVQPGGSLRLSCAASGFTLDYYNIGWFRQAPGKEREWVSCINSSDGSTYYADSVKGRFTISRDNAKNTVYLQMNSLKPEDTAVYYCAAPFAYYSNLCGVNGYDYWGQGTQVTVSS

SEQ ID NO: 9 - protein, 124 aa, Lama glama
EVQLVESGGGLVQAGGSLRLSCAASGFTFDDYAIGWFRQAPGKEREGVSCISSHDRTTYYADSVKGRFTISSDNAKNTVYLQMNSLKPEDTAVYYCAADPLVCGYNDPRLADYWGQGTQVTVSS

SEQ ID NO: 10 - protein, 123 aa, Lama glama
EVQLVESGGGLVQAGGSLRLSCAASGRTFSSYAMAWFRQAPGKEREFVAAIRWSGGTAYYADSVQGRFTISRDNAKNTVYLQMNSLKPEDTAVYYCANRAADTRLGPYEYDYWGQGTQVTVSS

SEQ ID NO: 11 - protein, 123 aa, Lama glama
EVQLVESGGGLVQAGGSLRLSCAASGFTFDDYAIGWFRQAPGKEREGVSCISSSDGSTYYADSVKGRFTISSDNAKNTVYLQMNSLKPEDTAVYYCATAWCDSSWYRSFVGYWGQGTQVTVSS

SEQ ID NO: 12 - protein, 128 aa, Lama glama
EVQLVESGGGLVQPGGSLRLSCAASGFTFGSYDMSWVRRSPGKGPEWVSSINSGGGSTYYADFVKGRFTISRDNAKNTLYLQMNSLKPEDTAVYYCAADRYIRARQGDYWGAYEYDYWGQGTQVTVSS

SEQ ID NO: 13 - protein, 117 aa, Lama glama
EVQLVESGGGLVQAEGSLRLSCAASGSTFSTYAMAWYRQAPGKQRELVAGISFDGSTHYAESVKGRFTISRDDAKNTVSLQMNSLKPEDAAVYYCYSVHPSTGFGSWGQGTQVTVSS

SEQ ID NO: 14 - protein, 120 aa, Lama glama
EVQLVESGGGLVQPGGSLRLSCAASGFTFDDYAIGWFRQAPGKEPEGISCISSSGGITYYADSVKGRFTISRDNAKNTVYLQMNSLKPEDTAVYYCATPGIAACRGIHYTGQGTQVTVSS

SEQ ID NO: 15 - protein, 120 aa, Lama glama
EVQLVESGGGLVQPGGSLRLSCAASGFTFDDYAIGWFRQAPGKEPEGISCISSSGGITYYADSVKGRFTTSRDNAKNTVYLQMNSLKPEDTAVYYCATPGIAACRGIHYTGQGTQVTVSS

SEQ ID NO: 16 - protein, 120 aa, Lama glama
EVQLVESGGGLVQPGGSLRLSCTASGFTFDVYAIGWFRQAPGKEPEGISCISSSGSITYYADSVKGRFTTSRDSAKNTVYLQMNSLKPEDTAVYYCATPGIAACRGIHYWGQGTQVTVSS

SEQ ID NO: 17 - protein, 120 aa, Lama glama
EVQLVESGGGLVQPGGSLRLSCAASGFTFDDYAIGWFRQAPGKEPEEISCISSSGGITYYADSVKGRFTISRDNAKNTVYLQMNSLKPEDTAVYYCATPGIAACRGIHYTGQGTQVTVSS

SEQ ID NO: 18 - protein, 116 aa, Lama glama
EVQLMESGGGLVQPGGSLRLSCVAAGFTFSNYYMSWVRQAPGKGLEWVSVISPDGSNTYYADTVKGRFTISRGNAKNTLFLQMTGLKSEDAAVYYCARGSGSWGVHGQGTQVTVSS

SEQ ID NO: 19 - protein, 123 aa, Lama glama
EVQLVESGGGLVQPGGSLRLSCAASGFTFGNYDMSWVRQAPGKGPEWVSAINSGGGDTYYADSVKGRFTISRDNAKNTLYLQMNSLKPEDTAVYYCATPRGWGPTGPHEYGYWGQGTQVTVSS

SEQ ID NO: 20 - protein, 120 aa, Lama glama
EVQLVESGGGLVQPGGSLRLSCTASGFTFDDYAIGWFRQAPGKEPEGISCISSSGSITYDADSVKGRFTISRDNAKNTVYLQMNSLKPEDTAVYYCATPGIAACRGIHYWGQGTQVTVSS

SEQ ID NO: 21 - protein, 123 aa, Artificial
  Mutated sequence from lama glama.
EVQLVESGGGLVQAGGSLRLSCAASGRTFSSYAMAWFRQAPGKEREYVAAIRWSGGTAYYADSVQGRFTISRDNAKNTVYLQMNSLRPEDTAVYYCANRAPDTRLRPYLYDYWGQGTQVTVSS

SEQ ID NO: 22 - protein, 123 aa, Artificial
  Mutated sequence from lama glama.
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYEYDHWGQGTQVTVSS

SEQ ID NO: 23 - protein, 123 aa, Artificial
  Mutated sequence from lama glama.
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLEPYLYDYWGQGTQVTVSS

SEQ ID NO: 24 - protein, 123 aa, Artificial
  Mutated sequence from lama glama.
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLRPYLYDYWGQGTQVTVSS

SEQ ID NO: 25 - protein, 123 aa, Artificial
  Mutated sequence from lama glama.
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLEPYEYDYWGQGTQVTVSS

SEQ ID NO: 26 - protein, 123 aa, Artificial
  Mutated sequence from lama glama.
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYLYDYWGQGTQVTVSS

SEQ ID NO: 27 - protein, 123 aa, Artificial
  Mutated sequence from lama glama.
EVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYEYDYWGQGTQVTVSS

SEQ ID NO: 28 - protein, 123 aa, Artificial; identical to SEQ ID NO: 27
  Mutated sequence from lama glama.

SEQ ID NO: 29 - protein, 128 aa, Artificial
  Mutated sequence from lama glama.
EVQLVESGGGLVQPGGSLRLSCAASGFTFGSYDMSWVRRSPGKGPEWVSAINSGGGSTYYADYVKGRFTISRDNAKNTLYLQMNSLKPEDTAVYYCAADRYIWARQGEYWGAYAYDYWGQGTQVTVSS

SEQ ID NO: 30 - protein, 128 aa, Artificial
  Mutated sequence from lama glama.
EVQLVESGGGLVQPGGSLRLSCAASGFTFGSYDMSWVRRSPGKGPEWVSAINSGGGSTYYTDYVKGRFTISRDNAKNTLYLQMNSLKPEDTAVYYCAADRYIWARQGEYWGAYAYDYWGQGTQVTVSS

SEQ ID NO: 31 - protein, 128 aa, Artificial
  Mutated sequence from lama glama.
EVQLVESGGGLVQPGGSLRLSCAASGFTIGSYDMSWVRRSPGKGPEWVSSINSGGGSTYYTDYVKGRFTISRDNAKNTLYLQMNSLKPEDTAVYYCAADRYIWARQGEYWGAYAYDYWGQGTQVTVSS

SEQ ID NO: 32 - protein, 128 aa, Artificial
  Mutated sequence from lama glama.
EVQLVESGGGLVQPGGSLRLSCAASGFTFGSYDMSWVRRSPGKGPEWVSSINSGGGSTYYADYVKGRFTISRDNAKNTLYLQMNSLKPEDTAVYYCAADRYIWARQGEYWGAYEYDYWGQGTQVTVSS

SEQ ID NO: 33 - protein, 128 aa, Artificial
  Mutated sequence from lama glama.
EVQLVESGGGLVQPGGSLRLSCAASGFTIGSYDMSWVRRSPGKGPEWVSAINSGGGSTYYADYVKGRFTISRDNAKNTLYLQMNSLKPEDTAVYYCAADRYIWARQGEYWGAYAYDYWGQGTQVTVSS

SEQ ID NO: 34 - protein, 123 aa, Artificial
  Mutated sequence from lama glama.
EVQLVESGGGLVQPGGSLRLSCAASGRTFSSYAMAWYRQAPGKEREYVAAIRWSGGTAYYADSVQGRFTISRDNAKNTVYLQMNSLRPEDTAVYYCANRAPDTRLAPYEYDHWGQGTLVTVSS

SEQ ID NO: 35 - protein, 123 aa, Artificial
  Mutated sequence from lama glama.
EVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 36 - protein, 128 aa, Artificial
  Mutated sequence from lama glama.
EVQLVESGGGLVQPGGSLRLSCAASGFTFGSYDMSWVRRSPGKGPEWVSSINSGGGSTYYADYVKGRFTISRDNAKNTLYLQMNSLRPEDTAVYYCAADRYIWARQGEYWGAYEYDYWGQGTLVTVSS

SEQ ID NO: 37 - protein, 128 aa, Artificial
  Mutated sequence from lama glama.
EVQLVESGGGLVQPGGSLRLSCAASGFTFGSYDMSWVRQSPGKGPEWVSSINSGGGSTYYADYVKGRFTISRDNAKNTLYLQMNSLRPEDTAVYYCAADRYIWARQGEYWGAYEYDYWGQGTLVTVSS

SEQ ID NO: 38 - protein, 128 aa, Artificial
  Mutated sequence from lama glama.
EVQLVESGGGLVQPGGSLRLSCAASGFTFGSYDMSWVRRAPGKGPEWVSSINSGGGSTYYADYVKGRFTISRDNAKNTLYLQMNSLRPEDTAVYYCAADRYIWARQGEYWGAYEYDYWGQGTLVTVSS

SEQ ID NO: 39 - protein, 128 aa, Artificial
  Mutated sequence from lama glama.
EVQLVESGGGLVQPGGSLRLSCAASGFTFGSYDMSWVRQAPGKGPEWVSSINSGGGSTYYADYVKGRFTISRDNAKNTLYLQMNSLRPEDTAVYYCAADRYIWARQGEYWGAYEYDYWGQGTLVTVSS

SEQ ID NO: 40 - protein, 128 aa, Artificial
  Mutated sequence from lama glama.
EVQLVESGGGLVQPGGSLRLSCAASGFTFGSYDMSWVRRAPGKGPEWVSSISSGGGSTYYADYVKGRFTISRDNAKNTLYLQMNSLRPEDTAVYYCAADRYIWARQGEYWGAYEYDYWGQGTLVTVSS

SEQ ID NO: 41 - protein, 128 aa, Artificial
  Mutated sequence from lama glama.
EVQLVESGGGLVQPGGSLRLSCAASGFTIGSYDMSWVRRAPGKGPEWVSSISSGGGSTYYADYVKGRFTISRDNAKNTLYLQMNSLRPEDTAVYYCAADRYIWARQGEYWGAYEYDYWGQGTLVTVSS

SEQ ID NO: 42 - protein, 125 aa, Lama glama
EVQLVESGGGLVQPGDSLKLSCAFSGRTFSSYSMGWFRQAQGKEREFVVAISSGGFIYDAVSLEGRFTISRDNTKNTVYLQTPSLKPEDTAVYYCAASRAYGSSRLRLADTYDYWGQGTQVTVSS

SEQ ID NO: 43 - protein, 125 aa, Lama glama
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 44 - protein, 125 aa, Lama glama
EVQLVESGGGLVQPGDSLKLSCVASGRTSSSYSMGWFRQAQGKEREFVVAISSGGYIYDSVSLQGRFTISRDNTKNTVYLQTPSLKPEDTAVYYCAASRAYGSSRLRLADTYDYWGQGTQVTVSS

SEQ ID NO: 45 - protein, 113 aa, Lama glama
EVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSS

SEQ ID NO: 46 - protein, 124 aa, Lama glama
EVQLVESGGGLVQAGGSLRLSCAASGRTFSNYAMGWFRQAPGKERVLVADISSSGINTYVADAVKGRFTISRDNAKNTVYLQMNSLKPEDTAVYYCAASAWWYSQMARDNYRYWGQGTQVTVSS

SEQ ID NO: 47 - protein, 123 aa, Lama glama
EVQLVESGGGLVQAGGSLRLSCTASGSAFKSYRMGWFRRTPGKEDEFVASISWTYGSTFYADSVKGRFTMSRDKAKNAGYLQMNSLKPEDTALYYCAAGAQSDRYNIRSYDYWGQGTQVTVSS

SEQ ID NO: 48 - protein, 285 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGSGGGGSGGGGSGGGGSGGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 49 - protein, 247 aa, Artificial
  Artificial polypeptide comprising lama glama sequences
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSS

SEQ ID NO: 50 - protein, 278 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGSGGGGSGGGGSGGGGSGGGGSGGGGSEVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSS

SEQ ID NO: 51 - protein, 289 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGSGGGGSGGGGSGGGGSGGGGSGGGGSEVQLVESGGGLVQAGGSLRLSCAASGRTFSNYAMGWFRQAPGKERVLVADISSSGINTYVADAVKGRFTISRDNAKNTVYLQMNSLKPEDTAVYYCAASAWWYSQMARDNYRYWGQGTQVTVSS

SEQ ID NO: 52 - protein, 288 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGSGGGGSGGGGSGGGGSGGGGSGGGGSEVQLVESGGGLVQAGGSLRLSCTASGSAFKSYRMGWFRRTPGKEDEFVASISWTYGSTFYADSVKGRFTMSRDKAKNAGYLQMNSLKPEDTALYYCAAGAQSDRYNIRSYDYWGQGTQVTVSS

SEQ ID NO: 53 - protein, 289 aa, Artificial
  Artificial polypeptide comprising lama glama sequences.
EVQLVESGGGLVQAGGSLRLSCAASGRTFSNYAMGWFRQAPGKERVLVADISSSGINTYVADAVKGRFTISRDNAKNTVYLQMNSLKPEDTAVYYCAASAWWYSQMARDNYRYWGQGTQVTVSSGGGGSGGGGSGGGGSGGGGSGGGGSGGGGSGGGGSGGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 54 - protein, 125 aa, Artificial
  Mutated sequence from lama glama.
EVQLVESGGGLVQTGGSLRLSCEASGRTFSSYSMGWFRQAPGKEREFVVAISKGGYKYDSVSLEGRFTISRDNAKNTVYLQINSLRPEDTAVYYCASSRAYGSSRLRLADTYEYWGQGTLVTVSS

SEQ ID NO: 55 - protein, 125 aa, Artificial
  Mutated sequence from lama glama.
EVQLVESGGGLVQPGGSLRLSCAASGRTFSSYSMGWFRQAPGKEREFVVAISKGGYKYDSVSLEGRFTISRDNAKNTVYLQMNSLRPEDTAVYYCASSRAYGSSRLRLADTYEYWGQGTLVTVSS

SEQ ID NO: 56 - protein, 125 aa, Artificial
  Mutated sequence from lama glama.
EVQLVESGGGLVQPGGSLRLSCEASGRTFSSYSMGWFRQAPGKEREFVVAISKGGYKYDSVSLEGRFTISRDNAKNTVYLQINSLRPEDTAVYYCASSRAYGSSRLRLADTYEYWGQGTLVTVSS

SEQ ID NO: 57 - protein, 125 aa, Artificial
  Mutated sequence from lama glama.
EVQLVESGGGLVQTGGSLRLSCEVSGRTFSSYSMGWFRQAPGKEREFVVAISKGGYKYDSVSLEGRFTISKDNAKNTVYLQINSLRPEDTAVYYCASSRAYGSSRLRLADTYEYWGQGTLVTVSS

SEQ ID NO: 58 - protein, 125 aa, Artificial
  Mutated sequence from lama glama.
EVQLVESGGGLVQTGDSLRLSCEVSGRTFSSYSMGWFRQAQGKEREFVVAISKGGYKYDSVSLEGRFTISKDNAKNTVYLQMNSLRPEDTAVYYCASSRAYGSSRLRLADTYEYWGQGTLVTVSS

SEQ ID NO: 59 - protein, 125 aa, Artificial
  Mutated sequence from lama glama.
EVQLVESGGGLVQPGDSLRLSCEVSGRTFSSYSMGWFRQAPGKEREFVVAISKGGYKYDSVSLEGRFTISKDNAKNTVYLQINSLRPEDTAVYYCASSRAYGSSRLRLADTYEYWGQGTLVTVSS

SEQ ID NO: 60 - protein, 125 aa, Artificial
  Mutated sequence from lama glama.
EVQLVESGGGLVQTGGSLRLSCEVSGRTFSSYSMGWFRQAPGKEREFVVAISKGGYKYDSVSLEGRFTISRDNAKNTVYLQINSLRPEDTAVYYCASSRAYGSSRLRLADTYEYWGQGTLVTVSS

SEQ ID NO: 61 - protein, 125 aa, Artificial
  Mutated sequence from lama glama.
EVQLVESGGGLVQTGDSLRLSCAVSGRTFSSYSMGWFRQAQGKEREFVVAISKGGYKYDSVSLEGRFTISKDNAKNTVYLQINSLRPEDTAVYYCASSRAYGSSRLRLADTYEYWGQGTLVTVSS

SEQ ID NO: 62 - protein, 125 aa, Artificial
  Mutated sequence from lama glama.
EVQLVESGGGLVQTGDSLRLSCEVSGRTFSSYSMGWFRQAQGKEREFVVAISKGGYKYDAVSLEGRFTISKDNAKNTVYLQINSLKPEDTAVYYCASSRAYGSSRLRLADTYEYWGQGTQVTVSS

SEQ ID NO: 63 - protein, 125 aa, Artificial
  Mutated sequence from lama glama.
DVQLVESGGGLVQPGGSLRLSCAASGRTFSSYSMGWFRQAPGKEREFVVAISKGGYKYDAVSLEGRFTISRDNAKNTVYLQMNSLRPEDTAVYYCASSRAYGSSRLRLADTYEYWGQGTLVTVSS

SEQ ID NO: 64 - protein, 125 aa, Artificial
  Mutated sequence from lama glama.
DVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSS

SEQ ID NO: 65 - protein, 113 aa, Artificial
  Mutated sequence from lama glama.
EVQLVESGGGLVQPGGSLRLSCAASGIRFMSMAWYRQAPGKQRELVARISSGGTTAYADSVKGRFTISRDNSKNTVYLQMNSLRAEDTAVYYCNTFSSRPNPWGQGTLVTVSS

SEQ ID NO: 66 - protein, 113 aa, Artificial
  Mutated sequence from lama glama.
EVQLVESGGGLVQPGGSLRLSCVASGIRFISMAWYRQAPGKHRELVARISSGGTTAYVDSVKGRFTISRDNSKNTVYLQMNSLKAEDTAVYYCNTFSSRPNPWGAGTQVTVSS

SEQ ID NO: 67 - protein, 113 aa, Artificial
  Mutated sequence from lama glama.
EVQLVESGGGLVQPGGSLRLSCAASGIRFISMAWYRQAPGKQRELVARISSGGTTAYADSVKGRFTISRDNSKNTVYLQMNSLRAEDTAVYYCNTFSSRPNPWGQGTLVTVSS

SEQ ID NO: 68 - protein, 283 aa, Artificial
  Artificial polypeptide comprising lama glama sequences.
EVQLVESGGGLVQAGGSLRLSCAASGRTFSSYAMAWFRQAPGKEREFVAAIRWSGGTAYYADSVQGRFTISRDNAKNTVYLQMNSLKPEDTAVYYCANRAADTRLGPYEYDYWGQGTQVTVSSGGGGSGGGGSGGGGCGGGGSGGGGSGGGGSGGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 69 - protein, 283 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGCGGGGSGGGGSGGGGSGGGGSEVQLVESGGGLVQAGGSLRLSCAASGRTFSSYAMAWFRQAPGKEREFVAAIRWSGGTAYYADSVQGRFTISRDNAKNTVYLQMNSLKPEDTAVYYCANRAADTRLGPYEYDYWGQGTQVTVSS

SEQ ID NO: 70 - protein, 402 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGSGGGGSGGGGSGGGGSEVQLVESGGGLVQAGGSLRLSCAASGRTFSSYAMAWFRQAPGKEREFVAAIRWSGGTAYYADSVQGRFTISRDNAKNTVYLQMNSLKPEDTAVYYCANRAADTRLGPYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 71 - protein, 381 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQAGGSLRLSCAASGRTFSSYAMAWFRQAPGKEREFVAAIRWSGGTAYYADSVQGRFTISRDNAKNTVYLQMNSLKPEDTAVYYCANRAADTRLGPYEYDYWGQGTQVTVSS

SEQ ID NO: 72 - protein, 402 aa, Artificial
  Artificial polypeptide comprising lama glama sequences.
EVQLVESGGGLVQAGGSLRLSCAASGRTFSSYAMAWFRQAPGKEREFVAAIRWSGGTAYYADSVQGRFTISRDNAKNTVYLQMNSLKPEDTAVYYCANRAADTRLGPYEYDYWGQGTQVTVSSGGGGSGGGGSGGGGSGGGGSGGGGSGGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 73 - protein, 381 aa, Artificial
  Artificial polypeptide comprising lama glama sequences.
EVQLVESGGGLVQAGGSLRLSCAASGRTFSSYAMAWFRQAPGKEREFVAAIRWSGGTAYYADSVQGRFTISRDNAKNTVYLQMNSLKPEDTAVYYCANRAADTRLGPYEYDYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSS

SEQ ID NO: 74 - protein, 381 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYEYDHWGQGTQVTVSS

SEQ ID NO: 75 - protein, 386 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGFTFGSYDMSWVRRSPGKGPEWVSSINSGGGSTYYADYVKGRFTISRDNAKNTLYLQMNSLKPEDTAVYYCAADRYIWARQGEYWGAYEYDYWGQGTQVTVSS

SEQ ID NO: 76 - protein, 288 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGCGGGGSGGGGSGGGGSGGGGSEVQLVESGGGLVQPGGSLRLSCAASGFTFGSYDMSWVRRSPGKGPEWVSSINSGGGSTYYADYVKGRFTISRDNAKNTLYLQMNSLKPEDTAVYYCAADRYIWARQGEYWGAYEYDYWGQGTQVTVSS

SEQ ID NO: 77 - protein, 283 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGCGGGGSGGGGSGGGGSGGGGSEVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYEYDHWGQGTQVTVSS

SEQ ID NO: 78 - protein, 405 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGSEVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSSGGGGSGGGGSGGGGCGGGGSGGGGSGGGGSGGGGSEVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYEYDHWGQGTQVTVSS

SEQ ID NO: 79 - protein, 431 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGCGGGGSGGGGSGGGGSGGGGSEVQLVESGGGLVQPGGSLRLSCVASGIRFMSMAWYRQAPGKHRELVARISSGGTTAYVDSVKGRFTISRDNSKNTVYLQMNSLKAEDTAVYYCNTFSSRPNPWGAGTQVTVSSGGGGSGGGGSGGGGSGGGGSGGGGSGGGGSGGGGSEVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYEYDHWGQGTQVTVSS

SEQ ID NO: 80 - protein, 407 aa, Artificial
  Artificial polypeptide comprising lama glama sequences.
EVQLVESGGGLVQTGDSLRLSCEVSGRTFSSYSMGWFRQAQGKEREFVVAISKGGYKYDSVSLEGRFTISKDNAKNTVYLQINSLKPEDTAVYYCASSRAYGSSRLRLADTYEYWGQGTQVTVSSGGGGSGGGGSGGGGSGGGGSGGGGSGGGGSGGGGSEVQLVESGGGLVQAGGSLRLSCAASGRTFSSYAMAWYRQAPGKEREYVAAIRWSGGTAYYADSVQGRFTISRDNAKNTVYLQMNSLKPEDTAVYYCANRAPDTRLAPYEYDHWGQGTQVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSS

SEQ ID NO: 81 - protein, 282 aa, Artificial
  Artificial polypeptide comprising sequences derived from lama glama.
VQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGGSGGGGCGGGGSGGGGSGGGGSGGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 82 - protein, 380 aa, Artificial
  Artificial polypeptide comprising sequences derived from lama glama.
VQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGSEVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 83 - protein, 430 aa, Artificial
  Artificial polypeptide comprising sequences derived from lama glama.
VQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGGSGGGGCGGGGSGGGGSGGGGSGGGGSEVQLVESGGGLVQPGGSLRLSCAASGIRFISMAWYRQAPGKQRELVARISSGGTTAYADSVKGRFTISRDNSKNTVYLQMNSLRAEDTAVYYCNTFSSRPNPWGQGTLVTVSSGGGGSGGGGSGGGGSGGGGSGGGGSGGGGSGGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSS

SEQ ID NO: 84 - protein, 868 aa, Artificial
  Artificial polypeptide comprising sequences derived from lama glama.
DVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGGSGGGGSGGGGSGGGGSGGGGSGGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 85 - protein, 990 aa, Artificial
  Artificial polypeptide comprising sequences derived from lama glama.
DVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGSEVQLVESGGGLVQPGGSLRLSCAASGIRFISMAWYRQAPGKQRELVARISSGGTTAYADSVKGRFTISRDNSKNTVYLQMNSLRAEDTAVYYCNTFSSRPNPWGQGTLVTVSSGGGGSGGGGSGGGGSGGGGSGGGGSGGGGSGGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 86 - protein, 995 aa, Artificial
  Artificial polypeptide comprising sequences derived from lama glama.
EVQLVESGGGLVQPGGSLRLSCAASGFTIGSYDMSWVRRAPGKGPEWVSSISSGGGSTYYADYVKGRFTISRDNAKNTLYLQMNSLRPEDTAVYYCAADRYIWARQGEYWGAYEYDYWGQGTLVTVSSGGGGSGGGGSGGGGSGGGGSGGGGSGGGGSGGGGSDVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGSEVQLVESGGGLVQPGGSLRLSCAASGIRFISMAWYRQAPGKQRELVARISSGGTTAYADSVKGRFTISRDNSKNTVYLQMNSLRAEDTAVYYCNTFSSRPNPWGQGTLVTVSSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 87 - protein, 903 aa, Artificial
  Artificial polypeptide comprising sequences derived from lama glama.
DVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGGSGGGGSGGGGSGGGGSGGGGSGGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGGSGGGGSGGGGSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 88 - protein, 1025 aa, Artificial
  Artificial polypeptide comprising sequences derived from lama glama.
DVQLVESGGGLVQPGGSLRLSCAASGRTFSSYSMGWFRQAPGKEREFVVAISKGGYKYDAVSLEGRFTISRDNAKNTVYLQINSLRPEDTAVYYCASSRAYGSSRLRLADTYEYWGQGTLVTVSSGGGGSGGGSEVQLVESGGGLVQPGGSLRLSCAASGIRFISMAWYRQAPGKQRELVARISSGGTTAYADSVKGRFTISRDNSKNTVYLQMNSLRAEDTAVYYCNTFSSRPNPWGQGTLVTVSSGGGGSGGGGSGGGGSGGGGSGGGGSGGGGSGGGGSEVQLVESGGGLVQPGGSLRLSCAASGRTFSSYAMAWYRQAPGKEREYVAAIRWSGGTAYYADSVKGRFTISRDNAKNTVYLQMNSLRPEDTAVYYCANRAPDTRLAPYEYDHWGQGTLVTVSSGGGGSGGGGSGGGGSGGGGSGGGGSGGGGSGGGGSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 89 - protein, 1030 aa, Artificial [sequence omitted]

SEQ ID NO: 90 - protein, 35 aa, Artificial
  Synthetic linker.
GGGGSGGGGSGGGGSGGGGSGGGGSGGGGSGGGGS

SEQ ID NO: 91 - protein, 9 aa, Artificial
  Synthetic linker.
GGGGSGGGS

SEQ ID NO: 92 - protein, 40 aa, Artificial
  Synthetic linker.
GGGGSGGGGSGGGGSGGGGSGGGGSGGGGSGGGGSGGGGS

SEQ ID NO: 93 - protein, 9 aa, Artificial
  Synthetic linker.
GGGGCGGGS

SEQ ID NO: 94 - protein, 25 aa, Artificial
  Synthetic linker.
GGGGCGGGGSGGGGSGGGGSGGGGS

SEQ ID NO: 95 - protein, 27 aa, Artificial
  Synthetic linker.
GGGSGGGGSGGGGCGGGGSGGGGSGGG

SEQ ID NO: 96 - protein, 35 aa, Artificial
  Synthetic linker.
GGGGSGGGGSGGGGCGGGGSGGGGSGGGGSGGGGS

SEQ ID NO: 97 - protein, 35 aa, Artificial
  Synthetic linker.
GGGGCGGGGSGGGGSGGGGSGGGGSGGGGSGGGGS

SEQ ID NO: 98 - protein, 115 aa, Artificial [sequence omitted]

SEQ ID NO: 99 - protein, 585 aa, Homo sapiens [sequence omitted]

SEQ ID NO: 100 - protein, 109 aa, Homo sapiens
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKWGQGTLVTVSS

SEQ ID NO: 101 - protein, 687 aa, Homo sapiens
HMMAAASRSASGWALLLLVALWQQRAAGSGVFQLQLQEFINERGVLASGRPCEPGCRTFFRVCLKHFQAVVSPGPCTFGTVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGANYACECPPNFTGSNCEKKVDRCTSNPCANGGQCLNRGPSRMCRCRPGFTGTYCELHVSDCARNPCAHGGTCHDLENGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

SEQ ID NO: 102 - protein, 707 aa, Macaca mulatta
MACACAMLATTARHESSMNKEYMAAASWSASGWALLLLVALWQQRAAGSGVFQLQLQEFVNERGVLASGRPCEPGCRTFFRVCLKHFQAVVSPGPCTFGSVSTPVLGTNSFAVRDDSSGGGRNPLQLPFNFTWPGTFSLIIEAWHAPGDDLRPEALPPDALISKIAIQGSLAVGQNWLLDEQTSTLTRLRYSYRVICSDNYYGDNCSRLCKKRNDHFGHYVCQPDGNLSCLPGWTGEYCQQPICLSGCHEQNGYCSKPAECLCRPGWQGRLCNECIPHNGCRHGTCSTPWQCTCDEGWGGLFCDQDLNYCTHHSPCKNGATCSNSGQRSYTCTCRPGYTGVDCELELSECDSNPCRNGGSCKDQEDGYHCLCPPGYYGLHCEHSTLSCADSPCFNGGSCRERNQGASYACECPPNFTGSNCEKKVDRCTSNPCANGGQCLNRGPSRMCRCRPGFTGTYCERHVSDCARNPCAHGGTCHDLESGLMCTCPAGFSGRRCEVRTSIDACASSPCFNRATCYTDLSTDTFVCNCPYGFVGSRCEFPVGLPPSFPWVAVSLGVGLAVLLVLLGMVAVAVRQLRLRRPDDGSREAMNNLSDFQKDNLIPAAQLKNTNQKKELEVDCGLDKSNCGKQQNHTLDYNLAPGPLGRGTMPGKFPHSDKSLGEKAPLRLHSEKPECRISAICSPRDSMYQSVCLISEERNECVIATEV

(19) United States
(12) Patent Application Publication
(10) Pub. No.: US 2011/0172398 A1
(43) Pub. Date: Jul. 14, 2011

(54) BISPECIFIC BINDING MOLECULES FOR ANTI-ANGIOGENESIS THERAPY

(75) Inventors: Eric BORGES, Maria Enzersdorf (AT); Andreas GSCHWIND, Wein (AT); Joachim BOUCNEAU, Brugge (BE); Evelyn DE TAVERNIER, Beervelde (BE); Joost KOLKMAN, Sint-Martens-Latem (BE); Pascal MERCHIERS, Kasterlee (BE); Diane VAN HOORICK, Laarne (BE)

(73) Assignee: BOEHRINGER INGELHEIM INTERNATIONAL GMBH, Ingelheim am Rhein (DE)

(21) Appl. No.: 12/894,989

(22) Filed: Sep. 30, 2010

(30) Foreign Application Priority Data
Oct. 2, 2009 (EP) 09172137.3
Sep. 3, 2010 (EP) 10175316.8

(51) Int. Cl. C07K 16/22; C07H 21/00; C12N 15/63; C12N 1/00

(52) U.S. Cl. 530/387.3; 536/23.53; 435/320.1; 435/243

(57) ABSTRACT

Bispecific binding molecules, in particular immunoglobulin single variable domains such as VHHs and domain antibodies, comprising a VEGF-binding component and a Dll4-binding component in one molecule. Pharmaceutical compositions containing same and their use in the treatment of diseases that are associated with VEGF- and Dll4-mediated effects on angiogenesis. Nucleic acids encoding the bispecific binding molecules, host cells and methods for preparing same.

### FIELD OF THE INVENTION

The invention relates to the field of human therapy, in particular cancer therapy and agents and compositions useful in such therapy.

### BACKGROUND OF THE INVENTION

As summarized in US 2008/0014196, angiogenesis is implicated in the pathogenesis of a number of disorders, including solid tumors and metastasis.

In the case of tumor growth, angiogenesis appears to be crucial for the transition from hyperplasia to neoplasia, and for providing nourishment for the growth and metastasis of the tumor (Folkman et al., Nature 339-58 (1989)), which allows the tumor cells to acquire a growth advantage compared to the normal cells. Therefore, anti-angiogenesis therapies have become an important treatment option for several types of tumors.

One of the most important pro-angiogenic factors is vascular endothelial growth factor (VEGF-A, in the following referred to as “VEGF”), which belongs to a gene family that includes placenta growth factor (PIGF), VEGF-B, VEGF-C, VEGF-D and VEGF-E and which exists in several isoforms that arise from alternative splicing of mRNA of a single gene, VEGF165 being the biologically most relevant isoform. Therefore, most anti-cancer therapies that rely on anti-angiogenesis have focused on blocking the VEGF pathway (Ferrara et al., Nat Rev Drug Discov. 2004 May; 3(5): 391-400).

Recently, Dll4 (or Delta like 4 or delta-like ligand 4) has been identified as a promising target for cancer therapy. Dll4 is a member of the Delta family of Notch ligands. Notch signaling is dysregulated in many cancers, e.g. in T-cell acute lymphoblastic leukemia and in solid tumors (Sharma et al. 2007, Cell Cycle 6 (8): 927-30; Shih et al., Cancer Res. 2007 Mar. 1; 67(5): 1879-82).

The extracellular domain of Dll4 is composed of an N-terminal domain, a Delta/Serrate/Lag-2 (DSL) domain, and a tandem of eight epidermal growth factor (EGF)-like repeats. Generally, the EGF domains are recognized as comprising amino acid residues 218-251 (EGF-1; domain 1), 252-282 (EGF-2; domain 2), 284-322 (EGF-3; domain 3), 324-360 (EGF-4; domain 4), and 362-400 (EGF-5; domain 5), with the DSL domain at about amino acid residues 173-217 and the N-terminal domain at about amino acid residues 27-172 of hDll4 (WO 2008/076379).

It has been reported that Dll4 exhibits highly selective expression by vascular endothelium, in particular in arterial endothelium (Shutter et al. (2000) Genes Develop. 14: 1313-1318). Recent studies in mice have shown that Dll4 is induced by VEGF and is a negative feedback regulator that restrains vascular sprouting and branching. Consistent with this role, the deletion or inhibition of Dll4 results in excessive angiogenesis (Scehnet et al., Blood. 2007 Jun. 1;109 (11): 4753-60). This unrestrained angiogenesis paradoxically decreases tumor growth due to the formation of non-productive vasculature, even in tumors resistant to anti-VEGF therapies (Thurston et al., Nat Rev Cancer. 2007 May; 7(5): 327-31; WO 2007/070671; Noguera-Troise et al., Nature. 2006 Dec. 21; 444(7122)). In addition to the effects on tumor angiogenesis, inhibition of Dll4 has been shown to reduce the frequency of cancer stem cells in preclinical tumor models (Hoey et al., Cell Stem Cell. 2009 Aug 7; 5(2): 168-77).

Several biological compounds that target Dll4 are in (pre-)clinical development have been described: REGN-421 (=SARI 53192; Regeneron, Sanofi-Aventis; WO2008076379) and OPM-21M18 (OncoMed) (Hoey et al., Cell Stem Cell. 2009 Aug. 7; 5(2): 168-77), both fully human Dll4 antibodies; YW152F (Genentech), a humanized Dll4 antibody (Ridgway et al., Nature. 2006 Dec. 21;444(7122): 1083-7); Dll4-Fc (Regeneron, Sanofi-Aventis), a recombinant fusion protein composed of the extracellular region of Dll4 and the Fc region of human IgG1 (Noguera-Troise et al., Nature. 2006 Dec. 21;444(7122)).

It has been shown that the combined inhibition of VEGF and Dll4 provides superior anti-tumor activity compared to anti-VEGF alone in xenograft models of multiple tumor types and in anti-VEGF resitant tumor models (Noguera-Troise et al., Nature. 2006 Dec. 21; 444(7122): 1032-7; Ridgway et al., Nature. 2006 Dec. 21; 444(7122): 1083-7; US 2008175847).

Monoclonal antibodies (MAbs) and fusion proteins have several shortcomings in view of their therapeutic application: To prevent their degradation, they must be stored at near freezing temperatures. Also, since they are quickly digested in the gut, they are not suited for oral administration. Another major restriction of MAbs for cancer therapy is poor transport, which results in low concentrations and a lack of targeting of all cells in a tumor.

Also, the state-of-the art therapies that are based on targeting both VEGF and Dll4, represent a combination therapy involving two individual inhibitors, i.e. an VEGF-binding molecule and a separate Dll4-binding molecule. However, these therapies have the drawbacks that development and production of two separate drugs involves high costs and many resources, two drugs may have different pharmacokinetic properties and that administration of two drugs is inconvenient for the patient.

In view of the above, it has been an object of the invention to provide improved molecules for human anti-tumor therapy.

The present invention is based on the concept of combining one or more VEGF-binding molecules with one or more Dll4-binding molecules in a single therapeutic agent.

Thus, the invention relates to bispecific binding molecules comprising one or more Dll4-binding molecules and one or more VEGF-binding molecules.

In the following, if not otherwise stated, the term “binding molecule” (or “antigen-binding molecule”) refers to either or both of a Dll4-binding molecule, in particular an immunoglobulin single variable domain, or a VEGF-binding molecule, in particular an immunoglobulin single variable domain. The term “bispecific binding molecule” refers to a molecule comprising at least one Dll4-binding molecule (or “binding component”) and at least one VEGF-binding molecule (or binding component). A bispecific binding molecule may contain more than one Dll4-binding molecule and/or more than one VEGF-binding molecule, i.e. in the case that the bispecific binding molecule contains a biparatopic (as defined below) Dll4-binding molecule and/or a biparatopic VEGF-binding molecule, in the part of the molecule that binds to Dll4 or to VEGF, i.e. in its “Dll4-binding component” (or anti-Dll4 component) or “VEGF-binding component” (or anti-VEGF component), respectively.

The bispecific binding molecules of the invention are useful as pharmacologically active agents in compositions in the prevention, treatment, alleviation and/or diagnosis of diseases or conditions that can be modulated by inhibition of Dll4, such as cancer.

It has been a further object of the invention to provide methods for the prevention, treatment, alleviation and/or diagnosis of such diseases, disorders or conditions, involving the use and/or administration of such agents and compositions.

In particular, it is has been an object of the invention to provide such pharmacologically active agents, compositions and/or methods that provide certain advantages compared to the agents, compositions and/or methods currently used and/or known in the art.

These advantages include improved therapeutic and/or pharmacological properties and/or other advantageous properties, e.g. for manufacturing purposes, especially as compared to conventional antibodies as those described above, or fragments thereof.

More in particular, it has been an object of the invention to provide novel molecules, and, specifically, molecules that bind to mammalian and, especially, human Dll4 and human VEGF, wherein such molecules are suitable for the therapeutic and diagnostic purposes as described herein.

### BRIEF SUMMARY OF THE INVENTION

According to a first aspect, there are provided bispecific binding molecules, comprising a Dll4-binding component and a VEGF-binding component in a single molecule.

More specifically, a bispecific binding molecule of the invention essentially comprises (i) a Dll4-binding component specifically binding to at least one epitope of Dll4 and (ii) a VEGF-binding component specifically binding to at least an epitope of VEGF, wherein the components are linked to each other in such a way that they simultaneously bind to Dll4 and VEGF or that they bind to either Dll4 or VEGF at a time.

According to preferred aspects of the invention, the two components comprise one or more immunoglobulin single variable domains that may be, independently of each other, VHHs or domain antibodies, and/or any other sort of immunoglobulin single variable domains, such as VL domains, as defined herein, provided that each of these immunoglobulin single variable domains will bind the antigen, i.e. Dll4 or VEGF, respectively.

According to a preferred embodiment, the immunoglobulin single variable domains are of the same type, in particular, all immunoglobulin single variable domains are VHHs or domain antibodies.

According to a particularly preferred embodiment, all immunoglobulin single variable domains are VHHs, preferably humanized (or “sequence-optimized”, as defined herein) VHHs. Accordingly, the invention relates to bispecific binding molecules comprising an (optionally humanized or sequence-optimized) anti-Dll4 VHH and an (optionally humanized or sequence-optimized) anti-VEGF VHH.

However, it will be clear to the skilled person that the teaching herein may be applied analogously to bispecific binding molecules including other anti-Dll4 or anti-VEGF immunoglobulin single variable domains, such as domain antibodies.

In another aspect, the invention relates to nucleic acids encoding the bispecific binding molecules of the invention as well as host cells containing same.

The invention further relates to a product or composition containing or comprising at least one bispecific binding molecule of the invention and optionally one or more further components of such compositions.

The invention further relates to methods for preparing or generating the bispecific binding molecules, nucleic acids, host cells, products and compositions described herein.

The invention further relates to applications and uses of the bispecific binding molecules, nucleic acids, host cells, products and compositions described herein, as well as to methods for the prevention and/or treatment for diseases and disorders that can be modulated by inhibition of Dll4.

These and other aspects, embodiments, advantages and applications of the invention will become clear from the further description hereinbelow.

### Definitions

Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Roitt et al., “Immunology” (2^{nd} Ed.), Gower Medical Publishing, London, New York (1989), as well as to the general background art cited herein; Furthermore, unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

Unless indicated otherwise, the terms “immunoglobulin” and “immunoglobulin sequence”—whether used herein to refer to a heavy chain antibody or to a conventional 4-chain antibody—are used as general terms to include both the full-size antibody, the individual chains thereof, as well as all parts, domains or fragments thereof (including but not limited to antigen-binding domains or fragments such as VHH domains or VH/VL domains, respectively). In addition, the term “sequence” as used herein (for example in terms like “immunoglobulin sequence”, “antibody sequence”, “(single) variable domain sequence”, “VHH sequence” or “protein sequence”), should generally be understood to include both the relevant amino acid sequence as well as nucleic acid sequences or nucleotide sequences encoding the same, unless the context requires a more limited interpretation.

The term “domain” (of a polypeptide or protein) as used herein refers to a folded protein structure which has the ability to retain its tertiary structure independently of the rest of the protein. Generally, domains are responsible for discrete functional properties of proteins, and in many cases may be added, removed or transferred to other proteins without loss of function of the remainder of the protein and/or of the domain.

The term “immunoglobulin domain” as used herein refers to a globular region of an antibody chain (such as e.g. a chain of a conventional 4-chain antibody or of a heavy chain antibody), or to a polypeptide that essentially consists of such a globular region. Immunoglobulin domains are characterized in that they retain the immunoglobulin fold characteristic of antibody molecules, which consists of a 2-layer sandwich of about 7 antiparallel beta-strands arranged in two beta-sheets, optionally stabilized by a conserved disulphide bond.

The term “immunoglobulin variable domain” as used herein means an immunoglobulin domain essentially consisting of four “framework regions” which are referred to in the art and hereinbelow as “framework region 1” or “FR1”; as “framework region 2” or“FR2”; as “framework region 3” or “FR3”; and as “framework region 4” or “FR4”, respectively; which framework regions are interrupted by three “complementarity determining regions” or “CDRs”, which are referred to in the art and hereinbelow as “complementarity determining region 1″or “CDR1”; as “complementarity determining region 2” or “CDR2”; and as “complementarity determining region 3” or “CDR3”, respectively. Thus, the general structure or sequence of an immunoglobulin variable domain can be indicated as follows: FR1-CDR1-FR2-CDR2-FR3-CDR3-FR4. It is the immunoglobulin variable domain(s) that confer specificity to an antibody for the antigen by carrying the antigen-binding site.

The term “immunoglobulin single variable domain” as used herein means an immunoglobulin variable domain which is capable of specifically binding to an epitope of the antigen without pairing with an additional variable immunoglobulin domain. One example of immunoglobulin single variable domains in the meaning of the present invention are “domain antibodies”, such as the immunoglobulin single variable domains VH and VL (VH domains and VL domains). Another example of immunoglobulin single variable domains are “VHH domains” (or simply “VHHs”) from camelids, as defined hereinafter.

In view of the above definition, the antigen-binding domain of a conventional 4-chain antibody (such as an IgG, IgM, IgA, IgD or IgE molecule; known in the art) or of a Fab fragment, a F(ab′)2 fragment, an Fv fragment such as a disulphide linked Fv or a scFv fragment, or a diabody (all known in the art) derived from such conventional 4-chain antibody, would normally not be regarded as an immunoglobulin single variable domain, as, in these cases, binding to the respective epitope of an antigen would normally not occur by one (single) immunoglobulin domain but by a pair of (associating) immunoglobulin domains such as light and heavy chain variable domains, i.e. by a VH-VL pair of immunoglobulin domains, which jointly bind to an epitope of the respective antigen.

“VHH domains”, also known as VHHs, V_{H}H domains, VHH antibody fragments, and VHH antibodies, have originally been described as the antigen binding immunoglobulin (variable) domain of “heavy chain antibodies” (i.e. of “antibodies devoid of light chains”; Hamers-Casterman C, Atarhouch T, Muyldermans S, Robinson G, Hamers C, Songa EB, Bendahman N, Hamers R.: “Naturally occurring antibodies devoid of light chains”; Nature 363, 446-448 (1993)). The term “VHH domain” has been chosen in order to distinguish these variable domains from the heavy chain variable domains that are present in conventional 4-chain antibodies (which are referred to herein as “V_{H} domains” or “VH domains”) and from the light chain variable domains that are present in conventional 4-chain antibodies (which are referred to herein as “V_{L} domains” or “VL domains”). VHH domains can specifically bind to an epitope without an additional antigen binding domain (as opposed to VH or VL domains in a conventional 4-chain antibody, in which case the epitope is recognized by a VL domain together with a VH domain). VHH domains are small, robust and efficient antigen recognition units formed by a single immunoglobulin domain.

In the context of the present invention, the terms VHH domain, VHH, V_{H}H domain, VHH antibody fragment, VHH antibody, as well as “Nanobody®” and “Nanobody® domain” (“Nanobody” being a trademark of the company Ablynx N.V.; Ghent; Belgium) are used interchangeably and are representatives of immunoglobulin single variable domains (having the structure FR1-CDR1-FR2-CDR2-FR3-CDR3-FR4 and specifically binding to an epitope without requiring the presence of a second immunoglobulin variable domain), and which are distinguished from VH domains by the so-called “hallmark residues”, as defined in e.g. WO2009/109635, FIG. 1.

The amino acid residues of a immunoglobulin single variable domain, e.g. a VHH, are numbered according to the general numbering for V_{H} domains given by Kabat et al. (“Sequence of proteins of immunological interest”, US Public Health Services, NIH Bethesda, Md., Publication No. 91), as applied to VHH domains from camelids, as shown e.g. in FIG. 2 of Riechmann and Muyldermans, J. Immunol. Methods 231, 25-38 (1999). According to this numbering,

FR1 comprises the amino acid residues at positions 1-30,

CDR1 comprises the amino acid residues at positions 31-35,

FR2 comprises the amino acids at positions 36-49,

CDR2 comprises the amino acid residues at positions 50-65,

FR3 comprises the amino acid residues at positions 66-94,

CDR3 comprises the amino acid residues at positions 95-102, and

FR4 comprises the amino acid residues at positions 103-113.

However, it should be noted that—as is well known in the art for V_{H} domains and for VHH domains—the total number of amino acid residues in each of the CDRs may vary and may not correspond to the total number of amino acid residues indicated by the Kabat numbering (that is, one or more positions according to the Kabat numbering may not be occupied in the actual sequence, or the actual sequence may contain more amino acid residues than the number allowed for by the Kabat numbering). This means that, generally, the numbering according to Kabat may or may not correspond to the actual numbering of the amino acid residues in the actual sequence.

Alternative methods for numbering the amino acid residues of V_{H} domains, which methods can also be applied in an analogous manner to VHH domains, are known in the art. However, in the present description, claims and figures, the numbering according to Kabat and applied to VHH domains as described above will be followed, unless indicated otherwise.

The total number of amino acid residues in a VHH domain will usually be in the range of from 110 to 120, often between 112 and 115. It should however be noted that smaller and longer sequences may also be suitable for the purposes described herein.

Immunoglobulin single variable domains (e.g. VHHs and domain antibodies), have a number of unique structural characteristics and functional properties which makes them highly advantageous for use in therapy as functional antigen-binding molecules. In particular, and without being limited thereto, VHH domains (which have been “designed” by nature to functionally bind to an antigen without pairing with a light chain variable domain) can function as single, relatively small, functional antigen-binding structural units.

Due to their unique properties, immunoglobulin single variable domains, as defined herein, like VHHs or VHs (or VLs) - either alone or as part of a larger polypeptide, e.g. a biparatopic molecule or a bispecific binding molecule, offer a number of significant advantages:
only a single domain is required to bind an antigen with high affinity and with high selectivity, so that there is no need to have two separate domains present, nor to assure that these two domains are present in the right spacial conformation and configuration (i.e. through the use of especially designed linkers, as with scFv's);
immunoglobulin single variable domains can be expressed from a single nucleic acid molecule and do not require any post-translational modification (like glycosylation;
immunoglobulin single variable domains can easily be engineered into multivalent and multispecific formats (as further discussed herein);
immunoglobulin single variable domains have high specificity and affinity for their target, low inherent toxicity and can be administered via alternative routes than infusion or injection;
immunoglobulin single variable domains are highly stable to heat, pH, proteases and other denaturing agents or conditions and, thus, may be prepared, stored or transported without the use of refrigeration equipments;
immunoglobulin single variable domains are easy and relatively inexpensive to prepare, both on small scale and on a manufacturing scale. For example, immunoglobulin single variable domains and polypeptides containing the same can be produced using microbial fermentation (e.g. as further described below) and do not require the use of mammalian expression systems, as with for example conventional antibodies;
immunoglobulin single variable domains are relatively small (approximately 15 kDa, or 10 times smaller than a conventional IgG) compared to conventional 4-chain antibodies and antigen-binding fragments thereof, and therefore show high(er) penetration into tissues (including but not limited to solid tumors and other dense tissues) and can be administered in higher doses than such conventional 4-chain antibodies and antigen-binding fragments thereof;
VHHs have specific so-called “cavity-binding properties” (inter alia due to their extended CDR3 loop, compared to VH domains from 4-chain antibodies) and can therefore also access targets and epitopes not accessible to conventional 4-chain antibodies and antigen-binding fragments thereof;
VHHs have the particular advantage that they are highly soluble and very stable and do not have a tendency to aggregate (as with the mouse-derived antigen-binding domains described by Ward et al., Nature 341: 544-546 (1989)).

The immunoglobulin single variable domains contained in the components of the bispecific binding molecules of the invention, are not limited with respect to a specific biological source from which they have been obtained or to a specific method of preparation. For example, obtaining VHHs may include the following steps:
(1) isolating the VHH domain of a naturally occurring heavy chain antibody; or screening a library comprising heavy chain antibodies or VHHs and isolating VHHs therefrom;
(2) expressing a nucleic acid molecule encoding a VHH with the naturally occurring sequence;
(3) “humanizing” (or sequence-optimizing) a VHH, optionally after affinity maturation, with a naturally occurring sequence or expressing a nucleic acid encoding such humanized VHH;
(4) “camelizing” (as described below) a immunoglobulin single variable heavy domain from a naturally occurring antibody from an animal species, in particular a species of mammal, such as from a human being, or expressing a nucleic acid molecule encoding such camelized domain;
(5) “camelizing” a VH, or expressing a nucleic acid molecule encoding such a camelized VH;
(6) using techniques for preparing synthetically or semi-synthetically proteins, polypeptides or other amino acid sequences;
(7) preparing a nucleic acid molecule encoding a VHH domain using techniques for nucleic acid synthesis, followed by expression of the nucleic acid thus obtained;
(8) subjecting heavy chain antibodies or VHHs to affinity maturation, to mutagenesis (e.g. random mutagenesis or site-directed mutagenesis) and/or any other technique(s) in order to increase the affinity and/or specificity of the VHH; and/or
(9) combinations or selections of the foregoing steps.

Suitable methods and techniques for performing the above-described steps are known in the art and will be clear to the skilled person.

According to a specific embodiment, the immunoglobulin single variable domains present in the bispecific binding molecules of the invention are VHHs with an amino acid sequence that essentially corresponds to the amino acid sequence of a naturally occurring VHH domain, but that has been humanized (sequence-optimized), optionally after affinity-maturation), i.e. by replacing one or more amino acid residues in the amino acid sequence of said naturally occurring VHH sequence by one or more of the amino acid residues that occur at the corresponding position(s) in a variable heavy domain of a conventional 4-chain antibody from a human being. This can be performed using methods known in the art, which can by routinely used by the skilled person.

A sequence-optimized VHH may contain one or more fully human framework region sequences, and, in an even more specific embodiment, may contain human framework region sequences derived from the human germline Vh3 sequences DP-29, DP-47, DP-51, or parts thereof, or be highly homologous thereto. Thus, a humanization protocol may comprise the replacement of any of the VHH residues with the corresponding framework 1, 2 and 3 (FR1, FR2 and FR3) residues of germline VH genes such as DP 47, DP 29 and DP 51) either alone or in combination. Suitable framework regions (FR) of the immunoglobulin single variable domains of the invention can be selected from those as set out e.g. in WO 2006/004678 and specifically, include the so-called “KERE” and “CLEW” classes. Particularly preferred are immunoglobulin single variable domains having the amino acid sequence G-L-E-W at about positions 44 to 47, and their respective humanized counterparts.

By way of example, a humanizing substitution for VHHs belonging to the 103 P,R,S-group and/or the CLEW-group (as defined below) is 108Q to 108L. Methods for humanizing immunoglobulin single variable domains are known in the art.

Binding immunoglobulin single variable domains with improved properties in view of therapeutic application, e.g. enhanced affinity or decreased immunogenicity, may be obtained from individual binding molecules by techniques known in the art, such as affinity maturation (for example, starting from synthetic, random or naturally occurring immunoglobulin sequences),

CDR grafting, humanizing, combining fragments derived from different immunoglobulin sequences, PCR assembly using overlapping primers, and similar techniques for engineering immunoglobulin sequences well known to the skilled person; or any suitable combination of any of the foregoing, also termed “sequence optimization”, as described herein. Reference is, for example, made to standard handbooks, as well as to the further description and Examples.

If appropriate, a binding molecule with increased affinity may be obtained by affinity-maturation of another binding molecule, the latter representing, with respect to the affinity-matured molecule, the “parent” binding molecule.

Methods of obtaining VHHs that bind to a specific antigen or epitope have been described earlier, e.g. in WO2006/040153 and WO2006/122786. As also described therein in detail, VHH domains derived from camelids can be “humanized” (also termed “sequence-optimized” herein, “sequence-optimizing” may, in addition to humanization, encompass an additional modification of the sequence by one or more mutations that furnish the VHH with improved properties, such as the removal of potential post translational modification sites) by replacing one or more amino acid residues in the amino acid sequence of the original VHH sequence by one or more of the amino acid residues that occur at the corresponding position(s) in a VH domain from a conventional 4-chain antibody from a human being. A humanized VHH domain may contain one or more fully human framework region sequences, and, in an even more specific embodiment, may contain human framework region sequences derived from DP-29, DP-47, DP-51, or parts thereof, optionally combined with JH sequences, such as JH5.

Domain antibodies, also known as “Dab”s and “dAbs” (the terms “Domain Antibodies” and “dAbs” being used as trademarks by the GlaxoSmithKline group of companies) have been described in e.g. Ward, E. S., et al.: “Binding activities of a repertoire of single immunoglobulin variable domains secreted from Escherichia coli”; Nature 341: 544-546 (1989); Holt, L. J. et al.: “Domain antibodies: proteins for therapy”; TRENDS in Biotechnology 21(11): 484-490 (2003); and WO2003/002609.

Domain antibodies essentially correspond to the VH or VL domains of antibodies from non-camelid mammals, in particular human 4-chain antibodies. In order to bind an epitope as a single antigen binding domain, i.e. without being paired with a VL or VH domain, respectively, specific selection for such antigen binding properties is required, e.g. by using libraries of human single VH or VL domain sequences.

Domain antibodies have, like VHHs, a molecular weight of approximately 13 to approximately 16 kDa and, if derived from fully human sequences, do not require humanization for e.g. therapeutical use in humans. As in the case of VHH domains, they are well expressed also in prokaryotic expression systems, providing a significant reduction in overall manufacturing cost.

Furthermore, it will also be clear to the skilled person that it is possible to “graft” one or more of the CDR's mentioned above onto other “scaffolds”, including but not limited to human scaffolds or non-immunoglobulin scaffolds. Suitable scaffolds and techniques for such CDR grafting are known in the art.

The terms “epitope” and “antigenic determinant”, which can be used interchangeably, refer to the part of a macromolecule, such as a polypeptide, that is recognized by antigen-binding molecules, such as conventional antibodies or the polypeptides of the invention, and more particularly by the antigen-binding site of said molecules. Epitopes define the minimum binding site for an immunoglobulin, and thus represent the target of specificity of an immunoglobulin.

A polypeptide (such as an immunoglobulin, an antibody, an immunoglobulin single variable domain of the invention, or generally a binding molecule or a fragment thereof) that can “bind to” or “specifically bind to”, that “has affinity for” and/or that “has specificity for a certain epitope, antigen or protein (or for at least one part, fragment or epitope thereof) is said to be “against” or “directed against' said epitope, antigen or protein or is a “binding” molecule with respect to such epitope, antigen or protein. In this context, a VEGF- or Dll4-binding molecule may also be referred to as “VEGF-neutralizing” or “Dll4-neutralizing”, respectively.

Generally, the term “specificity' refers to the number of different types of antigens or epitopes to which a particular antigen-binding molecule or antigen-binding protein (such as an immunoglobulin single variable domain) molecule can bind. The specificity of an antigen-binding molecule can be determined based on its affinity and/or avidity. The affinity, represented by the equilibrium constant for the dissociation of an antigen with an antigen-binding protein (KD), is a measure for the binding strength between an epitope and an antigen-binding site on the antigen-binding protein: the lesser the value of the KD, the stronger the binding strength between an epitope and the antigen-binding molecule (alternatively, the affinity can also be expressed as the affinity constant (KA), which is 1/KD). As will be clear to the skilled person (for example on the basis of the further disclosure herein), affinity can be determined in a manner known per se, depending on the specific antigen of interest. Avidity is the measure of the strength of binding between an antigen-binding molecule (such as an immunoglobulin, an antibody, an immunoglobulin single variable domain or a polypeptide containing it and the pertinent antigen. Avidity is related to both the affinity between an epitope and its antigen binding site on the antigen-binding molecule and the number of pertinent binding sites present on the antigen-binding molecule.

The part of an antigen-binding molecule that recognizes the epitope is called a paratope.

Unless indicated otherwise, the term “Dll4-binding molecule” or “VEGF-binding molecule” includes anti-Dll4 or anti-VEGF antibodies, anti-Dll4 antibody or anti-VEGF antibody fragments, “anti-Dll4 antibody-like molecules” or “anti-VEGF antibody-like molecules”, as defined herein, and conjugates with any of these. Antibodies include, but are not limited to, monoclonal and chimerized monoclonal antibodies. The term “antibody” encompasses complete immunoglobulins, like monoclonal antibodies produced by recombinant expression in host cells, as well as antibody fragments or “antibody-like molecules”, including single-chain antibodies and linear antibodies, so-called “SMIPs” (“Small Modular Immunopharmaceuticals”), as e.g described in WO 02/056910; Antibody-like molecules include immunoglobulin single variable domains, as defined herein. Other examples for antibody-like molecules are immunoglobulin super family antibodies (IgSF), or CDR-grafted molecules.

“VEGF-binding molecule” or “Dll4-binding molecule” respectively, refers to both monovalent target-binding molecules (i.e. molecules that bind to one epitope of the respective target) as well as to bi- or multivalent binding molecules (i.e. binding molecules that bind to more than one epitope, e.g. “biparatopic” molecules as defined hereinbelow). VEGF(or Dll4)-binding molecules containing more than one VEGF(or Dll4)-binding immunoglobulin single variable domain are also termed “formatted” binding molecules, they may, within the target-binding component, in addition to the immunoglobulin single variable domains, comprise linkers and/or moieties with effector functions, e.g. half-life-extending moieties like albumin-binding immunoglobulin single variable domains, and/or a fusion partner like serum albumin and/or an attached polymer like PEG.

The term “biparatopic VEGF(or Dll4)-binding molecule”or “biparatopic immunoglobulin single variable domain”as used herein shall mean a binding molecule comprising a first immunoglobulin single variable domain and a second immunoglobulin single variable domain as herein defined, wherein the two molecules bind to two non-overlapping epitopes of the respective antigen. The biparatopic binding molecules are composed of immunoglobulin single variable domains which have different specificities with respect to the epitope. The part of an antigen-binding molecule (such as an antibody or an immunoglobulin single variable domain of the invention) that recognizes the epitope is called a paratope.

A formatted binding molecule may, albeit less preferred, also comprise two identical immunoglobulin single variable domains or two different immunoglobulin single variable domains that recognize the same or overlapping epitopes or their respective antigen. In this case, with respect to VEGF, the two immunoglobulin single variable domains may bind to the same or an overlapping epitope in each of the two monomers that form the VEGF dimer.

Typically, the binding molecules of the invention will bind with a dissociation constant (K_{D}) of 10E-5 to 10E-14 moles/liter (M) or less, and preferably 10E-7 to 10E-14 moles/liter (M) or less, more preferably 10E-8 to 10E-14 moles/liter, and even more preferably 10E-11 to 10E-13, as measured e.g. in a Biacore or in a Kinexa assay), and/or with an association constant (K_{A}) of at least 10E7 ME-1, preferably at least 10E8 ME-1, more preferably at least 10E9 ME-1, such as at least 10E11 ME-1. Any K_{D} value greater than 10E-4 M is generally considered to indicate non-specific binding. Preferably, a polypeptide of the invention will bind to the desired antigen, i.e. VEGF or Dll4, respectively, with a K_{D} less than 500 nM, preferably less than 200 nM, more preferably less than 10 nM, such as less than 500 pM. Specific binding of an antigen-binding protein to an antigen or epitope can be determined in any suitable manner known per se, including, for example, the assays described herein, Scatchard analysis and/or competitive binding assays, such as radioimmunoassays (RIA), enzyme immunoassays (EIA) and sandwich competition assays, and the different variants thereof known per se in the art.

Amino acid residues will be indicated according to the standard three-letter or one-letter amino acid code, as generally known and agreed upon in the art. When comparing two amino acid sequences, the term “amino acid difference” refers to insertions, deletions or substitutions of the indicated number of amino acid residues at a position of the reference sequence, compared to a second sequence. In case of substitution(s), such substitution(s) will preferably be conservative amino acid substitution(s), which means that an amino acid residue is replaced with another amino acid residue of similar chemical structure and which has little or essentially no influence on the function, activity or other biological properties of the polypeptide. Such conservative amino acid substitutions are well known in the art, for example from WO 98/49185, wherein conservative amino acid substitutions preferably are substitutions in which one amino acid within the following groups (i)-(v) is substituted by another amino acid residue within the same group: (i) small aliphatic, nonpolar or slightly polar residues: Ala, Ser, Thr, Pro and Gly; (ii) polar, negatively charged residues and their (uncharged) amides: Asp, Asn, Glu and Gln; (iii) polar, positively charged residues: His, Arg and Lys; (iv) large aliphatic, nonpolar residues: Met, Leu, Ile, Val and Cys; and (v) aromatic residues: Phe, Tyr and Trp. Particularly preferred conservative amino acid substitutions are as follows: Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp or into Phe; Val into Ile or into Leu.

A polypeptide or nucleic acid molecule is considered to be “(in) essentially isolated (form)”—for example, when compared to its native biological source and/or the reaction medium or cultivation medium from which it has been obtained—when it has been separated from at least one other component with which it is usually associated in said source or medium, such as another protein/polypeptide, another nucleic acid, another biological component or macromolecule or at least one contaminant, impurity or minor component. In particular, a polypeptide or nucleic acid molecule is considered “essentially isolated” when it has been purified at least 2-fold, in particular at least 10-fold, more in particular at least 100-fold, and up to 1000-fold or more. A polypeptide or nucleic acid molecule that is “in essentially isolated form” is preferably essentially homogeneous, as determined using a suitable technique, such as a suitable chromatographical technique, such as polyacrylamide gel electrophoresis.

“Sequence identity' between two VEGF-binding molecule sequences indicates the percentage of amino acids that are identical between the sequences. It may be calculated or determined as described in paragraph f) on pages 49 and 50 of WO 08/020079. “Sequence similarity” indicates the percentage of amino acids that are either identical or that represent conservative amino acid substitutions.

Alternative methods for numbering the amino acid residues of V_{H} domains, which methods can also be applied in an analogous manner to VHH domains, are known in the art. However, in the present description, claims and figures, the numbering according to Kabat and applied to VHH domains as described above will be followed, unless indicated otherwise.

An “affinity-matured” binding molecule, in particular a VHH or a domain antibody, has one or more alterations in one or more CDRs which result in an improved affinity forits target, as compared to the respective parent binding molecule. Afffinity-matured binding molecules may be prepared by methods known in the art, for example, as described by Marks et al., 1992, Biotechnology 10: 779-783, or Barbas, et al., 1994, Proc. Nat. Acad. Sci, USA 91: 3809-3813.; Shier et al., 1995, Gene 169: 147-155; Yelton et al., 1995, Immunol. 155: 1994-2004; Jackson et al., 1995, J. Immunol. 154(7): 3310-9; and Hawkins et al., 1992, J. Mol. Biol. 226(3): 889 896; K S Johnson and R E Hawkins, “Affinity maturation of antibodies using phage display”, Oxford University Press 1996.

For the present invention, an “amino acid sequences of SEQ ID NO: x”: includes, if not otherwise stated, an amino acid sequence that is 100% identical with the sequence shown in the respective SEQ ID NO: x;
a) amino acid sequences that have at least 80% amino acid identity with the sequence shown in the respective SEQ ID NO: x;
b) amino acid sequences that have 3, 2, or 1 amino acid differences with the sequence shown in the respective SEQ ID NO: x.

The terms “cancer” and “cancerous” refer to or describe the physiological condition in mammals that is typically characterized by unregulated cell growth/proliferation. Examples of cancer to be treated with a bispecific binding molecule of the invention, include but are not limited to carcinoma, lymphoma, blastoma, sarcoma, and leukemia. More particular examples of such cancers, as suggested for treatment with Dll4 antagonists in US 2008/0014196, include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney cancer, liver cancer, prostate cancer, vulval cancer, thyroid cancer, hepatic carcinoma, gastric cancer, melanoma, and various types of head and neck cancer. Dysregulation of angiogenesis can lead to many disorders that can be treated by compositions and methods of the invention. These disorders include both non-neoplastic and neoplastic conditions. Neoplasties include but are not limited those described above. Non-neoplastic disorders include, but are not limited to, as suggested for treatment with Dll4 antagonists in US 2008/0014196, undesired or aberrant hypertrophy, arthritis, rheumatoid arthritis (RA), psoriasis, psoriatic plaques, sarcoidosis, atherosclerosis, atherosclerotic plaques, diabetic and other proliferative retinopathies including retinopathy of prematurity, retrolental fibroplasia, neovascular glaucoma, age-related macular degeneration, diabetic macular edema, corneal neovascularization, corneal graft neovascularization, corneal graft rejection, retinal/choroidal neovascularization, neovascularization of the angle (rubeosis), ocular neovascular disease, vascular restenosis, arteriovenous malformations (AVM), meningioma, hemangioma, angiofibroma, thyroid hyperplasias (including Grave's disease), corneal and other tissue transplantation, chronic inflammation, lung inflammation, acute lung injury/ARDS, sepsis, primary pulmonary hypertension, malignant pulmonary effusions, cerebral edema (e.g., associated with acute stroke/closed head injury/trauma), synovial inflammation, pannus formation in RA, myositis ossificans, hypertropic bone formation, osteoarthritis (OA), refractory ascites, polycystic ovarian disease, endometriosis, 3^{rd} spacing of fluid diseases (pancreatitis, compartment syndrome, burns, bowel disease), uterine fibroids, premature labor, chronic inflammation such as IBD (Crohn's disease and ulcerative colitis), renal allograft rejection, inflammatory bowel disease, nephrotic syndrome, undesired or aberrant tissue mass growth (non-cancer), hemophilic joints, hypertrophic scars, inhibition of hair growth, Osier-Weber syndrome, pyogenic granuloma retrolental fibroplasias, scleroderma, trachoma, vascular adhesions, synovitis, dermatitis, preeclampsia, ascites, pericardial effusion (such as that associated with pericarditis), and pleural effusion.

### DETAILED DESCRIPTION OF THE INVENTION

In a first aspect, the present invention relates to a bispecific binding molecule comprising a Dll4-binding component and a VEGF-binding component.

According to preferred embodiments, said Dll4-binding component and said VEGF-binding component comprise at least one Dll4-binding immunoglobulin single variable domain and at least one VEGF-binding immunoglobulin single variable domain, respectively.

In a preferred aspect, said Dll4-binding component and said VEGF-binding component each comprise at least one VEGF-binding immunoglobulin single variable domain and at least one Dll4-binding immunoglobulin single variable domain, respectively, wherein each of said immunoglobulin single variable domains has four framework regions and three complementarity determining regions CDR1, CDR2 and CDR3, respectively, wherein
a) a CDR3 of said at least one Dll4-binding immunoglobulin single variable domain has an amino acid sequence selected from
   i) Arg Ala Pro Asp Thr Arg Leu Xaa Pro Tyr Xaa Tyr Asp Xaa as shown in SEQ ID NO: 1, wherein
      Xaa at position 8 is Arg, Ala or Glu;
      Xaa at position 11 is Leu or Glu; and
      Xaa at position 14 is Tyr or His; and
   ii) Asp Arg Tyr Ile Trp Ala Arg Gln Gly Glu Tyr Trp Gly Ala Tyr Xaa Asp Tyr as shown in SEQ ID NO: 2, wherein
      Xaa is Gln, Ala or Tyr; and wherein
b) a CDR3 of said at least one VEGF-binding immunoglobulin single variable domain has the amino acid sequence Ser Arg Ala Tyr Gly Ser Ser Arg Leu Arg Leu Ala Asp Thr Tyr Xaa Tyr, as shown in SEQ ID NO: 3, wherein Xaa is Asp or Glu,
   wherein said VEGF-binding immunoglobulin single variable domain is capable of blocking the interaction of human recombinant VEGF165 with the human recombinant VEGFR-2 with an inhibition rate of ≧60%.

According to preferred embodiments, the immunoglobulin single variable domains are VHHs.

In preferred embodiments, a bispecific binding molecule of the invention contains immunoglobulin single variable domains, in particular VHHs, that have been obtained by sequence optimization, optionally after affinity maturation, of a parent immunoglobulin single variable domain.

By way of example, the Dll4-binding molecules contained in the bispecific binding molecules have been obtained from parent Dll4-binding molecules that are VHHs with amino acid sequences shown in Table 5 and SEQ ID NOs: 4-20.

Preferred immunoglobulin single variable domains contained in the Dll4-binding component are derived from a VHH with an amino acid sequence shown in SEQ ID NO: 10.

In certain embodiments, said preferred Dll4-binding immunoglobulin single variable domains have been obtained by sequence optimization of affinity-matured VHHs derived from the VHH with the sequence shown in SEQ ID NO: 10, wherein said affinity-matured VHHs have amino acid sequences shown in SEQ ID NOs: 21-27 and in Table 16.

In a preferred embodiment, said affinity-matured VHH has an amino acid sequence selected from sequences shown in SEQ ID NO: 22.

In preferred embodiments, the VHH has been obtained by sequence optimization of a VHH with an amino acid sequence shown in SEQ ID NO: 22. Preferred sequence-optimized VHHs have amino acid sequences selected from sequences shown in SEQ ID NOs: 34 and 35 and in Table 23.

Another group of preferred immunoglobulin single variable domains contained in the Dll4-binding component are derived from a VHH with an amino acid sequence shown in SEQ ID NO: 12.

In certain embodiments, said preferred Dll4-binding immunoglobulin single variable domains have been obtained by sequence optimization of affinity-matured VHHs derived from the VHH with the sequence shown in SEQ ID NO: 12, wherein said affinity-matured VHHs have amino acid sequences shown in SEQ ID NOs: 28-33 and in Table 17.

In a preferred embodiment, said affinity-matured VHH has an amino acid sequence selected from sequences shown in SEQ ID NOs: 30, 32 and 33.

In an even more preferred embodiment, the VHH has been obtained by sequence optimization of a VHH with an amino acid sequence shown in SEQ ID NO: 32. Examples of sequence-optimized VHHs are those with sequences shown in SEQ ID NOs: 36-39 and Table 24, and, particularly preferred, those with SEQ ID NOs: 40 and 41, shown in Table 25.

Examples for VEGF-binding immunoglobulin single variable domains capable of blocking the interaction of human recombinant VEGF165 with the human recombinant VEGFR-2 with an inhibition rate of ≧60° /o are VHHs shown in SEQ ID NOs: 42-44 and Table 32.

Preferably, a VEGF-binding immunoglobulin single variable domain contained in the VEGF-binding component has been obtained by sequence optimization of a VHH with an amino acid sequence shown in SEQ ID NO: 43. Preferred VHHs have sequences as shown in SEQ ID NOs: 54-62, particularly preferred receptor-blocking VHHs have sequences shown in SEQ ID NOs: 63 and 64 and Table 59.

In a further embodiment, the invention relates to bispecific binding molecules, wherein the Dll4-binding component and/or the VEGF-binding component comprise(s) two or more binding molecules in the form of immunoglobulin single variable domains that bind to the antigen Dll4, or VEGF, respectively, at different non-overlapping epitopes on the respective antigen. Such binding molecules contained in the bispecific binding molecules of the invention comprise immunoglobulin single variable domains that are directed against at least two non-overlapping epitopes present in Dll4 or VEGF, respectively, wherein said individual immunoglobulin single variable domains are linked to each other in such a way that they are capable of simultaneously binding to their respective epitope.

Thus, the anti-Dll4 and/or the anti-VEGF component contained in the bispecific binding molecules of the invention may include two (or more) anti-Dll4 (or anti-VEGF, respectively) immunoglobulin single variable domains, wherein the immunoglobulin single variable domains are directed against different epitopes within the Dll4 (or VEGF) target. Thus, the two immunoglobulin single variable domains in a bispecific binding molecule will have different antigen specificity and therefore different CDR sequences.

Such bivalent binding molecules are also named “biparatopic single domain antibody constructs” (if the immunoglobulin single variable domains consist or essentially consist of single domain antibodies), or “biparatopic VHH constructs” (if the immunoglobulin single variable domains consist or essentially consist of VHHs), respectively, as the two immunoglobulin single variable domains will include two different paratopes.

In the bispecific binding molecule of the invention, one or both of the binding molecules may be bivalent; e.g. the VEGF-binding component may be biparatopic and the Dll4-binding component may be one immunoglobulin single variable domain, or the VEGF-binding component may be one immunoglobulin single variable domain and the Dll4-binding component may be biparatopic.

In bispecific binding molecules of the invention, it is preferably the VEGF-binding component that contains a bivalent VEGF-binding immunoglobulin single variable domain, e.g. a biparatopic VHH.

Such VEGF-binding immunoglobulin single variable domain may be two or more VEGF-binding VHHs, which are
a. identical VHHs that are capable of blocking the interaction between recombinant human VEGF and the recombinant human VEGFR-2 with an inhibition rate of ≧60% or
b. different VHHs that bind to non-overlapping epitopes of VEGF, wherein at least one VHH is capable of blocking the interaction between recombinant human VEGF and the recombinant human VEGFR-2 with an inhibition rate of ≧60% and wherein at least one VHH is capable of blocking said interaction with an inhibition rate of ≦60%.

Examples for VHHs capable of blocking said interaction with an inhibition rate of ≦60% (“non-receptor blocking” VHHs) are listed in SEQ ID Nos: 45-47 and Table 33; a preferred VHH of this type has the sequence shown in SEQ ID NO: 45. Suitable VHHs of this type as components in bispecific binding molecules for human therapy are sequence-optimized variants of VHH with a sequence shown in SEQ ID NO: 45, in particular VHHs with sequences shown in SEQ ID Nos: 65 and 66 and in Table 61, a particularly preferred binding partner in a bivalent VEGF-binding VHH has a sequence shown in SEQ ID NO: 67 (Table 63).

Bivalent anti-VEGF VHH constructs are exemplified in SEQ ID NOs: 48-53 and Table 45; bispecific binding molecules for human therapy will contain the respective sequence-optimized variants of these VHHs. Bispecific binding molecules are exemplified in SEQ ID NOs: 68-73 (see also Table 66 and FIG. 39) and SEQ ID NO: 74-80 (see also Table 68 and FIG. 40); the examples shown contain parental and affinity-matured VHHs as buildings blocks; bispecific binding molecules for human therapy will contain the respective sequence-optimized variants of these VHHs (as exemplified in SEQ ID NOs: 81-89 and FIG. 48).

Preferred bispecific binding molecules of the invention comprise
a) as the Dll4-binding component a VHH with a sequence selected from sequences in SEQ ID NO: 35 or 41, and
b) as the VEGF-binding component
   i) a VHH with a sequence shown in SEQ ID NO: 64 or
   ii) a biparatopic VHH comprising a VHH with a sequence shown in SEQ ID NO: 64 and a VHH with a sequence shown in SEQ ID NO:67.

According to preferred embodiments, the VEGF-binding component is located at the N-terminus.

In bispecific binding molecules of the invention that start with EVQ, the N-terminal E of a VHH may be replaced by a D (which is often a result of sequence-optimization) or it may be missing (as for expression in E.coli). This usually applies only to the VHH that is situated N-terminally. Examples for bispecific binding molecules in which the N-terminal E is missing, are given in FIG. 48 for the compounds A1, A2 and A3 (SEQ ID Nos: 81-83).

According to preferred embodiments, the binding molecules present in the bispecific binding molecules (the Dll4-binding molecules within the Dll4-binding component or the VEGF-binding molecules within the VEGF-binding component or the two adjacent Dll4- and VEGF-binding components) may be connected with each other directly (i.e. without use of a linker) or via a linker. The linker is preferably a linker peptide and will be selected so as to allow binding of the two different binding molecules to each of non-overlapping epitopes of the targets, either within one and the same target molecule, or within two different molecules.

In the case of biparatopic binding molecules, selection of linkers within the D1114- or the VEGF-binding component will inter alia depend on the epitopes and, specifically, the distance between the epitopes on the target to which the immunoglobulin single variable domains bind, and will be clear to the skilled person based on the disclosure herein, optionally after some limited degree of routine experimentation.

Two binding molecules (two VHHs or domain antibodies or VHH and a domain antibody), or two binding components, may be linked to each other via an additional VHH or domain antibody, respectively (in such binding molecules, the two or more immunoglobulin single variable domains may be linked directly to said additional immunoglobulin single variable domain or via suitable linkers). Such an additional VHH or domain antibody may for example be a VHH or domain antibody that provides for an increased half-life. For example, the latter VHH or domain antibody may be one that is capable of binding to a (human) serum protein such as (human) serum albumin or (human) transferrin.

Alternatively, the two or more immunoglobulin single variable domains that bind to the respective target may be linked in series (either directly or via a suitable linker) and the additional VHH or domain antibody (which may provide for increased half-life) may be connected directly or via a linker to one of these two or more aforementioned immunoglobulin sequences.

Suitable linkers are described herein in connection with specific polypeptides of the invention and may—for example and without limitation—comprise an amino acid sequence, which amino acid sequence preferably has a length of 9 or more amino acids, more preferably at least 17 amino acids, such as about 20 to 40 amino acids. However, the upper limit is not critical but is chosen for reasons of convenience regarding e.g. biopharmaceutical production of such polypeptides.

The linker sequence may be a naturally occurring sequence or a non-naturally occurring sequence. If used for therapeutic purposes, the linker is preferably non-immunogenic in the subject to which the bispecific binding molecule of the invention is administered.

One useful group of linker sequences are linkers derived from the hinge region of heavy chain antibodies as described in WO 96/34103 and WO 94/04678.

Other examples are poly-alanine linker sequences such as Ala-Ala-Ala.

Further preferred examples of linker sequences are Gly/Ser linkers of different length such as (glyₓser_{y}), linkers, including (gly₄ser)₃, (gly₄ser)₄, (gly₄ser), (gly₃ser), gly₃, and (gly₃ser₂)₃.

Some non-limiting examples of linkers are shown in FIGS. 40 and 48, e.g. the linkers

(35GS; SEQ ID NO: 90)

GGGGSGGGGSGGGGSGGGGSGGGGSGGGGSGGGGS;

(9GS; SEQ ID NO: 91)

GGGGSGGGS;

(40GS; SEQ ID NO: 92)

GGGGSGGGGSGGGGSGGGGSGGGGSGGGGSGGGGSGGGGS.

If a bispecific binding molecule is modified by the attachment of a polymer, for example of a polyethylene glycol PEG (polyethylene glycol) moiety, the linker sequence preferably includes an amino acid residue, such as a cysteine or a lysine, allowing such modification, e.g. PEGylation, in the linker region.

Examples of linkers useful for PEGylation are:

(“GS9, C5”, SEQ ID NO: 93)

GGGGCGGGS;

(“GS25, C5, SEQ ID NO: 94)

GGGGCGGGGSGGGGSGGGGSGGGGS

(“GS27, C14”, SEQ ID NO: 95)

GGGSGGGGSGGGGCGGGGSGGGGSGGG,

(“GS35, C15”, SEQ ID NO: 96)

GGGGSGGGGSGGGGCGGGGSGGGGSGGGGSGGGGS,
and

(“GS35, C5”, SEQ ID NO: 97)

GGGGCGGGGSGGGGSGGGGSGGGGSGGGGSGGGGS.

Furthermore, the linker may also be a poly(ethylene glycol) moiety, as shown in e.g. WO 04/081026.

In another embodiment, the immunoglobulin single variable domains are linked to each other via another moiety (optionally via one or two linkers), such as another polypeptide which, in a preferred but non-limiting embodiment, may be a further immunoglobulin single variable domain as described above. Such moiety may either be essentially inactive or may have a biological effect such as improving the desired properties of the polypeptide or may confer one or more additional desired properties to the polypeptide. For example, and without limitation, the moiety may improve the half-life of the protein or polypeptide, and/or may reduce its immunogenicity or improve any other desired property.

According to a preferred embodiment, a bispecific binding molecule of the invention includes, especially when intended for use or used as a therapeutic agent, a moiety which extends the half-life of the polypeptide of the invention in serum or other body fluids of a patient. The term “half-life” is defined as the time it takes for the serum concentration of the (modified) polypeptide to reduce by 50%, in vivo, for example due to degradation of the polypeptide and/or clearance and/or sequestration by natural mechanisms.

More specifically, such half-life extending moiety can be covalently linked to or fused to an immunoglobulin single variable domain and may be, without limitation, an Fc portion, an albumin moiety, a fragment of an albumin moiety, an albumin binding moiety, such as an anti-albumin immunoglobulin single variable domain, a transferrin binding moiety, such as an anti-transferrin immunoglobulin single variable domain, a polyoxyalkylene molecule, such as a polyethylene glycol molecule, an albumin binding peptide or a hydroxyethyl starch (HES) derivative.

In another embodiment, the bispecific binding molecule of the invention comprises a moiety which binds to an antigen found in blood, such as serum albumin, serum immunoglobulins, thyroxine-binding protein, fibrinogen or transferrin, thereby conferring an increased half-life in vivo to the resulting polypeptide of the invention. According to a specifically preferred embodiment, such moiety is an albumin-binding immunoglobulin and, especially preferred, an albumin-binding immunoglobulin single variable domain such as an albumin-binding VHH domain.

If intended for use in humans, such albumin-binding immunoglobulin single variable domain preferably binds to human serum albumin and preferably is a humanized albumin-binding VHH domain.

Immunoglobulin single variable domains binding to human serum albumin are known in the art and are described in further detail in e.g. WO 2006/122786. Specifically, useful albumin binding VHHs are ALB 1 and its humanized counterpart, ALB 8 (WO 2009/095489). Other albumin binding VHH domains mentioned in the above patent publication may, however, be used as well.

A specifically useful albumin binding VHH domain is ALB8 which consists of or contains the amino acid sequence shown in SEQ ID NO: 98.

According to a further embodiment of the invention, the two immunoglobulin single variable domains, in preferably VHHs, may be fused to a serum albumin molecule, such as described e.g. in WO01/79271 and WO03/59934. As e.g. described in WO01/79271, the fusion protein may be obtained by conventional recombinant technology: a DNA molecule coding for serum albumin, or a fragment thereof, is joined to the DNA coding for the VEGF-binding molecule, the obtained construct is inserted into a plasmid suitable for expression in the selected host cell, e.g. a yeast cell like Pichia pastoris or a bacterial cell, and the host cell is then transfected with the fused nucleotide sequence and grown under suitable conditions. The sequence of a useful HSA is shown in SEQ ID NO: 99.

According to another embodiment, a half-life extending modification of a polypeptide of the invention (such modification also reducing immunogenicity of the polypeptide) comprises attachment of a suitable pharmacologically acceptable polymer, such as straight or branched chain poly(ethylene glycol) (PEG) or derivatives thereof (such as methoxypoly(ethylene glycol) or mPEG). Generally, any suitable form of PEGylation can be used, such as the PEGylation used in the art for antibodies and antibody fragments (including but not limited to domain antibodies and scFv's); reference is made, for example, to: Chapman, Nat. Biotechnol., 54, 531-545 (2002); Veronese and Harris, Adv. Drug Deliv. Rev. 54, 453-456 (2003); Harris and Chess, Nat. Rev. Drug. Discov. 2 (2003); and WO04/060965.

Various reagents for PEGylation of polypeptides are also commercially available, for example from Nektar Therapeutics, USA, or NOF Corporation, Japan, such as the Sunbright® EA Series, SH Series, MA Series, CA Series, and ME Series, such as Sunbright® ME-100MA, Sunbright® ME-200MA, and Sunbright® ME-400MA.

Preferably, site-directed PEGylation is used, in particular via a cysteine-residue (see for example Yang et al., Protein Engineering 16, 761-770 (2003)). For example, for this purpose, PEG may be attached to a cysteine residue that naturally occurs in a polypeptide of the invention, a polypeptide of the invention may be modified so as to suitably introduce one or more cysteine residues for attachment of PEG, or an amino acid sequence comprising one or more cysteine residues for attachment of PEG may be fused to the N- and/or C-terminus of a polypeptide of the invention, all using techniques of protein engineering known per se to the skilled person.

Preferably, for the polypeptides of the invention, a PEG is used with a molecular weight of more than 5 kDa, such as more than 10 kDa and less than 200 kDa, such as less than 100 kDa; for example in the range of 20 kDa to 80 kDa.

With regard to PEGylation, its should be noted that generally, the invention also encompasses any bispecific binding molecule that has been PEGylated at one or more amino acid positions, preferably in such a way that said PEGylation either (1) increases the half-life in vivo; (2) reduces immunogenicity; (3) provides one or more further beneficial properties known per se for PEGylation; (4) does not essentially affect the affinity of the polypeptide for its target (e.g. does not reduce said affinity by more than 50%, and more preferably not by more than 10%, as determined by a suitable assay described in the art); and/or (4) does not affect any of the other desired properties of the bispecific binding molecules of the invention. Suitable PEG-groups and methods for attaching them, either specifically or non-specifically, will be clear to the skilled person. Various reagents for PEGylation of polypeptides are also commercially available, for example from Nektar Therapeutics, USA, or NOF Corporation, Japan, such as the Sunbright® EA Series, SH Series, MA Series, CA Series, and ME Series, such as Sunbright® ME-100MA, Sunbright® ME-200MA, and Sunbright® ME-400MA.

According to an especially preferred embodiment of the invention, a PEGylated polypeptide of the invention includes one PEG moiety of linear PEG having a molecular weight of 40 kDa or 60 kDa, wherein the PEG moiety is attached to the polypeptide in a linker region and, specifially, at a Cys residue at position 5 of a GS9-linker peptide as shown in SEQ ID NO:93, at position 14 of a GS27-linker peptide as shown in SEQ ID NO:95, or at position 15 of a GS35-linker peptide as shown in SEQ ID NO:96, or at position 5 of a 35GS-linker peptide as shown in SEQ ID NO:97.

A bispecific binding molecule of the invention may be PEGylated with one of the PEG reagents as mentioned above, such as “Sunbright® ME-400MA”, as shown in the following chemical formula:

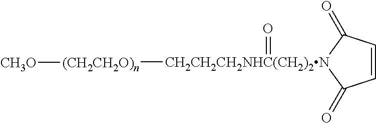

Bispecific binding molecules that contain linkers and/or half-life extending functional groups are shown in SEQ ID NO: 81 and in FIG. 48.

According to another embodiment, the immunoglobulin single variable domains are domain antibodies, as defined herein.

Immunoglobulin single variable domains present in the bispecific binding molecules of the invention may also have sequences that correspond to the amino acid sequence of a naturally occurring VH domain that has been “camelized”, i.e. by replacing one or more amino acid residues in the amino acid sequence of a naturally occurring variable heavy chain from a conventional 4-chain antibody by one or more amino acid residues that occur at the corresponding position(s) in a VHH domain of a heavy chain antibody. This can be performed in a manner known per se, which will be clear to the skilled person, and reference is additionally be made to WO 94/04678. Such camelization may preferentially occur at amino acid positions which are present at the VH-VL interface and at the so-called Camelidae Hallmark residues (see for example also WO 94/04678). A detailled description of such “humanization” and “camelization” techniques and preferred framework region sequences consistent therewith can additionally be taken from e.g. pp. 46 and pp. 98 of WO 2006/040153 and pp. 107 of WO 2006/122786.

The binding molecules have specificity for Dll4 or VEGF, respectively, in that they comprise one or more immunoglobulin single variable domains specifically binding to one or more epitopes within the Dll4 molecule or within the VEGF molecule, respectively.

Specific binding of a binding molecule to its antigen Dll4 or VEGF can be determined in any suitable manner known per se, including, for example, the assays described herein, Scatchard analysis and/or competitive binding assays, such as radioimmunoassays (RIA), enzyme immunoassays (EIA and ELISA) and sandwich competition assays, and the different variants thereof known per se in the art.

With regard to the antigen Dll4 or VEGF, respectively, an immunoglobulin single variable domain is not limited with regard to the species. Thus, the immunoglobulin single variable domains preferably bind to human Dll4 or to human VEGF, respectively, if intended for therapeutic purposes in humans. However, immunoglobulin single variable domains that bind to Dll4 or VEGF, respectively, from another mammalian species, or polypeptides containing them, are also within the scope of the invention. An immunoglobulin single variable domain binding to one species form of Dll4 or VEGF may cross-react with the respective antigen from one or more other species. For example, immunoglobulin single variable domains binding to the human antigen may exhibit cross reactivity with the respective antigen from one or more other species of primates and/or with the antigen from one or more species of animals that are used in animal models for diseases, for example monkey (in particular Cynomolgus or Rhesus), mouse, rat, rabbit, pig, dog or) and in particular in animal models for diseases and disorders that can be modulated by inhibition of Dll4 (such as the species and animal models mentioned herein). Immunoglobulin single variable domains of the invention that show such cross-reactivity are advantageous in a research and/or drug development, since it allows the immunoglobulin single variable domains of the invention to be tested in acknowledged disease models such as monkeys, in particular Cynomolgus or Rhesus, or mice and rats.

Also, the binding molecules are not limited to or defined by a specific domain or an antigenic determinant of the antigen against which they are directed. Preferably, in view of cross-reactivity with one or more antigen molecules from species other than human that is/are intended for use as an animal model during development of a therapeutic Dll4/VEGF antagonist, a binding molecule recognizes an epitope in a region of the the respective antigen that has a high degree of identity with the human antigen. By way of example, in view of using a mouse model, an anti-Dll4 immunoglobulin single variable domain contained in the bispecific binding molecules of the invention recognizes an epitope which is, totally or in part, located within the EGF-2 domain of Dll4, which shows a high identity between human and mouse.

Therefore, according to a preferred embodiment, the bispecific binding molecule of the invention comprises a Dll4-binding molecule which is an immunoglobulin single variable domain that is selected from the group that binds to an epitope that is totally or partially contained within the EGF-2 domain that corresponds to amino acid residues 252-282 of SEQ ID NO:101.

If a bispecific binding molecule of the invention contains a biparatopic

Dll4-binding molecule, which contains more than one immunoglobulin single variable domain, at least one of the immunoglobulin single variable domain components binds to the epitope within the EGF-2 domain, as defined above. Preferably, the VEGF-binding component binds to the VEGF isoforms VEGF165 and/or VEGF121.

Preferably, an immunoglobulin single variable domain that is a component of a bispecific binding molecule of the invention binds to Dll4 or to VEGF, respectively, with an affinity less than 500 nM, preferably less than 200 nM, more preferably less than 10 nM, such as less than 500 pM (as determined by Surface Plasmon Resonance analysis, as described in Example 5.7).

Preferably, immunoglobulin single variable domains contained in the bispecific binding molecules of the invention have IC₅₀ values, as measured in a competition ELISA assay as described in Example 5.1. in the range of 10⁻⁶ to 10⁻¹⁰ moles/litre or less, more preferably in the range of 10⁻⁸ to 10⁻¹⁰ moles/litre or less and even more preferably in the range of 10⁻⁹ to 10⁻¹⁰ moles/litre or less.

According to a non-limiting but preferred embodiment of the invention, Dll4- or VEGF-binding immunoglobulin single variable domains contained in the bispecific binding molecules of the invention bind to Dll4 or VEGF, respectively, with an dissociation constant (K_{D}) of 10⁻⁵ to 10⁻¹² moles/liter (M) or less, and preferably 10⁻⁷ to 10⁻¹² moles/liter (M) or less and more preferably 10⁻⁸ to 10⁻¹² moles/liter (M), and/or with an association constant (K_{A}) of at least 10⁷ M⁻¹, preferably at least 10⁸ M⁻¹, more preferably at least 10⁹ M⁻¹, such as at least 10¹² M⁻¹; and in particular with a K_{D} less than 500 nM, preferably less than 200 nM, more preferably less than 10 nM, such as less than 500 pM. The K_{D} and K_{A} values of the immunoglobulin single variable domain of the invention against Dll4 can be determined.

In another aspect, the invention relates to nucleic acid molecules that encode bispecific binding molecules of the invention. Such nucleic acid molecules will also be referred to herein as “nucleic acids of the invention” and may also be in the form of a genetic construct, as defined herein. A nucleic acid of the invention may be genomic DNA, cDNA or synthetic DNA (such as DNA with a codon usage that has been specifically adapted for expression in the intended host cell or host organism). According to one embodiment of the invention, the nucleic acid of the invention is in essentially isolated form, as defined hereabove.

The nucleic acid of the invention may also be in the form of, may be present in and/or may be part of a vector, such as for example a plasmid, cosmid or YAC. The vector may especially be an expression vector, i.e. a vector that can provide for expression of the Dll4-binding molecule in vitro and/or in vivo (i.e. in a suitable host cell, host organism and/or expression system). Such expression vector generally comprises at least one nucleic acid of the invention that is operably linked to one or more suitable regulatory elements, such as promoter(s), enhancer(s), terminator(s), and the like. Such elements and their selection in view of expression of a specific sequence in a specific host are common knowledge of the skilled person. Specific examples of regulatory elements and other elements useful or necessary for expressing Dll4-binding molecules of the invention, such as promoters, enhancers, terminators, integration factors, selection markers, leader sequences, reporter genes, and the like, are disclosed e.g. on pp. 131 to 133 of WO 2006/040153.

The nucleic acids of the invention may be prepared or obtained in a manner known per se (e.g. by automated DNA synthesis and/or recombinant DNA technology), based on the information on the amino acid sequences for the polypeptides of the invention given herein, and/or can be isolated from a suitable natural source.

In another aspect, the invention relates to host cells that express or that are capable of expressing one or more bispecific binding molecules of the invention; and/or that contain a nucleic acid of the invention. According to a particularly preferred embodiment, said host cells are bacterial cells; other useful cells are yeast cells, fungal cells or mammalian cells.

Suitable bacterial cells include cells from gram-negative bacterial strains such as strains of Escherichia coli, Proteus, and Pseudomonas, and gram-positive bacterial strains such as strains of Bacillus, Streptomyces, Staphylococcus, and Lactococcus. Suitable fungal cell include cells from species of Trichoderma, Neurospora, and Aspergillus. Suitable yeast cells include cells from species of Saccharomyces (for example Saccharomyces cerevisiae), Schizosaccharomyces (for example Schizosaccharomyces pombe), Pichia (for example Pichia pastoris and Pichia methanolica), and Hansenula.

Suitable mammalian cells include for example CHO cells, BHK cells, HeLa cells, COS cells, and the like. However, amphibian cells, insect cells, plant cells, and any other cells used in the art for the expression of heterologous proteins can be used as well.

The invention further provides methods of manufacturing a bispecific binding molecule of the invention, such methods generally comprising the steps of:

culturing host cells comprising a nucleic acid capable of encoding a bispecific binding molecule under conditions that allow expression of the bispecific binding molecule of the invention; and

recovering or isolating the polypeptide expressed by the host cells from the culture; and

optionally further purifying and/or modifying and/or formulating the bispecific binding molecule of the invention.

For production on an industrial scale, preferred host organisms include strains of E. coli, Pichia pastoris, and S. cerevisiae that are suitable for large scale expression, production and fermentation, and in particular for large scale pharmaceutical expression, production and fermentation.

The choice of the specific expression system depends in part on the requirement for certain post-translational modifications, more specifically glycosylation. The production of a bispecific binding molecule of the invention for which glycosylation is desired or required would necessitate the use of mammalian expression hosts that have the ability to glycosylate the expressed protein. In this respect, it will be clear to the skilled person that the glycosylation pattern obtained (i.e. the kind, number and position of residues attached) will depend on the cell or cell line that is used for the expression.

Bispecific binding molecules of the invention may be produced either in a cell as set out above intracellullarly (e.g. in the cytosol, in the periplasma or in inclusion bodies) and then isolated from the host cells and optionally further purified; or they can be produced extracellularly (e.g. in the medium in which the host cells are cultured) and then isolated from the culture medium and optionally further purified.

Methods and reagents used for the recombinant production of polypeptides, such as specific suitable expression vectors, transformation or transfection methods, selection markers, methods of induction of protein expression, culture conditions, and the like, are known in the art. Similarly, protein isolation and purification techniques useful in a method of manufacture of a polypeptide of the invention are well known to the skilled person.

In a further aspect, the invention relates to a peptide with an amino acid sequence selected from amino acid sequences shown in SEQ ID NOs: 1 to 166, SEQ ID NOs: 333 to 353, or SEQ ID NOs: 375 to 395, respectively, and a nucleic acid molecule encoding same.

These peptides correspond to CDR3s derived from the VHHs of the invention. They, in particular the nucleic acid molecules encoding them, are useful for CDR grafting in order to replace a CDR3 in an immunoglobulin chain, or for insertion into a non-immunoglobulin scaffold, e.g. a protease inhibitor, DNA-binding protein, cytochrome b562, a helix-bundle protein, a disulfide-bridged peptide, a lipocalin or an anticalin, thus conferring target-binding properties to such scaffold. The method of CDR-grafting is well known in the art and has been widely used, e.g. for humanizing antibodies (which usually comprises grafting the CDRs from a rodent antibody onto the Fv frameworks of a human antibody).

In order to obtain an immunoglobulin or a non-immunoglobulin scaffold containing a CDR3 of the invention, the DNA encoding such molecule may be obtained according to standard methods of molecular biology, e.g. by gene synthesis, by oligonucleotide annealing or by means of overlapping PCR fragments, as e.g. described by Daugherty et al., 1991, Nucleic Acids Research, Vol. 19, 9, 2471-2476. A method for inserting a VHH CDR3 into a non-immunoglobulin scaffold has been described by Nicaise et al., 2004, Protein Science, 13, 1882-1891.

The invention further relates to a product or composition containing or comprising at least one bispecific binding molecule of the invention and optionally one or more further components of such compositions known per se, i.e. depending on the intended use of the composition.

For pharmaceutical use, a bispecific binding molecule of the invention or a polypeptide containing same may be formulated as a pharmaceutical preparation or composition comprising at least one bispecific binding molecule of the invention and at least one pharmaceutically acceptable carrier, diluent or excipient and/or adjuvant, and optionally one or more further pharmaceutically active polypeptides and/or compounds. By means of non-limiting examples, such a formulation may be in a form suitable for oral administration, for parenteral administration (such as by intravenous, intramuscular or subcutaneous injection or intravenous infusion), for topical administration, for administration by inhalation, by a skin patch, by an implant, by a suppository, etc. Such suitable administration forms—which may be solid, semi-solid or liquid, depending on the manner of administration—as well as methods and carriers for use in the preparation thereof, will be clear to the skilled person, and are further described herein.

Thus, in a further aspect, the invention relates to a pharmaceutical composition that contains at least one bispecific binding molecule, in particular one immunoglobulin single variable domain of the invention or a polypeptide containing same and at least one suitable carrier, diluent or excipient (i.e. suitable for pharmaceutical use), and optionally one or more further active substances.

The bispecific binding molecules of the invention may be formulated and administered in any suitable manner known per se: Reference, in particular for the immunoglobulin single variable domains, is for example made to WO 04/041862, WO 04/041863, WO 04/041865, WO 04/041867 and WO 08/020079, as well as to the standard handbooks, such as Remington's Pharmaceutical Sciences, 18^{th} Ed., Mack Publishing Company, USA (1990), Remington, the Science and Practice of Pharmacy, 21^{th} Edition, Lippincott Williams and Wilkins (2005); or the Handbook of Therapeutic Antibodies (S. Dubel, Ed.), Wiley, Weinheim, 2007 (see for example pages 252-255).

For example, an immunoglobulin single variable domain of the invention may be formulated and administered in any manner known per se for conventional antibodies and antibody fragments (including ScFv's and diabodies) and other pharmaceutically active proteins. Such formulations and methods for preparing the same will be clear to the skilled person, and for example include preparations suitable for parenteral administration (for example intravenous, intraperitoneal, subcutaneous, intramuscular, intraluminal, intra-arterial or intrathecal administration) or for topical (i.e. transdermal or intradermal) administration.

Preparations for parenteral administration may for example be sterile solutions, suspensions, dispersions or emulsions that are suitable for infusion or injection. Suitable carriers or diluents for such preparations for example include, without limitation, sterile water and pharmaceutically acceptable aqueous buffers and solutions such as physiological phosphate-buffered saline, Ringer's solutions, dextrose solution, and Hank's solution; water oils; glycerol; ethanol; glycols such as propylene glycol or as well as mineral oils, animal oils and vegetable oils, for example peanut oil, soybean oil, as well as suitable mixtures thereof. Usually, aqueous solutions or suspensions will be preferred.

Thus, the bispecific binding molecule of the invention may be systemically administered, e.g., orally, in combination with a pharmaceutically acceptable vehicle such as an inert diluent or an assimilable edible carrier. For oral therapeutic administration, the bispecific binding molecule of the invention may be combined with one or more excipients and used in the form of ingestible tablets, buccal tablets, troches, capsules, elixirs, suspensions, syrups, wafers, and the like. Such compositions and preparations should contain at least 0.1% of the bispecific binding molecule of the invention. Their percentage in the compositions and preparations may, of course, be varied and may conveniently be between about 2 to about 60% of the weight of a given unit dosage form. The amount of the bispecific binding molecule of the invention in such therapeutically useful compositions is such that an effective dosage level will be obtained.

The tablets, pills, capsules, and the like may also contain binders, excipients, disintegrating agents, lubricants and sweetening or flavouring agents, for example those mentioned on pages 143-144 of WO 08/020079. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the bispecific binding molecules of the invention, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the bispecific binding molecules of the invention may be incorporated into sustained-release preparations and devices.

Preparations and formulations for oral administration may also be provided with an enteric coating that will allow the constructs of the invention to resist the gastric environment and pass into the intestines. More generally, preparations and formulations for oral administration may be suitably formulated for delivery into any desired part of the gastrointestinal tract. In addition, suitable suppositories may be used for delivery into the gastrointestinal tract.

The bispecific binding molecules of the invention may also be administered intravenously or intraperitoneally by infusion or injection, as further described on pages 144 and 145 of WO 08/020079.

For topical administration of the bispecific binding molecules of the invention, it will generally be desirable to administer them to the skin as compositions or formulations, in combination with a dermatologically acceptable carrier, which may be a solid or a liquid, as further described on page 145 of WO 08/020079.

Generally, the concentration of the bispecific binding molecules of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the bispecific binding molecules of the invention required for use in treatment will vary not only with the particular bispecific binding molecule selected, but also with the route of administration, the nature of the condition being treated and the age and condition of the patient and will be ultimately at the discretion of the attendant physician or clinician. Also, the dosage of the bispecific binding molecules of the invention varies depending on the target cell, tumor, tissue, graft, or organ.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

An administration regimen may include long-term, daily treatment. By “long-term” is meant at least two weeks and preferably, several weeks, months, or years of duration. Necessary modifications in this dosage range may be determined by one of ordinary skill in the art using only routine experimentation given the teachings herein. See Remington's Pharmaceutical Sciences (Martin, E. W., ed. 4), Mack Publishing Co., Easton, Pa. The dosage can also be adjusted by the individual physician in the event of any complication.

According to a further embodiment, the invention relates to the use of bispecific binding molecules, e.g. immunoglobulin single variable domains or polypeptides containing them, for therapeutic purposes, such as
for the prevention, treatment and/or alleviation of a disorder, disease or condition, especially in a human being, that is associated with Dll4-mediated effects on angiogenesis or that can be prevented, treated or alleviated by modulating the Notch signaling pathway with a Dll4-binding molecule,
in a method of treatment of a patient in need of such therapy, such method comprising administering, to a subject in need thereof, a pharmaceutically active amount of at least one bispecific binding molecule of the invention, e.g. an immunoglobulin single variable domain, or a pharmaceutical composition containing same;
for the preparation of a medicament for the prevention, treatment or alleviation of disorders, diseases or conditions associated with Dll4-mediated effects on angiogenesis;
as an active ingredient in a pharmaceutical composition or medicament used for the above purposes.

According to a specific aspect, said disorder disorder, disease or condition is a cancer or cancerous disease, as defined herein.

According to another aspect, the disease is an eye disease associated with associated with Dll4-mediated effects on angiogenesis or which can be treated or alleviated by modulating the Notch signaling pathway with a Dll4-binding molecule.

Depending on the cancerous disease to be treated, a bispecific binding molecule of the invention may be used on its own or in combination with one or more additional therapeutic agents, in particular selected from chemotherapeutic agents like DNA damaging agents or therapeutically active compounds that inhibit angiogenesis, signal transduction pathways or mitotic checkpoints in cancer cells.

The additional therapeutic agent may be administered simultaneously with, optionally as a component of the same pharmaceutical preparation, or before or after administration of the bispecific binding molecule.

In certain embodiments, the additional therapeutic agent may be, without limitation (and in the case of the receptors, including the respective ligands), one or more inhibitors selected from the group of inhibitors of EGFR, VEGFR, HER2-neu, Her3, AuroraA, AuroraB, PLK and PI3 kinase, FGFR, PDGFR, Raf, KSP, PDK1, PTK2, IGF-R or IR.

Further examples of additional therapeutic agents are inhibitors of CDK, Akt, src/bcr abl, cKit, cMet/HGF, c-Myc, Flt3, HSP90, hedgehog antagonists, inhibitors of JAK/STAT, Mek, mTor, NFkappaB, the proteasome, Rho, an inhibitor of wnt signaling or an inhibitor of the ubiquitination pathway or another inhibitor of the Notch signaling pathway.

Examples for Aurora inhibitors are, without limitation, PHA-739358, AZD-1152, AT 9283, CYC-116, R-763, VX-680, VX-667, MLN-8045, PF-3814735.

An example for a PLK inhibitor is GSK-461364.

Examples for raf inhibitors are BAY-73-4506 (also a VEGFR inhibitor), PLX 4032, RAF-265 (also in addition a VEGFR inhibitor), sorafenib (also in addition a VEGFR inhibitor), and XL 281.

Examples for KSP inhibitors are ispinesib, ARRY-520, AZD-4877, CK-1122697, GSK 246053A, GSK-923295, MK-0731, and SB-743921.

Examples for a src and/or bcr-abl inhibitors are dasatinib, AZD-0530, bosutinib, XL 228 (also an IGF-1R inhibitor), nilotinib (also a PDGFR and cKit inhibitor), imatinib (also a cKit inhibitor), and NS-187.

An example for a PDK1 inhibitor is BX-517.

An example for a Rho inhibitor is BA-210.

Examples for PI3 kinase inhibitors are PX-866, BEZ-235 (also an mTor inhibitor), XL 418 (also an Akt inhibitor), XL-147, and XL 765 (also an mTor inhibitor).

Examples for inhibitors of cMet or HGF are XL-184 (also an inhibitor of VEGFR, cKit, Flt3), PF-2341066, MK-2461, XL-880 (also an inhibitor of VEGFR), MGCD-265 (also an inhibitor of VEGFR, Ron, Tie2), SU-11274, PHA-665752, AMG-102, and AV-299.

An example for a c-Myc inhibitor is CX-3543.

Examples for Flt3 inhibitors are AC-220 (also an inhibitor of cKit and PDGFR), KW 2449, lestaurtinib (also an inhibitor of VEGFR, PDGFR, PKC), TG-101348 (also an inhibitor of JAK2), XL-999 (also an inhibitor of cKit, FGFR, PDGFR and VEGFR), sunitinib (also an inhibitor of PDGFR, VEGFR and cKit), and tandutinib (also an inhibitor of PDGFR, and cKit).

Examples for HSP90 inhibitors are tanespimycin, alvespimycin, IPI-504 and CNF 2024.

Examples for JAK/STAT inhibitors are CYT-997 (also interacting with tubulin), TG 101348 (also an inhibitor of Flt3), and XL-019.

Examples for Mek inhibitors are ARRY-142886, PD-325901, AZD-8330, and XL 518.

Examples for mTor inhibitors are temsirolimus, AP-23573 (which also acts as a VEGF inhibitor), everolimus (a VEGF inhibitor in addition). XL-765 (also a PI3 kinase inhibitor), and BEZ-235 (also a PI3 kinase inhibitor).

Examples for Akt inhibitors are perifosine, GSK-690693, RX-0201, and triciribine.

Examples for cKit inhibitors are AB-1010, OSI-930 (also acts as a VEGFR inhibitor), AC-220 (also an inhibitor of Flt3 and PDGFR), tandutinib (also an inhibitor of Flt3 and PDGFR), axitinib (also an inhibitor of VEGFR and PDGFR), XL-999 (also an inhibitor of Flt3, PDGFR, VEGFR, FGFR), sunitinib (also an inhibitor of Flt3, PDGFR, VEGFR), and XL-820 (also acts as a VEGFR- and PDGFR inhibitor), imatinib (also a bcr-abl inhibitor), nilotinib (also an inhibitor of bcr-abl and PDGFR).

Examples for hedgehog antagonists are IPl-609 and CUR-61414.

Examples for CDK inhibitors are seliciclib, AT-7519, P-276, ZK-CDK (also inhibiting VEGFR2 and PDGFR), PD-332991, R-547, SNS-032, PHA-690509, and AG 024322.

Examples for proteasome inhibitors are bortezomib, carfilzomib, and NPI-0052 (also an inhibitor of NFkappaB).

An example for an NFkappaB pathway inhibitor is NPI-0052.

An example for an ubiquitination pathway inhibitor is HBX-41108.

In preferred embodiments, the additional therapeutic agent is an anti-angiogenic agent.

Examples for anti-angiogenic agents are inhibitors of the FGFR, PDGFR and VEGFR or the respective ligands (e.g VEGF inhibitors like pegaptanib or the anti-VEGF antibody bevacizumab), and thalidomides, such agents being selected from, without limitation, bevacizumab, motesanib, CDP-791, SU-14813, telatinib, KRN-951, ZK-CDK (also an inhibitor of CDK), ABT-869, BMS-690514, RAF-265, IMC-KDR, IMC-18F1, IMiDs (immunomodulatory drugs), thalidomide derivative CC-4047, lenalidomide, ENMD 0995, IMC-D11, Ki 23057, brivanib, cediranib, XL-999 (also an inhibitor of cKit and Flt3), 1B3, CP 868596, IMC 3G3, R-1530 (also an inhibitor of Flt3), sunitinib (also an inhibitor of cKit and Flt3), axitinib (also an inhibitor of cKit), lestaurtinib (also an inhibitor of Flt3 and PKC), vatalanib, tandutinib (also an inhibitor of Flt3 and cKit), pazopanib, GW 786034, PF-337210, IMC-1121B, AVE-0005, AG-13736, E-7080, CHIR 258, sorafenib tosylate (also an inhibitor of Raf), RAF-265 (also an inhibitor of Raf), vandetanib, CP-547632, OSI-930, AEE-788 (also an inhibitor of EGFR and Her2), BAY-57-9352 (also an inhibitor of Raf), BAY-73-4506 (also an inhibitor of Raf), XL 880 (also an inhibitor of cMet), XL-647 (also an inhibitor of EGFR and EphB4), XL 820 (also an inhibitor of cKit), and nilotinib (also an inhibitor of cKit and brc-abl).

The additional therapeutic agent may also be selected from EGFR inhibitors, it may be a small molecule EGFR inhibitor or an anti-EGFR antibody. Examples for anti-EGFR antibodies, without limitation, are cetuximab, panitumumab, matuzumab; an example for a small molecule EGFR inhibitor is gefitinib. Another example for an EGFR modulator is the EGF fusion toxin.

Among the EGFR and Her2 inhibitors useful for combination with the bispecific binding molecule of the invention are lapatinib, gefitinib, erlotinib, cetuximab, trastuzumab, nimotuzumab, zalutumumab, vandetanib (also an inhibitor of VEGFR), pertuzumab, XL-647, HKI-272, BMS-599626 ARRY-334543, AV 412, mAB-806, BMS-690514, JNJ-26483327, AEE-788 (also an inhibitor of VEGFR), ARRY-333786, IMC-11F8, Zemab.

Other agents that may be advantageously combined in a therapy with the bispecific binding molecule of the invention are tositumumab and ibritumomab tiuxetan (two radiolabelled anti-CD20 antibodies), alemtuzumab (an anti-CD52 antibody), denosumab, (an osteoclast differentiation factor ligand inhibitor), galiximab (a CD80 antagonist), ofatumumab (a CD20 inhibitor), zanolimumab (a CD4 antagonist), SGN40 (a CD40 ligand receptor modulator), rituximab (a CD20 inhibitor) or mapatumumab (a TRAIL-1 receptor agonist).

Other chemotherapeutic drugs that may be used in combination with the bispecific binding molecules of the present invention are selected from, but not limited to hormones, hormonal analogues and antihormonals (e.g. tamoxifen, toremifene, raloxifene, fulvestrant, megestrol acetate, flutamide, nilutamide, bicalutamide, cyproterone acetate, finasteride, buserelin acetate, fludrocortisone, fluoxymesterone, medroxyprogesterone, octreotide, arzoxifene, pasireotide, vapreotide), aromatase inhibitors (e.g. anastrozole, letrozole, liarozole, exemestane, atamestane, formestane), LHRH agonists and antagonists (e.g. goserelin acetate, leuprolide, abarelix, cetrorelix, deslorelin, histrelin, triptorelin), antimetabolites (e.g. antifolates like methotrexate, pemetrexed, pyrimidine analogues like 5 fluorouracil, capecitabine, decitabine, nelarabine, and gemcitabine, purine and adenosine analogues such as mercaptopurine thioguanine, cladribine and pentostatin, cytarabine, fludarabine); antitumor antibiotics (e.g. anthracyclines like doxorubicin, daunorubicin, epirubicin and idarubicin, mitomycin-C, bleomycin dactinomycin, plicamycin, mitoxantrone, pixantrone, streptozocin); platinum derivatives (e.g. cisplatin, oxaliplatin, carboplatin, lobaplatin, satraplatin); alkylating agents (e.g. estramustine, meclorethamine, melphalan, chlorambucil, busulphan, dacarbazine, cyclophosphamide, ifosfamide, hydroxyurea, temozolomide, nitrosoureas such as carmustine and lomustine, thiotepa); antimitotic agents (e.g. vinca alkaloids like vinblastine, vindesine, vinorelbine, vinflunine and vincristine; and taxanes like paclitaxel, docetaxel and their formulations, larotaxel; simotaxel, and epothilones like ixabepilone, patupilone, ZK-EPO); topoisomerase inhibitors (e.g. epipodophyllotoxins like etoposide and etopophos, teniposide, amsacrine, topotecan, irinotecan) and miscellaneous chemotherapeutics such as amifostine, anagrelide, interferone alpha, procarbazine, mitotane, and porfimer, bexarotene, celecoxib.

The efficacy of bispecific binding molecules of the invention or polypeptides containing them, and of compositions comprising the same, can be tested using any suitable in vitro assay, cell-based assay, in vivo assay and/or animal model known per se, or any combination thereof, depending on the specific disease or disorder of interest. Suitable assays and animal models will be clear to the skilled person, and for example include the assays described herein and used in the Examples below, e.g. a proliferation assay.

### BRIEF DESCRIPTION OF THE FIGURES

FIG. 1: Amino acid sequence alignment of human, rhesus and cynomolgus DLL4

FIG. 2: Human and mouse DLL4 deletion mutants (amino acid domain boundaries in superscript).

FIG. 3: Purified VHHs block the hDLL4/hNotch1-Fc interaction (ELISA).

FIG. 4: Purified VHHs block the hDLL4/hNotch1-Fc interaction (AlphaScreen).

FIG. 5: Purified VHHs block the CHO-hDLL4/hNotch1-Fc and CHO-mDLL4/hNotch1-Fc interaction (FMAT).

FIG. 6: Purified VHHs block the DLL4 mediated Notch1 cleavage (reporter).

FIG. 7: Binding of purified VHHs to recombinant human and mouse DLL4 (ELISA).

FIG. 8: Binding of purified VHHs to recombinant human DLL1 and human Jagged-1 (ELISA).

FIG. 9: Binding of purified VHHs to human/mouse/cynomolgus DLL4 (FACS).

FIG. 10: Affinity-matured VHHs block the hDLL4/hNotch1-Fc interaction (ELISA).

FIG. 11: Affinity-matured VHHs block the CHO-hDLL4/hNotch1-Fc and CHO-mDLL4/hNotch1-Fc interaction (FMAT).

FIG. 12: Binding of purified VHHs to human/mouse DLL4 (ELISA)

FIG. 13: Binding of purified affinity-matured VHHs to recombinant human DLL1 and human Jagged-1 (ELISA).

FIG. 14: Binding of purified VHHs to human/mouse/cynomolgus DLL4 (FACS).

FIG. 15: Evaluation of VHHs effects on Dll4-mediated inhibition of HUVEC proliferation.

FIG. 16: Affinity matured VHHs in DLL4-mediated reporter assay

FIG. 17: A) Sequence alignment of VHH DLLBII129B05 to the human VH3/JH germline sequence.
B) Sequence alignment of VHH DLLBII136C07 to the human VH3/JH5 germline sequence.

FIG. 18: A) Purified sequence optimized VHH variants of DLLBII129B05 blocking CHO-hDLL4/hNotch1-Fc and CHO-mDLL4/hNotch1-Fc interaction (FMAT)
B) Purified sequence optimized VHH variants of DLLBII136C07 blocking CHO-hDLL4/hNotch1-Fc and CHO-mDLL4/hNotch1-Fc interaction (FMAT)

FIG. 19: Purified sequence optimized VHHs blocking DLL4 mediated Notch1 cleavage (reporter assay)

FIG. 20: Purified monovalent VHHs block the hVEGF165/hVEGFR2-Fc interaction (ELISA)

FIG. 21: Purified monovalent VHHs block the hVEGF165/hVEGFR1-Fc interaction (ELISA)

FIG. 22: Purified monovalent VHHs block the hVEGF165/hVEGFR2-Fc interaction (AlphaScreen)

FIG. 23: Purified monovalent VHHs block the hVEGF165/hVEGFR1-Fc interaction (AlphaScreen)

FIG. 24: Binding of monovalent VHHs to recombinant human and mouse VEGF (ELISA)

FIG. 25: Binding of monovalent VHHs to human VEGF121

FIG. 26: Purified VHHs do not bind to VEGFB, VEGFC, VEGFD and PIGF

FIG. 27: Formatted VHHs block hVEGF165/hVEGFR2-Fc interaction (ELISA)

FIG. 28: Formatted VHHs block hVEGF165/hVEGFR1-Fc interaction (ELISA)

FIG. 29: Formatted VHHs block hVEGF165/hVEGFR2-Fc interaction (AlphaScreen)

FIG. 30: Formatted VHHs block hVEGF165/hVEGFR1-Fc interaction (AlphaScreen)

FIG. 31: Formatted VHHs block mVEGF164/mVEGFR2-Fc interaction (AlphaScreen)

FIG. 32: Formatted VHHs bind to mouse and human VEGF

FIG. 33: Formatted VHHs do not bind to VEGFB, VEGFC, VEGFD and PIGF

FIG. 34: Formatted VHHs bind to VEGF121

FIG. 35: Sequence alignment of VHH VEGFBII23B04 with human VH3/JH germline consensus sequence

FIG. 36: VHH variants of VEGFBII23B4 block the hVEGF165/hVEGFR2-Fc interaction(AlphaScreen)

FIG. 37: Sequence-optimized clones of VEGFBII23B4 block the hVEGF165/hVEGFR2-Fc interaction (AlphaScreen)

FIG. 38: Sequence alignment of VHH VEGFBII5B5 with human VH3/JH germline consensus sequence

FIG. 39: Format of cycle 1 bispecific VEGF-DLL4 VHHs.

FIG. 40: Format of cycle 2 bispecific VEGF-DLL4 VHHs.

FIG. 41: Bispecific VHHs (cycle 1) in the VEGF/VEGFR2 AlphaScreen assay (in the presence or absence of 5 μM HSA)

FIG. 42: Bispecific VHHs (cycle 1) in the VEGF/VEGFR1 AlphaScreen assay (in presence or absence of 5 μM HSA)

FIG. 43: Bispecific VHHs (cycle 1) in the CHO-hDLL4/hNotch1-Fc FMAT assay (in presence or absence of 25 μM HSA)

FIG. 44: Bispecific VHHs (cycle 2) in the VEGF/VEGFR2 AlphaScreen assay (in presence or absence of 5 μM HSA)

FIG. 45: Bispecific VHHs (cycle 2) in the VEGF/VEGFR1 AlphaScreen assay (in the presence or absence of 5 μM HSA)

FIG. 46: Bispecific VHHs (cycle 2) in the CHO-hDLL4/hNotch1-Fc and CHO-mDLL4/hNotch1-Fc FMAT assay (in the presence or absence of 25 μM HSA)

FIG. 47: Bispecific VHHs (cycle 2) in the DLL4 mediated reporter assay (in the presence or absence of 175 μM HSA)

FIG. 48: Format of sequence-optimized bispecific VEGF-DLL4 VHHs

FIG. 49: Bispecific VHHs (cycle 3) in the VEGF/VEGFR2 AlphaScreen assay (in presence or absence of 5 μM HSA)

FIG. 50: Bispecific VHHs (cycle 3) in the VEGF/VEGFR1 AlphaScreen assay (in presence or absence of 5 μM HSA)

FIG. 51: Bispecific VHHs (cycle 3) in the CHO-hDLL4/hNotch1-Fc and CHO-mDLL4/hNotch1-Fc FMAT assay (in thr presence or absence of 25 μM HSA)

FIG. 52: Efficacy of selected VHHs in a mouse model of human colon cancer (SW620 model)
A: SW620 tumor growth kinetics
B: Absolute tumor volumes at the end of the study on day 21
C: change of body weight over time

Materials and Methods

a) Generation of CHO and HEK293 Cell Lines Overexpressing Human, Mouse and Cynomolgus D114

The cDNAs encoding human (SEQ ID NO: 101; NM_{—}019074.2) and mouse D114 (NM_{—}019454.3) are amplified from a Human Adult Normal Tissue Heart cDNA library (BioChain, Hayward, Calif., USA) and a Mouse Heart Tissue cDNA library (isolated from C57/B16 strain), respectively, using oligonucleotides designed in the 5′ and 3′ UTR of the corresponding sequence. Amplicons are cloned into the mammalian expression vector pCDNA3.1(+)-neo (Invitrogen, Carlsbad, Calif., USA).

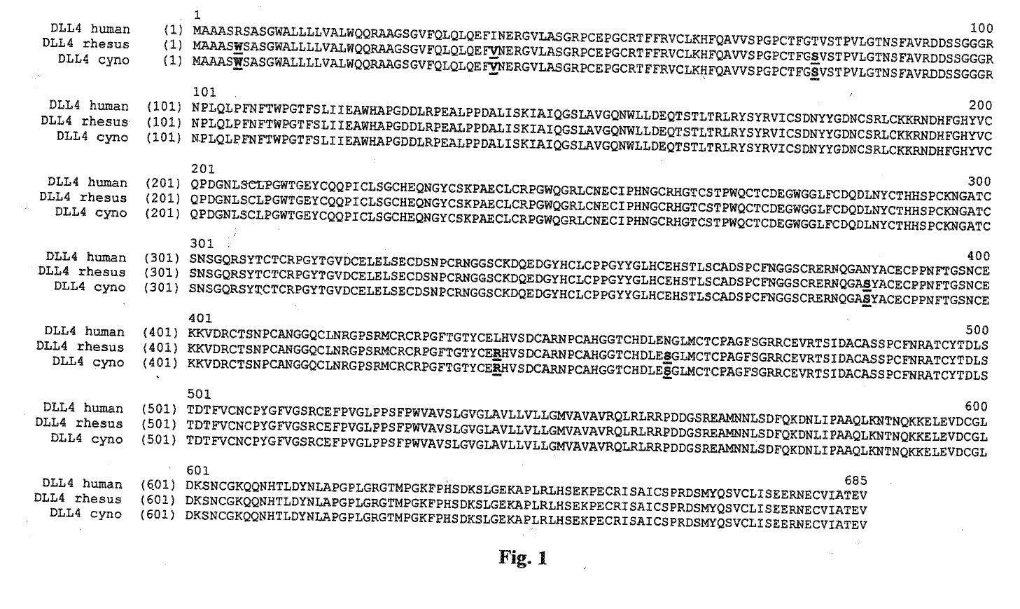

Cynomolgus D114 cDNA is amplified from a Cynomolgus Normal Tissue Heart cDNA library (BioChain, Hayward, Calif., USA), using primers designed on the 5′ and 3′ UTR of the Dll4 encoding sequence of the closely related species rhesus (Macaca mulatta Dll4, SEQ ID NO:102; XM_{—}001099250.1) (see FIG. 1). The final amplicon is cloned in the mammalian expression vector pCDNA3.1(+)-neo (Invitrogen, Carlsbad, Calif., USA). The amino acid sequence of cynomolgus Dll4 is shown to be 100% identical to rhesus, and 99% identical to human (see FIG. 1; differences from the human sequence are indicated as bold-underlined).

To establish Chinese Hamster Ovary (CHO) cells overexpressing human Dll4, mouse Dll4 or cynomolgus Dll4, parental CHO cells are electroporated with pCDNA3.1(+)-neo-hDll4, pcDNA3.1(+)-neo-mDll4 or pcDNA3.1(+)-neo-cDll4, respectively. Human Embyonic Kidney (HEK293) cells overexpressing human Dll4 and mouse Dll4 are generated by lipid-mediated transfection with Fugene (Roche) of pCDNA3.1(+)-neo-hDll4 or mDll4 plasmids, respectively, in the HEK293 parental cell line. For all conditions, transfectants are selected by adding 1 mg/mL geneticin (Invitrogen, Carlsbad, Calif., USA).

b) Generation of Monoclonal Anti-Dll4 IgG and Fab Fragment

In US 2008/0014196; Genentech) a human/mouse cross-reactive Dll4 mAb is described that is used by Ridgway et al. (2006) to show additive effects of VEGF mAb and Dll4 mAb on tumor growth in a number of xenograft models. This anti-Dll4 mAb and its corresponding Fab are purified to assess the properties of this antibody (fragment) in biochemical/cellular assays and xenograft models and for specific elutions during phage selections. The published variable heavy and light chain sequences of Dll4 mAb are cloned into a hIgG2aK framework, transiently expressed in HEK293 cells and purified from supernatants using protein A chromatography. Purified Dll4 mAb shows binding to human Dll4 and mouse Dll4 in ELISA and FACS (using CHO-mDll4 and CHO-hDll4 cells), sub-nanomolar affinities to both growth factor orthologues in Biacore.

The corresponding Dll4 Fab fragment is constructed via gene assembly based on back-translation and codon optimization for expression in E. coli using Leto's Gene Optimization software (www.entechelon.com). Oligonucleotide primers for the assembly of the variable light chain (V_{L}), variable heavy chain (V_{H}), constant light chain (CO and constant domain 1 of the heavy chain (C_{H1}) are designed and an assembly PCR is performed. The cDNA segments encoding V_{L}+C_{L} and V_{H}+C_{H1} are cloned into a pUC119-derived vector, which contains the LacZ promotor, a resistance gene for kanamycin, a multiple cloning site and a hybrid glll-pelB leader sequence, using the restriction sites SfiI and AscI and the restriction sites KpnI and NotI, respectively. In frame with the Fab coding sequence, the expression vector encodes a C-terminal HA and His6-tag. The Fab fragment is expressed in E. coli as His6-tagged protein and subsequently purified from the culture medium by immobilized metal affinity chromatography (IMAC) and size exclusion chromatography (SEC). Relevant amino acid sequences of the variable heavy and variable light chain are depicted (SEQ ID NO: 1 and SEQ ID NO: 2; respectively, of US 2008/0014196); the amino acid sequences of the complete heavy and light chain are shown in SEQ ID NOs: 419 and 420, respectively.

c) Generation of Dll4 Mutants for Epitope Mapping

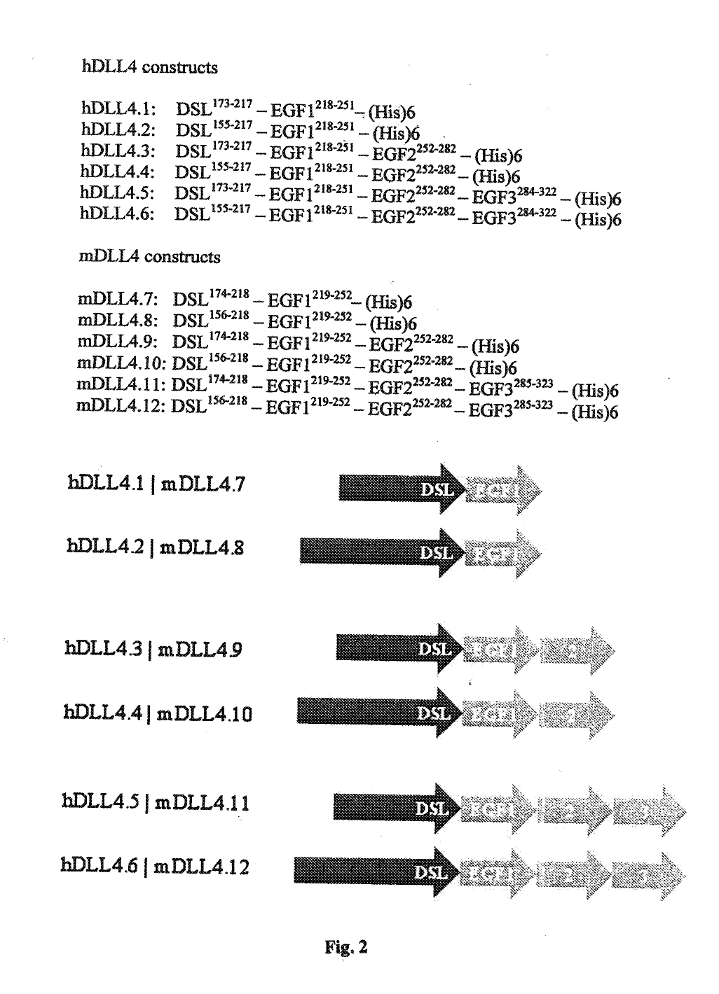

To identify the region in the extracellular domain (ECD) of Dll4 that comprises the epitope recognized by the anti-Dll4 VHHs, progressive deletion mutants of the Dll4 ECD are generated. The mammalian expression vector pSecTag2/Hygro (Invitrogen, Carlsbad, Calif., USA) comprising a CMV promotor upstream of polynucleotides encoding a nested series of deletion fragments of the Dll4 ECD fused to a polyHis-tag are generated using standard recombinant DNA technology (see FIG. 2; amino acid domain boundaries in superscript).). These recombinant proteins are expressed in transiently transfected HEK293 cells using the Freestyle 293 Expression System (Invitrogen, Carlsbad, Calif., USA) from which conditioned medium is collected and purified via IMAC. Only Dll4 mutants lacking the EGF2-like domain showed impaired binding to the humanized human/mouse cross-reactive anti-Dll4 mAb described above (immobilized via a capturing anti-human IgG coated Biacore sensor chip). This IgG is known to have a specific binding epitope in this Dll4 domain (patent application Genentech, US 2008/0014196A1). d) Generation of Dll4 Reporter Assay Plasmids

A reporter assay is developed based on the γ-secretase mediated cleavage of Notch1 and nuclear translocation of the intracellular domain of Notch1 (NICD) upon stimulation with Dll4, essentially as described (Struhl and Adachi, Cell. May 15, 1998; 93(4):649-60). Gal4/VP16 coding sequences are inserted into the NICD-coding sequence. The potent hybrid transcriptional activator GAL4-VP16, which consists of a DNA binding fragment of yeast GAL4 fused to a Herpes simplex viral transcriptional activator domain VP16, is inserted carboxy-terminal to the transmembrane domain of Notch1. Cleavage of this construct by γ-secretase results in the release of the Gal4/VP16 NICD fusion protein which will translocate to the nucleus where it will bind to and transcriptionally activate a co-transfected luciferase reporter plasmid, containing a strong GAL4-UAS promoter sequence (Struhl, G. and Adachi, A., Cell, vol. 93, 649-660, 1998). The human Notch1-Gal4/VP16 expression cassette is cloned in pcDNA3.1(+)-neo (Invitrogen, Carlsbad, Calif., USA). The pGL4.31[Luc2P/Gal4UAS/Hygro] vector (Promega, Madison, Wis., USA) is used as luciferase reporter plasmid.

e) Production and Functionality-Testing of VEGF109

A cDNA encoding the receptor binding domain of human vascular endothelial growth factor isoform VEGF165 (GenBank: AAM03108.1; AA residues 27-135) is cloned into pET28a vector (Novagen, Madison, Wis.) and overexpressed in E.coli (BL21 Star DE3) as a His-tagged insoluble protein. Expression is induced by addition of 1 mM IPTG and allowed to continue for 4 hours at 37° C. Cells are harvested by centrifugation and lysed by sonication of the cell pellet. Inclusion bodies are isolated by centrifugation. After a washing step with 1% Triton X 100 (Sigma-Aldrich), proteins are solubilized using 7.5M guanidine hydrochloride and refolded by consecutive rounds of overnight dialysis using buffers with decreasing urea concentrations from 6M till 0M. The refolded protein is purified by ion exchange chromatography using a MonoQ5/50GL (Amersham BioSciences) column followed by gel filtration with a Superdex75 10/300 GL column (Amersheim BioSciences). The purity and homogeneity of the protein is confirmed by SDS-PAGE and Westen blot. In addition, binding activity to VEGFR1, VEGFR2 and Bevacizumab is monitored by ELISA. To this end, 1 μg/mL of recombinant human VEGF109 is immobilized overnight at 4° C. in a 96-well MaxiSorp plate (Nunc, Wiesbaden, Germany). Wells are blocked with a casein solution (1%). Serial dilutions of VEGFR1, VEGFR2 or Bevacizumab are added to the VEGF109 coated plate and binding is detected using alkaline phosphatase (AP) conjugated goat anti-human IgG, Fc specific (Jackson Immuno Research Laboratories Inc., West Grove, Pa., USA) and a subsequent enzymatic reaction in the presence of the substrate PNPP (p-nitrophenylphosphate) (Sigma-Aldrich). VEGF109 could bind to VEGFR1, VEGFR2 and Bevacizumab, indicating that the produced VEGF109 is active.

f) KLH Conjugation of VEGF165 and Functionality-Testing of KLH-Conjugated VEGF165

Recombinant human VEGF165 (R&D Systems, Minneapolis, Minn., USA) is conjugated to mariculture keyhole limpet hemocyanin (mcKLH) using the lmject Immunogen EDC kit with mcKLH (Pierce, Rockford, Ill., USA) according to the manufacturer's instructions. Efficient conjugation of the polypeptide to mcKLH is confirmed by SDS-PAGE. Functionality of the conjugated protein is checked by ELISA: 2 μg/mL of KLH conjugated VEGF165 is immobilized overnight at 4° C. in a 96-well MaxiSorp plate (Nunc, Wiesbaden, Germany). Wells are blocked with a casein solution (1%). Serial dilutions of VEGFR1 or VEGFR2 are added and binding is detected using a horseradish peroxidase (HRP)-conjugated goat anti-human IgG, Fc specific (Jackson Immuno Research Laboratories Inc., West Grove, Pa., USA) and a subsequent enzymatic reaction in the presence of the substrate TMB (3,3′,5,5′-tetramentylbenzidine) (Pierce, Rockford, Ill., USA). The KLH conjugated protein could still interact with VEGFR1, VEGFR2 and Bevacizumab, confirming that the relevant epitopes on VEGF165 are still accessible.

### EXAMPLE 1

Immunization with Dll4 from Different Species Induces a Humoral Immune Response in Llama 1.1. Immunizations

After approval of the Ethical Committee of the faculty of Veterinary Medicine (University Ghent, Belgium), 4 llamas (designated No. 208, 209, 230, 231) are immunized with 6 intramuscular injections (100 or 50 μg/dose at weekly intervals) of recombinant human Dll4 (R&D Systems, Minneapolis, Minn., US). The Dll4 antigen is formulated in Stimune (Cedi Diagnostics BV, Lelystad, The Netherlands). Three additional llamas (designated No. 127b, 260, 261) are immunized according to standard protocols with 4 subcutaneous injections of alternating human Dll4 and mouse Dll4 overexpressing CHO cells which are established as described above. Cells are re-suspended in D-PBS and kept on ice prior to injection. Furthermore, three additional llamas (designated No. 282, 283, 284) are immunized according to standard protocols with 4 intramuscular injections (100 or 50 μg/dose at biweekly intervals) of alternating recombinant human Dll4 and mouse Dll4 (R&D Systems, Minneapolis, Minn., US). The first injection at day 0 with human Dll4 is formulated in Complete Freund's Adjuvant (Difco, Detroit, Mich., USA), while the subsequent injections with human and mouse Dll4 are formulated in Incomplete Freund's Adjuvant (Difco, Detroit, Mich., USA). 1.2. Evaluation of Induced Immune Responses in Llama

To evaluate the induction of an immune responses in the animals against human Dll4 by ELISA, sera are collected from llamas 208, 209, 230 and 231 at day 0 (pre-immune), day 21 and day 43 (time of peripheral blood lymphocyte [PBL] collection), from llamas 127b, 260 and 261 at day 0 and day 51, and from llamas 282, 283 and 284 at day 0, day 28 and day 50. In short, 2 μg/mL of recombinant human Dll4 or mouse Dll4 (R&D Systems, Minneapolis, Minn., USA) are immobilized overnight at 4° C. in a 96-well MaxiSorp plate (Nunc, Wiesbaden, Germany). Wells are blocked with a casein solution (1%). After addition of serum dilutions, specifically bound immunoglobulins are detected using a horseradish peroxidase (HRP)-conjugated goat anti-llama immunoglobulin (Bethyl Laboratories Inc., Montgomery, Tex., USA) and a subsequent enzymatic reaction in the presence of the substrate TMB (3,3′,5,5′-tetramentylbenzidine) (Pierce, Rockford, Ill., USA), showing that a significant antibody-dependend immune response against Dll4 is induced. The antibody response is mounted both by conventional and heavy-chain only antibody expressing B-cell repertoires since specifically bound immunoglobulins can be detected with antibodies specifically recognizing the conventional llama IgG1 antibodies or the heavy chain only llama IgG2 or IgG3 antibodies (Table 2-A). In all llamas injected with mouse Dll4, an antibody response is mounted by conventional and heavy chain only antibody expressing B-cells specifically against mouse Dll4. Additionally, serum titers of cell immunized animals are confirmed by FACS analysis on human and mouse Dll4 overexpressing HEK293 cells (Table 2-B). The Dll4 serum titer responses for each llama are depicted in Table 2.

TABLE 2
Antibody-mediated specific serum response against DLL4

| | | | | | | | | | |
|---|---|---|---|---|---|---|---|---|---|
| | | Total | | | | Total | | | |
| Llama | Immunogen | IgG | IgG1 | IgG2 | IgG3 | IgG | IgG1 | IgG2 | IgG3 |

A) ELISA (recombinant protein, coated on solid phase)

| | | Recombinant human DLL4 | | | | Recombinant mouse DLL4 | | | |
|---|---|---|---|---|---|---|---|---|---|
| 208 | rec. human | + | + | +/− | +/− | n/d | n/d | n/d | n/d |
| | DLL4 | | | | | | | | |
| 209 | rec. human | + | + | +/− | +/− | n/d | n/d | n/d | n/d |
| | DLL4 | | | | | | | | |
| 230 | rec. human | ++ | ++ | +/− | +/− | n/d | n/d | n/d | n/d |
| | DLL4 | | | | | | | | |
| 231 | rec. human | ++ | ++ | ++ | ++ | n/d | n/d | n/d | n/d |
| | DLL4 | | | | | | | | |
| 127b | CHO- | ++ | ++ | +/− | +/− | + | ++ | +/− | +/− |
| | hDLL4 + | | | | | | | | |
| | CHO- | | | | | | | | |
| | mDLL4 | | | | | | | | |
| 260 | CHO- | ++ | ++ | + | + | ++ | ++ | + | ++ |
| | hDLL4 + | | | | | | | | |
| | CHO- | | | | | | | | |
| | mDLL4 | | | | | | | | |
| 261 | CHO- | ++ | ++ | +/− | +/− | + | + | +/− | +/− |
| | hDLL4 + | | | | | | | | |
| | CHO- | | | | | | | | |
| | mDLL4 | | | | | | | | |
| 282 | rec. human | ++ | ++ | ++ | ++ | ++ | ++ | + | + |
| | DLL4 + | | | | | | | | |
| | mouse | | | | | | | | |
| | DLL4 | | | | | | | | |
| 283 | rec. human | ++ | ++ | ++ | ++ | ++ | ++ | ++ | ++ |
| | DLL4 + | | | | | | | | |
| | mouse | | | | | | | | |
| | DLL4 | | | | | | | | |
| 284 | rec. human | + | + | + | + | + | ++ | + | ++ |
| | DLL4 + | | | | | | | | |
| | mouse | | | | | | | | |
| | DLL4 | | | | | | | | |

B) FACS (natively expressed protein on HEK293 cells)

| | | human DLL4 | | | | mouse DLL4 | | | |
|---|---|---|---|---|---|---|---|---|---|
| 208 | rec. human DLL4 | n/d | n/d | n/d | n/d | n/d | n/d | n/d | n/d |
| 209 | rec. human DLL4 | n/d | n/d | n/d | n/d | n/d | n/d | n/d | n/d |
| 230 | rec. human DLL4 | n/d | n/d | n/d | n/d | n/d | n/d | n/d | n/d |
| 231 | rec. human DLL4 | n/d | n/d | n/d | n/d | n/d | n/d | n/d | n/d |
| 127b | CHO-hDLL4 + | + | n/d | n/d | n/d | + | n/d | n/d | n/d |
| | CHO-mDLL4 | | | | | | | | |
| 260 | CHO-hDLL4 + | ++ | n/d | n/d | n/d | ++ | n/d | n/d | n/d |
| | CHO-mDLL4 | | | | | | | | |
| 261 | CHO-hDLL4 + | + | n/d | n/d | n/d | + | n/d | n/d | n/d |
| | CHO-mDLL4 | | | | | | | | |
| 282 | rec. human DLL4 + | n/d | n/d | n/d | n/d | n/d | n/d | n/d | n/d |
| | mouse DLL4 | | | | | | | | |
| 283 | rec. human DLL4 + | n/d | n/d | n/d | n/d | n/d | n/d | n/d | n/d |
| | mouse DLL4 | | | | | | | | |
| 284 | rec. human DLL4 + | n/d | n/d | n/d | n/d | n/d | n/d | n/d | n/d |
| | mouse DLL4 | | | | | | | | |

n/d, not determined

### EXAMPLE 2

Cloning of the Heavy-Chain Only Antibody Fragment Repertoires and Preparation of Phage

Following the final immunogen injection, immune tissues as the source of B-cells that produce the heavy-chain antibodies are collected from the immunized llamas. Typically, two 150-ml blood samples, collected 4 and 8 days after the last antigen injection, and one lymph node biopsy, collected 4 days after the last antigen injection are collected per animal. From the blood samples, peripheral blood mononuclear cells (PBMCs) are prepared using Ficoll-Hypaque according to the manufacturer's instructions (Amersham Biosciences, Piscataway, N.J., USA). From the PBMCs and the lymph node biopsy, total RNA is extracted, which is used as starting material for RT-PCR to amplify the VHH encoding DNA segments, as described in WO 05/044858. For each immunized llama, a library is constructed by pooling the total RNA isolated from all collected immune tissues of that animal. In short, the PCR amplified VHH repertoire is cloned via specific restriction sites into a vector designed to facilitate phage display of the VHH library. The vector is derived from pUC119 and contains the LacZ promoter, a M13 phage glll protein coding sequence, a resistance gene for ampicillin or carbenicillin, a multiple cloning site and a hybrid glll-pelB leader sequence (pAX050). In frame with the VHH coding sequence, the vector encodes a C-terminal c-myc tag and a His6 tag. Phage are prepared according to standard protocols and stored after filter sterilization at 4° C. for further use.

### EXAMPLE 3

Selection of Dll4-Specific VHHs via Phage Display

VHH repertoires obtained from all llamas and cloned as phage library are used in different selection strategies, applying a multiplicity of selection conditions. Variables include i) the Dll4 protein format (C-terminally His-tagged recombinantly expressed extracellular domain of human Dll4 (Met1-Pro524) and mouse Dll4 (Met1-Pro525) (R&D Systems, Minneapolis, Minn., USA), or full length human Dll4 and mouse Dll4 present on Dll4-overexpressing CHO or HEK293 cells, ii) the antigen presentation method (plates directly coated with Dll4 or Neutravidin plates coated with Dll4 via a biotin-tag; solution phase: incubation in solution followed by capturing on Neutravidin-coated plates), iii) the antigen concentration and iv) different elution methods (non-specific via trypsin or specfic via cognate receptor Notch1/Fc chimera or anti-Dll4 IgG/Fab). All selections are done in Maxisorp 96-well plates (Nunc, Wiesbaden, Germany).

Selections are performed as follows: Dll4 antigen preparations for solid and solution phase selection formats are presented as described above at multiple concentrations. After 2 h incubation with the phage libraries followed by extensive washing, bound phage are eluted with trypsin (1 mg/mL) for 30 minutes. In case trypsin is used for phage elution, the protease activity is immediately neutralized applying 0.8 mM protease inhibitor ABSF. As control, selections w/o antigen are performed in parallel. Phage outputs that show enrichment over background (non-antigen control) are used to infect E. coli. Infected E. coli cells are either used to prepare phage for the next selection round (phage rescue) or plated on agar plates (LB+amp+glucose^{2%}) for analysis of individual VHH clones. In order to screen a selection output for specific binders, single colonies are picked from the agar plates and grown in 1 mL 96-deep-well plates. LacZ-controlled VHH expression is induced by adding IPTG (0.1-1 mM final) in the absence of glucose. Periplasmic extracts (in a volume of ˜80 uL) are prepared according to standard protocols

### EXAMPLE 4

Screening of Periplasmic Extracts in Dll4-Notch1 AlphaScreen and FMAT Competition Assay

Periplasmic extracts are screened in a human Dll4/human Notch1 AlphaScreen assay to assess the blocking capacity of the expressed VHHs. Human Dll4 is biotinylated using biotin (Sigma, St Louis, Mo., USA) and biotinamidohexanoic acid 3-sulfo-N-hydroxysuccinimide ester sodium salt (Sigma, St Louis, Mo., USA). Notch1/Fc chimera (R&D Systems, Minneapolis, Minn., USA) is captured using an anti-Fc VHH which is coupled to acceptor beads according to the manufacturer's instructions (Perkin Elmer, Waltham, Mass., US). To evaluate the neutralizing capacity of the VHHs, dilution series of the periplasmic extracts are pre-incubated with biotinylated human Dll4. To this mixture, the acceptor beads and the streptavidin donor beads are added and further incubated for 1 hour at room temperature. Fluorescence is measured by reading plates on the Envision Multilabel Plate reader (Perkin Elmer, Waltham, Mass., USA) using an excitation wavelength of 680 nm and an emission wavelength of 520 nm. Decrease in fluorescence signal indicates that the binding of biotinylated human Dll4 to the human Notch1/Fc receptor is blocked by the VHH expressed in the periplasmic extract.

Alternatively, CHO-hDll4 and CHO-mDll4 cells are used in a human Notch1/Fc FMAT (Fluorometric Microvolume Assay Technology) competition assay. Recombinant human Notch1/Fc chimera (R&D Systems, Minneapolis, Minn., USA) is randomly labeled with Alexa-647 (Invitrogen, Carlsbad, Calif., USA). In brief, 5 μL periplasmic material is added to 100 pM or 175 pM labeled human Notch1/Fc together with 7,500 CHO-hDll4 or CHO-mDll4 overexpressing cells, respectively, and readout is performed after 2 hours of incubation. To set the no-competition baseline, at least 30 replicates of cells with human Notch1/Fc˜Alexa647 are included and the percentage of inhibition is calculated from this baseline. All calculations are based on the FL1_total signal which comprises the average of the fluorescence per well times the number of counts per well.

From this screening, inhibiting VHHs are selected and sequenced. Sequence analysis revealed 166 unique VHHs belonging to 40 different B-cell lineages. The total number of variants found for each B-cell lineage is depicted in Table 3. An overview of periplasmic screening data is given in Table 4. The amino acid sequences of unique VHHs selected for further characterization are shown in the Sequence Listing (SEQ ID NOs: 4-20) and in Table 5 (CDRs and framework regions are indicated).

TABLE 3
Selection parameters used for the identification of DLL4-specific VHH B-cell lineages

| B-cell | | # | | selection | phage | selection |
|---|---|---|---|---|---|---|
| lineage | VHH ID | variants | library | format | elution | rounds |
| 1 | DLLBII8A09 | 31 | 231 | rhDLL4 (3 nM) | trypsin | 1 |
| 2 | DLLBII5B11 | 1 | 231 | rhDLL4 (3 nM) | trypsin | 1 |
| 3 | DLLBII7B05 | 21 | 231 | RI: biot-rhDLL4 (3 nM) | trypsin | 2 |
| | | | | RII: biot-rhDLL4 (0.03 | | |
| | | | | nM) | | |
| 4 | DLLBII6B11 | 13 | 231 | biot-rhDLL4 (3M) | trypsin | 1 |
| 5 | DLLBII8C11 | 5 | 231 | RI: biot-rhDLL4 (3 nM) | trypsin | 2 |
| | | | | RII: biot-rhDLL4 (3 nM) | | |
| 6 | DLLBII19D10 | 1 | 231 | biot-rhDLL4 (3 nM) | trypsin | 1 |
| 7 | DLLBII33C05 | 2 | 231 | CHO-hDLL4 (2E6/mL) | trypsin | 1 |
| 8 | DLLBII28B06 | 2 | 231 | rmDLL4 (0.5 ug/mL) | trypsin | 1 |
| 9 | DLLBII17G10 | 1 | 231 | biot-rhDLL4 (3 nM) | trypsin | 1 |
| 10 | DLLBII17C01 | 8 | 231 | biot-rhDLL4 (3 nM) | trypsin | 1 |
| 11 | DLLBII19F04 | 1 | 231 | biot-rhDLL4 (3 nM) | trypsin | 1 |
| 12 | DLLBII17F10 | 1 | 231 | biot-rhDLL4 (3 nM) | trypsin | 1 |
| 13 | DLLBII17B03 | 5 | 231 | biot-rhDLL4 (3 nM) | trypsin | 1 |
| 14 | DLLBII19F12 | 2 | 231 | biot-rhDLL4 (3 nM) | trypsin | 1 |
| 15 | DLLBII42B07 | 1 | 231 | RI: biot-rhDLL4 (3 nM) | rhNotch1/ | 2 |
| | | | | RII: biot-rhDLL4 (3 nM) | Fc | |
| 16 | DLLBII47D01 | 1 | 230 | RI: biot-rhDLL4 (3 nM) | rhNotch1/ | 2 |
| | | | | RII: biot-rhDLL4 (3 nM) | Fc | |
| 17 | DLLBII56A09 | 15 | 230 | RI: CHO-mDLL4 | rhNotch1/ | 2 |
| | | | | (2E6/mL) | Fc | |
| | | | | RII: CHO-mDLL4 | | |
| | | | | (2E6/mL) | | |
| 18 | DLLBII95F02 | 5 | 230 | RI: CHO-mDLL4 | trypsin | 2 |
| | | | | (2E6/mL) | | |
| | | | | RII: CHO-mDLL4 | | |
| | | | | (2E6/mL) | | |
| 19 | DLLBII96C03 | 20 | 230 | RI: CHO-mDLL4 | trypsin | 2 |
| | | | | (2E6/mL) | | |
| | | | | RII: CHO-mDLL4 | | |
| | | | | (2E6/mL) | | |
| 20 | DLLBII104G01 | 1 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 | (RIII) | |
| | | | | (+rhDLL4) | | |
| 21 | DLLBII102F08 | 3 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (0.01 | (RIII) | |
| | | | | nM) | | |
| 22 | DLLBII112A03 | 1 | 209 | RI: CHO-mDLL4 | trypsin | 2 |
| | | | | (2E6/mL) | | |
| | | | | RII: CHO-mDLL4 | | |
| | | | | (2E6/mL) | | |
| 23 | DLLBII102G04 | 2 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (0.01 | (RIII) | |
| | | | | nM) | | |
| 24 | DLLBII101G08 | 1 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (0.1 | (RIII) | |
| | | | | nM) | | |
| 25 | DLLBII112A04 | 1 | 209 | RI: CHO-mDLL4 | trypsin | 2 |
| | | | | (2E6/mL) | | |
| | | | | RII: CHO-mDLL4 | | |
| | | | | (2E6/mL) | | |
| 26 | DLLBII101H09 | 1 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (0.1 | (RIII) | |
| | | | | nM) | | |
| 27 | DLLBII101H05 | 1 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (1 nM) | (RIII) | |
| 28 | DLLBII112E07 | 1 | 209 | RI: CHO-mDLL4 | trypsin | 2 |
| | | | | (2E6/mL) | | |
| | | | | RII: CHO-mDLL4 | | |
| | | | | (2E6/mL) | | |
| 29 | DLLBII101F01 | 1 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (1 nM) | (RIII) | |
| 30 | DLLBII104A03 | 1 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (1 nM) + | (RIII) | |
| | | | | rhDLL4 | | |
| 31 | DLLBII104C04 | 1 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (1 nM) + | (RIII) | |
| | | | | rhDLL4 | | |
| 32 | DLLBII104B05 | 1 | 230 | RI: CHO-mDLL4 | rhNotch1/ | 3 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (1 nM) + | (RIII) | |
| | | | | rhDLL4 | | |
| 33 | DLLBII107C03 | 1 | 208 | RI: CHO-mDLL4 | rhNotch1/ | 2 |
| | | | | (2E6/mL) | Fc | |
| | | | | RII: CHO-mDLL4 | | |
| | | | | (2E6/mL) | | |
| 34 | DLLBII58A11 | 4 | 260 | RI: biot-rhDLL4 (3 nM) | rhNotch1/ | 2 |
| | | | | RII: biot-rmDLL4 (3 nM) | Fc | |
| 35 | DLLBII61F05 | 1 | 260 | RI: HEK293H-hDLL4 | trypsin | 2 |
| | | | | (2E6/mL) | | |
| | | | | RII: HEK293H-hDLL4 | | |
| | | | | (2E6/mL) | | |
| 36 | DLLBII61F07 | 1 | 260 | RI: HEK293H-hDLL4 | trypsin | 2 |
| | | | | (2E6/mL) | | |
| | | | | RII: HEK293H-hDLL4 | | |
| | | | | (2E6/mL) | | |
| 37 | DLLBII62C11 | 1 | 260 | RI: HEK293H-hDLL4 | trypsin | 2 |
| | | | | (2E6/mL) | | |
| | | | | RII: HEK293H-mDLL4 | | |
| | | | | (2E6/mL) | | |
| 38 | DLLBII115A05 | 1 | 230 | RI: CHO-mDLL4 | rhNotch1/ | 4 |
| | | | | (2E6/mL) | Fc | |
| | | | | RII: CHO-mDLL4 | (RI-RII) | |
| | | | | (2E6/mL) | trypsin | |
| | | | | RIII: biot-rhDLL4 (1 nM) | (RIII) | |
| | | | | RIV: CHO-mDLL4 | trypsin | |
| | | | | (2E6/mL) | (RIV) | |
| 39 | DLLBII83G01 | 4 | 284 | RI: CHO-mDLL4 | DLL4 | 2 |
| | | | | (2E6/mL) | IgG | |
| | | | | RI: CHO-hDLL4 | | |
| | | | | (2E6/mL) | | |
| 40 | DLLBII80E08 | 1 | 283 | RI: CHO-hDLL4 | DLL4 | 2 |
| | | | | (2E6/mL) | IgG | |
| | | | | RI: CHO-hDLL4 | | |
| | | | | (2E6/mL) | | |

TABLE 4
Screening of periplasmic extracts containing expressed anti-DLL4 VHH

| | | | | Alpha | | | |
|---|---|---|---|---|---|---|---|
| | | | ELISA | Screen | FMAT | FMAT | |
| B-cell | Representative | # unique | hDLL4 | hDLL4 | hDLL4 | mDLL4 | Biacore ^{(a)} |
| lineage | VHH ID | sequences | % inh | % inh | % inh | % inh | k_{d} (s⁻¹) |
| 1 | DLLBII8A09 | 31 | 96 | — | — | — | (1.2^{E-03}-2.4^{E-04}) |
| 2 | DLLBII5B11 | 1 | 98 | — | — | — | — |
| 3 | DLLBII7B05 | 21 | 84 | — | — | — | (2.4^{E-04}) |
| 4 | DLLBII6B11 | 13 | 98 | — | — | — | (9.4^{E-04}-3.7^{E-04}) |
| 5 | DLLBII8C11 | 5 | 57 | — | — | — | (7.3^{E-04}-6.0^{E-04}) |
| 6 | DLLBII19D10 | 1 | 98 | 85 | — | — | 1.3^{E-03} |
| 7 | DLLBII33C05 | 2 | 86 | 75 | — | — | 9.2^{E-04} |
| | | | | | | | (2.1^{E-03}) |
| 8 | DLLBII28B06 | 2 | 23 | 54 | — | — | 7.5^{E-03} |
| | | | | | | | (1.6^{E-04}) |
| 9 | DLLBII17G10 | 1 | 93 | 82 | — | — | 1.5^{E-03} |
| 10 | DLLBII17C01 | 8 | 82 | 84 | — | — | 5.6^{E-04} |
| | | | | | | | (5.6^{E-04}-5.3^{E-04}) |
| 11 | DLLBII19F04 | 1 | 98 | 95 | — | — | 1.1^{E-03} |
| 12 | DLLBII17F10 | 1 | 98 | 88 | — | — | 1.1^{E-03}/ |
| | | | | | | | 3.1^{E-04} ^{(b)} |
| 13 | DLLBII17B03 | 5 | 76 | 77 | — | — | 1.2^{E-03}/ |
| | | | | | | | 2.2^{E-04} ^{(b)} |
| 14 | DLLBII19F12 | 2 | 98 | 98 | — | — | 4.9^{E-04} |
| | | | | | | | (1.0^{E-03}) |
| 15 | DLLBII42B07 | 1 | — | — | — | — | — |
| 16 | DLLBII47D01 | 1 | — | — | 87 | — | — |
| 17 | DLLBII56A09 | 15 | — | — | — | — | 1.1^{E-03} |
| | | | | | | | (9.5^{E-03}-1.1^{E-03}) |
| 18 | DLLBII95F02 | 5 | — | — | 81 | 71 | 6.7^{E-04} |
| 19 | DLLBII96C03 | 20 | — | — | 75 | 83 | — |
| 20 | DLLBII104G01 | 1 | — | — | 94 | 86 | 1.2^{E-03} |
| | | | | | | | (1.4^{E-03}-9.4^{E-04}) |
| 21 | DLLBII102F08 | 3 | — | — | 85 | 75 | — |
| 22 | DLLBII112A03 | 1 | — | — | 72 | 97 | — |
| 23 | DLLBII102G04 | 2 | — | — | 86 | 82 | — |
| 24 | DLLBII101G08 | 1 | — | — | 91 | 92 | 2.1^{E-03} |
| 25 | DLLBII112A04 | 1 | — | — | 75 | 90 | — |
| 26 | DLLBII101H09 | 1 | — | — | 87 | 75 | — |
| 27 | DLLBII101H05 | 1 | — | — | 85 | 83 | — |
| 28 | DLLBII112E07 | 1 | — | — | 80 | 85 | — |
| 29 | DLLBII101F01 | 1 | — | — | 85 | 78 | 2.0^{E-02} |
| 30 | DLLBII104A03 | 1 | — | — | 86 | 83 | — |
| 31 | DLLBII104C04 | 1 | — | — | 87 | 83 | 1.0^{E-03} |
| 32 | DLLBII104B05 | 1 | — | — | 86 | 78 | — |
| 33 | DLLBII107C03 | 1 | — | — | 75 | 80 | — |
| 34 | DLLBII58A11 | 4 | — | — | 95 | 73 | 1.6^{E-03} |
| | | | | | | | (1.7^{E-03}-1.6^{E-03}) |
| 35 | DLLBII61F05 | 1 | — | — | 74 | 76 | — |
| 36 | DLLBII61F07 | 1 | — | — | 79 | 77 | — |
| 37 | DLLBII62C11 | 1 | — | — | 74 | 71 | — |
| 38 | DLLBII115A05 | 1 | — | — | 74 | 84 | 3.1^{E-03} |
| 39 | DLLBII83G01 | 4 | — | — | 87 | 93 | 4.1^{E-04} |
| 40 | DLLBII80E08 | 1 | — | — | 71 | 82 | — |

^{(a)} if multiple unique variants within a B-cell lineage are identified, the range (max-min) in off-rate or the off-rate of a lineage member is given between brackets in italics).
^{(b)} heterogeneous fit: fast and slow off-rate determined.

TABLE 5
Sequence IDs and AA sequences of selected monovalent anti-DLL4 VHHs
(FR, framework; CDR, complementary determining region)

| VHH ID/ | | | | | | | |
|---|---|---|---|---|---|---|---|
| SEQ ID | | | | | | | |
| NO | Framework 1 | CDR 1 | Framework 2 | CDR 2 | Framework 3 | CDR 3 | Framework 4 |
| DLLBII05B11 4 | EVQLVESGGGLVQP | LHVIG | WLRQAPGKEREWVS | CISSSDGS | RFTISRDNAKNTVYLQMN | PWDSWYCGIGNDY | WGQGTQVTVSS |
| | GGSLRLSCAISGFT | | | TYYADSVK | SLKPEDTAVYYCAA | DY | |
| | LD | | | G | | | |
| DLLBII06B1 5 | EVQLVESEGGLVQA | SYAMG | WYRQAPGKQRELVA | VISNGGIT | RFTISRDNAKNTVYLQMN | SGSYYYPTDVHEY | WGQGTQVTVSS |
| | GGSLRLSCAASGST | | | NYPNSVKG | SLKPEDTAVYYCFY | DY | |
| | FS | | | | | | |
| DLLBII07A02 6 | EVQLVESGGGLVQA | SYAMG | WYRQAPGKQREWVA | AFSTGGST | RFTISRDNAKNTVYLQMN | SGSYYYPTDVFEY | WGQGTQVTVSS |
| | GGSLRLSCAASGST | | | NYADSVKG | SLKPEDTAVYYCFY | DY | |
| | FN | | | | | | |
| DLLBII07B05 7 | EVQLVESGGGLVQA | YYAVG | WFRQAPGKEREGVS | CISSRGGS | RFTTSRNNAKNTVYLQMN | HPLQNCCGGSAYA | WGQGTQVTVSS |
| | GGSLRLSCAASGFA | | | TFYADSVK | SLKPEDTAVYYCAA | SPEAVYEY | |
| | LD | | | G | | | |
| DLLBII08A09 8 | EVQLVESGGGLVQP | YYNIG | WFRQAPGKEREWVS | CINSSDGS | RFTISRDNAKNTVYLQMN | PFAYYSNLCGVNG | WGQGTQVTVSS |
| | GGSLRLSCAASGFT | | | TYYADSVK | SLKPEDTAVYYCAA | YDY | |
| | LD | | | G | | | |
| DLLBII08C11 9 | EVQLVESGGGLVQA | DYAIG | WFRQAPGKEREGVS | CISSHDRT | RFTISSDNAKNTVYLQMN | DPLVCGYNDPRLA | WGQGTQVTVSS |
| | GGSLRLSCAASGFT | | | TYYADSVK | SLKPEDTAVYYCAA | DY | |
| | FD | | | G | | | |
| DLLBII101G08 | EVQLVESGGGLVQA | SYAMA | WFRQAPGKEREFVA | AIRWSGGT | RFTISRDNAKNTVYLQMN | RAADTRLGPYEYD | WGQGTQVTVSS |
| 10 | GGSLRLSCAASGRT | | | AYYADSVQ | SLKPEDTAVYYCAN | Y | |
| | FS | | | G | | | |
| DLLBII104G01 | EVQLVESGGGLVQA | DYAIG | WFRQAPGKEREGVS | CISSSDGS | RFTISSDNAKNTVYLQMN | AWCDSSWYRSFVG | WGQGTQVTVSS |
| 11 | GGSLRLSCAASGFT | | | TYYADSVK | SLKPEDTAVYYCAT | Y | |
| | FD | | | G | | | |
| DLLBII115A05 | EVQLVESGGGLVQP | SYDMS | WVRRSPGKGPEWVS | SINSGGGS | RFTISRDNAKNTLYLQMN | DRYIRARQGDYWG | WGQGTQVTVSS |
| 12 | GGSLRLSCAASGFT | | | TYYADFVK | SLKPEDTAVYYCAA | AYEYDY | |
| | FG | | | G | | | |
| DLLBII19F04 | EVQLVESGGGLVQA | TYAMA | WYRQAPGKQRELVA | GISFDGST | RFTISRDDAKNTVSLQMN | VHPSTGFGS | WGQGTQVTVSS |
| 13 | EGSLRLSCAASGST | | | HYAESVKG | SLKPEDAAVYYCYS | | |
| | FS | | | | | | |
| DLLBII55D12 | EVQLVESGGGLVQP | DYAIG | WFRQAPGKEPEGIS | CISSSGGI | RFTISRDNAKNTVYLQMN | PGIAACRGIHY | TGQGTQVTVSS |
| 14 | GGSLRLSCAASGFT | | | TYYADSVK | SLKPEDTAVYYCAT | | |
| | FD | | | G | | | |
| DLLBII56A09 | EVQLVESGGGLVQP | DYAIG | WFRQAPGKEPEGIS | CISSSGGI | RFTTSRDNAKNTVYLQMN | PGIAACRGIHY | TGQGTQVTVSS |
| 15 | GGSLRLSCAASGFT | | | TYYADSVK | SLKPEDTAVYYCAT | | |
| | FD | | | G | | | |
| DLLBII56C04 | EVQLVESGGGLVQP | VYAIG | WFRQAPGKEPEGIS | CISSSGSI | RFTTSRDSAKNTVYLQMN | PGIAACRGIHY | WGQGTQVTVSS |
| 16 | GGSLRLSCTASGFT | | | TYYADSVK | SLKPEDTAVYYCAT | | |
| | FD | | | G | | | |
| DLLBII56H08 | EVQLVESGGGLVQP | DYAIG | WFRQAPGKEPEEIS | CISSSGGI | RFTISRDNAKNTVYLQMN | PGIAACRGIHY | TGQGTQVTVSS |
| 17 | GGSLRLSCAASGFT | | | TYYADSVK | SLKPEDTAVYYCAT | | |
| | FD | | | G | | | |
| DLLBII62C11 | EVQLMESGGGLVQP | NYYMS | WVRQAPGKGLEWVS | VISPDGSN | RFTISRGNAKNTLFLQMT | GSGSWGV | HGQGTQVTVSS |
| 18 | GGSLRLSCVAAGFT | | | TYYADTVK | GLKSEDAAVYYCAR | | |
| | FS | | | G | | | |
| DLLBII96C03 | EVQLVESGGGLVQP | NYDMS | WVRQAPGKGPEWVS | AINSGGGD | RFTISRDNAKNTLYLQMN | PRGWGPTGPHEYG | WGQGTQVTVSS |
| 19 | GGSLRLSCAASGFT | | | TYYADSVK | SLKPEDTAVYYCAT | Y | |
| | FG | | | G | | | |
| DLLBII57C11 | EVQLVESGGGLVQP | DYAIG | WFRQAPGKEPEGIS | CISSSGSI | RFTISRDNAKNTVYLQMN | PGIAACRGIHY | WGQGTQVTVSS |
| 20 | GGSLRLSCTASGFT | | | TYDADSVK | SLKPEDTAVYYCAT | | |
| | FD | | | G | | | |

### EXAMPLE 5

Characterization of Purified Anti-Dll4 VHHs

Inhibitory anti-Dll4 VHHs selected from the screening described in Example 4 are further purified and characterized. Selected VHHs are expressed in E. coli TG1 as c-myc, His6-tagged proteins. Expression is induced by addition of 1 mM IPTG and allowed to continue for 4 hours at 37° C. After spinning the cell cultures, periplasmic extracts are prepared by freeze-thawing the pellets. These extracts are used as starting material and VHHs are purified via IMAC and size exclusion chromatography (SEC) resulting in 95% purity as assessed via SDS-PAGE.

5.1. Evaluation of Dll4 Blocking VHHs in ELISA

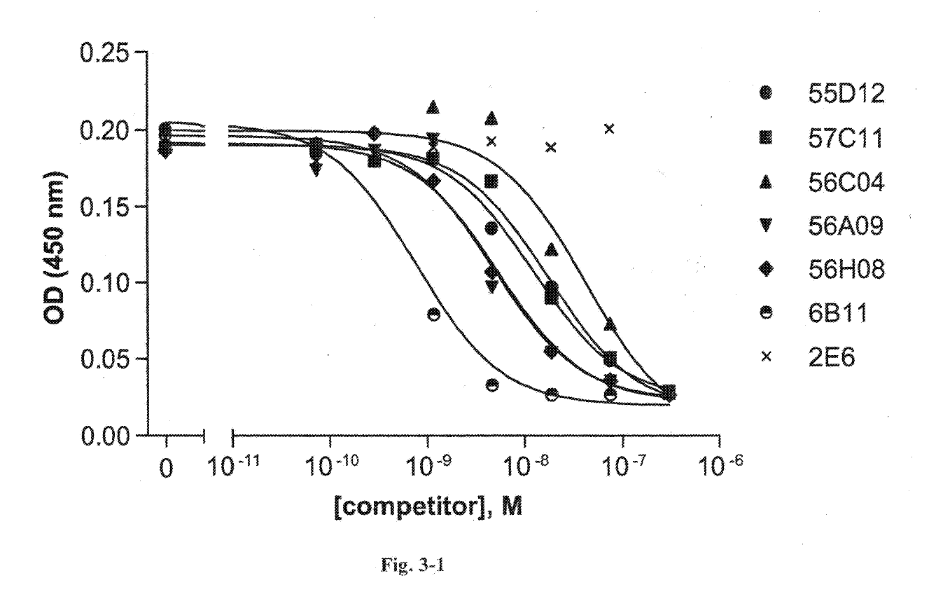

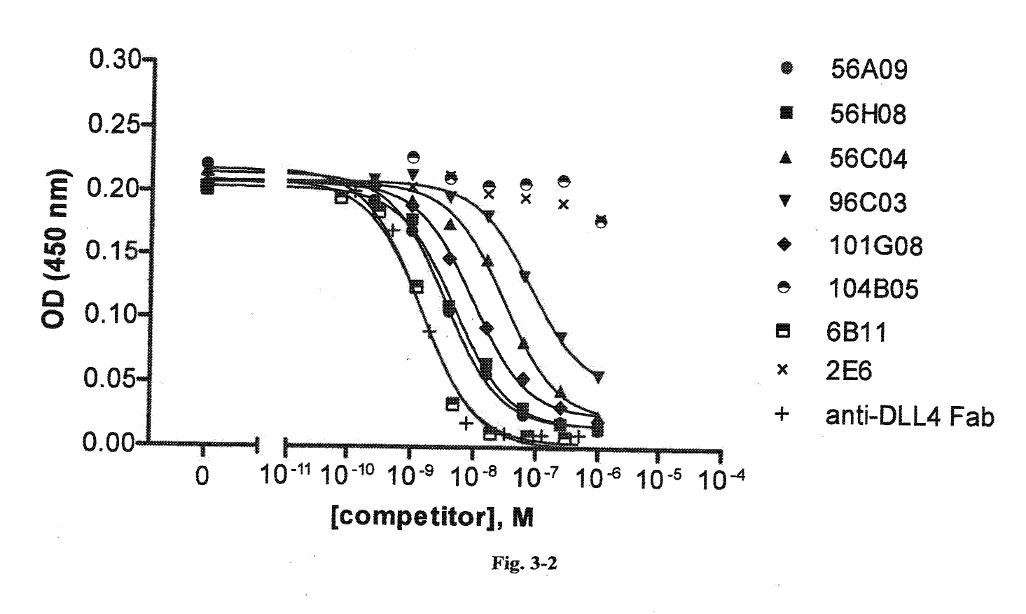

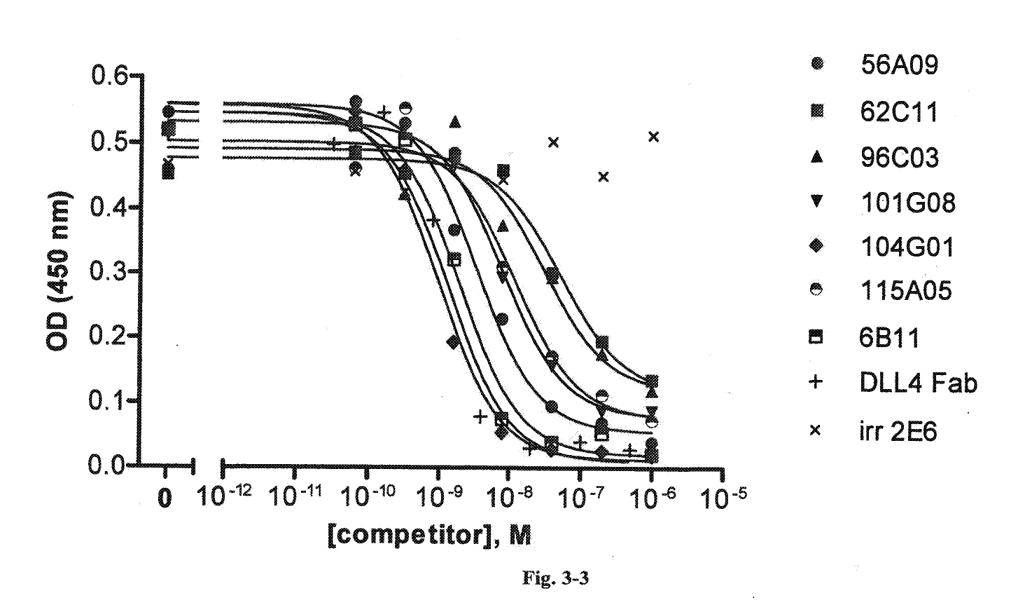

The blocking capacity of the VHHs is evaluated in a human Dll4—human Notch1/Fc blocking ELISA. In brief, 1 μg/mL of human Notch1/Fc chimera (R&D Systems, Minneapolis, Minn., USA) is coated in a 96-well MaxiSorp plate (Nunc, Wiesbaden, Germany). A fixed concentration of 15 nM biotinylated human Dll4 is preincubated with a dilution series of the VHH for 1 hour, after which the mixture is incubated on the coated Notch1 receptor for an additional hour. Residual binding of biotinylated human Dll4 is detected using horseradish peroxidase (HRP) conjugated extravidin (Sigma, St. Louis, Mo., USA) (FIG. 3). Human Dll4 is biotinylated as described above. The IC₅₀ values for VHHs blocking the human Dll4—human Notch1/Fc interaction are depicted in Table 6.

TABLE 6
IC₅₀ (nM) values for VHHs in hDLL4/hNotch1-Fc competition ELISA

| VHH ID | IC₅₀ (nM) |
|---|---|
| 6B11 | 1.5 |
| 55D12 | 12.3 |
| 56A09 | 4.9 |
| 56C04 | 33.9 |
| 56H08 | 6.9 |
| 57C11 | 17.3 |
| 62C11 | 72.0 |
| 96C03 | 38.4 |
| 101G08 | 9.5 |
| 104G01 | 1.1 |
| 115A05 | 9.1 |
| antiDLL4 Fab | 0.7 |

5.2. Evaluation of Dll4-Blocking VHHs in AlphaScreen

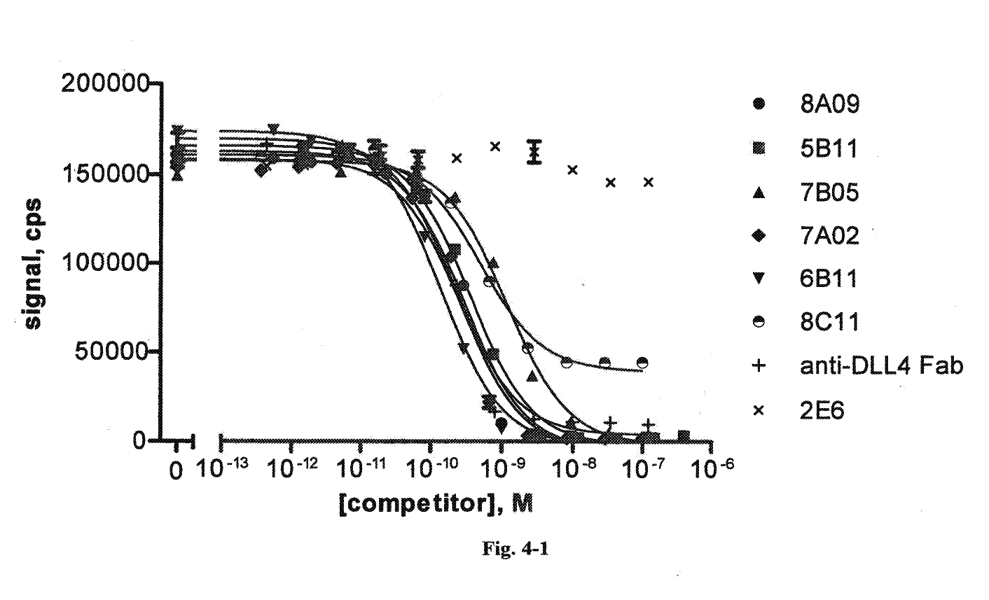

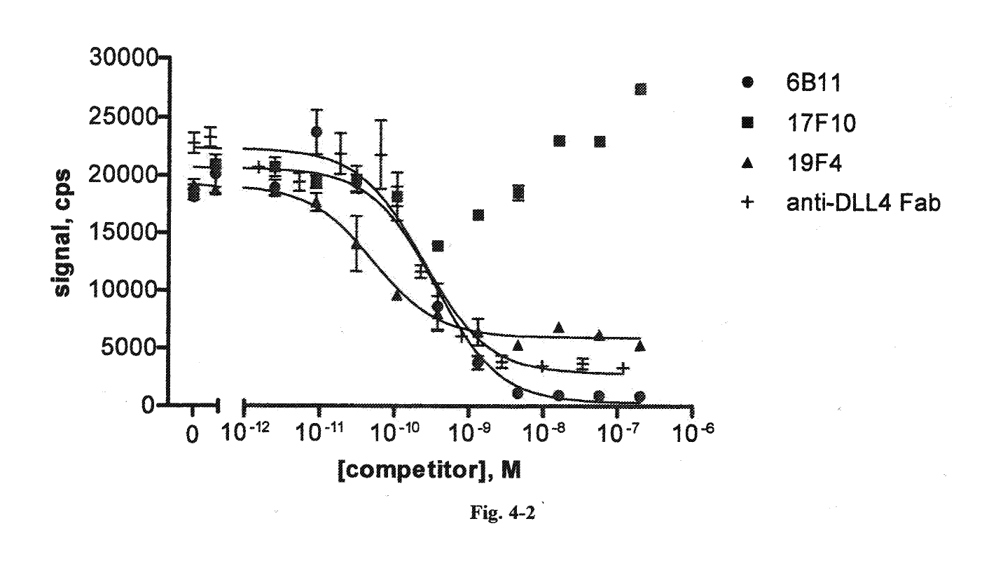

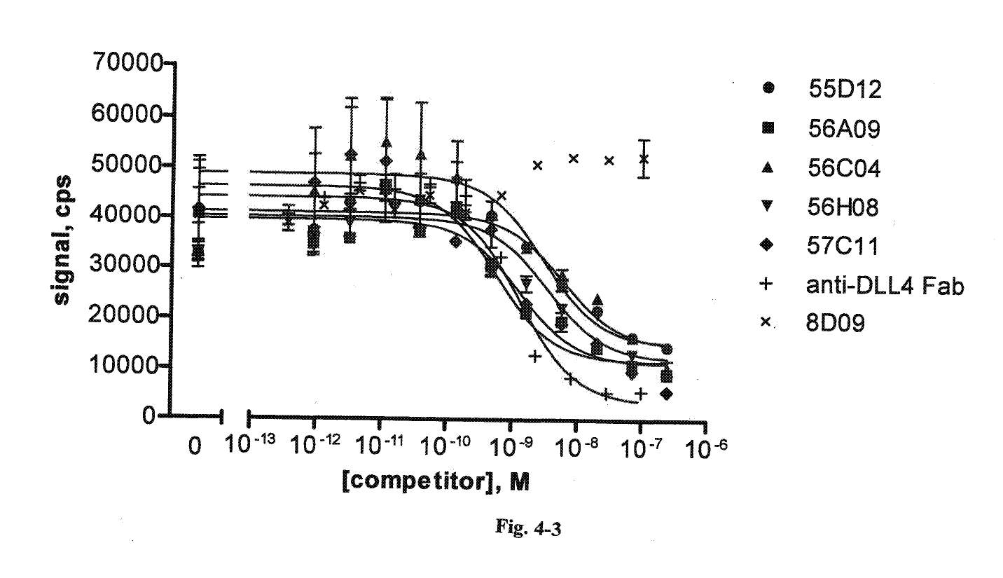

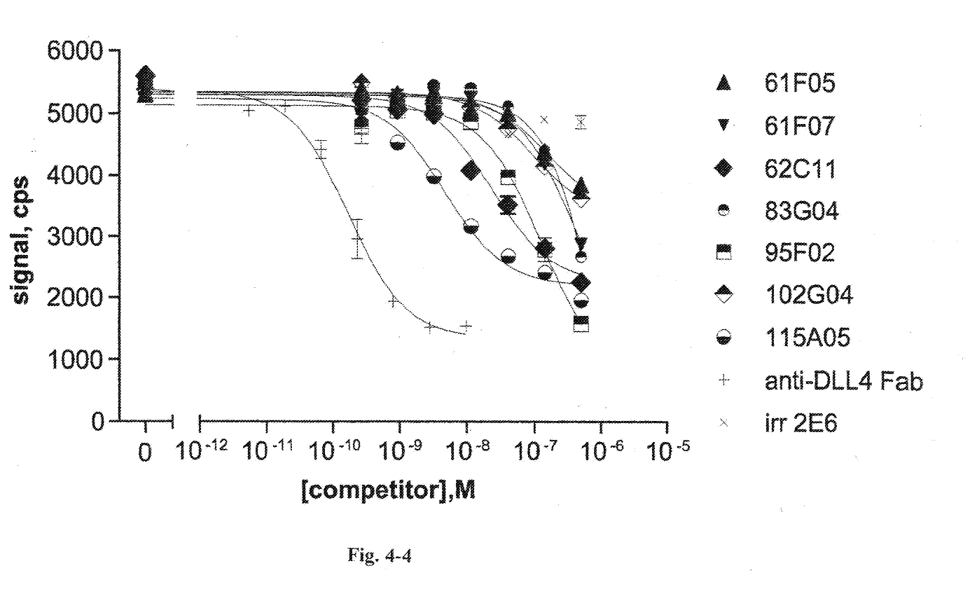

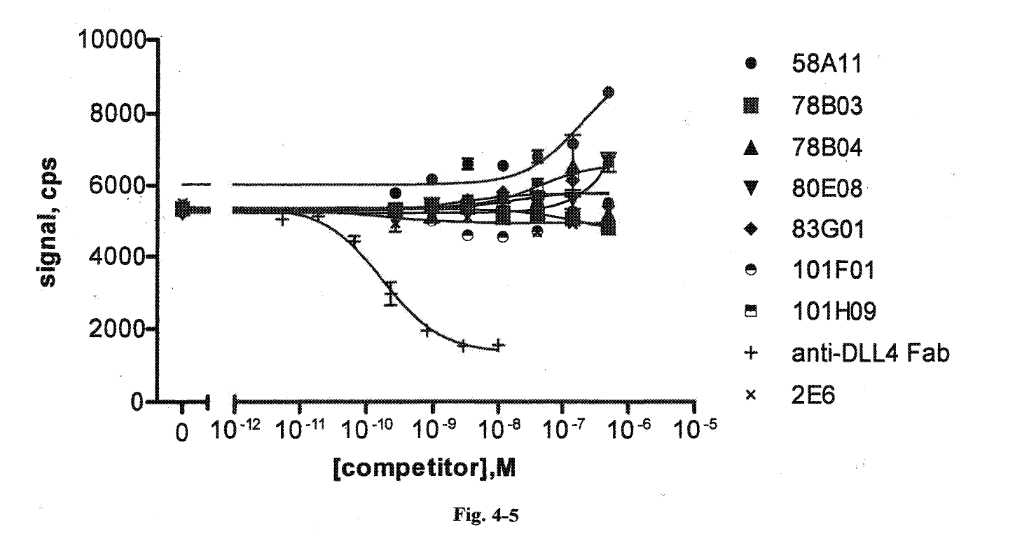

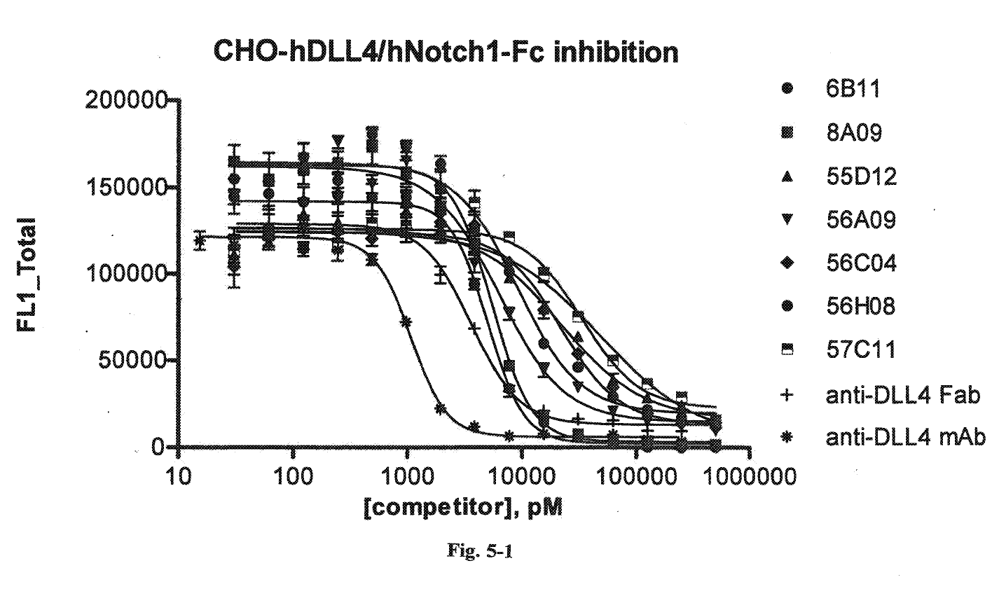

In brief, 1 nM biotinylated human Dll4 is captured on streptavidin-coated donor beads (20 μg/mL), while 0.4 nM of the receptor human Notch1 (as a Fc fusion protein) is captured on anti-human Fc VHH-coated acceptor beads (20 μg/mL). Both loaded beads are incubated together with a dilution range of the competing VHH (FIG. 4). The IC₅₀ values for VHHs blocking the human Dll4—human Notch1/Fc interaction are depicted in Table 7.

TABLE 7
IC₅₀ (nM) values for VHHs in hDLL4/hNotch1 competition
AlphaScreen

| VHH ID | IC₅₀ (nM) |
|---|---|
| 5B11 | 0.7 |
| 6B11 | 0.3 |
| 7A02 | 0.4 |
| 7B05 | 1.1 |
| 8A09 | 0.4 |
| 8C11 | 0.7^{(a)} |
| 19F04 | 0.05^{(a)} |
| 55D12 | 2.3 |
| 56A09 | 1.2 |
| 56C04 | 5.4 |
| 56H08 | 1.6 |
| 57C11 | 2.2 |
| 62C11 | 24.1 |
| 115A05 | 5.0 |
| antiDLL4 | 0.3 |
| Fab | |

^{(a)}partial inhibitor

5.3. Inhibition by Anti-Dll4 VHHs of Human Notch 1/Fc Binding to Human or Mouse Dll4 Expressed on the CHO Cells

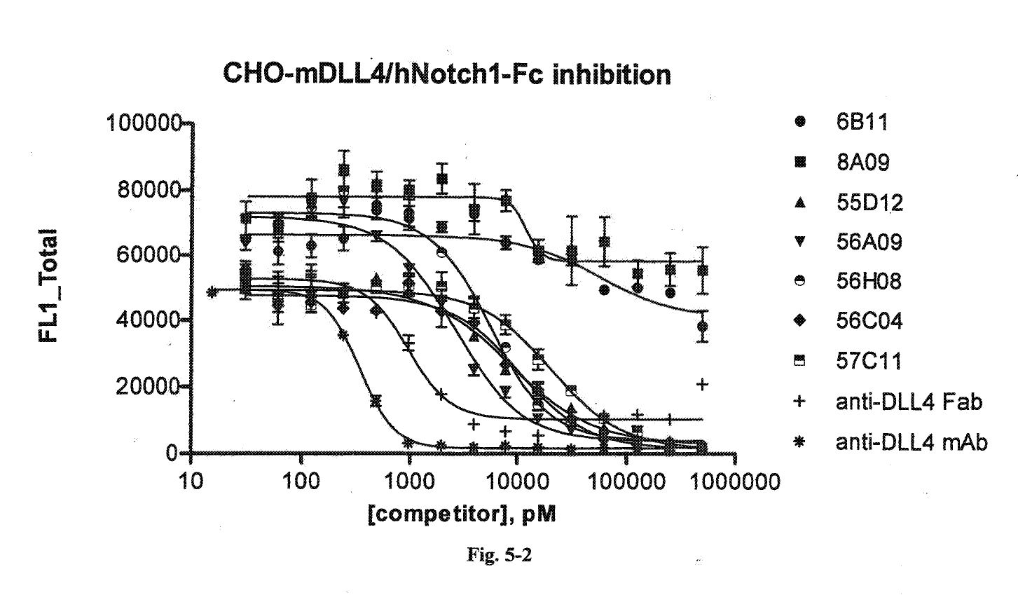

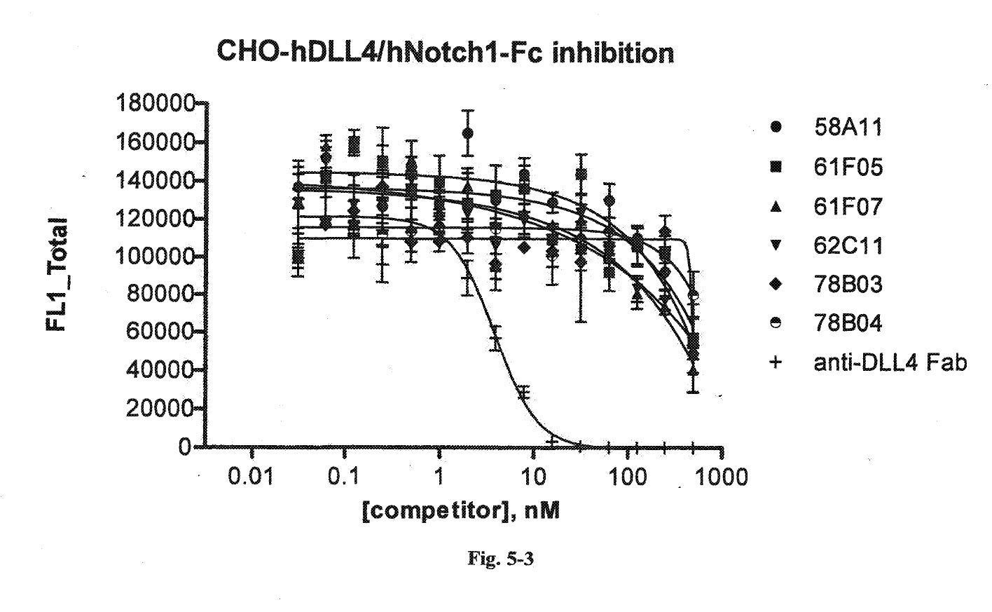

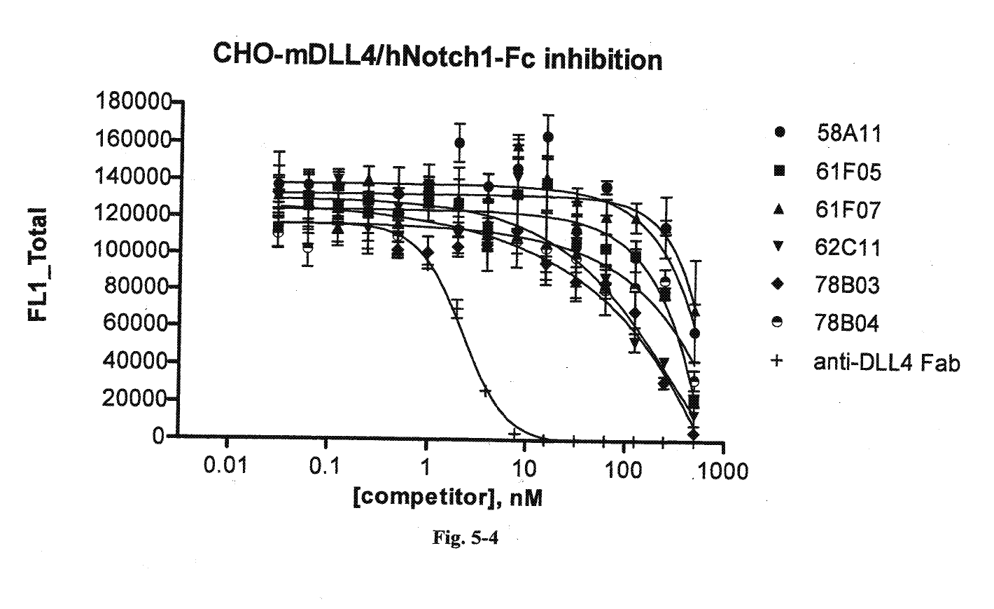

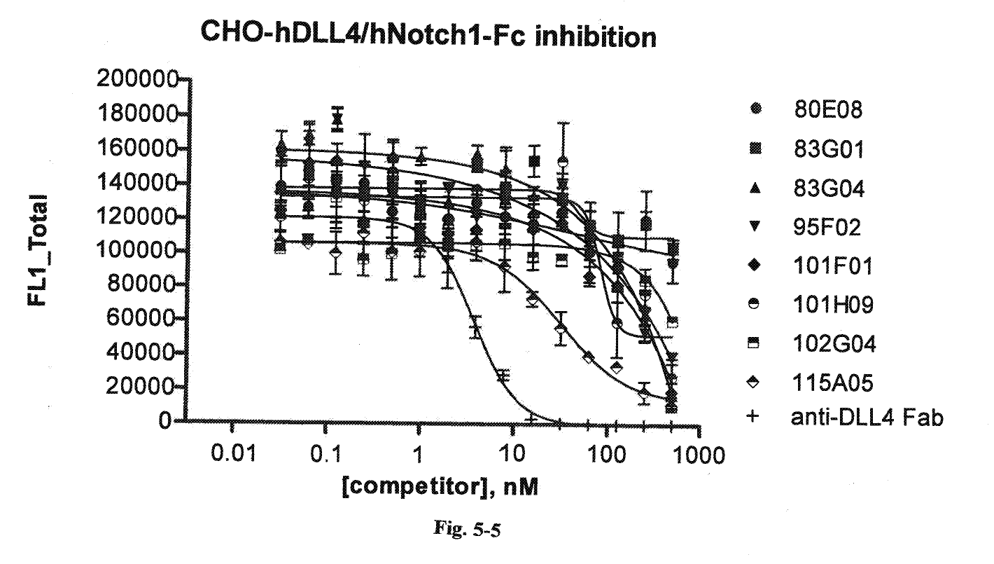

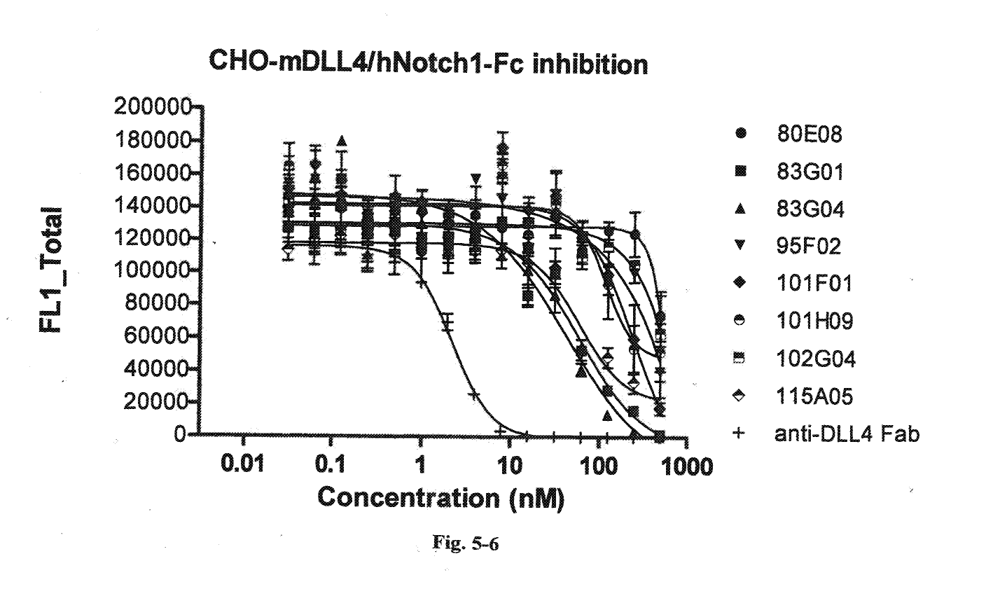

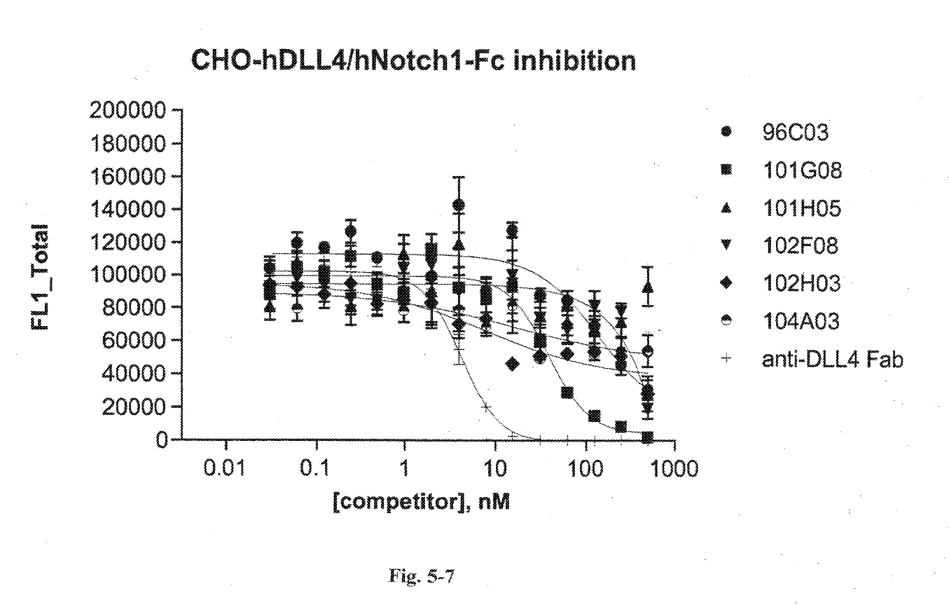

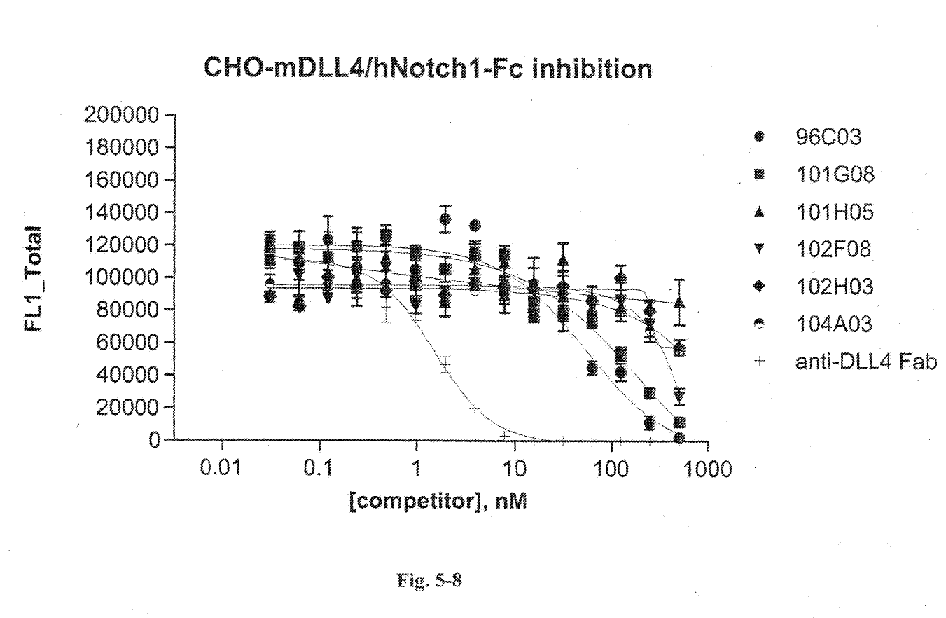

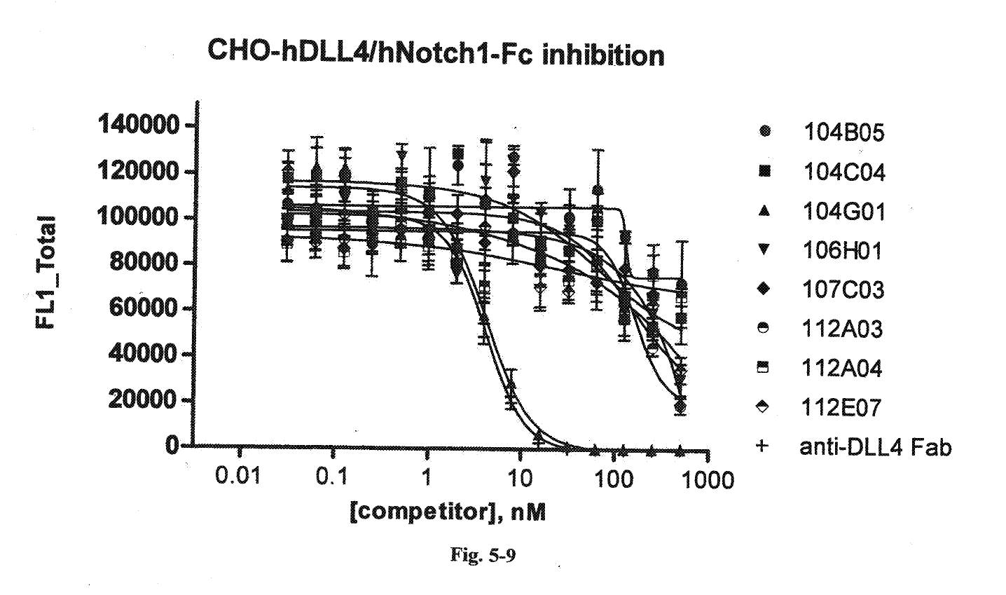

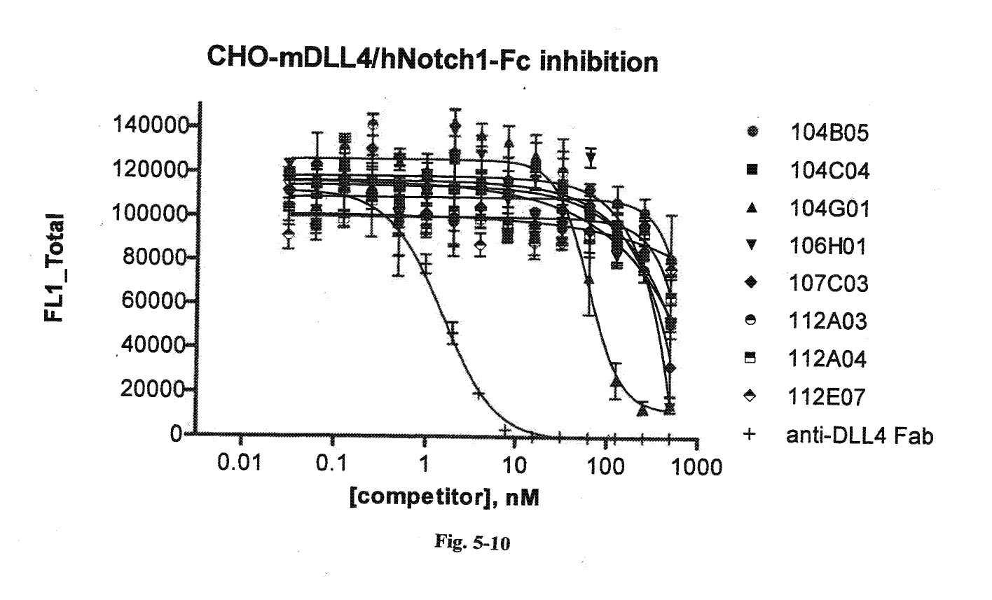

The blocking capacity of the VHHs is evaluated in a human and mouse Dll4—human Notch1/Fc competitive FMAT assay (FIG. 5) as outlined in Example 4. The IC₅₀ values for VHHs blocking the interaction of human Notch1/Fc to human or mouse Dll4 expressed on CHO cells are depicted in Table 8.

TABLE 8
(Mean) IC₅₀ values (nM) of purified VHHs blocking the interaction of
human Notch1/Fc to human or mouse DLL4 expressed on CHO cells
(FMAT)

| | hDLL4 | mDLL4 |
|---|---|---|
| VHH ID | IC₅₀ (nM) | IC₅₀ (nM) |
| 6B11 | 8.9 | — |
| 8A09 | 5.5 | — |
| 19F04 | 33.0 | — |
| 55D12 | 39.1 | 41.0 |
| 56A09 | 10.6 | 15.0 |
| 56C04 | 28.7 | 49.6 |
| 56H08 | 22.0 | 33.7 |
| 57C11 | 53.9 | 49.5 |
| 62C11 | 172.2 | 106.3 |
| 96C03 | 160.8 | 28.8 |
| 101G08 | 24.6 | 92.1 |
| 104G01 | 2.5 | — |
| 115A05 | 22.0 | 43.0 |
| antiDLL4 Fab | 5.4 | 2.3 |

5.4. Evaluation of Dll4-Blocking VHHs in Reporter Assay

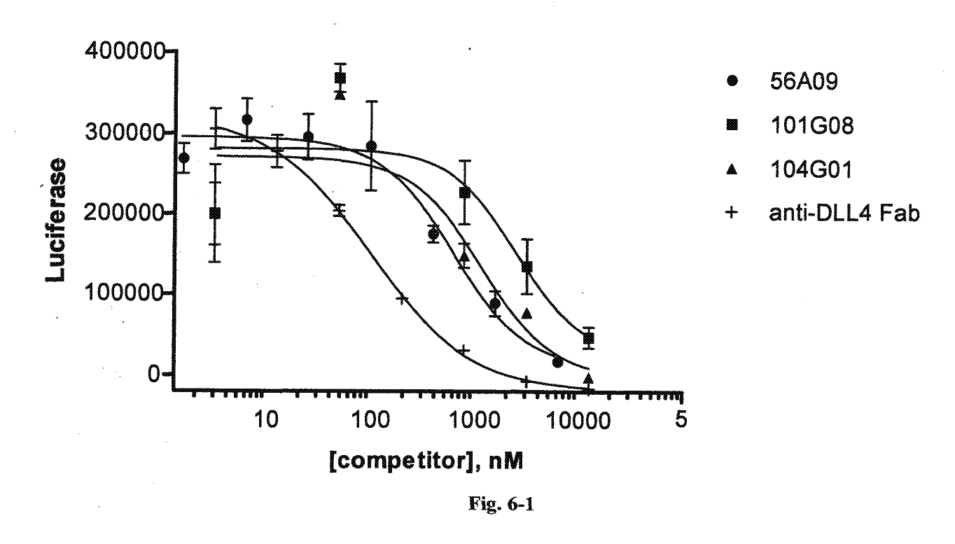

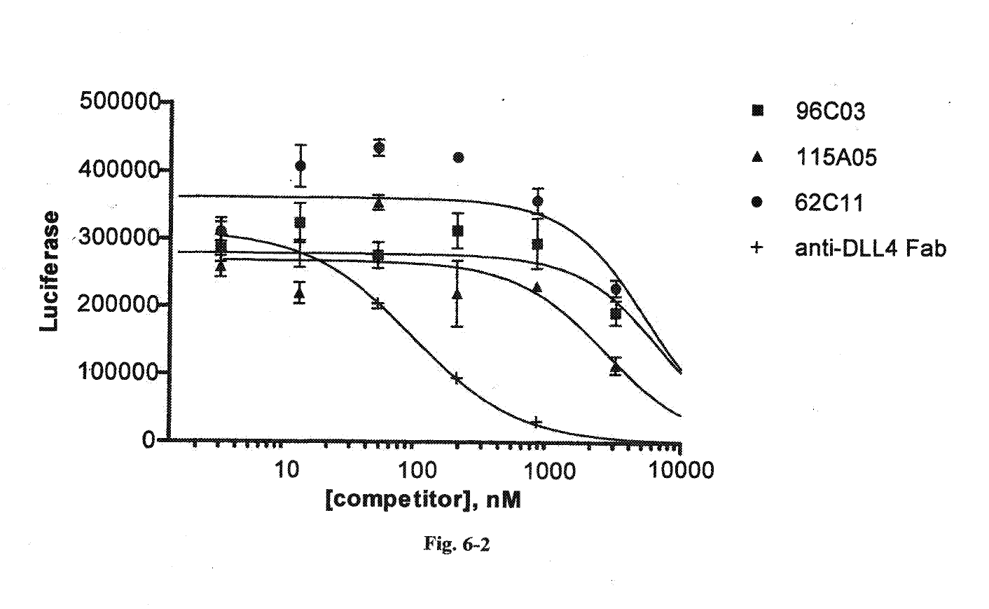

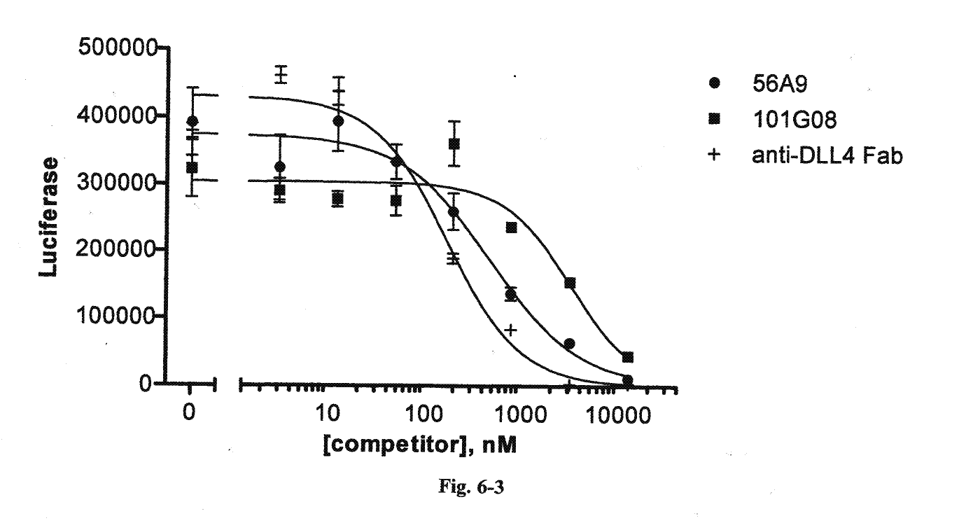

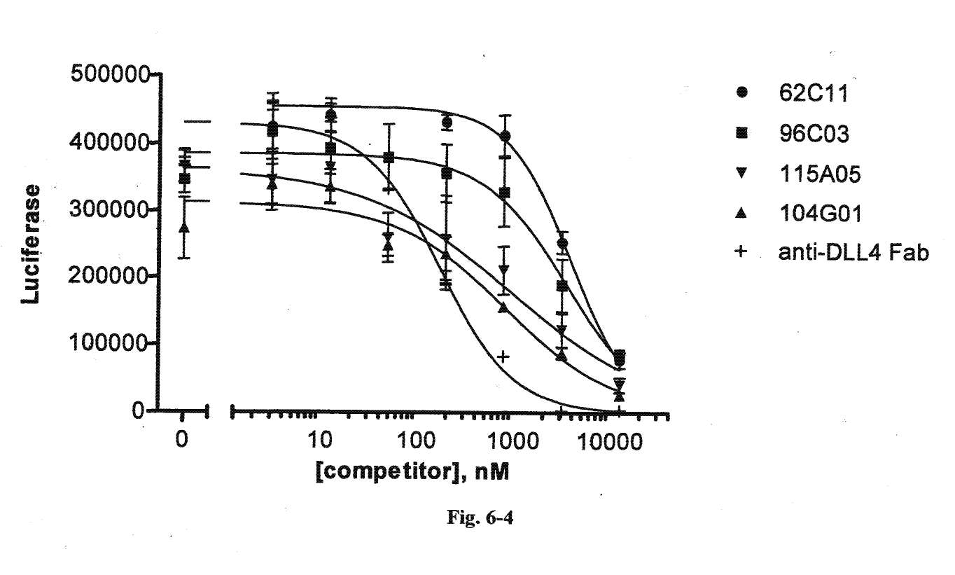

To evaluate the potency of the selected VHHs, a reporter assay is set up which is based on the y-secretase mediated cleavage of Notch1 and release of the intracellular domain of Notch1 (NICD) upon stimulation with Dll4. The Notch1-GAL4/VP16 construct is cotransfected with the pGL4.31[Luc2P/Gal4UAS/Hygro] reporter plasmid in HEK cells resulting in a transient expression of the fusion protein. These transiently transfected cells are stimulated for 24 hours by co-culture with a HEK293-hDll4 stable cell line. Forty-eight hours post-transfection, the readout is performed. The VHHs are preincubated with the HEK293-hDll4 cells 1 hour before the start of the co-culture and are included during the co-culture (FIG. 6). The IC₅₀ values of the VHHs for blocking the Dll4-mediated cleavage of Notch1 and subsequent translocation of its NICD to the nucleus of the receptor cell are depicted in Table 9.

TABLE 9
(Mean) IC₅₀ values (nM) of purified VHHs in a
DLL4/Notch1 reporter assay

| VHH ID | IC₅₀ |
|---|---|
| 56A09 | 540 |
| 62C11 | 4663 |
| 96C03 | 5156 |
| 101G08 | 2760 |
| 104G01 | 964 |
| 115A05 | 1740 |
| anti-DLL4 Fab | 133 |

5.5. Epitope Binning

In order to determine whether VHHs can bind simultaneously to Dll4 when e.g. a benchmark antibody is bound, epitope binning experiments are carried out (via Surface Plasmon Resonance (SPR) on a Biacore T100 instrument). Anti-Dll4 Fab fragment is irreversibly immobilized on the reference and on the active flow cell of a CM5 sensor chip. For each sample (cycle), human Dll4 is injected on the active and reference flow cell and reversibly captured by anti-Dll4 Fab. Additional binding of VHHs is evaluated by injection over the immobilized surface. All VHHs and anti-Dll4 Fab are injected at 100 nM with a surface contact time of 120 seconds and a flow rate of 10 uL/minute. Surface is regenerated using 10 mM glycine (pH1.5). Processed curves are evaluated with Biacore T100 Evaluation software. Table 10-A represents the sequential injection/regeneration path of analysed VHHs and controls. VHHs DLLBII56A09 (SEQ ID NO:15), DLLBII96CO3 (SEQ ID NO:19), DLLBII101G08 (SEQ ID NO: 10) and DLLBII115A05 (SEQ ID NO: 112) are shown not to additionally bind to human Dll4 captured by Dll4 Fab. Injection of Dll4 Fab also failed to additionally bind human Dll4 indicating that all epitopes are saturated. Therefore, it can be concluded that these VHHs recognize an epitope overlapping with Dll4 Fab for binding human Dll4. Human-only VHHs DLLBII6B11 (SEQ ID NO:5) and DLLBII104G01 (SEQ ID NO:11) show additional binding on Dll4 Fab captured human Dll4, indicating that these VHHs that are specific for human Dll4 recognize a different epitope than the human/mouse cross-reactive VHHs.

TABLE 10-A
Epitope-binning of anti-DLL4 VHHs - simultaneous binding with
DLL4 Fab

| Injection | Binding/ | | Binding level |
|---|---|---|---|
| step | Regeneration | [sample] | (RU) |
| 1 | hDLL4 | 100 nM | 1727 |
| 2 | DLL4 Fab | 100 nM | no binding |
| 3 | 59A9 | 100 nM | no binding |
| 4 | 6B11 | 100 nM | 405 |
| 5 | Glycine pH 1.5 | 10 mM | 90 |
| 6 | hDLL4 | 100 nM | 1349 |
| 7 | 104G1 | 100 nM | 276 |
| 8 | Glycine pH 1.5 | 10 mM | 87 |
| 9 | hDLL4 | 100 nM | 1336 |
| 10 | Glycine pH 1.5 | 10 mM | 70 |
| 11 | hDLL4 | 100 nM | 1333 |
| 12 | 96C3 | 100 nM | no binding |
| 13 | 101G8 | 100 nM | no binding |
| 14 | 115A05 | 100 nM | no binding |
| 15 | Glycine pH 1.5 | 10 mM | 70 |

5.6. Epitope Mapping Using Dll4 Deletion Mutants

Binding of the VHHs to these Dll4 mutants is assessed in Biacore. In brief, VHHs DLLBII101G08 (SEQ ID NO:10) and DLLBII115A5 (SEQ ID NO:12) are coated on a CM4 Sensorchip and 200 nM of each deletion mutant is injected across the chip. Binding is qualitatively assessed. No binding of DLLBII56A09 (SEQ ID NO:15), DLLBII101 G08 (SEQ ID NO: 10) and DLLBII115A05 (SEQ ID NO: 12) is observed to human and mouse Dll4 mutants hDll4.1 and mDll4.8, respectively, lacking EGF-like 2 domain (Table 10-B). Indirect evidence using a hDll4/Dll4 IgG competitive ELISA already pointed to this observation. In brief, 1 μg/mL of Dll4 IgG is coated in a 96-well MaxiSorp plate (Nunc, Wiesbaden, Germany). A fixed concentration of 6 nM biotinylated human Dll4 is preincubated with a dilution series of the VHH for 1 hour, after which the mixture is incubated on the coated IgG for an additional hour. Residual binding of biotinylated human Dll4 is detected using horseradish peroxidase conjugated extravidin (Sigma, St. Louis, Mo., USA) (data not shown). Human Dll4 is biotinylated as described above. It is known from patent literature that the monoclonal anti-Dll4 IgG (Genentech, US 2008/0014196A1) binds to an epitope within the EGF-like 2 domain of Dll4.

TABLE 10-B
Epitope mapping of anti-DLL4 VHHs - binding to DLL4 deletion mutants

| | DLLBII56A9 | | DLLBII101G8 | | DLLBII115A5 | |
|---|---|---|---|---|---|---|
| | Binding | | Binding | | Binding | |
| sample | (RU) | kd (1/s) | (RU) | Kd (1/s) | (RU) | kd (1/s) |
| hDLL4 | 281 | 9.5E−04 | 373 | 2.0E−03 | 324 | 3.5E−03 |
| mDLL4 | 389 | 1.9E−03 | 502 | 6.0E−03 | 344 | 6.5E−03 |
| hDLL4.1 | no | | no | | no | |
| | binding | | binding | | binding | |
| hDLL4.3 | 125 | 7.4E−04 | 198 | 4.65E−03 | 137 | 3.5E−03 |
| hDLL4.5 | 143 | 1.2E−03 | 266 | 2.19E−03 | 162 | 4.2E−03 |
| hDLL4.6 | 136 | 1.1E−03 | 229 | 2.20E−03 | 152 | 4.1E−03 |
| mDLL4.8 | no | | no | | no | |
| | binding | | binding | | binding | |
| mDLL4.10 | 141 | 1.1E−03 | 189 | 5.14E−03 | 121 | 3.8E−03 |
| mDLL4.11 | 132 | 1.6E−03 | 210 | 6.16E−03 | 121 | 6.6E−03 |
| mDLL4.12 | 161 | 1.3E−03 | 244 | 4.52E−03 | 152 | 3.1E−03 |

5.7. Determining the Affinity of the hDll4—VHH Interaction

Kinetic analysis to determine the affinity of the Dll4—VHH interaction is performed by Surface Plasmon Resonance (SPR) on a Biacore T100 instrument. Recombinant human Dll4 is immobilized onto a CM5 chip via amine coupling using EDC and NHS) or biotinylated human Dll4 is captured on a SA chip (streptavidin surface). Purified VHHs or Fab fragment are injected for 2 minutes at different concentrations (between 10 and 300 nM) and allowed to dissociate for 20 min at a flow rate of 45 μl/min. Between sample injections, the surfaces are regenerated with 10 mM glycine pH1.5 and 100 mM HCl. HBS-N (Hepes buffer pH7.4) is used as running buffer. If possible, data are evaluated by fitting a 1:1 interaction model (Langmuir binding) onto the binding curves. The affinity constant K_{D} is calculated from resulting association and dissociation rate constants (kₐ) and (k_{d}). The affinities of the anti-Dll4 VHHs are depicted in Table 11.

TABLE 11
Affinity K_{D} (nM) of purified VHHs for recombinant human DLL4

| | rhDLL4 | | |
|---|---|---|---|
| VHH ID | kₐ (M⁻¹ · s⁻¹) | k_{d} (s⁻¹) | K_{D} (nM) |
| 56A09 | 1.7E+05 | 9.3E−04 | 5.6 |
| 56C04 | 1.1E+05 | 4.9E−03 | 45 |
| 56H08 | 1.2E+05 | 1.1E−03 | 9.4 |
| 62C11 | 1.2E+06 | 1.3E−01 | 120 |
| 96C03 | 1.6E+05 | 4.8E−02 | 310 |
| 101G08 | 4.3E+04 | 2.2E−03 | 52 |
| 104G01^{(a)} | 1.2E+05-1.5E+05 | 3E−03-6E−04 | 4-24 |
| 115A05 | 1.5E+05 | 3.9E−03 | 25 |
| antiDLL4 Fab | 2.3E+05 | 3.4E−04 | 1.5 |

^{(a)}heterogeneous binding curve resulting in no 1:1 fit

5.8. Binding to Orthologues (mDll4, cDll4) and Family Members (hJagged-1, hDLL1)

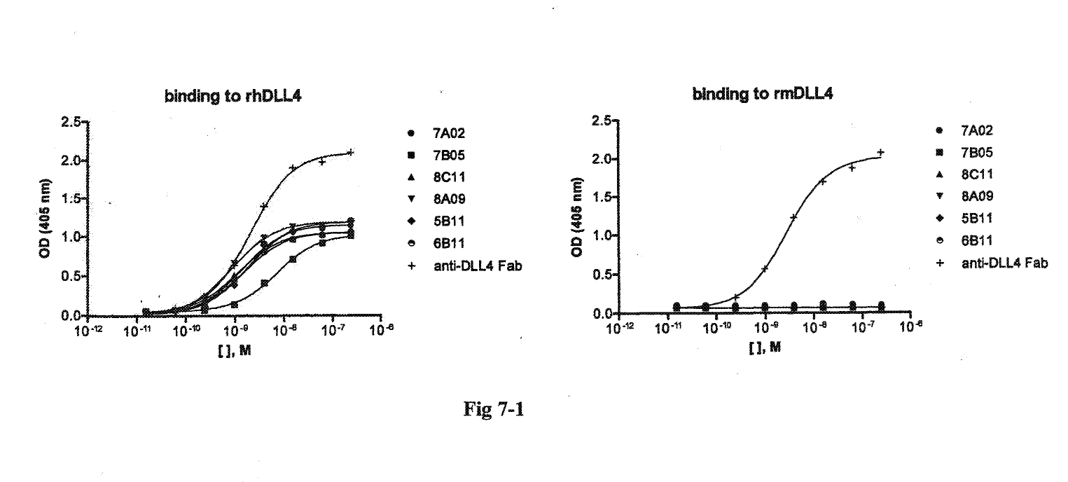

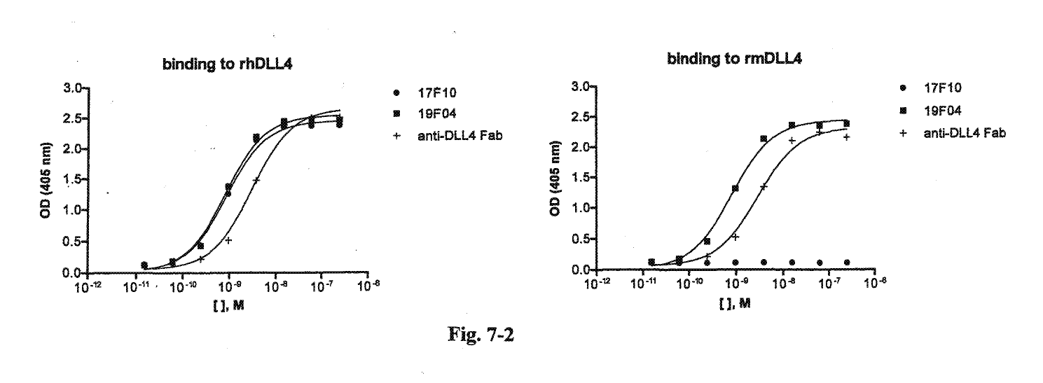

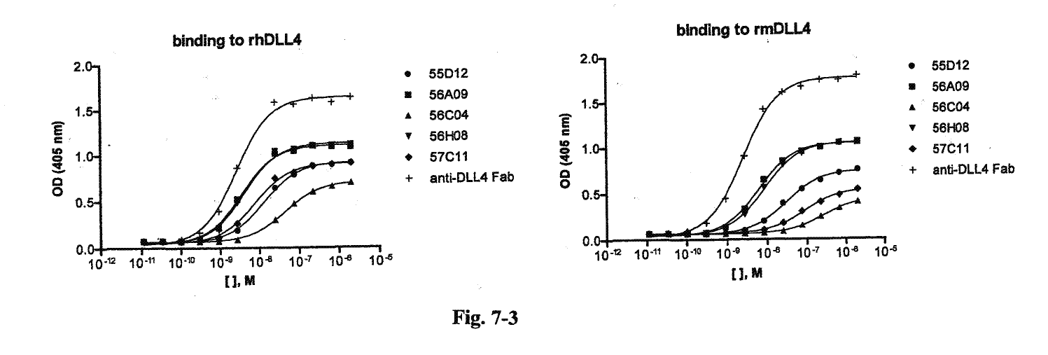

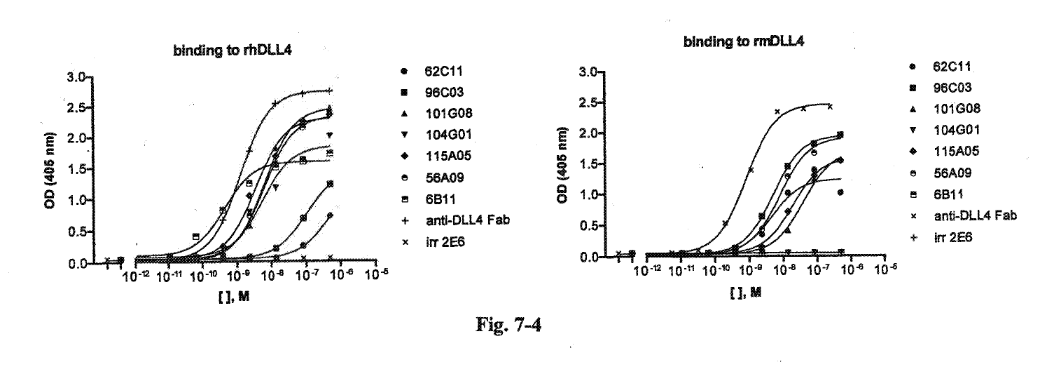

In order to determine cross-reactivity to mouse Dll4 a binding ELISA is performed. In brief, recombinant mouse Dll4 (R&D Systems, Minneapolis, Miss., USA) is coated overnight at 4° C. at 1 μg/mL in a 96-well MaxiSorp plate (Nunc, Wiesbaden, Germany). Wells are blocked with a casein solution (1% in PBS). VHHs are applied as dilution series and binding is detected using a mouse anti-myc (Roche) and an anti-mouse-AP conjugate (Sigma, St Louis, Mo., USA) (FIG. 7). As reference, binding to human Dll4 is measured. EC₅₀ values are summarized in Table 12.

TABLE 12
EC₅₀ (nM) values for VHHs in a recombinant human DLL4- and
mouse DLL4-binding ELISA

| | rhDLL4 | rmDLL4 |
|---|---|---|
| VHH ID | EC₅₀ (nM) | EC₅₀ (nM) |
| 5B11 | 1.8 | — |
| 6B11 | 1.4 | — |
| 7A02 | 1.4 | — |
| 7B05 | 7.2 | — |
| 8A09 | 0.9 | — |
| 8C11 | 1.1 | — |
| 17F10 | 0.9 | — |
| 19F04 | 0.9 | 0.8 |
| 55D12 | 13.1 | 30.0 |
| 56A09 | 3.6 | 6.3 |
| 56C04 | 44.3 | 244.0 |
| 56H08 | 4.1 | 8.7 |
| 57C11 | 7.9 | 83.4 |
| 62C11 | 137.0 | 13.1 |
| 96C03 | 86.5 | 8.7 |
| 101G08 | 8.9 | 53.9 |
| 104G01 | 8.4 | — |
| 115A05 | 5.0 | 33.4 |
| antiDLL4 Fab | 3.0 | 3.0 |

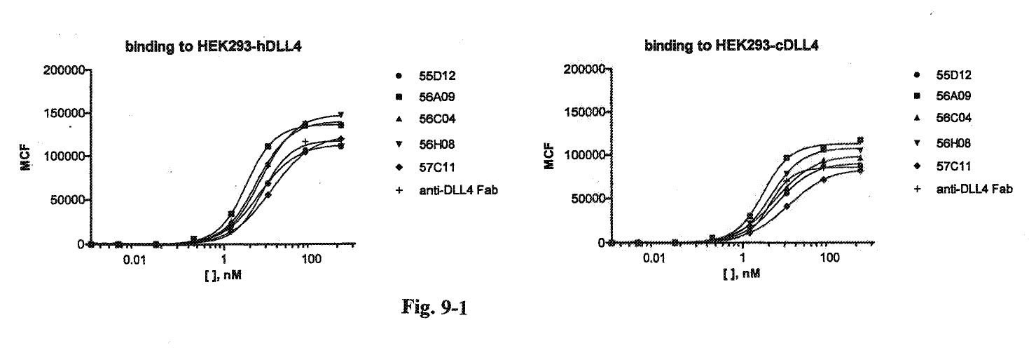

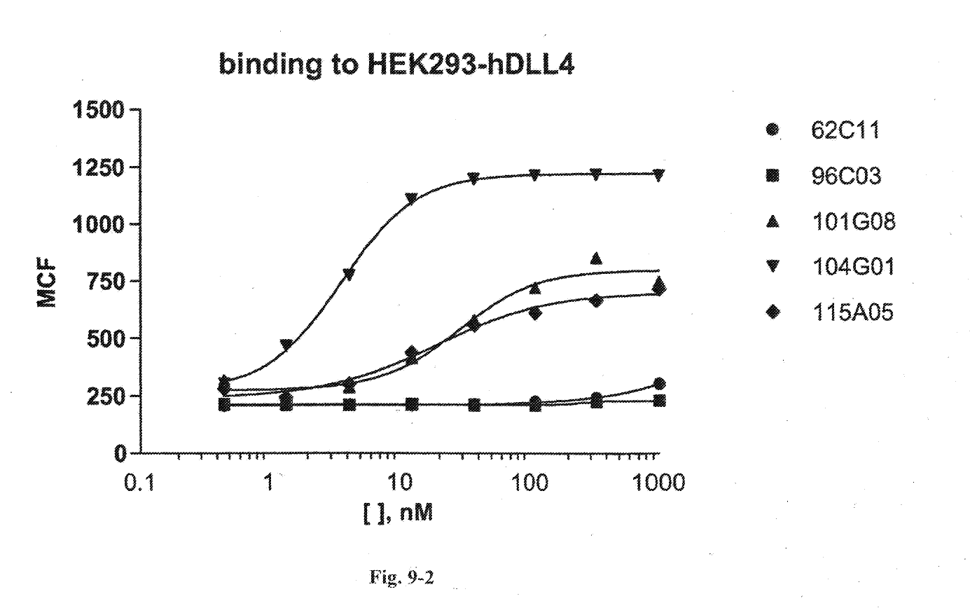

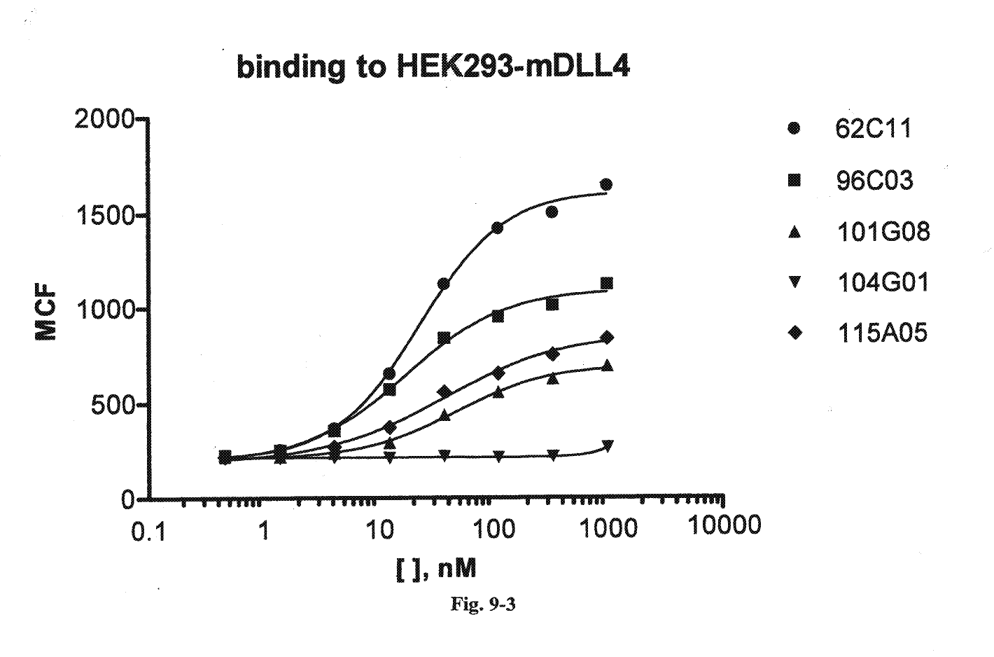

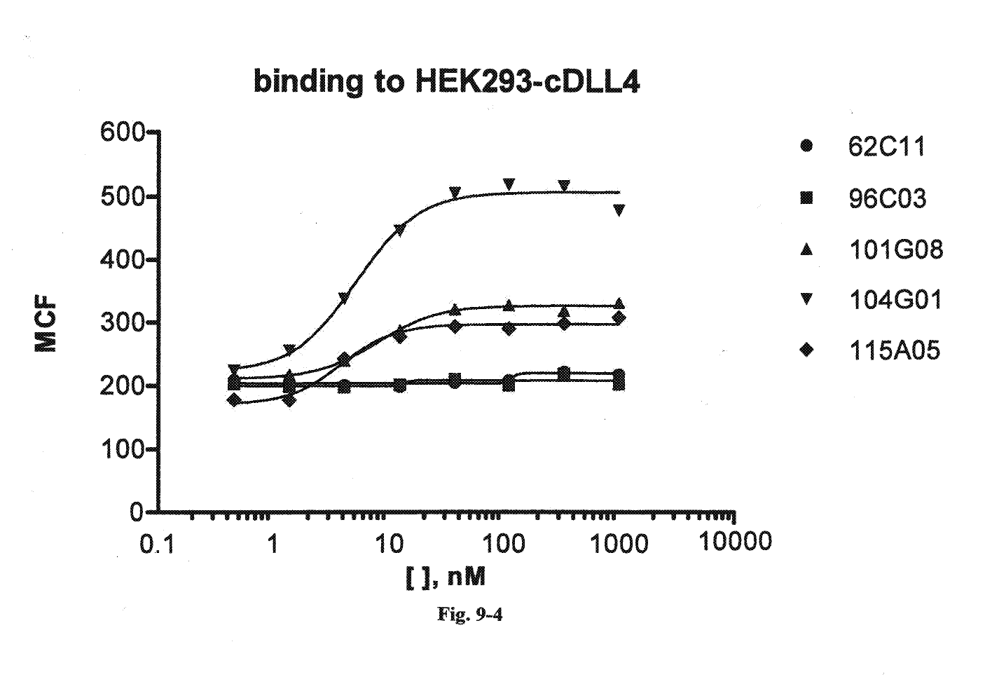

In order to determine the cynomologus cross-reactivity of the VHHs, a FACS binding experiment is performed. Cynomolgus Dll4 expressing HEK293 cells (transient or stable transfection) are used for a titration binding experiment of the VHHs. After a 30 minutes incubation on ice, all samples are washed and detection is performed by applying anti-c-myc-Alexa647 (Santa Cruz Biotechnology, Santa Cruz, Calif., USA). Human and mouse Dll4 overexpressing HEK293 cells are taken as reference. The mean MCF value is determined on the FACS Array and used for calculation of the EC₅₀ value (see FIG. 9).

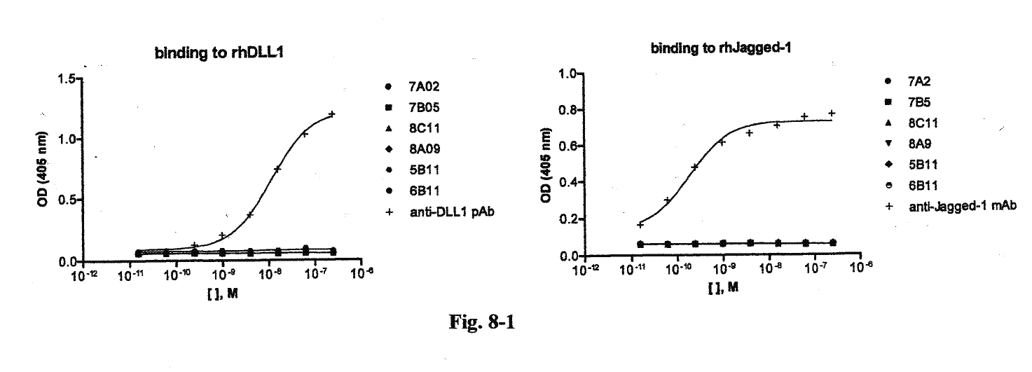

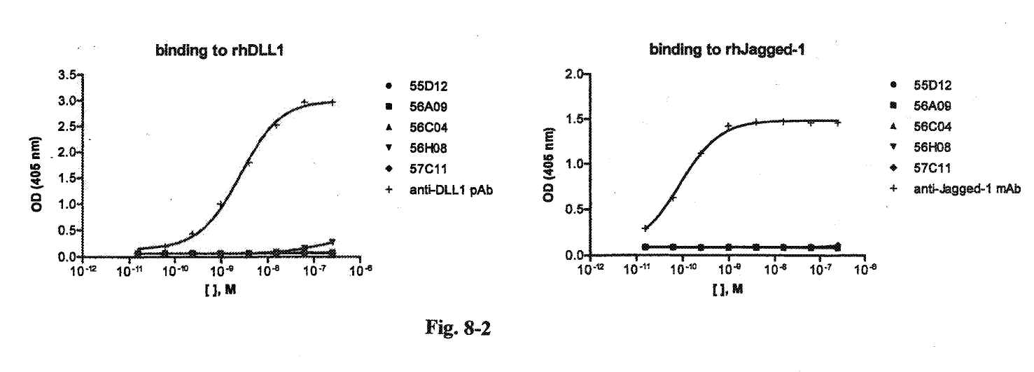

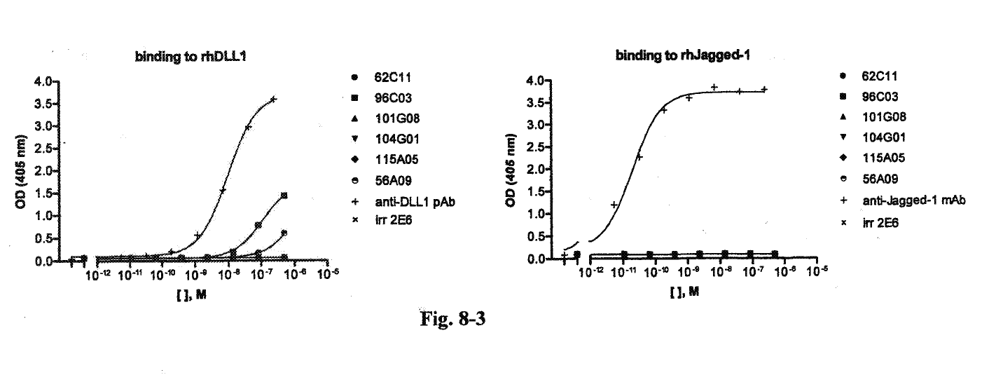

Absence of binding to homologous ligands human DLL1 and human Jagged-1 is assessed via solid phase binding assay (ELISA). In brief, human DLL1 (Alexis, San Diego, Calif., USA) and human Jagged-1 (Alexis, San Diego, Calif., USA) are coated overnight at 4° C. at 1 μg/mL in a 96-well MaxiSorp plate (Nunc, Wiesbaden, Germany). Wells are blocked with a casein solution (1% in PBS). VHHs are applied as dilution series and binding is detected using a mouse anti-myc (Roche) and an anti-mouse-AP conjugate (Sigma, St. Louis, Mo., USA). All anti-Dll4 VHHs are considered as being non-cross reactive to these homologous ligands (FIG. 8).

5.9. Evaluation of Anti-Dll4 VHHs in Blocking Dll4-Mediated HUVEC Proliferation

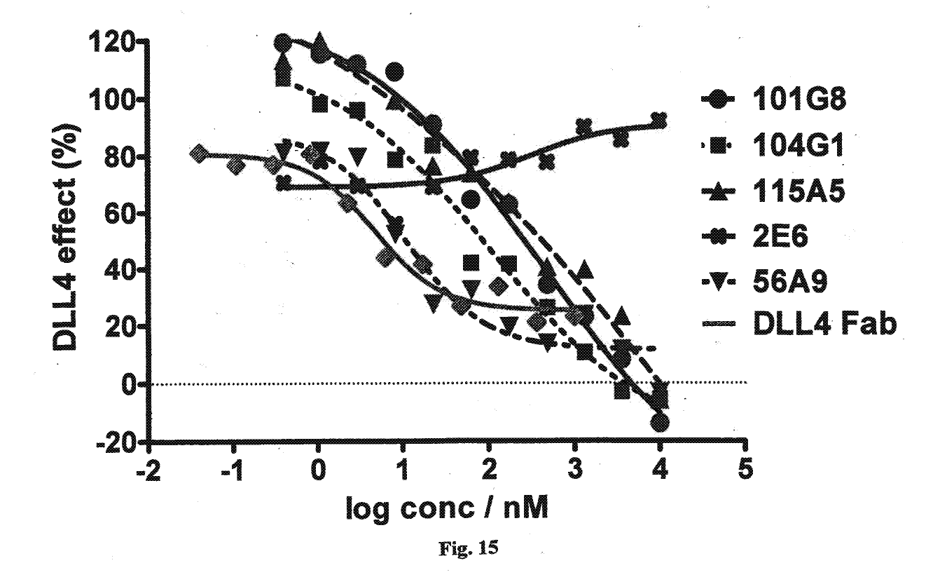

The potency of the selected VHHs is evaluated in a proliferation assay, as described by Ridgway et al., Nature. Dec. 21, 2006; 444(7122):1083-7), in modified form. In brief, 96-well tissue culture plates are coated with purified Dll4-His (RnD Systems; C-terminal His-tagged human Dll4, amino acid 27-524, 0.75 ml/well, 10 ng/ml) in coating buffer (PBS, 0.1% BSA). Wells are washed in PBS before 4000 HUVEC cells/well are seeded in quadruplicate. Cell proliferation is measured by [³H]-Thymidine incorporation on day 4. The results, shown in FIG. 15, demonstrate that the DLL4 VHHs DLLBII101G08, DLLBII104G01, DLLBII115A05, DLLBII56A09 and the DLL4 Fab inhibit the DLL4-dependent effect on HUVEC proliferation in a dose-dependent manner, the IC₅₀ values are summarized in Table 13. The tested VHHs achieve complete inhibition of the DLL4-dependent effect at 10 μM.

TABLE 13
IC₅₀ values obtained in the DLL4 proliferation assay

| VHH/Fab | Fab | 56A9 | 104G1 | 101G8 | 115A5 |
|---|---|---|---|---|---|
| IC₅₀ (nM) (experiment 1) | 4.9 | 11.0 | 103 | 401 | 10002 |
| IC₅₀ (nM) (experiment 2) | 5.6 | 6.8 | 32 | 112 | N.D. |
| n | 2 | 2 | 2 | 2 | 1 |

### EXAMPLE 6

Affinity Maturation of Selected Anti-Dll4 VHHs

VHHs DLLBII101G08 and DLLBII115A05 are subjected to two cycles of affinity maturation.

In a first cycle, amino acid substitutions are introduced randomly in both framework (FW) and complementary determining regions (CDR) using the error-prone PCR method. Mutagenesis is performed in a two-round PCR-based approach (Genemorph II Random Mutagenesis kit obtained from Stratagene, La Jolla, Calif., USA) using 1 ng of the DLLBII101G08 or DLLBII115A05 cDNA template, followed by a second error-prone PCR using 0.1 ng of product of round 1. After a polish step, PCR products are inserted via unique restriction sites into a vector designed to facilitate phage display of the VHH library. Consecutive rounds of in-solution selections are performed using decreasing concentrations of biotinylated recombinant human DLL4 (biot-rhDLL4) and trypsin elutions. Affinity-driven selections in a third round using cold rhDLL4 (at least 100× excess over biot-rhDLL4) are also performed. No selections on murine DLL4 are included as (conservation of) cross-reactivity is assessed at the screening level. Individual mutants are produced as recombinant protein using an expression vector derived from pUC119, which contains the LacZ promoter, a resistance gene for ampicillin, a multiple cloning site and an ompA leader sequence (pAX50). E. coli TG1 cells are transformed with the expression vector library and plated on agar plates (LB+Amp+2% glucose). Single colonies are picked from the agar plates and grown in 1 mL 96-deep-well plates. VHH expression is induced by adding IPTG (1 mM). Periplasmic extracts (in a volume of ˜80 uL) are prepared according to standard methods and screened for binding to recombinant human and mouse Dll4 in a ProteOn (BioRad, Hercules, Calif., USA) off-rate assay. In brief, a GLC ProteOn Sensor chip is coated with recombinant human Dll4 on the “ligand channels” L2 and L4 (with L1/L3 as reference channel), while “ligand channels” L3 and L6 is coated with mouse Dll4. Periplasmic extract of affinity-matured clones is diluted 1/10 and injected across the “analyte channels” A1-A6. An average off-rate is calculated of the wild type clones present in the plate and served as a reference to calculate off-rate improvements.

In a second cycle, a combinatorial library is created by simultaneously randomising the susceptible positions identified in cycle one. For this, the full length DLLBII101G8 or DLLBII115A05 cDNA is synthesized by overlap PCR using oligonucleotides degenerated (NNS) at the randomisation positions and a rescue PCR is performed. The randomised VHH genes are inserted into a phage display vector (pAX50) using specific restriction sites as described above (Example 2). Preparation of periplasmic extracts of individual VHH clones is performed as described before.

Screening for binding to recombinant human Dll4 in a PrateOn off-rate assay identifies clones with up to 38-fold (DLLBII101G08) and 11-fold (DLLBII115A05) improved off-rates (Table 15).

TABLE 15
Off-rate screening of DLLBII101G08 and DLLBII15A05 affinity-
matured clones.

| | hDLL4 | | mDLL4 | |
|---|---|---|---|---|
| | k_{d} (s⁻¹) | fold | k_{d} (s⁻¹) | fold |
| DLLBII101G08 | 2.2E−03 | 1 | 6.7E−03 | 1 |
| DLLBII129D08 | 5.9E−05 | 38 | 1.9E−04 | 35 |
| DLLBII129H04 | 6.8E−05 | 33 | 2.5E−04 | 27 |
| DLLBII129G10 | 7.3E−05 | 31 | 2.6E−04 | 26 |
| DLLBII129H07 | 7.4E−05 | 30 | 2.5E−04 | 27 |
| DLLBII129B02 | 7.6E−05 | 30 | 2.6E−04 | 26 |
| DLLBII129E11 | 8.0E−05 | 28 | 2.5E−04 | 26 |
| DLLBII130F06 | 6.5E−05 | 27 | 2.6E−04 | 19 |
| DLLBII130B03 | 6.7E−05 | 27 | 2.4E−04 | 20 |
| DLLBII129D01 | 8.5E−05 | 26 | 2.6E−04 | 26 |
| DLLBII130D06 | 6.9E−05 | 26 | 3.1E−04 | 16 |
| DLLBII129G09 | 8.8E−05 | 26 | 3.4E−04 | 20 |
| DLLBII129B05 | 9.3E−05 | 24 | 3.4E−04 | 20 |
| DLLBII130E03 | 7.5E−05 | 24 | 2.7E−04 | 18 |
| DLLBII129H05 | 9.4E−05 | 24 | 3.5E−04 | 19 |
| DLLBII130A05 | 7.5E−05 | 24 | 3.0E−04 | 17 |
| DLLBII130B02 | 7.8E−05 | 23 | 2.9E−04 | 17 |
| DLLBII129H02 | 9.9E−05 | 23 | 3.4E−04 | 19 |
| DLLBII130B04 | 8.3E−05 | 22 | 2.9E−04 | 17 |
| DLLBII129E07 | 1.1E−04 | 21 | 2.8E−04 | 24 |
| DLLBII129E03 | 1.1E−04 | 20 | 3.6E−04 | 18 |
| DLLBII129A03 | 1.2E−04 | 19 | 3.8E−04 | 18 |

The best variants of DLLBII101G08 and DLLBII115A05 variants are cloned into expression vector pAX100 in frame with a C-terminal c-myc tag and a (His)6 tag. Off-rates on recombinant mouse Dll4 are also improved. VHHs are produced in E. coli as His6-tagged proteins and purified by IMAC and SEC. Sequences of VHHs selected for further characterization are represented in Tables 16 (DLLBII101G08) and 17 (DLLBII115A05), respectively.

TABLE 16
Affinity-matured variants of 101G08

| VHH ID | | | | | | | |
|---|---|---|---|---|---|---|---|
| SEQ ID | | | | | | | |
| NO | FR1 | CDR1 | FR2 | CDR2 | FR3 | CDR3 | FR4 |
| DLLBII129A03 | EVQLVESGGGLVQAGG | SYAMA | WFRQAPGKERE | AIRWSGGTAY | RFTISRDNAKNTVYLQMNS | RAPDTRLRPY | WGQGTQVTVSS |
| 21 | SLRLSCAASGRTFS | | YVA | YADSVQG | LRPEDTAVYYCAN | LYDY | |
| DLLBII129B05 | EVQLVESGGGLVQAGG | SYAMA | WYRQAPGKERE | AIRWSGGTAY | RFTISRDNAKNTVYLQMNS | RAPDTRLAPY | WGQGTQVTVSS |
| 22 | SLRLSCAASGRTFS | | YVA | YADSVQG | LKPEDTAVYYCAN | EYDH | |
| DLLBII129D08 | EVQLVESGGGLVQAGG | SYAMA | WYRQAPGKERE | AIRWSGGTAY | RFTISRDNAKNTVYLQMNS | RAPDTRLEPY | WGQGTQVTVSS |
| 23 | SLRLSCAASGRTFS | | YVA | YADSVQG | LKPEDTAVYYCAN | LYDY | |
| DLLBII129E11 | EVQLVESGGGLVQAGG | SYAMA | WYRQAPGKERE | AIRWSGGTAY | RFTISRDNAKNTVYLQMNS | RAPDTRLRPY | WGQGTQVTVSS |
| 24 | SLRLSCAASGRTFS | | YVA | YADSVQG | LKPEDTAVYYCAN | LYDY | |
| DLLBII129H07 | EVQLVESGGGLVQAGG | SYAMA | WYRQAPGKERE | AIRWSGGTAY | RFTISRDNAKNTVYLQMNS | RAPDTRLEPY | WGQGTQVTVSS |
| 25 | SLRLSCAASGRTFS | | YVA | YADSVQG | LKPEDTAVYYCAN | EYDY | |
| DLLBII130B03 | EVQLVESGGGLVQAGG | SYAMA | WYRQAPGKERE | AIRWSGGTAY | RFTISRDNAKNTVYLQMNS | RAPDTRLAPY | WGQGTQVTVSS |
| 26 | SLRLSCAASGRTFS | | YVA | YADSVQG | LKPEDTAVYYCAN | LYDY | |
| DLLBII130F06 | EVQLVESGGGLVQAGG | SYAMA | WYRQAPGKERE | AIRWSGGTAY | RFTISRDNAKNTVYLQMNS | RAPDTRLAPY | WGQGTQVTVSS |
| 27 | SLRLSCAASGRTFS | | YVA | YADSVQG | LKPEDTAVYYCAN | EYDY | |

TABLE 17
Affinity-matured variants of 115A05

| VHH ID | | | | | | | |
|---|---|---|---|---|---|---|---|
| SEQ ID | | | | | | | |
| NO | FR1 | CDR1 | FR2 | CDR2 | FR3 | CDR3 | FR4 |
| DLLBII133A05 | EVQLVESGGGLVQPG | SYDMS | WVRRSPGKGP | AINSGGGSTFY | RFTISRDNAKNTLYLQ | DRYIWARQGEYWG | WGQGTQ |
| 28 | GSLRLSCAASGFTFG | | EWVS | TDYVKG | MNSLKPEDTAVYYCAA | AYQYDY | VTVSS |
| DLLBII133A09 | EVQLVESGGGLVQPG | SYDMS | WVRRSPGKGP | AINSGGGSTYY | RFTISRDNAKNTLYLQ | DRYIWARQGEYWG | WGQGTQ |
| 29 | GSLRLSCAASGFTFG | | EWVS | ADYVKG | MNSLKPEDTAVYYCAA | AYAYDY | VTVSS |
| DLLBII133G05 | EVQLVESGGGLVQPG | SYDMS | WVRRSPGKGP | AINSGGGSTYY | RFTISRDNAKNTLYLQ | DRYIWARQGEYWG | WGQGTQ |
| 30 | GSLRLSCAASGFTFG | | EWVS | TDYVKG | MNSLKPEDTAVYYCAA | AYAYDY | VTVSS |
| DLLBII134D10 | EVQLVESGGGLVQPG | SYDMS | WVRRSPGKGP | SINSGGGSTYY | RFTISRDNAKNTLYLQ | DRYIWARQGEYWG | WGQGTQ |
| 31 | GSLRLSCAASGFTIG | | EWVS | TDYVKG | MNSLKPEDTAVYYCAA | AYAYDY | VTVSS |
| DLLBII136C07 | EVQLVESGGGLVQPG | SYDMS | WVRRSPGKGP | SINSGGGSTYY | RFTISRDNAKNTLYLQ | DRYIWARQGEYWG | WGQGTQ |
| 32 | GSLRLSCAASGFTFG | | EWVS | ADYVKG | MNSLKPEDTAVYYCAA | AYEYDY | VTVSS |
| DLLBII015 | EVQLVESGGGLVQPG | SYDMS | WVRRSPGKGP | AINSGGGSTYY | RFTISRDNAKNTLYLQ | DRYIWARQGEYWG | WGQGTQ |
| 33 | GSLRLSCAASGFTIG | | EWVS | ADYVKG | MNSLKPEDTAVYYCAA | AYAYDY | VTVSS |

### EXAMPLE 7

Characterization of Affinity-Matured Purified Anti-Dll4 VHHs

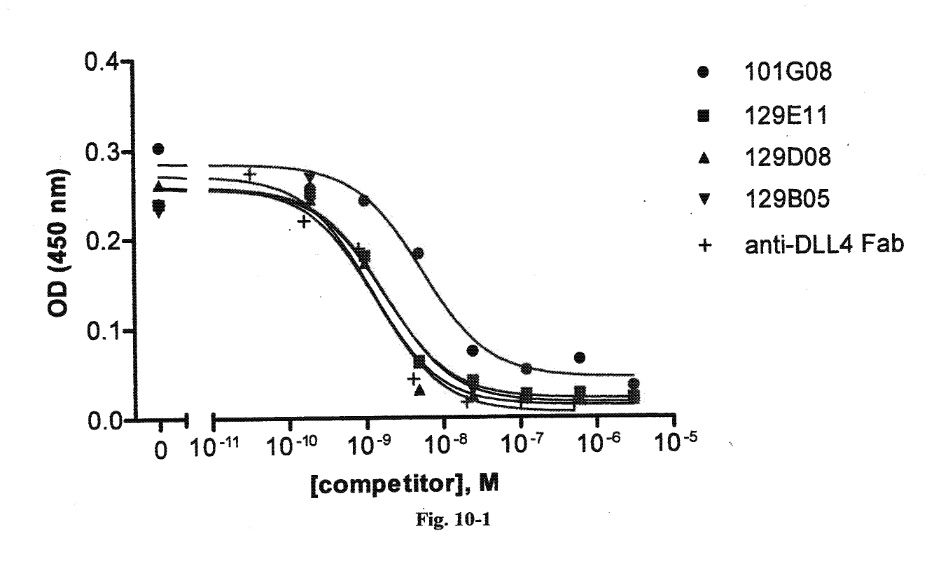

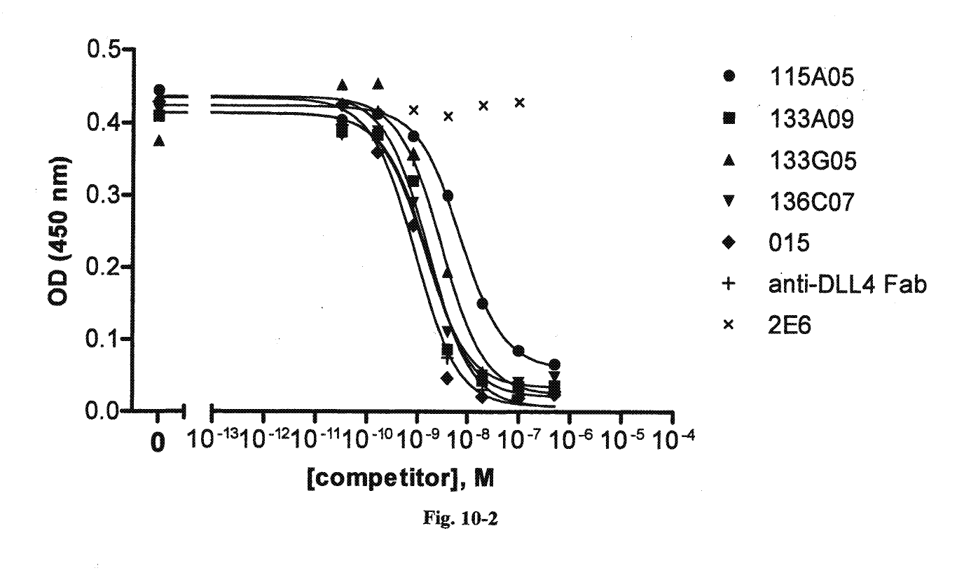

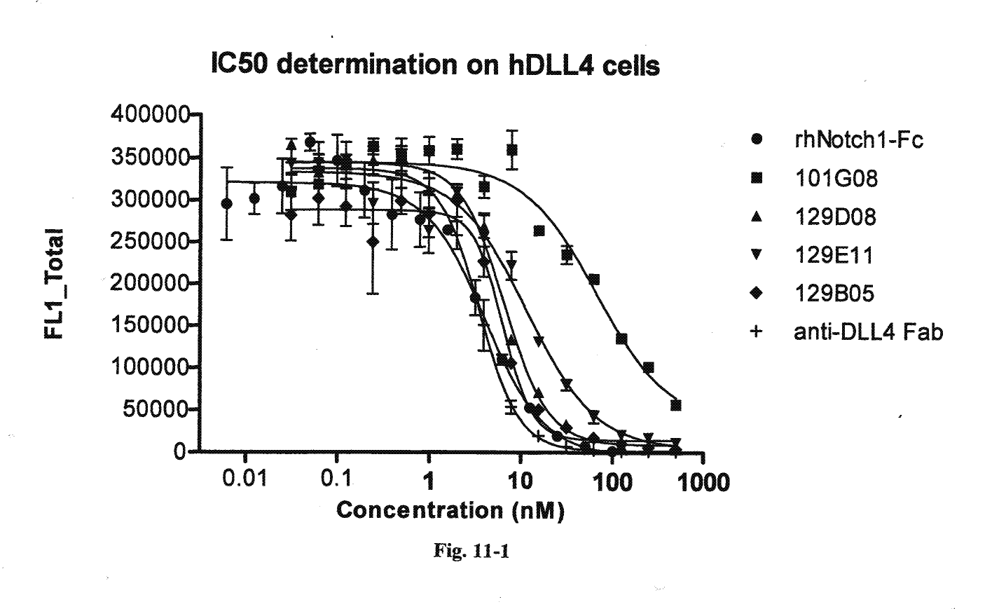

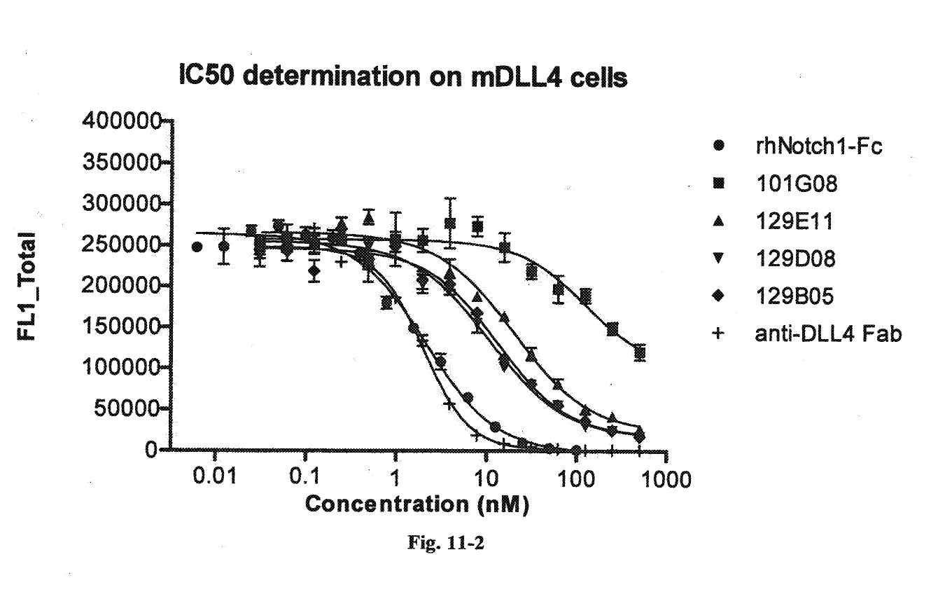

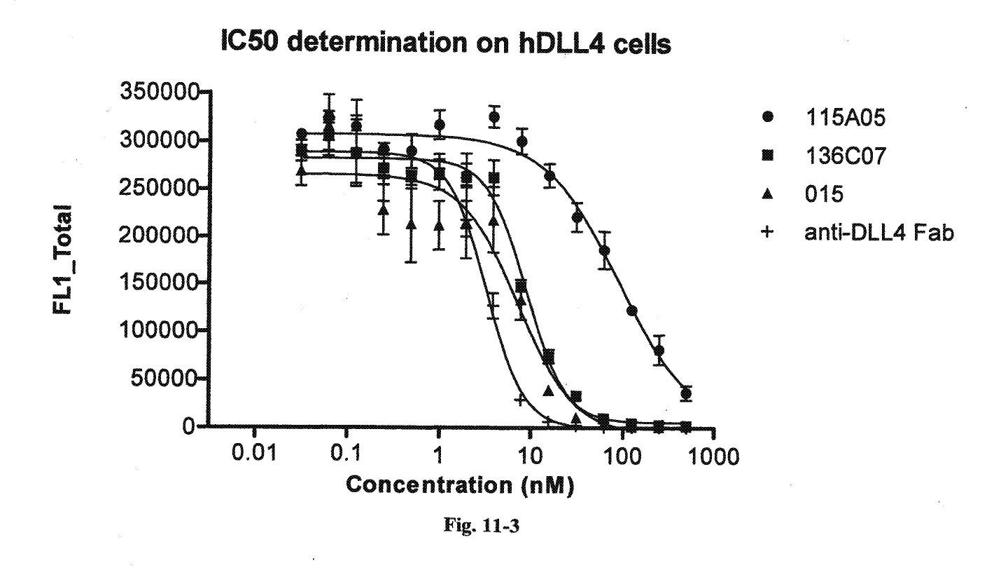

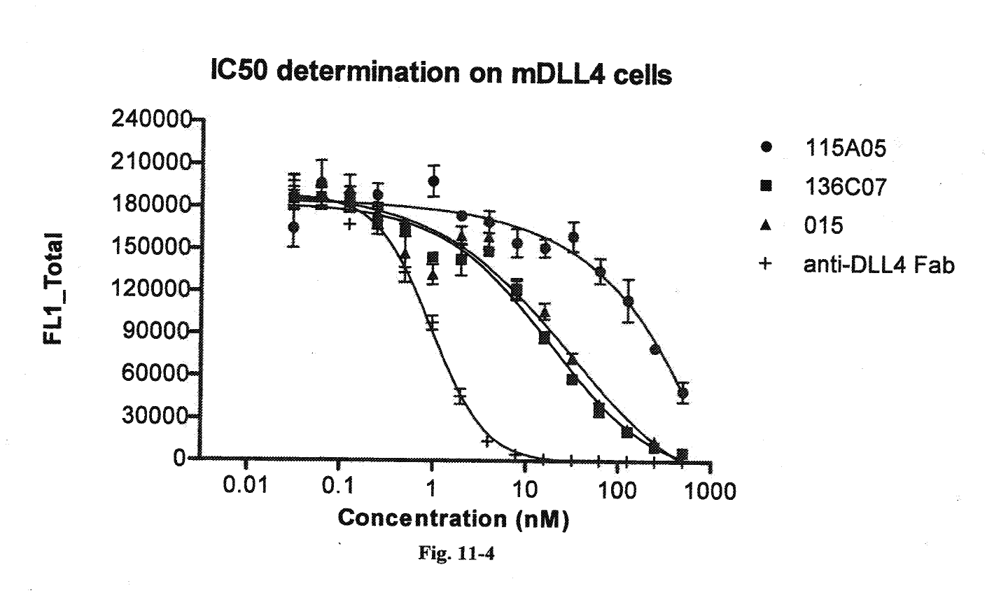

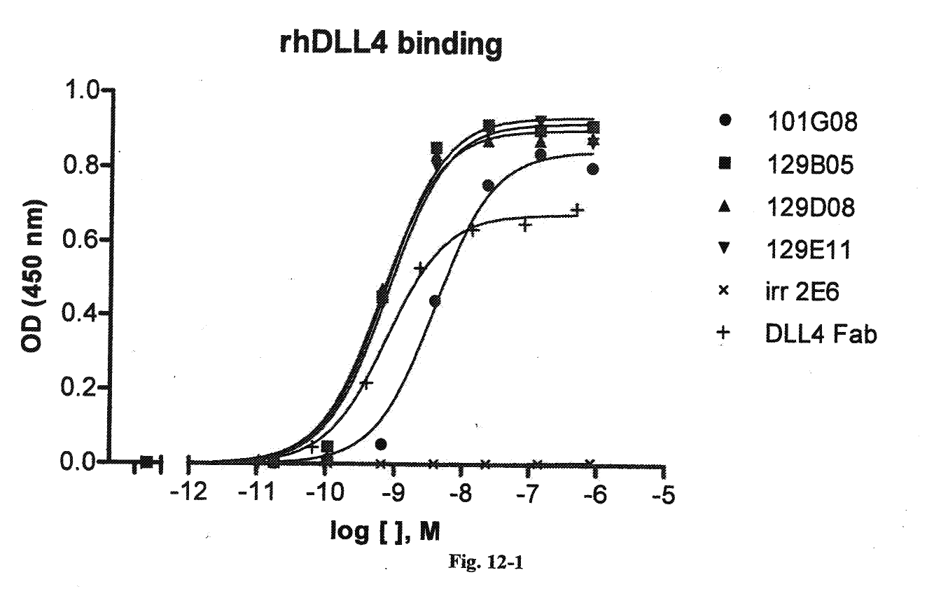

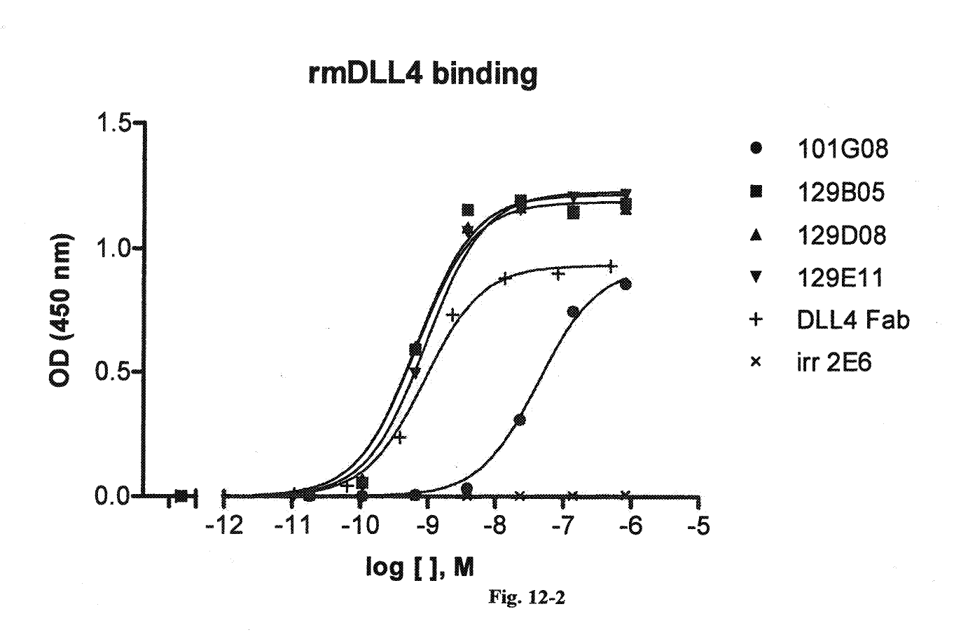

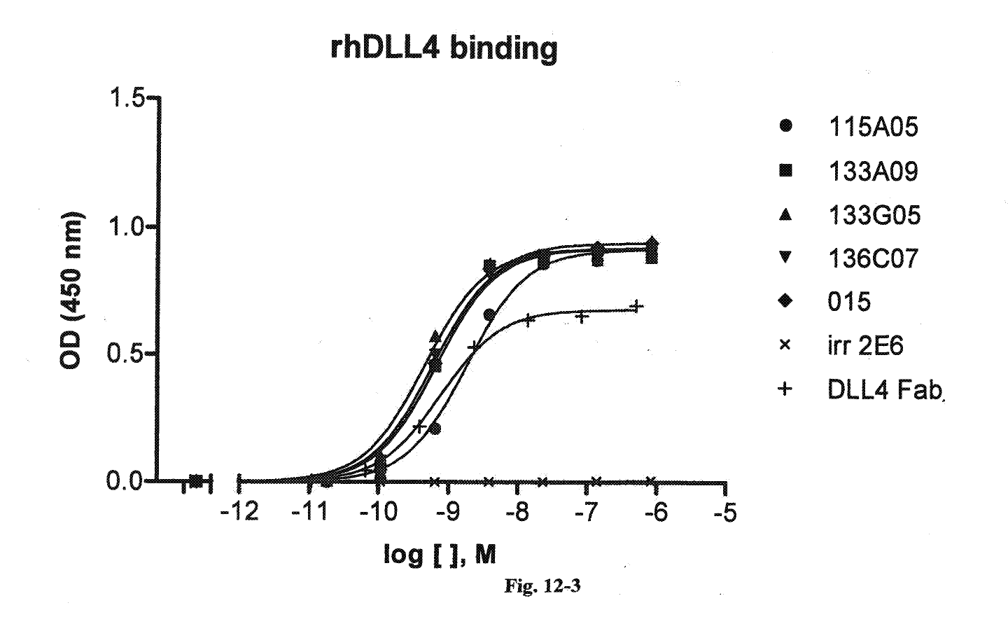

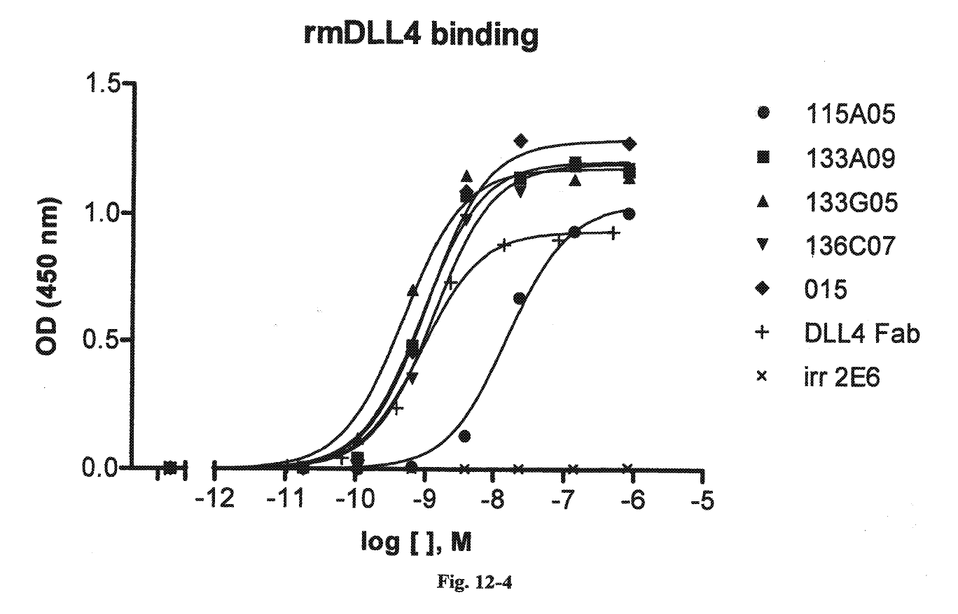

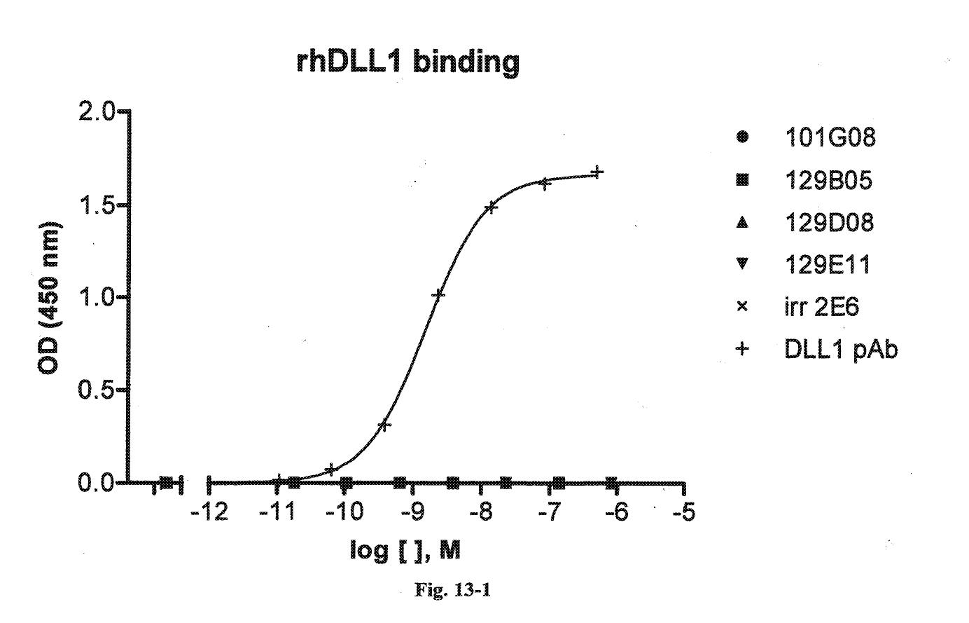

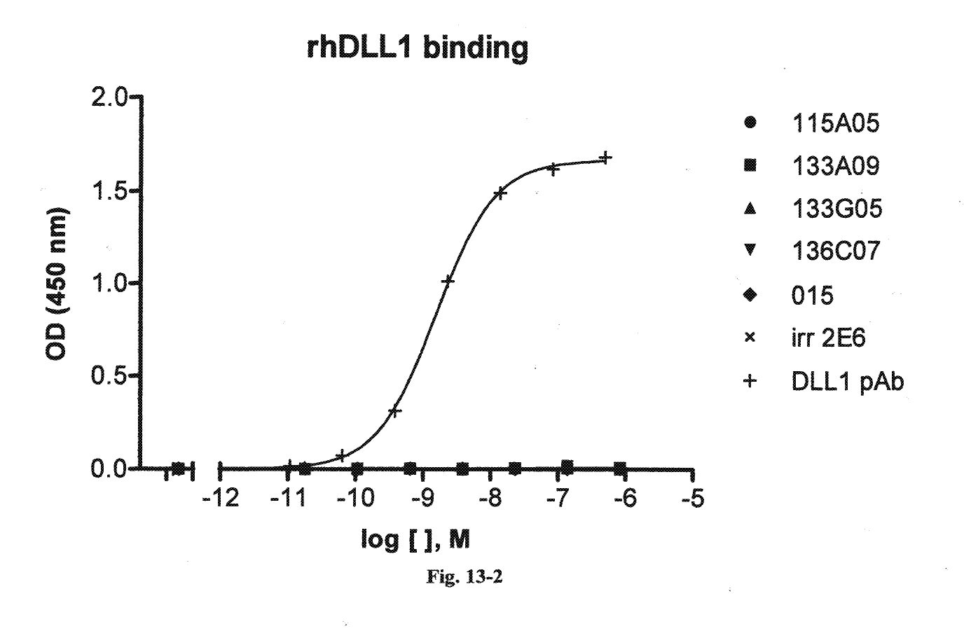

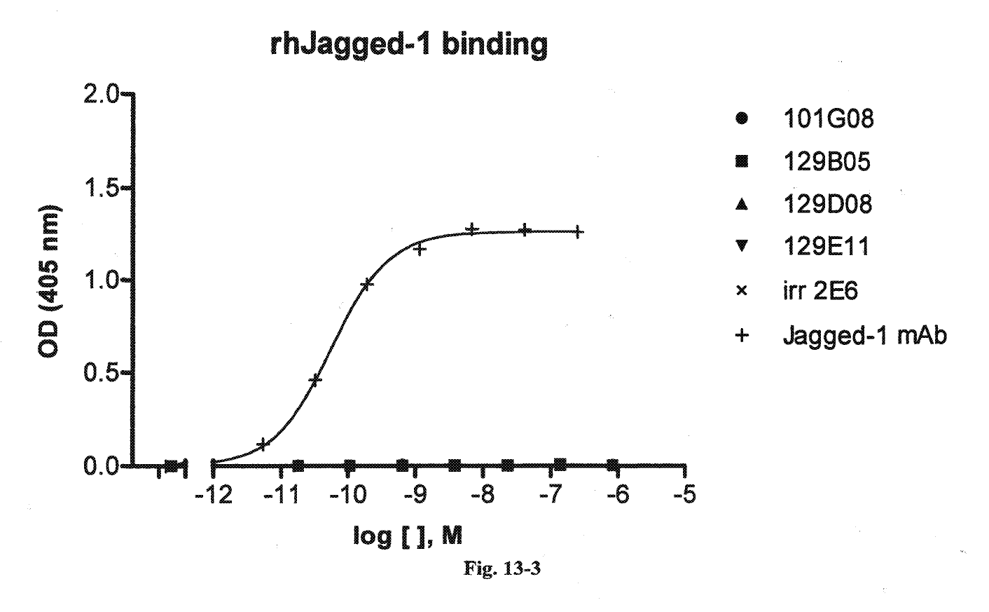

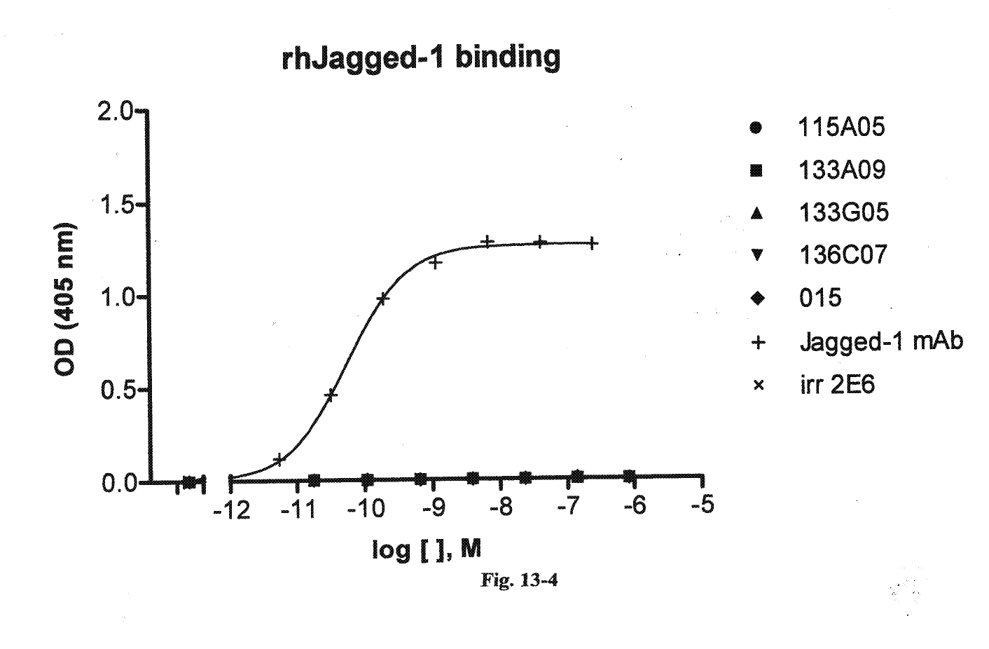

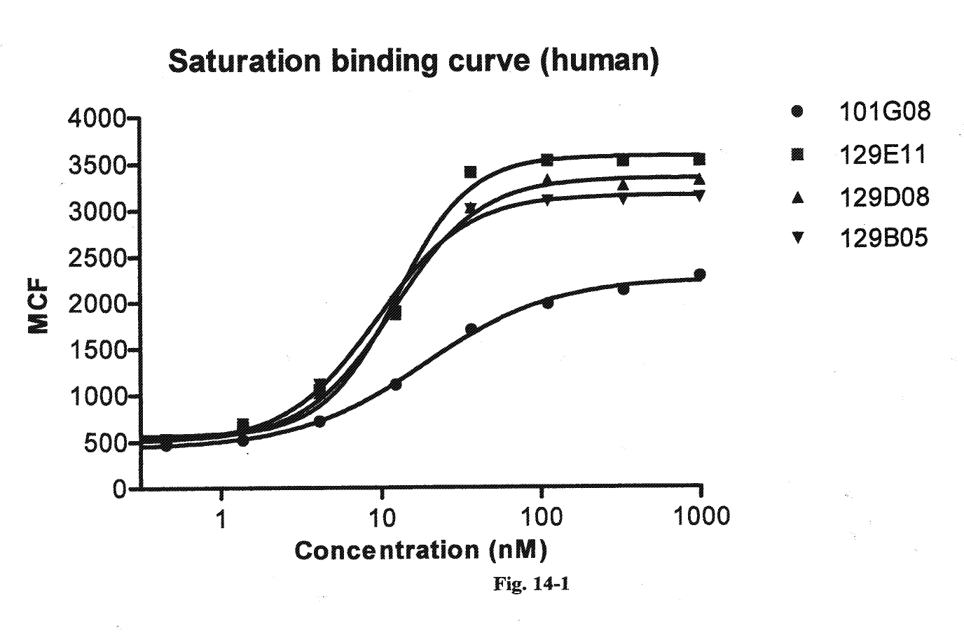

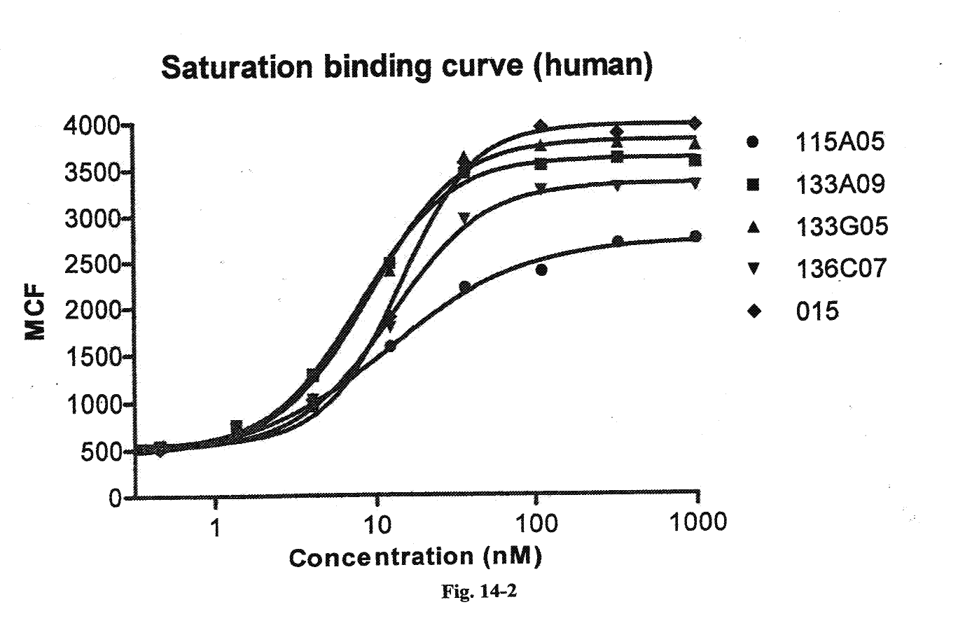

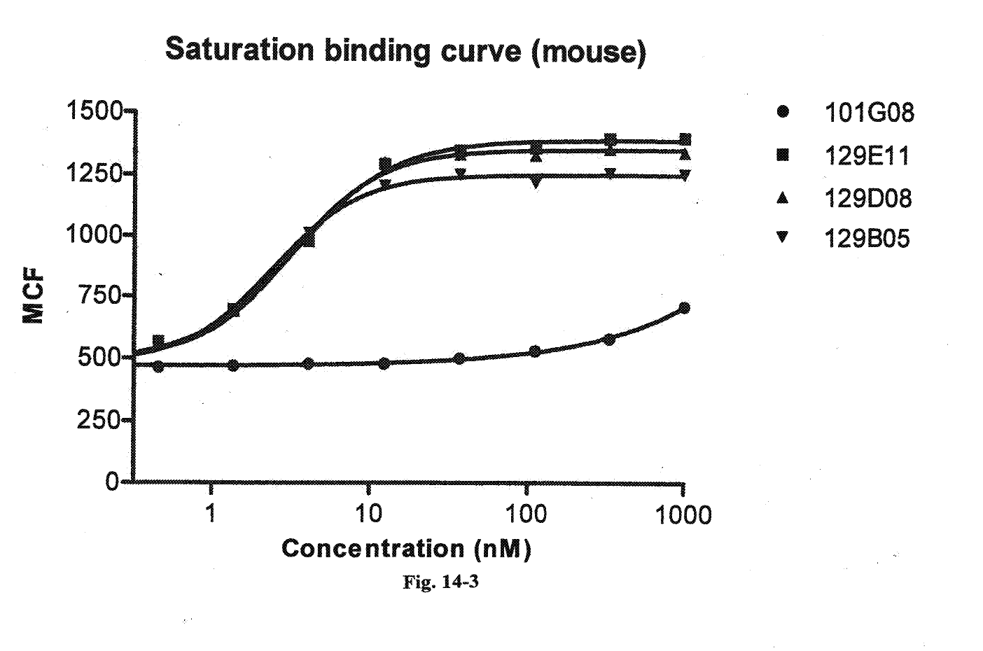

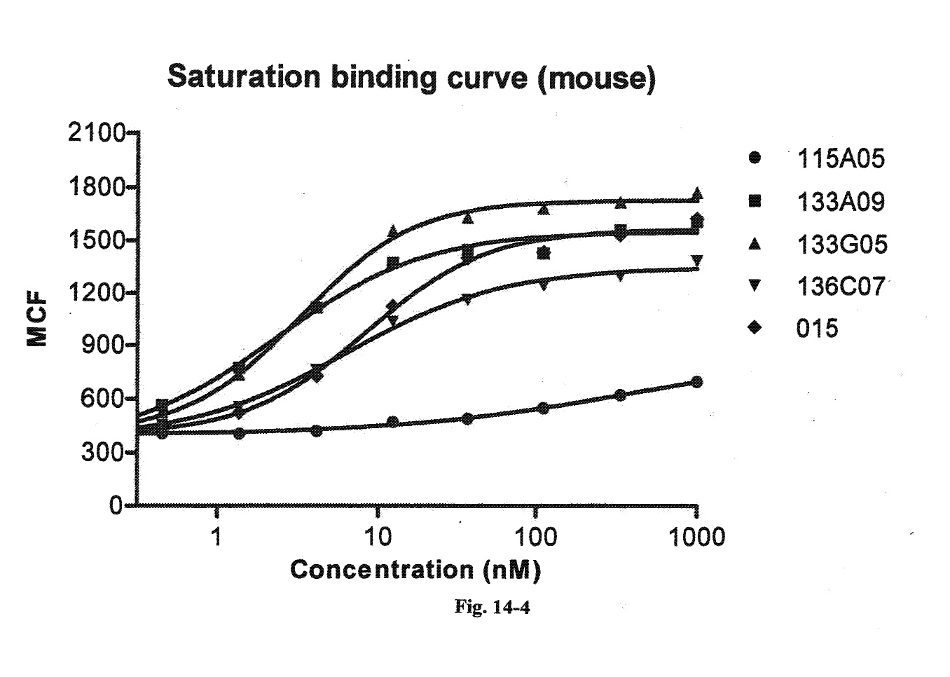

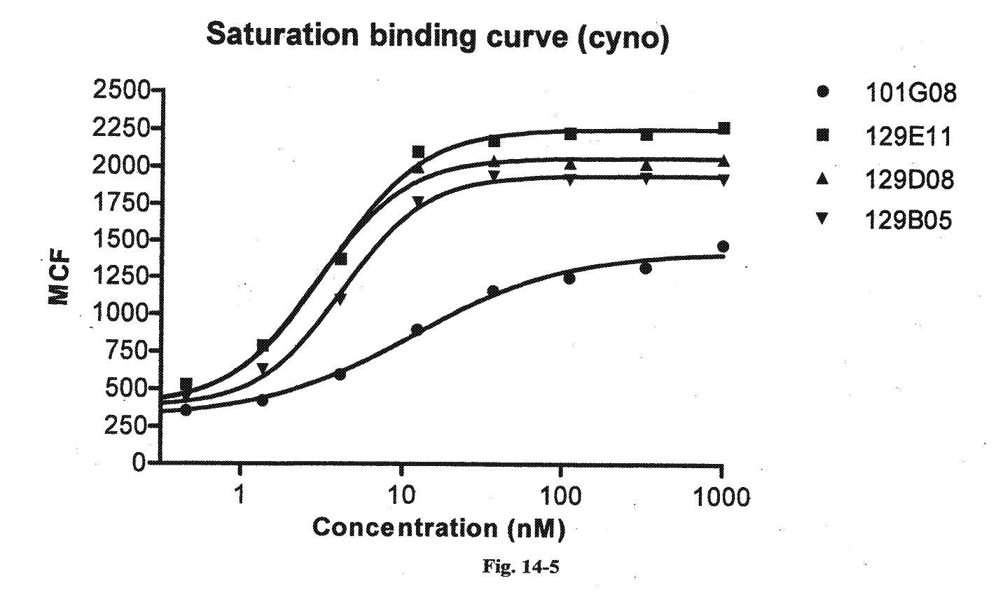

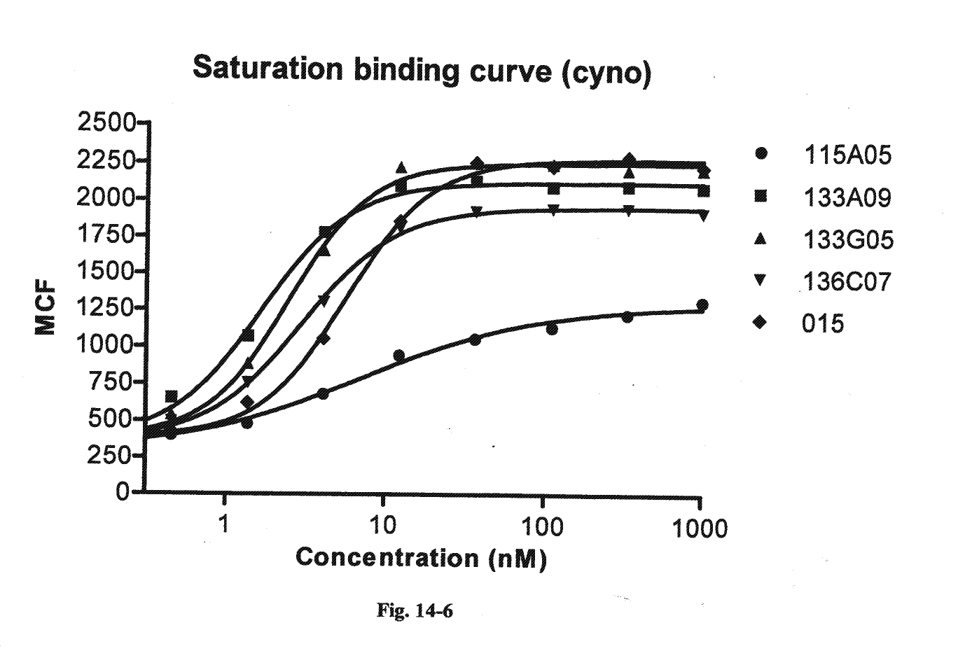

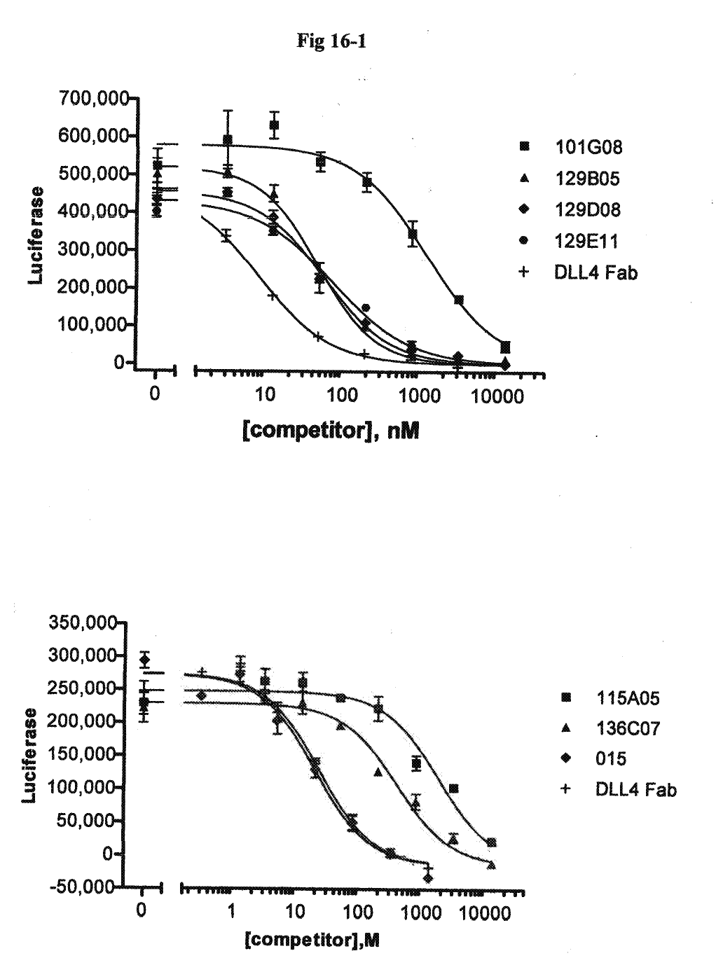

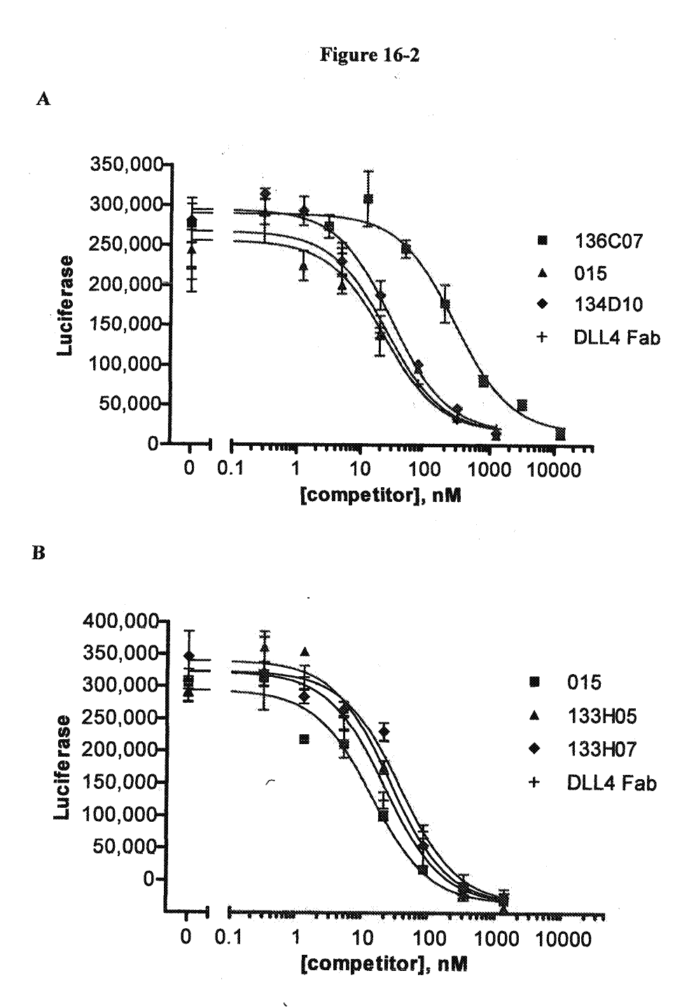

Affinity-matured variants of VHHs DLLBII101G08 and DLLBII115A05 are expressed and purified as described above (Example 5). VHHs are characterized in the hDll4—hNotch1 competition ELISA (Example 5.1; Table 17; FIG. 10), the CHO-hDll4/hNotch1-Fc and CHO-mDll4/hNotch1-Fc competition FMAT (Example 5.3; Table 18; FIG. 11), the hDLL1 and hJAG1 binding ELISA and hDll4/mDll4/cynoDll4 FACS (Example 5.8; Table 19; FIGS. 13 and 14, Table 20), determination of binding affinity on hDLL4 and mDLL4 in Biacore (Example 5.7; Table 19, FIG. 12) and the DLL4-mediated reporter assay (Example 5.4; Table 21; FIG. 16).

Characterization data are summarized in Table 22. Overall, the affinity-matured VHHs show clear improvements in affinity and potency, while their binding to mDll4 and cyno Dll4 is maintained and no binding to hDLL1 or hJAG1 is observed.

TABLE 17
IC₅₀ (nM) values for affinity-matured VHHs in hDLL4/hNotch1-Fc
competition ELISA

| VHH ID | IC₅₀ (nM) |
|---|---|
| 101G08 | 10.0 |
| 129A03 | 1.8 |
| 129B05 | 0.9 |
| 129D08 | 1.2 |
| 129E11 | 1.3 |
| 129H07 | 1.0 |
| 130B03 | 1.5 |
| 130F06 | 1.3 |
| anti-DLL4 Fab | 1.5 |
| 115A05 | 7.5 |
| 133A05 | 2.1 |
| 133A09 | 1.5 |
| 133G05 | 2.0 |
| 134D10 | 1.3 |
| 136C07 | 1.4 |
| 015 | 0.9 |
| anti-DLL4 Fab | 1.2 |

TABLE 18
IC₅₀ values (nM) of purified affinity-matured VHHs blocking the
interaction of human Notch1/Fc to human or mouse DLL4
expressed on CHO cells (FMAT)

| | hDLL4 | mDLL4 |
|---|---|---|
| | IC₅₀ | IC₅₀ |
| VHH ID | (nM) | (nM) |
| 101G08 | 69.3 | 140.5 |
| (wt) | | |
| 129B05 | 7.4 | 14.4 |
| 129D08 | 7.8 | 11.0 |
| 129E11 | 8.1 | 12.3 |
| DLL4 Fab | 5.5 | 3.0 |
| 115A05 | 106.7 | 348.9 |
| (wt) | | |
| 133A09 | 6.6 | 18.6 |
| 133G05 | 5.9 | 12.0 |
| 136C07 | 8.0 | 31.2 |
| 015 | 5.7 | 21.2 |
| DLL4 Fab | 3.4 | 1.6 |

TABLE 19
Affinity K_{D} (nM) of purified affinity-matured
VHHs on recombinant human DLL4 and mouse DLL4

| | rhDLL4 | | | rmDLL4 | | |
|---|---|---|---|---|---|---|
| | kₐ | k_{d} | K_{D} | kₐ | k_{d} | K_{D} |
| VHH ID | (M⁻¹ s⁻¹) | (s⁻¹) | (nM) | (M⁻¹ s⁻¹) | (s⁻¹) | (nM) |
| 101G08 | 4.8E+04 | 2.3E−03 | 48.0 | 9.4E+04 | 5.6E−03 | 60.0 |
| (wt) | | | | | | |
| 129A03 | 2.1E+05 | 1.2E−04 | 0.5 | | | |
| 129B05 | 2.3E+05 | 7.9E−05 | 0.3 | 2.7E+05 | 3.1E−04 | 1.1 |
| 129D08 | 1.8E+05 | 6.4E−05 | 0.4 | 2.7E+05 | 2.0E−04 | 0.8 |
| 129E11 | 1.9E+05 | 9.0E−05 | 0.5 | 2.5E+05 | 2.9E−04 | 1.2 |
| 129H07 | 1.6E+05 | 7.3E−05 | 0.5 | | | |
| 130B03 | 2.2E+05 | 6.8E−05 | 0.3 | | | |
| 130F06 | 2.0E+05 | 8.0E−05 | 0.4 | | | |
| anti- | 2.3E+05 | 3.4E−04 | 1.5 | | | |
| DLL4 | | | | | | |
| Fab | | | | | | |
| 115A05 | 2.5E+05 | 4.0E−03 | 16.0 | 1.7E+05 | 9.1E−03 | 53.0 |
| (wt) | | | | | | |
| 133A09 | 4.4E+05 | 9.0E−04 | 2.1 | 3.5E+05 | 2.7E−03 | 7.8 |
| 133G05 | 5.9E+05 | 4.7E−04 | 0.8 | 4.7E+05 | 1.6E−03 | 3.4 |
| 136C07 | 6.2E+05 | 3.9E−04 | 0.6 | 5.0E+05 | 1.3E−03 | 2.6 |
| 015 | 4.5E+05 | 4.7E−04 | 1.0 | 3.5E+05 | 1.5E−03 | 4.3 |
| anti- | 2.3E+05 | 3.4E−04 | 1.5 | | | |
| DLL4 | | | | | | |
| Fab | | | | | | |

TABLE 20
EC₅₀ (nM) values of affinity-matured VHHs for binding on CHO-
hDLL4, CHO-mDLL4 and CHO-cDLL4 (FACS)

| | hDLL4 | mDLL4 | cDLL4 |
|---|---|---|---|
| VHH ID | EC₅₀ (nM) | EC₅₀ (nM) | EC₅₀ (nM) |
| 101G08(wt) | 17.5 | | 11.2 |
| 129B05 | 9.7 | 3.9 | 3.9 |
| 129D08 | 9.6 | 3.7 | 3.8 |
| 129E11 | 1.4 | 4.1 | 4.2 |
| anti-DLL4 Fab | 5.6 | 2.1 | 2.5 |
| 115A05(wt) | 11.3 | | 13.8 |
| 133A09 | 7.2 | 1.7 | 2.3 |
| 133G05 | 8.5 | 2.8 | 2.7 |
| 136C07 | 10.9 | 8.3 | 3.5 |
| 015 | 14.8 | 7.0 | 5.1 |
| anti-DLL4 Fab | 5.6 | 2.1 | 2.5 |

TABLE 21
IC₅₀ (nM) values of affinity-matured VHHs in DLL4-mediated
reporter assay

| VHH ID | IC₅₀ (nM) |
|---|---|
| 101G08 | 1940 |
| (wt) | |
| 129B05 | 60 |
| 129D08 | 77 |
| 129E11 | 98 |
| DLL4 Fab | 16 |
| 115A05 | 1340 |
| (wt) | |
| 133A09 | 87 |
| 133G05 | 104 |
| 133H05 | 25 |
| 133H07 | 35 |
| 134D10 | 18 |
| 136C07 | 226 |
| 015 | 18 |
| DLL4 Fab | 16 |

TABLE 22
Characteristics of affinity-matured VHHs derived from DLLBII101G08 and DLLBII115A05

| | | | | FMAT | FMAT | | | | | |
|---|---|---|---|---|---|---|---|---|---|---|
| | K_{D} | | ELISA | hDLL4 | mDLL4 | FACS | FACS | FACS | | |
| | (nM) | K_{D} (nM) | IC₅₀ | IC₅₀ | IC₅₀ | EC₅₀ | EC₅₀ | EC₅₀ | ELISA | ELISA |
| | hDLL4 | mDLL4 | (nM) | (nM) | (nM) | (nM) | (nM) | (nM) | hDLL1 | hJag-1 |
| 101G08 | 48.0 | 60.0 | 10.0 | 69.3 | 140.5 | 17.5 | NF | 11.2 | nb | nb |
| 129A03 | 0.5 | | 1.8 | | | | | | | |
| 129B05 | 0.3 | 1.1 | 0.9 | 7.4 | 14.4 | 9.7 | 3.9 | 3.9 | nb | nb |
| 129D08 | 0.4 | 0.8 | 1.2 | 7.8 | 11.0 | 9.6 | 3.7 | 3.8 | nb | nb |
| 129E11 | 0.5 | 1.2 | 1.3 | 8.1 | 12.3 | 10.4 | 4.1 | 4.2 | nb | nb |
| 129H07 | 0.5 | | 1.0 | | | | | | | |
| 130B03 | 0.3 | | 1.5 | | | | | | | |
| 130F06 | 0.4 | | 1.3 | | | | | | | |
| DLL4 | 1.5 | | 1.5 | 5.5 | 3.0 | 5.6 | 2.1 | 2.5 | | |
| Fab | | | | | | | | | | |
| 115A05 | 16.0 | 53.0 | 7.5 | 106.7 | 348.9 | 11.3 | NF | 13.8 | nb | nb |
| 133A05 | | | 2.1 | | | | | | | |
| 133A09 | 2.1 | 7.8 | 1.5 | 6.6 | 18.6 | 7.2 | 1.7 | 2.3 | nb | nb |
| 133G05 | 0.8 | 3.4 | 2.0 | 5.9 | 12.0 | 8.5 | 2.8 | 2.7 | nb | nb |
| 134D10 | | | 1.3 | | | | | | | |
| 136C07 | 0.6 | 2.6 | 1.4 | 8.0 | 31.2 | 10.9 | 8.3 | 3.5 | nb | nb |
| 015 | 1.0 | 4.3 | 0.9 | 5.7 | 21.2 | 14.8 | 7.0 | 5.1 | nb | nb |
| DLL4 | 1.5 | | 1.2 | 3.4 | 1.6 | 5.6 | 2.1 | 2.5 | | |
| Fab | | | | | | | | | | |

nb: no binding

### EXAMPLE 8

Sequence Optimization of VHH DLLBII129B05 and DLLBII136C07

The amino acid sequence of DLLBII129B05 (FIG. 17-A) and DLLBII136C07 (FIG. 17-B) is aligned to the human germline VH3/JH consensus sequence. Residues are numbered according to Kabat, CDRs are shown in grey according to AbM definition (Oxford Molecular's AbM antibody modelling software).

Residues to be mutated to their human counterpart are underlined.

The alignment shows that DLLBII129B05 contains 4 framework mutations relative to the reference germline sequence. Non-human residues at positions 14, 64, 83 and 108 are selected for substitution with their human germline counterparts. A set of 2 DLLBII129B05 variants (DLLBII017 and DLLBII018) carrying different combinations of human residues on these positions is constructed and produced (Example 6; AA sequences are listed in Table 23).

For DLLBII136C07, the VHH contains 4 framework mutations relative to the reference germline sequence. Non-human residues at positions 39, 40, 83 and 108 are selected for substitution with their human germline counterparts. A set of 4 DLLBII136C07 variants (DLLBII019, DLLBII020, DLLBII021, DLLBII022) is generated carrying different combinations of human residues at these positions (Example 6; AA sequences are listed in Table 24). In parallel, a potential Asn deamidation site at position N52-S52a (CDR2 region, see FIG. 17-B boxed residues) is removed by introducing a N52S mutation. In a second cycle, tolerated mutations from the humanization effort and the N52S substitution are combined, resulting in sequence-optimized variant DLLBII036. One additional sequence-optimized variant (DLLBII039) is constructed including a F291 mutation in CDR1, which is shown to increase the potency of DLLBII136C07 in the DLL4-mediated reporter assay (Table 21; FIG. 16). Sequences of both sequence-optimized variants of DLLBII136C07 are listed in Table 25.

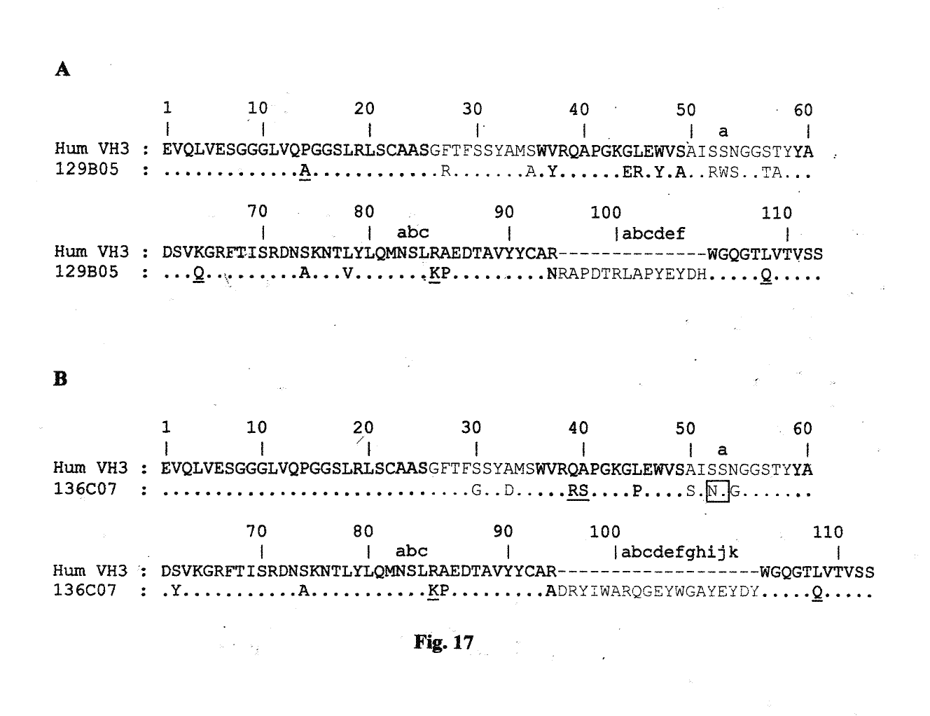

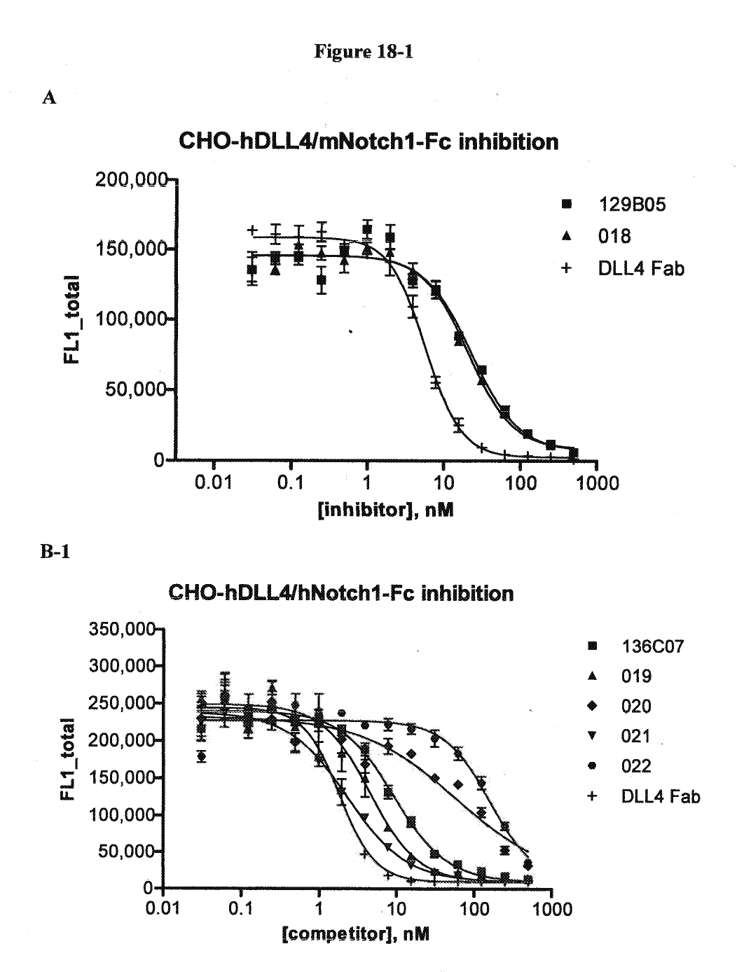

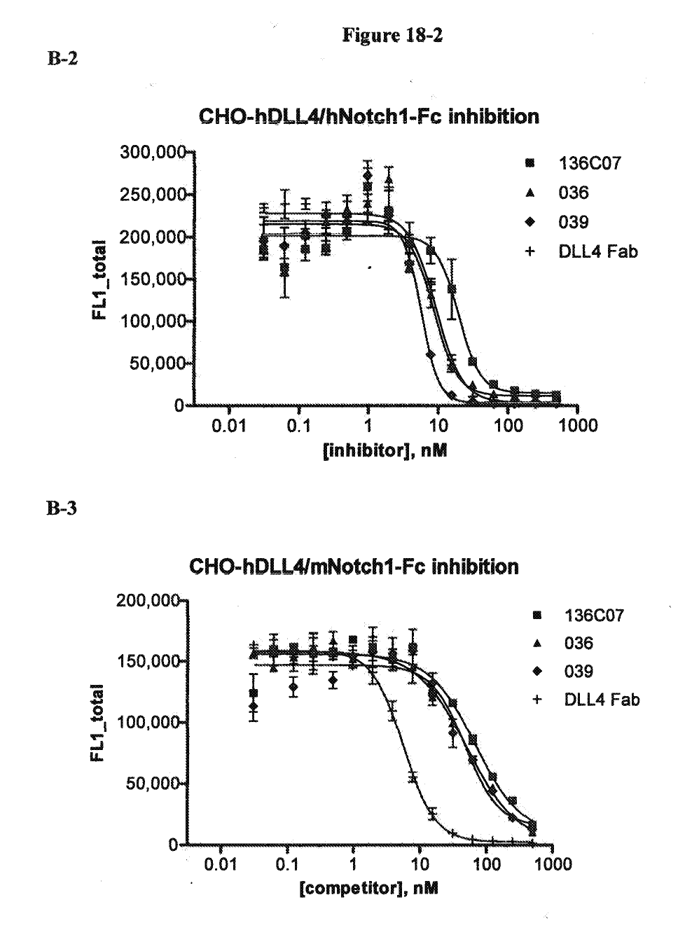

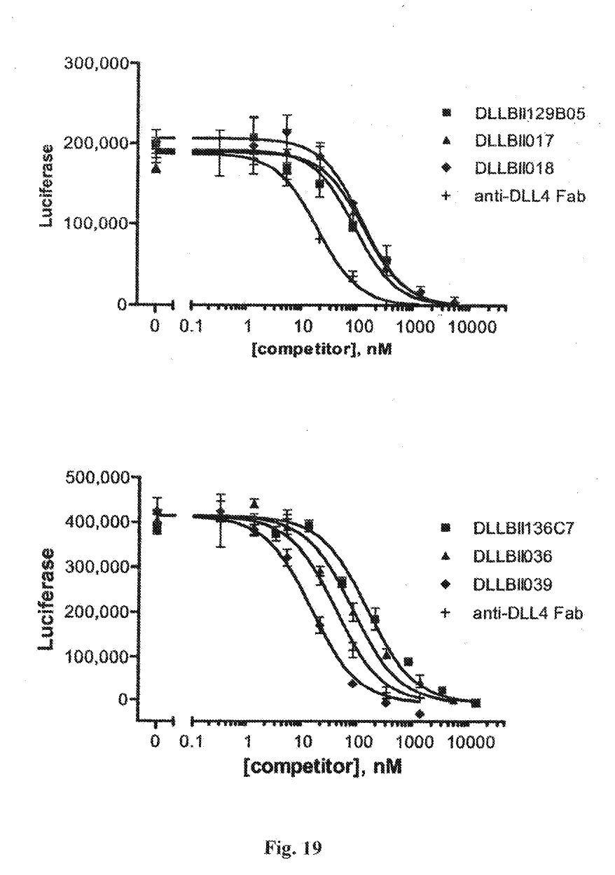

All these variants are characterized as purified protein in the CHO-hDLL4/hNotch1-Fc and CHO-mDLL4/hNotch1-Fc competitive FMAT assay (example 5.3; Table 26; FIG. 18), the DLL4 mediated reporter assay (example 5.4; Table 27; FIG. 19), the DLL4 HUVEC proliferation assay (example 5.9; Table 28) and in Biacore for affinity determination (example 5.7; Table 29). Additionally, the melting temperature (Tₘ) of each clone is determined in a thermal shift assay, which is based on the increase in fluorescence signal upon incorporation of Sypro Orange (Invitrogen) (Ericsson et al, Anal. Biochem. 357 (2006), pp 289-298). All variants displayed similar Tₘ values when compared to the parental DLLBII129B05. Table 30 summarizes Tₘ values at pH 7 for these clones.

TABLE 23

Sequence IDs and AA sequences of monovalent sequence-
optimized anti-DLL4 VHHs of parental DLLBII129BO5
(FR, framework; CDR, complementary determining region)

| VHH ID | | | | | | | |
|---|---|---|---|---|---|---|---|
| SEQ ID | | | | | | | |
| NO | FR1 | CDR1 | FR2 | CDR2 | FR3 | CDR3 | FR4 |
| DLLBII017 | EVQLVES | SYAMA | WYRQA | AIRWSGG | RFTISRDNA | RAPDTRLAP | WGQGTLVT |
| 34 | GGGLVQP | | PGKER | TAYYADS | KNTVYLQMN | YEYDH | VSS |
| | GGSLRLS | | EYVA | VQG | SLRPEDTAV | | |
| | CAASGRT | | | | YYCAN | | |
| | FS | | | | | | |
| DLLBII018 | EVQLVES | SYAMA | WYRQA | AIRWSGG | RFTISRDNA | RAPDTRLAP | WGQGTLVT |
| 35 | GGGLVQP | | PGKER | TAYYADS | KNTVYLQMN | YEYDH | VSS |
| | GGSLRLS | | EYVA | VKG | SLRPEDTAV | | |
| | CAASGRT | | | | YYCAN | | |
| | FS | | | | | | |

TABLE 24

Sequence IDs and AA sequences of monovalent sequence-
optimized anti-DLL4 VHHs of parental DLLBII136C07 (cycle 1)
(FR, framework; CDR, complementary determining region)

| VHH ID | | | | | | | |
|---|---|---|---|---|---|---|---|
| SEQ ID | | | | | | | |
| NO | FR1 | CDR1 | FR2 | CDR2 | FR3 | CDR3 | FR4 |
| DLLBII019 | EVQLVESGGG | SYDMS | WVRRSP | SINSGGG | RFTISRDNA | DRYIWARQG | WGQGTLVT |
| 36 | LVQPGGSLRL | | GKGPEW | STYYADY | KNTLYLQMN | EYWGAYEYD | VSS |
| | SCAASGFTFG | | VS | VKG | SLRPEDTAV | Y | |
| | | | | | YYCAA | | |
| DLLBII020 | EVQLVESGGG | SYDMS | WVRQSP | SINSGGG | RFTISRDNA | DRYIWARQG | WGQGTLVT |
| 37 | LVQPGGSLRL | | GKGPEW | STYYADY | KNTLYLQMN | EYWGAYEYD | VSS |
| | SCAASGFTFG | | VS | VKG | SLRPEDTAV | Y | |
| | | | | | YYCAA | | |
| DLLBII021 | EVQLVESGGG | SYDMS | WVRRAP | SINSGGG | RFTISRDNA | DRYIWARQG | WGQGTLVT |
| 38 | LVQPGGSLRL | | GKGPEW | STYYADY | KNTLYLQMN | EYWGAYEYD | VSS |
| | SCAASGFTFG | | VS | VKG | SLRPEDTAV | Y | |
| | | | | | YYCAA | | |
| DLLBII022 | EVQLVESGGG | SYDMS | WVRQAP | SINSGGG | RFTISRDNA | DRYIWARQG | WGQGTLVT |
| 39 | LVQPGGSLRL | | GKGPEW | STYYADY | KNTLYLQMN | EYWGAYEYD | VSS |
| | SCAASGFTFG | | VS | VKG | SLRPEDTAV | Y | |
| | | | | | YYCAA | | |

TABLE 25

Sequence IDs and AA sequences monovalent sequence-
optimized anti-DLL4 VHHs of parental DLLBII136C07
(cycle 2) (FR, framework; CDR, complementary
determining region)

| VHH ID | | | | | | | |
|---|---|---|---|---|---|---|---|
| SEQ ID | | | | | | | |
| NO | FR1 | CDR1 | FR2 | CDR2 | FR3 | CDR3 | FR4 |
| DLLBII036 | EVQLVESG | SYDMS | WVRRAPG | SISSG | RFTISRDN | DRYIWAR | WGQG |
| 40 | GGLVQPGG | | KGPEWVS | GGSTY | AKNTLYLQ | QGEYWGA | TLVT |
| | SLRLSCAA | | | YADYV | MNSLRPED | YEYDY | VSS |
| | SGFTFG | | | KG | TAVYYCAA | | |
| DLLBII039 | EVQLVESG | SYDMS | WVRRAPG | SISSG | RFTISRDN | DRYIWAR | WGQG |
| 41 | GGLVQPGG | | KGPEWVS | GGSTY | AKNTLYLQ | QGEYWGA | TLVT |
| | SLRLSCAA | | | YADYV | MNSLRPED | YEYDY | VSS |
| | SGFTIG | | | KG | TAVYYCAA | | |

TABLE 26
IC₅₀ (nM) values of sequence-optimized VHHs CHO-hDLL4 and
CHIO-mDLL4 competition FMAT

| | hDLL4 | mDLL4 |
|---|---|---|
| VHH ID | IC₅₀ (nM) | IC₅₀ (nM) |
| 129B05 | 8.2 | 15.9 |
| 017 | 12.1 | n/d |
| 018 | 11.0 | 15.4 |
| DLL4 Fab | 5.8 | 4.3 |
| 136C07 | 11.4 | 50.8 |
| 019 | 3.0 | n/d |
| 020 | 44.6 | n/d |
| 021 | 2.1 | n/d |
| 022 | 95.4 | n/d |
| 036 | 9.7 | 44.7 |
| 039 | 6.2 | 43.8 |
| DLL4 Fab | 5.4 | 4.3 |

n/d, not determined

TABLE 27
IC₅₀ (nM) values of sequence-optimized VHHs in DLL4-mediated
reporter assay

| | hDLL4 |
|---|---|
| VHH ID | IC₅₀ (nM) |
| 129B05 | 108 |
| 017 | 126 |
| 018 | 136 |
| DLL4 Fab | 23 |
| 136C07 | 112 |
| 019 | n/d |
| 020 | n/d |
| 021 | n/d |
| 022 | n/d |
| 036 | 78 |
| 039 | 16 |
| DLL4 Fab | 24 |

n/d, not determined

TABLE 28
IC₅₀ (nM) values of sequence-optimized VHHs in DLL4-mediated
HUVEC proliferation assay

| | | Inhibition |
|---|---|---|
| VHH ID | IC₅₀ (nM) | (%) |
| 129B05 | 3.7 | 100 |
| 018 | 5.3 | 100 |
| DLL4 Fab | 4.7 | 100 |
| 136C07 | 14.5 | 100 |
| 036 | 7.6 | 100 |
| 039 | 14.4 | 100 |
| DLL4 Fab | 4.7 | 100 |

TABLE 29
Affinity of sequence-optimized VHHs (Biacore) (for reference, DLL4
Fab has an affinity of 1.5 nM)

| VHH ID | kₐ (M⁻¹s⁻¹) | k_{d} (s⁻¹) | K_{D} (nM) |
|---|---|---|---|
| 129B05 | 3.4E+05 | 7.9E−05 | 0.2 |
| 017 | 3.7E+05 | 8.0E−05 | 0.2 |
| 018 | 4.5E+05 | 9.4E−05 | 0.2 |
| 136C07 | 5.5E+05 | 5.2E−04 | 1.0 |
| 019 | 5.7E+05 | 7.4E−04 | 1.3 |
| 020 | 3.4E+05 | 9.3E−03 | 27 |
| 021 | 5.6E+05 | 5.7E−04 | 1.0 |
| 022 | 4.7E+05 | 2.2E−02 | 46 |
| 036 | 6.6E+05 | 5.5E−04 | 0.8 |
| 039 | 4.5E+05 | 8.1E−04 | 1.8 |

TABLE 30
Tₘ values (° C.) at pH7 of sequence-optimized VHHs

| VHH ID | Tm (° C.) |
|---|---|
| 129B05 | 67.3 |
| 017 | 68.1 |
| 018 | 71.0 |
| 136C07 | 68.1 |
| 019 | 69.0 |
| 020 | 69.0 |
| 021 | 69.0 |
| 022 | 70.3 |
| 036 | 71.4 |
| 039 | 69.4 |

### EXAMPLE 9

Immunization with Different VEGF Formats Induces a Humoral Immune Response in Llama

9.1 Immunizations

After approval of the Ethical Committee of the faculty of Veterinary Medicine (University Ghent, Belgium), 4 llamas (designated No. 264, 265, 266, 267) are immunized according to standard protocols with 6 intramuscular injections (100 or 50 μg/dose at weekly intervals) of recombinant human VEGF109. The first injection at day 0 is formulated in Complete Freund's Adjuvant (Difco, Detroit, Mich., USA), while the subsequent injections are formulated in Incomplete Freund's Adjuvant (Difco, Detroit, Mich., USA). In addition, four llamas (designated No. 234, 235, 280 and 281) are immunized according to the following protocol: 5 intramuscular injections with KLH-conjugated human VEGH165 (100 or 50 μg/dose at biweekly intervals) followed by 4 intramuscular injections of human VEGF109 (first dose of 100 μg, followed 2 weeks later with three 50 μg/dose at weekly interval).

9.2 Evaluation of VEGF-Induced Immune Responses in Llama

To monitor VEGF specific serum titers, an ELISA assay is set up in which 2 μg/mL of recombinant human VEGF165 or VEGF109 is immobilized overnight at 4° C. in a 96-well MaxiSorp plate (Nunc, Wiesbaden, Germany). Wells are blocked with a casein solution (1%). After addition of serum dilutions, bound total IgG is detected using horseradish peroxidase (HRP)-conjugated goat anti-llama immunoglobulin (Bethyl Laboratories Inc., Montgomery, Tex., USA) and a subsequent enzymatic reaction in the presence of the substrate TMB (3,3′,5,5′-tetramentylbenzidine) (Pierce, Rockford, Ill., USA). For llamas 264, 265, 266 and 267 an additional ELISA is performed in which the isotype specific responses against VEGF165 and VEGF109 are evaluated. Isotype specific responses are detected using mouse mAbs specifically recognizing conventional llama IgG1 and the heavy-chain only llama IgG2 and IgG3 [Daley et al. (2005). Clin. Diagn. Lab. Imm. 12:380-386] followed by a rabbit anti-mouse-HRP conjugate (DAKO). ELISAs are developed using TMB as chromogenic substrate and absorbance is measured at 450 nm. The serum titers for each llama are depicted in Table 31.

TABLE 31
Antibody-mediated specific serum response against VEGF165 and
VEGF109. ELISA (solid phase coated with recombinant protein)

| | | Recombinant human VEGF165 | | | | Recombinant human VEGF109 | | | |
|---|---|---|---|---|---|---|---|---|---|
| | | Total | | | | Total | | | |
| Llama | Immunogen | IgG | IgG1 | IgG2 | IgG3 | IgG | IgG1 | IgG2 | IgG3 |
| 234 | VEGF165-KLH + | ++ | n/d | n/d | n/d | ++ | n/d | n/d | n/d |
| | VEGF109 | | | | | | | | |
| 235 | VEGF165-KLH + | ++ | n/d | n/d | n/d | ++ | n/d | n/d | n/d |
| | VEGF109 | | | | | | | | |
| 280 | VEGF165-KLH + | + | n/d | n/d | n/d | + | n/d | n/d | n/d |
| | VEGF109 | | | | | | | | |
| 281 | VEGF165-KLH + | + | n/d | n/d | n/d | + | n/d | n/d | n/d |
| | VEGF109 | | | | | | | | |
| 264 | VEGF109 | n/d | ++ | + | + | ++ | ++ | + | + |
| 265 | VEGF109 | n/d | ++ | + | + | + | ++ | + | + |
| 266 | VEGF109 | n/d | ++ | + | +/− | ++ | ++ | + | +/− |
| 267 | VEGF109 | n/d | +/− | − | − | +/− | +/− | − | − |

n/d, not determined

### EXAMPLE 10

Selection of VEGF-Specific VHHs Via Phage Display

The cloning of the heavy-chain only antibody fragment repertoires and preparation of phage is performed as described in Example 2. VHH phage libraries are used in different selection strategies applying a multiplicity of selection conditions. Variables include i) the VEGF protein format (rhVEGF165, rhVEGF109 or rmVEGF164), ii) the antigen presentation method (solid phase: directly coated or via a biotin-tag onto Neutravidin-coated plates; solution phase: incubation in solution followed by capturing on Neutravidin-coated plates), iii) the antigen concentration and iv) the elution method (trypsin or competitive elution using VEGFR2). All selections are carried out in Maxisorp 96-well plates (Nunc, Wiesbaden, Germany).

Selections are performed as follows: Phage libraries are incubated at RT with variable concentrations of VEGF antigen, either in solution or immobilized on a solid support. After 2 hrs of incubation and extensive washing, bound phage are eluted. In case trypsin is used for phage elution, the protease activity is immediately neutralized by addition of 0.8 mM protease inhibitor AEBSF. Phage outputs that show enrichment over background are used to infect E. coli. Infected E. coli cells are either used to prepare phage for the next selection round (phage rescue) or plated on agar plates (LB+amp+glucose^{2%}) for analysis of individual VHH clones. In order to screen a selection output for specific binders, single colonies are picked from the agar plates and grown in 1 mL 96-deep-well plates. The lacZ-controlled VHH expression is induced by adding IPTG (0.1-1 mM final). Periplasmic extracts (in a volume of ˜80 uL) are prepared according to standard methods.

### EXAMPLE 11

Identification of VEGF-Binding (Non-Receptor Blocking) and VEGF-Blocking (Receptor-Blocking) VHHs

Periplasmic extracts are tested for binding to human VEGF165 by ELISA. In brief, 2 μg/mL of recombinant human VEGF165 is immobilized overnight at 4° C. in a 96-well MaxiSorp plate (Nunc, Wiesbaden, Germany). Wells are blocked with a casein solution (1%). After addition of typically a 10-fold dilution of the periplasmic extracts, VHH binding is detected using a mouse anti-myc (Roche) and an anti-mouse-HRP conjugate (DAKO). Clones showing ELISA signals of >3-fold above background are considered as VEGF binding VHHs.

In addition, periplasmic extracts are screened in a human VEGF165/human VEGFR2 AlphaScreen assay to assess the blocking capacity of the VHHs. Human VEGF165 is biotinylated using Sulfo-NHS-LC-Biotin (Pierce, Rockford, Ill., USA). Human VEGFR2/Fc chimera (R&D Systems, Minneapolis, Minn., USA) is captured using an anti-humanFc VHH which is coupled to acceptor beads according to the manufacturer's instructions (Perkin Elmer, Waltham, Mass., US). To evaluate the neutralizing capacity of the VHHs, periplasmic extracts are diluted 1/25 in PBS buffer containing 0.03% Tween 20 (Sigma-Aldrich) and preincubated with 0.4 nM biotinylated human VEGF165 for 15 minutes at room temperature (RT). To this mixture the acceptor beads (10 μg/ml) and 0.4 nM VEGFR2-huFc are added and further incubated for 1 hour at RT in the dark. Subsequently donor beads (10 μg/ml) are added followed by incubation of 1 hour at RT in the dark. Fluorescence is measured by reading plates on the Envision Multi label Plate reader (Perkin Elmer, Waltham, Mass., USA) using an excitation wavelength of 680 nm and an emission wavelength between 520 nm and 620 nm. Periplasmic extract containing irrelevant VHH is used as negative control. Periplasmic extracts containing anti-VEGF165 VHHs which are able to decrease the fluorescence signal with more than 60% relative to the signal of the negative control are identified as a hit. All hits identified in the AlphaScreen are confirmed in a competition ELISA. To this end, 1 μg/mL of human VEGFR2 chimera (R&D Systems, Minneapolis, Minn., USA) is coated in a 96-well MaxiSorp plate (Nunc, Wiesbaden, Germany). Fivefold dilutions of the periplasmic extracts are incubated in the presence of a fixed concentration (4 nM) of biotinylated human VEGF165 in PBS buffer containing 0.1% casein and 0.05% Tween 20 (Sigma-Aldrich). Binding of these VHH/bio-VEGF165 complexes to the human VEGFR2 chimera coated plate is detected using horseradish peroxidase (HRP) conjugated extravidin (Sigma, St Louis, Mo., USA). VHH sequence IDs and the corresponding AA sequences of inhibitory (receptor-blocking) VHHs and VEGF-binding (non-receptor-blocking) VHHs selected for further characterization are listed in Table 32 and Table 33, respectively.

TABLE 32

Sequence IDs and AA sequences of monovalent
receptor-blocking anti-VEGF VHHs selected for
further characterization (FR, framework; CDR,
complementary determining region)

| VHH ID/ | | | | | | | |
|---|---|---|---|---|---|---|---|
| SEQ ID | | | | | | | |
| NO: | FR1 | CDR1 | FR2 | CDR2 | FR3 | CDR3 | FR4 |
| VEGFBII23A06 | EVQLVES | SYSMG | WFR | AISSG | RFTISRDN | SRAYGSSR | WGQGT |
| 42 | GGGLVQP | | QAQ | GFIYD | TKNTVYLQ | LRLADTYD | QVTVS |
| | GDSLKLS | | GKE | AVSLE | TPSLKPED | Y | S |
| | CAFSGRT | | REF | G | TAVYYCAA | | |
| | FS | | VV | | | | |
| VEGFBII23B04 | EVQLVES | SYSMG | WFR | AISKG | RFTISKDN | SRAYGSSR | WGQGT |
| 43 | GGGLVQT | | QAQ | GYKYD | AKNTVYLQ | LRLADTYE | QVTVS |
| | GDSLRLS | | GKE | SVSLE | INSLKPED | Y | S |
| | CEVSGRT | | REF | G | TAVYYCAS | | |
| | FS | | VV | | | | |
| VEGFBII24C04 | EVQLVES | SYSMG | WFR | AISSG | RFTISRDN | SRAYGSSR | WGQGT |
| 44 | GGGLVQP | | QAQ | GYIYD | TKNTVYLQ | LRLADTYD | QVTVS |
| | GDSLKLS | | GKE | SVSLQ | TPSLKPED | Y | S |
| | CVASGRT | | REF | G | TAVYYCAA | | |
| | SS | | VV | | | | |

TABLE 33

Sequence IDs and AA sequences of monovalent non-receptor-
blocking anti-VEGF VHHs selected for further
characterization (FR, framework; CDR, complementary
determining region)

| VHH ID/ | | | | | | | |
|---|---|---|---|---|---|---|---|
| SEQ ID | | | | | | | |
| NO: | FR1 | CDR1 | FR2 | CDR2 | FR3 | CDR3 | FR4 |
| VEGFBII05B05 | EVQLVE | SMA | WYRQA | RISSGGT | RFTISRDNS | FSSRPNP | WGAGT |
| 45 | SGGGLV | | PGKHR | TAYVDSV | KNTVYLQMN | | QVTVS |
| | QPGGSL | | ELVA | KG | SLKAEDTAV | | S |
| | RLSCVA | | | | YYCNT | | |
| | SGIRFM | | | | | | |
| VEGFBII10E07 | EVQLVE | NYAM | WFRQA | DISSSGI | RFTISRDNA | SAWWYSQM | WGQGT |
| 46 | SGGGLV | G | PGKER | NTYVADA | KNTVYLQMN | ARDNYRY | QVTVS |
| | QAGGSL | | VLVA | VKG | SLKPEDTAV | | S |
| | RLSCAA | | | | YYCAA | | |
| | SGRTFS | | | | | | |
| VEGFBII86H09 | EVQLVE | SYRM | WFRRT | SISWTYG | RFTMSRDKA | GAQSDRYN | WGQGT |
| 47 | SGGGLV | G | PGKED | STFYADS | KNAGYLQMN | IRSYDY | QVTVS |
| | QAGGSL | | EFVA | VKG | SLKPEDTAL | | S |
| | RLSCTA | | | | YYCAA | | |
| | SGSAFK | | | | | | |

Dissociation rates of receptor-blocking VHHs are analyzed on Biacore (Biacore T100 instrument, GE Healthcare). HBS-EP+ buffer is used as running buffer and experiments are performed at 25° C. Recombinant human VEGF165 is irreversibly captured on a CM5 sensor chip via amine coupling (using EDC and NHS) up to a target level of +/−1500 RU. After immobilization, surfaces are deactivated with 10 min injection of 1M ethanolamine pH8.5. A reference surface is activated and deactivated with respectively EDC/NHS and ethanolamine. Periplasmic extracts of VHHs are injected at a 10-fold dilution in running buffer for 2 min at 45 μl/min and allowed to dissociate for 10 or 15 min. Between different samples, the surfaces are regenerated with regeneration buffer. Data are double referenced by subtraction of the curves on the reference channel and of a blank running buffer injection. The dissociation phase of the processed curves is evaluated by fitting a two phase decay model in the Biacore T100 Evaluation software v2.0.1. Values for k_{d}-fast, k_{d}-slow and % fast are listed in Table 34.

TABLE 34
Off-rate determination of receptor-blocking VHHs with Biacore

| | | | | Binding level |
|---|---|---|---|---|
| VHH ID | k_{d} (fast) | k_{d} (slow) | % fast | (RU) |
| VEGFBII23B04 | 8.80E−03 | 4.00E−05 | 12 | 768 |
| VEGFBII24C04 | 1.30E−02 | 3.40E−05 | 17 | 456 |
| VEGFBII23A06 | 1.70E−02 | 3.70E−05 | 13 | 547 |

### EXAMPLE 12

Characterization of Purified Anti-VEGF VHHs

Three inhibitory anti-VEGF VHHs are selected for further characterization as purified proteins: VEGFBII23B04, VEGFBII24C04 and VEGFBII23A06. These VHHs are expressed in E. coli TG1 as c-myc, His6-tagged proteins. Expression is induced by addition of 1 mM IPTG and allowed to continue for 4 hours at 37° C. After spinning the cell cultures, periplasmic extracts are prepared by freeze-thawing the pellets. These extracts are used as starting material for VHH purification via IMAC and size exclusion chromatography (SEC). Final VHH preparations show 95% purity as assessed via SDS-PAGE.

12.1 Evaluation of Human VEGF165/VEGFR2 Blocking VHHs in Human VEGF165/Human VEGFR2-Fc Blocking ELISA

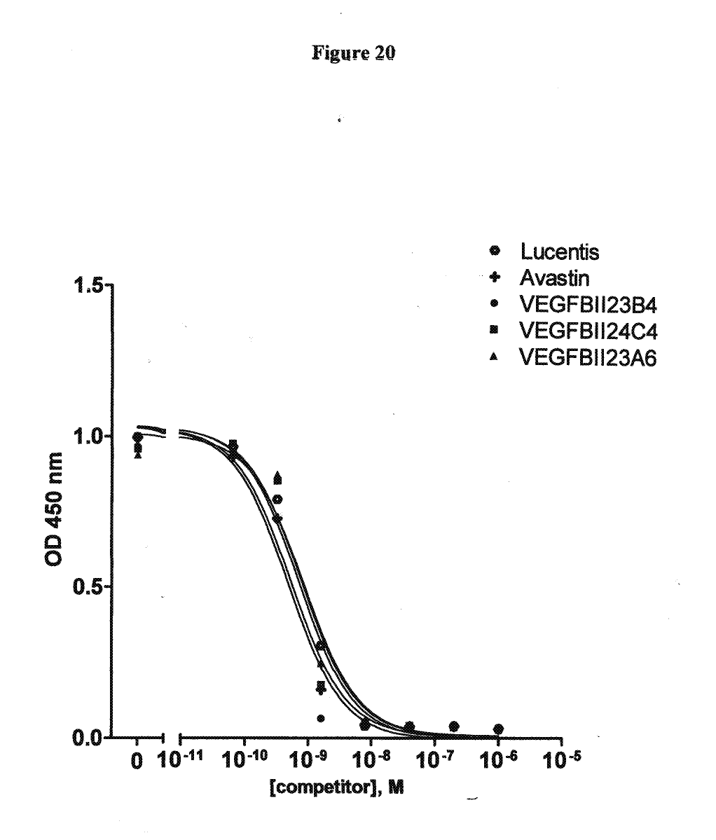

The blocking capacity of the VHHs is evaluated in a human VEGF165/human VEGFR2-Fc blocking ELISA. In brief, 1 μg/mL of VEGFR2-Fc chimera (R&D Systems, Minneapolis, Minn., USA) is coated in a 96-well MaxiSorp plate (Nunc, Wiesbaden, Germany). Dilution series (concentration range 1 mM-64 pM) of the purified VHHs in PBS buffer containing 0.1% casein and 0.05% Tween 20 (Sigma) are incubated in the presence of 4 nM biotinlyated VEGF165. Residual binding of bio-VEGF165 to VEGFR2 is detected using horseradish peroxidase (HRP) conjugated extravidin (Sigma, St Louis, Mo., USA) and TMB as substrate. As controls Bevacizumab (Avastin® and Ranibizumab (Lucentis) are taken along. Dose inhibition curves are shown in FIG. 20, the corresponding IC₅₀ values and % inhibition are summarized in Table 35.

TABLE 35
IC₅₀ (nM) values and % inhibition for monovalent VHHs in
hVEGF165/hVEGFR2-Fc competition ELISA

| | | % |
|---|---|---|
| VHH ID | IC₅₀ (nM) | inhibition |
| VEGFBII23B04 | 2.1 | 100 |
| VEGFBII23A06 | 3.0 | 100 |
| VEGFBII24C04 | 2.5 | 100 |
| Ranibizumab | 1.6 | 100 |
| Bevacizumab | 1.7 | 100 |

12.2 Evaluation of Human VEGF165/VEGFR2 Blocking VHHs in Human VEGF165/Human VEGFR1-Fc Blocking ELISA

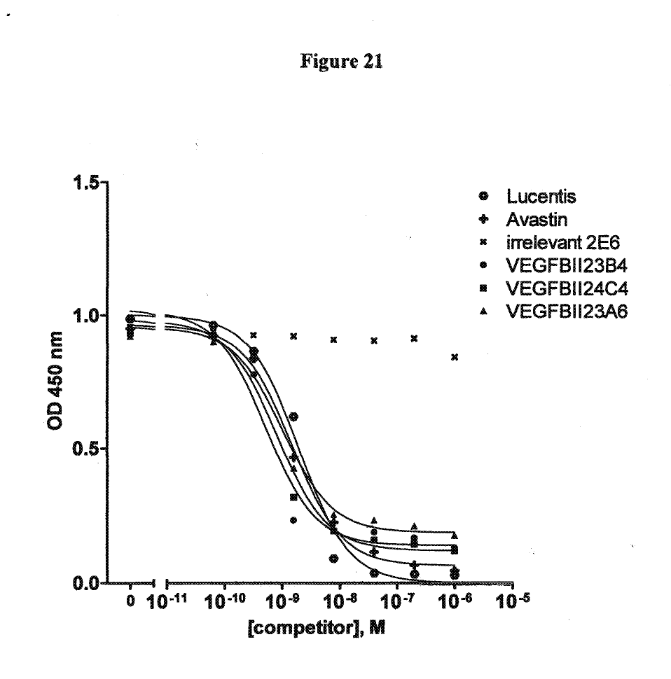

VHHs are also evaluated in a human VEGF165/human VEGFR1-Fc blocking ELISA. In brief, 2 μg/mL of VEGFR1-Fc chimera (R&D Systems, Minneapolis, Minn., USA) is coated in a 96-well MaxiSorp plate (Nunc, Wiesbaden, Germany). Dilution series (concentration range 1 mM-64 pM) of the purified VHHs in PBS buffer containing 0.1% casein and 0.05% Tween 20 (Sigma) are incubated in the presence of 0.5nM biotinlyated VEGF165. Residual binding of bio-VEGF165 to VEGFR1 is detected using horseradish peroxidase (HRP) conjugated extravidin (Sigma, St Louis, Mo., USA) and TMB as substrate. As controls Bevacizumab, Ranibizumab and an irrelevant VHH (2E6) are taken along. Dose inhibition curves are shown in FIG. 21, the corresponding IC₅₀ values and % inhibition are summarized in Table 36.

TABLE 36
IC₅₀ (nM) values and % inhibition of monovalent VHHs in
hVEGF165/hVEGFR1-Fc competition ELISA

| VHH ID | IC₅₀ (nM) | % inhibition |
|---|---|---|
| VEGFBII23B04 | 0.5 | 64 |
| VEGFBII23A06 | 0.9 | 55 |
| VEGFBII24C04 | 0.8 | 71 |
| Ranibizumab | 1.2 | 91 |
| Bevacizumab | 1.5 | 96 |

12.3 Evaluation of the Anti-VEGF165 VHHs in the Human VEGF165/Human VEGFR2-Fc Blocking AlphaScreen

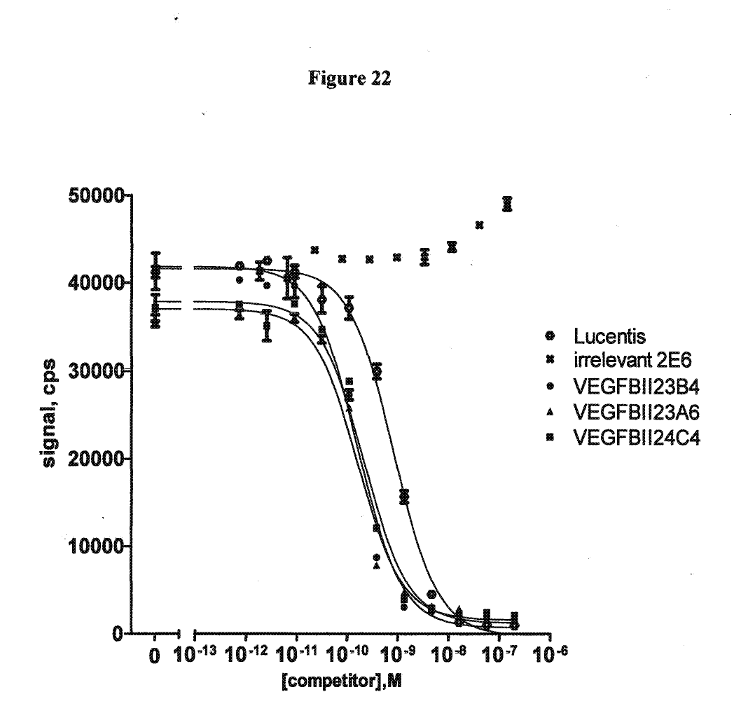

The blocking capacity of the VHHs is also evaluated in a human VEGF165/human VEGFR2-Fc blocking AlphaScreen. Briefly, serial dilutions of purified VHHs (concentration range: 200 nM-0.7 pM) in PBS buffer containing 0.03% Tween 20 (Sigma) are added to 4 pM bio-VEGF165 and incubated for 15 min. Subsequently VEGFR2-Fc (0.4 nM) and anti-Fc VHH-coated acceptor beads (20 μg/ml) are added and this mixture is incubated for 1 hour in the dark. Finally, streptavidin donor beads (20 μg/ml) are added and after 1 hour of incubation in the dark, fluorescence is measured on the Envision microplate reader. Dose-response curves are shown in the FIG. 22. The IC₅₀ values for VHHs blocking the human VEGF165-human VEGFR2-Fc interaction are summarized in Table 37.

TABLE 37
IC₅₀ (pM) values and % inhibition for VHHs in hVEGF165/hVEGFR2-
Fc competition AlphaScreen

| VHH ID | IC₅₀ (pM) | % inhibition |
|---|---|---|
| VEGFBII23B04 | 160 | 100 |
| VEGFBII23A06 | 250 | 100 |
| VEGFBII24C04 | 250 | 100 |
| Ranibizumab | 860 | 100 |

12.4 Evaluation of the Anti-VEGF165 VHHs in the Human VEGF165/Human VEGFR1-Fc Blocking AlphaScreen

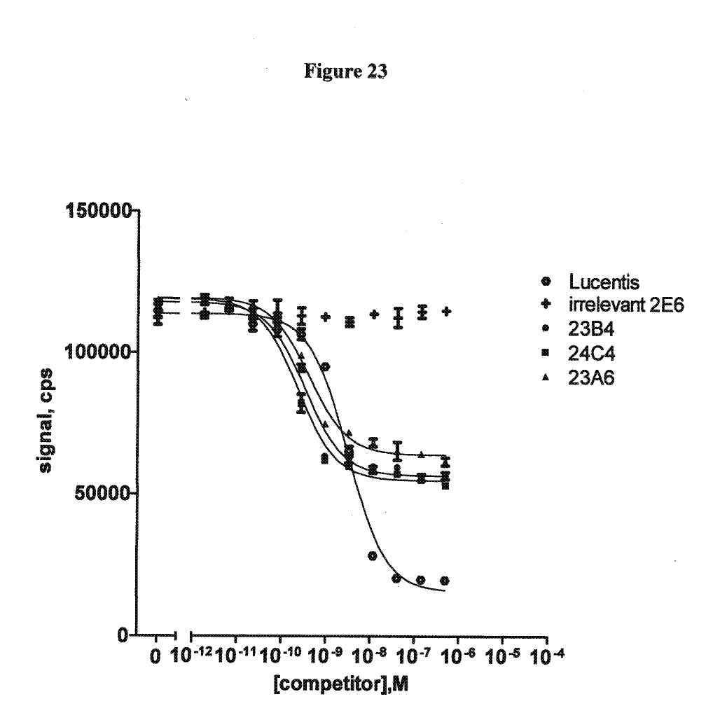

The blocking capacity of the VHHs is also evaluated in a human VEGF165/human VEGFR1-Fc blocking AlphaScreen. Briefly, serial dilutions of purified VHHs (concentration range: 500 nM-1.8 pM)) in PBS buffer containing 0.03% Tween 20 (Sigma) are added to 0.4 nM bio-VEGF165 and incubated for 15 min. Subsequently VEGFR1-Fc (1 nM) and anti-Fc VHH-coated acceptor beads (20 μg/ml) are added and this mixture is incubated for 1 hour in the dark. Finally, streptavidin donor beads (20 μg/ml) are added and after 1 hour of incubation in the dark, fluorescence is measured on the Envision microplate reader. Dose-response curves are shown in the FIG. 23. The IC₅₀ values and % inhibition for VHHs blocking the human VEGF165-human VEGFR1-Fc interaction are summarized in Table 38.

TABLE 38
IC₅₀ (nM) values and % inhibiton for VHHs in hVEGF165/hVEGFR1-
Fc competition AlphaScreen

| VHH ID | IC₅₀ (nM) | % inhibition |
|---|---|---|
| VEGFBII23B04 | 0.9 | 41 |
| VEGFBII23A06 | 0.4 | 46 |
| VEGFBII24C04 | 0.2 | 53 |
| Ranibizumab | 3.3 | 79 |

12.5 Determination of the Affinity of the Human VEGF165-VHH Interaction

Binding kinetics of VHH VEGFBII23B4 with hVEGF165 is analyzed by SPR on a Biacore T100 instrument. Recombinant human VEGF165 is immobilized directly on a CM5 chip via amine coupling (using EDC and NHS). VHHs are analyzed at different concentrations between 10 and 360 nM. Samples are injected for 2 min and allowed to dissociate up to 20 min at a flow rate of 45 μl/min. In between sample injections, the chip surface is regenerated with 100 mM HCl. HBS-EP+ (Hepes buffer pH7.4+EDTA) is used as running buffer. Binding curves are fitted using a Two State Reaction model by Biacore T100 Evaluation Software v2.0.1. The calculated affinities of the anti-VEGF VHHs are listed in Table 39.

TABLE 39
Affinity K_{D} (nM) of purified VHHs for recombinant human VEGF165

| | VEGF165 | | | | | | |
|---|---|---|---|---|---|---|---|
| | kₐ | kₐ₁ | kₐ₂ | k_{d} | k_{d1} | k_{d2} | K_{D} |
| VHH ID | (M⁻¹ · s⁻¹) | (M⁻¹ · s⁻¹) | (M⁻¹ · s⁻¹) | (s⁻¹) | (s⁻¹) | (s⁻¹) | (nM) |
| VEGFBII23B04^{(a)} | — | 2.1E+05 | 1.4E−02 | — | 8.6E−03 | 2.4E−04 | 0.7 |
| VEGFBII23A06^{(a)} | — | 4.2E+05 | 2.0E−02 | — | 5.7E−02 | 1.0E−04 | 0.7 |
| VEGFBII24C04^{(a)} | — | 3.2E+05 | 1.8E−02 | — | 2.6E−02 | 9.6E−05 | 0.4 |

^{(a)}Heterogeneous binding curve resulting in no 1:1 fit, curves are fitted using a Two State Reaction model by Biacore T100 Evaluation Software v2.0.1

12.6 Binding to Mouse VEGF164

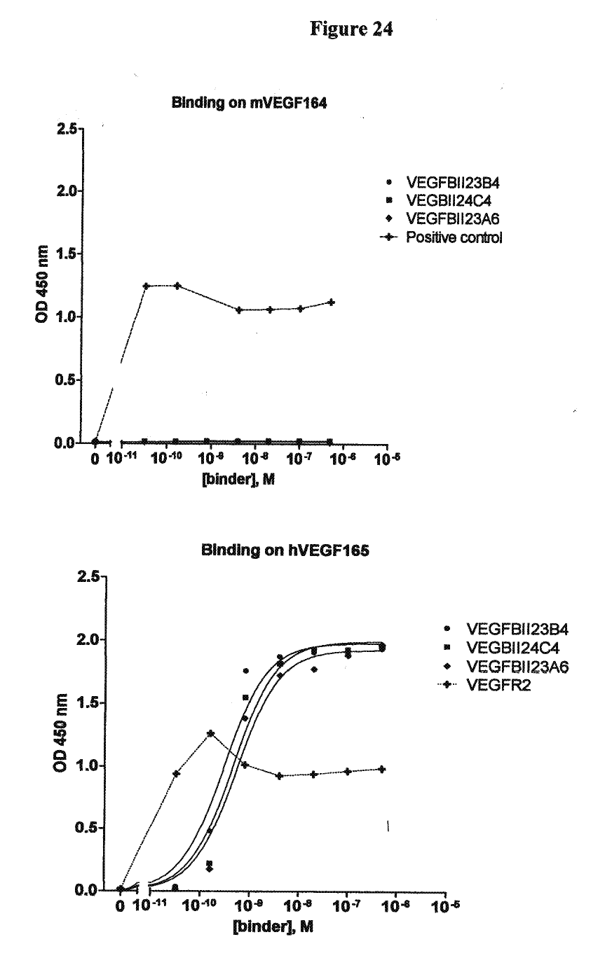

Cross-reactivity to mouse VEGF164 is determined using a binding ELISA. In brief, recombinant mouse VEGF164 (R&D Systems, Minneapolis, MS, USA) is coated overnight at 4° C. at 1 μg/mL in a 96-well MaxiSorp plate (Nunc, Wiesbaden, Germany). Wells are blocked with a casein solution (1% in PBS). VHHs are applied as dilution series (concentration range: 500nM-32 pM) in PBS buffer containing 0.1% casein and 0.05% Tween 20 (Sigma) and binding is detected using a mouse anti-myc (Roche) and an anti-mouse-HRP conjugate (DAKO) and a subsequent enzymatic reaction in the presence of the substrate TMB (3,3′,5,5′-tetramentylbenzidine) (Pierce, Rockford, Ill., USA) (FIG. 24). A mouse VEGF164 reactive mAb is included as positive control. As reference, binding to human VEGF165 is also measured. EC₅₀ values are summarized in Table 40.

TABLE 40
EC₅₀ (pM) values for VHHs in a recombinant human VEGF165 and
mouse 164 binding ELISA

| | rhVEGF165 | rmVEGF164 |
|---|---|---|
| VHH ID | EC₅₀ (pM) | EC₅₀ (pM) |
| VEGFBII23B04 | 297 | NB |
| VEGFBII24C04 | 453 | NB |
| VEGFBII23A06 | 531 | NB |

NB, no binding

12.7 Binding to VEGF121

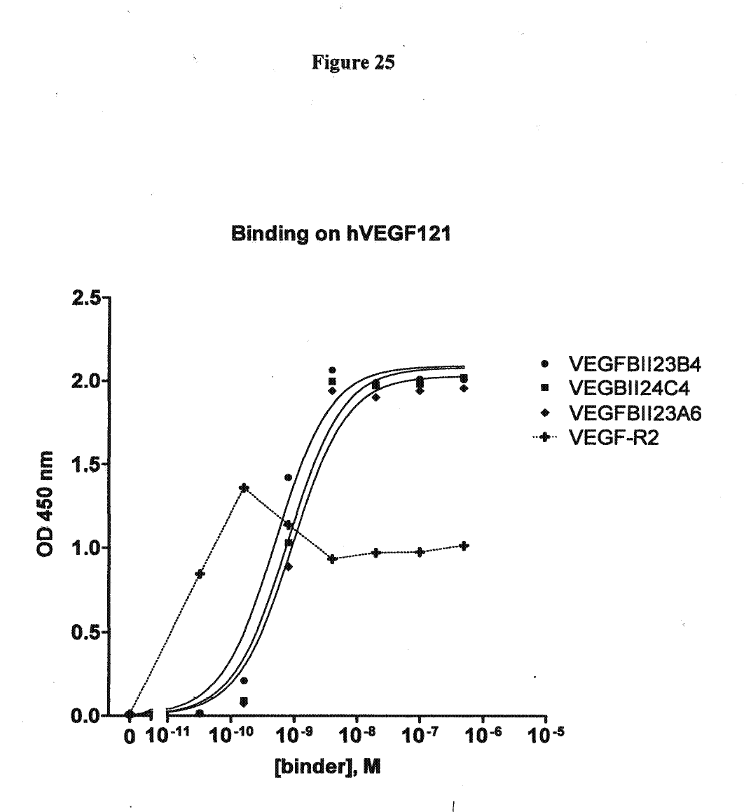

Binding to recombinant human VEGF121 is assessed via a solid phase binding ELISA. Briefly, recombinant human VEGF121 (R&D Systems, Minneapolis, MS, USA) is coated overnight at 4° C. at 1 μg/mL in a 96-well MaxiSorp plate (Nunc, Wiesbaden, Germany). Wells are blocked with a casein solution (1% in PBS). VHHs are applied as dilution series (concentration range: 500 nM-32 pM) in PBS buffer containing 0.1% casein and 0.05% Tween 20 (Sigma) and binding is detected using a mouse anti-myc (Roche) and an anti-mouse-HRP conjugate (DAKO) and a subsequent enzymatic reaction in the presence of the substrate TMB (3,3′,5,5′-tetramentylbenzidine) (Pierce, Rockford, Ill., USA) (FIG. 25). As positive control serial dilutions of the VEGFR2 is taken along. EC₅₀ values are summarized in Table 41.

TABLE 41
EC₅₀ (pM) values for monovalent VHHs in a recombinant human
VEGF121 binding ELISA

| VHH ID | EC₅₀ (pM) |
|---|---|
| VEGFBII23B04 | 510 |
| VEGFBII24C04 | 792 |
| VEGFBII23A06 | 928 |

12.8 Binding to VEGF Family Members VEGFB, VEGFC, VEGFD and PIGF

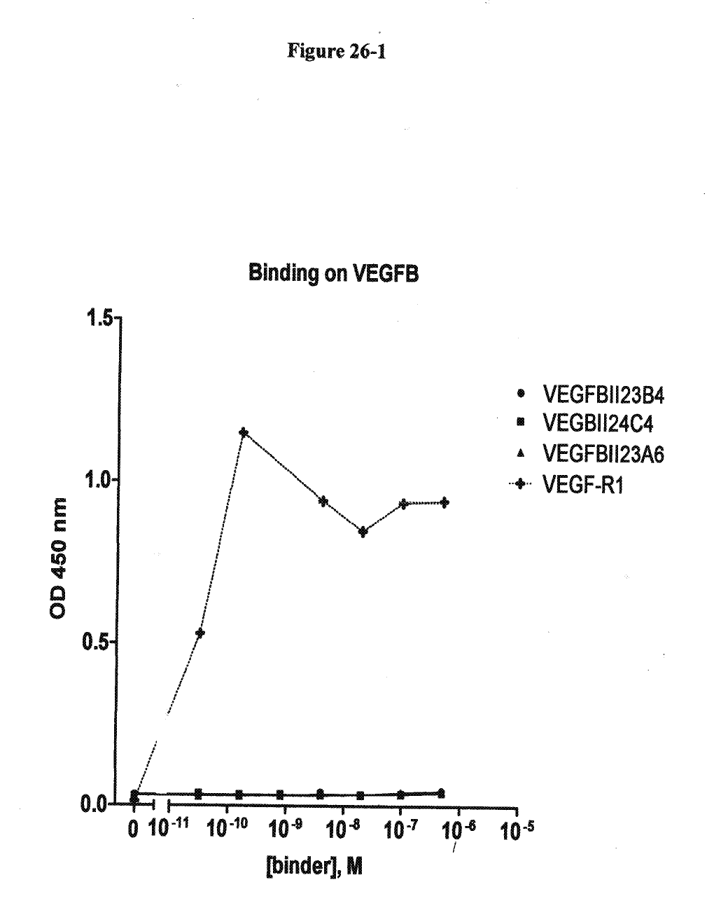

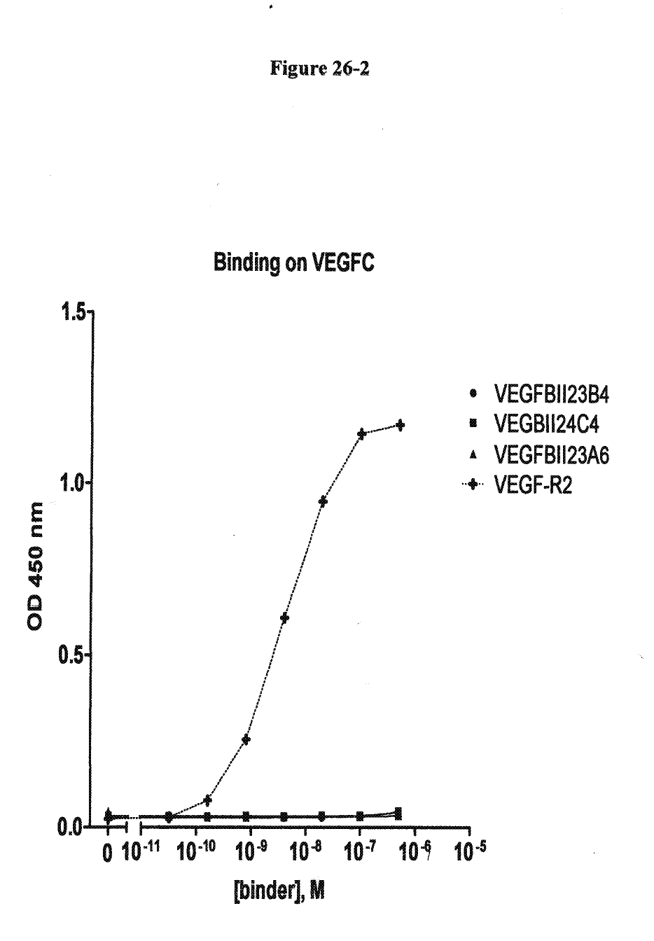

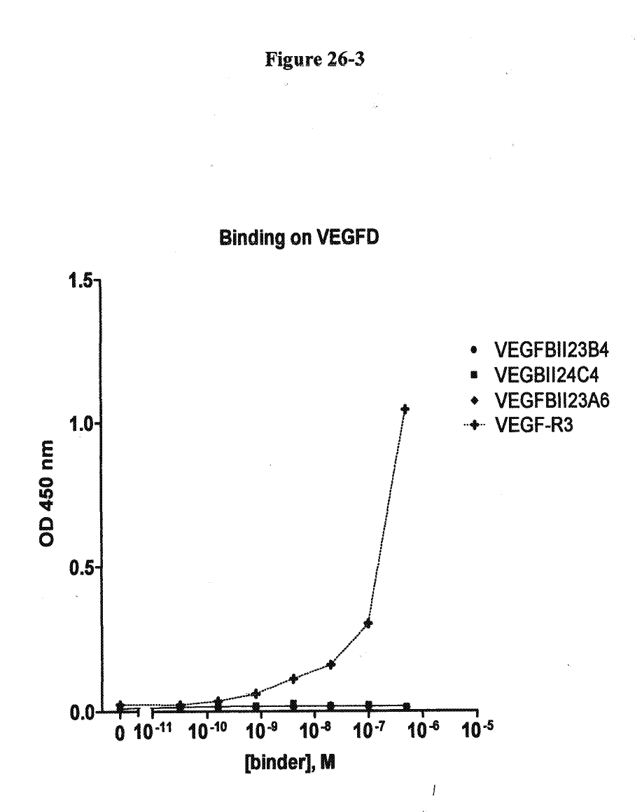

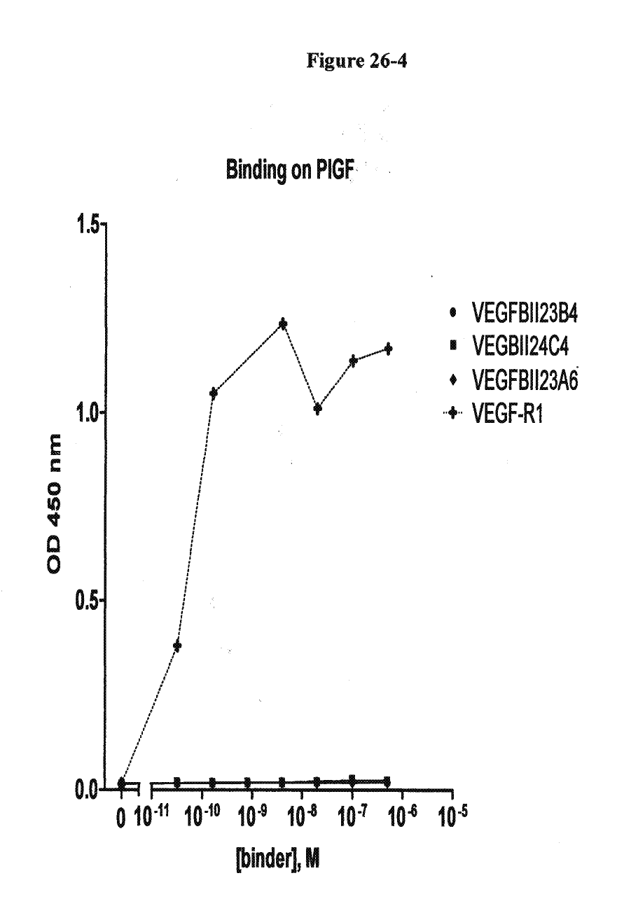

Binding to VEGFB, VEGFC, VEGFD and PIGF is assessed via a solid phase binding ELISA. In brief, VEGFB, VEGFC, VEGFD and PIGF (R&D Systems, Minneapolis, MS, USA) are coated overnight at 4° C. at 1 μg/mL in a 96-well MaxiSorp plate (Nunc, Wiesbaden, Germany). Wells are blocked with a casein solution (1% in PBS). VHHs are applied as dilution series (concentration range: 500 nM-32 pM) and binding is detected using a mouse anti-myc (Roche) and an anti-mouse-AP conjugate (Sigma, St Louis, Mo., USA). As positive controls serial dilutions of the appropriate receptors are taken along and detected with horseradish peroxidase (HRP)-conjugated goat anti-human IgG, Fc specific antibody (Jackson Immuno Research Laboratories Inc., West Grove, Pa., USA) and a subsequent enzymatic reaction in the presence of the substrate TMB (3,3′,5,5′-tetramentylbenzidine) (Pierce, Rockford, Ill., USA). Dose-response curves of VHHs and controls are shown in FIG. 26. The results show that there is no detectable binding of the selected VHHs to VEGFB, VEGFC, VEGFD or PIGF.

12.9 Epitope Binning

Biacore-based epitope binning experiments are performed to investigate which VEGF binders bind to a similar or overlapping epitope as VEGFBII23B04. To this end, VEGFBII23B04 is immobilized on a CM5 sensor chip. For each sample, human VEGF165 is passed over the chip surface and reversibly captured by VEGFBII23B4. Purified VHHs (100 nM) or periplasmic extracts (1/10 diluted) are then injected with a surface contact time of 240 seconds and a flow rate of 10 uL/minute. Between different samples, the surface is regenerated with regeneration buffer (100 mM HCl). Processed curves are evaluated with Biacore T100 Evaluation software. VHHs could be divided within two groups: group one which gave additional binding to VEGFBII23B04 captured VEGF165 and a second group which is not able to simultaneously bind to VEGFBII23B04 captured VEGF165 (the selected VHHs 24C04, 23A06 and 23B04 are in this group).

The same assay set-up is used to assess whether VEGFR1, VEGFR2, Ranibizumab and Bevacizumab are able to bind to human VEGF-165 simultaneously with VEGFBII23B04. Table 42 presents the additional binding responses to VEGFBII23B04 captured VEGF165. Only VEGFR2 is not able to bind to VEGFBII23B04 captured VEGF165, underscoring the blocking capacity of VEGFBII23B04 for the VEGF-VEGFR2 interaction. In addition, these data show that the VEGFBII23B04 epitope does not correspond to the Bevacizumab and Ranibizumab epitope.

TABLE 42
Epitope binding of VEGFBII23B04-binding of benchmark inhibitors
or cognate receptors to VEGFBII23B04-captured VEGF165

| | | | Binding |
|---|---|---|---|
| Injection | | | level |
| step | Binding | [sample] | (RU) |
| 1 | VEGF165 | 100 nM | 1727 |
| 2 | VEGFBII23B04 | 100 nM | — |
| 3 | Ranibizumab | 100 nM | 763 |
| 4 | Bevacizumab | 100 nM | 1349 |
| 5 | VEGFR1 | 100 nM | 1011 |
| 6 | VEGFR2 | 100 nM | — |

12.10 Characterization of the Anti-VEGF VHHs in the HUVEC Proliferation Assay

The potency of the selected VHHs is evaluated in a proliferation assay. In brief, primary HUVEC cells (Technoclone) are supplement-starved over night and then 4000 cells/well are seeded in quadruplicate in 96-well tissue culture plates. Cells are stimulated in the absence or presence of VHHs with 33 ng/mL VEGF. The proliferation rates are measured by [³H] Thymidine incorporation on day 4. The results of the HUVEC proliferation assay shown in Table 43 demonstrate that VEGFBII23B04 and Bevacizumab inhibit the VEGF-induced HUVEC proliferation by more than 90%, with IC50s<1 nM.

TABLE 43
IC₅₀ (nM) values and % inhibition of monovalent VEGFBII23B04,
VEGFBII23A06 and VEGFBII24C04 in the VEGF HUVEC proliferation
assay

| | | % |
|---|---|---|
| VHH ID | IC₅₀ (nM) | inhibition |
| VEGFBII23B04 | 0.36 | 91 |
| Bevacizumab | 0.21 | 92 |
| VEGFBII23A06 | 4.29 | 73 |
| VEGFBII24C04 | 3.8 | 79 |
| Bevacizumab | 0.78 | 78 |

12.11 Characterization of the Anti-VEGF VHHs in the HUVEC Erk Phosphorylation Assay

The potency of the selected VHHs is assessed in the HUVEC Erk phosphorylation assay. In brief, primary HUVEC cells are serum-starved over night and then stimulated in the absence or presence of VHHs with 10 ng/mL VEGF for 5 min. Cells are fixed with 4% Formaldehyde in PBS and ERK phosphorylation levels are measured by ELISA using phosphoERK-specific antibodies (anti-phosphoMAP Kinase pERK1&2, M8159, Sigma) and polyclonal Rabbit Anti-Mouse-Immunoglobulin-HRP conjugate (PO161, Dako). As shown in Table 44, VEGFBII23B4 and Bevacizumab inhibit the VEGF induced Erk phosphorylation by at least 90%, with IC₅₀s<1 nM.

TABLE 44
IC₅₀ (nM) values and % inhibition of monovalent VEGFBII23B04 in
VEGF HUVEC Erk phosphorylation assay

| | IC₅₀ | % |
|---|---|---|
| VHH ID | (nM) | inhibition |
| VEGFBII23B04 | 0.37 | 90 |
| Bevacizumab | 0.63 | 98 |

### EXAMPLE 13

Generation of Multivalent Anti-VEGF Blocking VHHs

VHH VEGFBII23B04 is genetically fused to either VEGFBII23B04 resulting in a homodimeric VHH or different VEGF-binding VHHs resulting in heterodimeric (bivalent) VHHs. To generate the bivalent VHHs, a panel of 10 unique VEGF-binding VHHs are linked via a 9 or 40 Gly-Ser flexible linker in two different orientations to VEGFBII23B04. Homodimeric VEGFBII23B04 (VEGFBII010) and the 40 heterodimeric bivalent VHHs are expressed in E. coli TG1 as c-myc, His6-tagged proteins. Expression is induced by addition of 1 mM IPTG and allowed to continue for 4 hours at 3TC. After spinning the cell cultures, periplasmic extracts are prepared by freeze-thawing the pellets. These extracts are used as starting material and VHHs are purified via IMAC and desalting resulting in 90% purity as assessed via SDS-PAGE. AA sequences the homodimeric and selected bivalent VEGF-binding VHHs are shown in SEQ ID NO: 48-53 and in Table 45.

TABLE 45
Sequence ID, VHH ID and AA sequence of selected bivalent anti-VEGF VHHs

| VHH ID/ | | |
|---|---|---|
| SEQ ID NO: | VHH ID | AA sequence |
| VEGFBII23B04- | VEGFBII010 | EVQLVESGGGLVQTGDSLRLSCEVSGRTFSSYSMGWFRQAQGKEREFVVAISKG |
| 35GS-23B04 | | GYKYDSVSLEGRFTISKDNAKNTVYLQINSLKPEDTAVYYCASSRAYGSSRLRLAD |
| 48 | | TYEYWGQGTQVTVSSGGGGSGGGGSGGGGSGGGGSGGGGSGGGGSGGGGS |
| | | EVQLVESGGGLVQTGDSLRLSCEVSGRTFSSYSMGWFRQAQGKEREFVVAISKG |
| | | GYKYDSVSLEGRFTISKDNAKNTVYLQINSLKPEDTAVYYCASSRAYGSSRLRLAD |
| | | TYEYWGQGTQVTVSS |
| VEGFBII23B04- | VEGFBII022 | EVQLVESGGGLVQTGDSLRLSCEVSGRTFSSYSMGWFRQAQGKEREFVVAISKG |
| 9GS-5B05 | | GYKYDSVSLEGRFTISKDNAKNTVYLQINSLKPEDTAVYYCASSRAYGSSRLRLAD |
| 49 | | TYEYWGQGTQVTVSSGGGGSGGGSEVQLVESGGGLVQPGGSLRLSCVASGIRF |
| | | MSMAWYRQAPGKHRELVARISSGGTTAYVDSVKGRFTISRDNSKNTVYLQMNSLK |
| | | AEDTAVYYCNTFSSRPNPWGAGTQVTVSS |
| VEGFBII23B04- | VEGFBII021 | EVQLVESGGGLVQTGDSLRLSCEVSGRTFSSYSMGWFRQAQGKEREFVVAISKG |
| 40GS-5B05 | | GYKYDSVSLEGRFTISKDNAKNTVYLQINSLKPEDTAVYYCASSRAYGSSRLRLAD |
| 50 | | TYEYWGQGTQVTVSSGGGGSGGGGSGGGGSGGGGSGGGGSGGGGSGGGGS |
| | | GGGGSEVQLVESGGGLVQPGGSLRLSCVASGIRFMSMAWYRQAPGKHRELVARI |
| | | SSGGTTAYVDSVKGRFTISRDNSKNTVYLQMNSLKAEDTAVYYCNTFSSRPNPWG |
| | | AGTQVTVSS |
| VEGFBII23B04- | VEGFBII023 | EVQLVESGGGLVQTGDSLRLSCEVSGRTFSSYSMGWFRQAQGKEREFVVAISKG |
| 40GS-10E07 | | GYKYDSVSLEGRFTISKDNAKNTVYLQINSLKPEDTAVYYCASSRAYGSSRLRLAD |
| 51 | | TYEYWGQGTQVTVSSGGGGSGGGGSGGGGSGGGGSGGGGSGGGGSGGGGS |
| | | GGGGSEVQLVESGGGLVQAGGSLRLSCAASGRTFSNYAMGWFRQAPGKERVLV |
| | | ADISSSGINTYVADAVKGRFTISRDNAKNTVYLQMNSLKPEDTAVYYCAASAWWYS |
| | | QMARDNYRYWGQGTQVTVSS |
| VEGFBII23B04- | VEGFBII024 | EVQLVESGGGLVQTGDSLRLSCEVSGRTFSSYSMGWFRQAQGKEREFVVAISKG |
| 40GS-86H09 | | GYKYDSVSLEGRFTISKDNAKNTVYLQINSLKPEDTAVYYCASSRAYGSSRLRLAD |
| 52 | | TYEYWGQGTQVTVSSGGGGSGGGGSGGGGSGGGGSGGGGSGGGGSGGGGS |
| | | GGGGSEVQLVESGGGLVQAGGSLRLSCTASGSAFKSYRMGWFRRTPGKEDEFV |
| | | ASISWTYGSTFYADSVKGRFTMSRDKAKNAGYLQMNSLKPEDTALYYCAAGAQSD |
| | | RYNIRSYDYWGQGTQVTVSS |
| VEGFBII10E07- | VEGFBII025 | EVQLVESGGGLVQAGGSLRLSCAASGRTFSNYAMGWFRQAPGKERVLVADISSS |
| 40GS-23B04 | | GINTYVADAVKGRFTISRDNAKNTVYLQMNSLKPEDTAVYYCAASAWWYSQMARD |
| 53 | | NYRYWGQGTQVTVSSGGGGSGGGGSGGGGSGGGGSGGGGSGGGGSGGGGS |
| | | GGGGSEVQLVESGGGLVQTGDSLRLSCEVSGRTFSSYSMGWFRQAQGKEREFV |
| | | VAISKGGYKYDSVSLEGRFTISKDNAKNTVYLQINSLKPEDTAVYYCASSRAYGSSR |
| | | LRLADTYEYWGQGTQVTVSS |

The panel of 40 bivalent VHHs is tested in the VEGFR2 and VEGFR1 blocking AlphaScreen assay, as described in Example 12.3 and 12.4, respectively. Based on potency and maximum level of inhibition, the best five bivalent VHHs (VEGFBII021, VEGFBII022, VEGFBI023, VEGFBI024 and VEGFBII025—see Table 45) are chosen for further characterization. An overview of the screening results for the selected five bivalent VHHs in the competitive VEGFR2 and VEGFR1 AlphaScreen is shown in Table 46.

TABLE 46
Potency and efficacy of the five selected bispecific bivalent VHHs in
the VEGF/VEGFR1 and VEGF/VEGFR2 competition AlphaScreen assay

| | |
|---|---|
| VEGFR2 | VEGFR1 |

| VHH ID | IC₅₀ (pM) | IC₅₀ (pM) | % inhibition |
|---|---|---|---|
| VEGFBII021 | 9 | 16 | 100 |
| VEGFBII022 | 7 | 8 | 100 |
| VEGFBII023 | 38 | 44 | 91 |
| VEGFBII024 | 12 | 46 | 100 |
| VEGFBII025 | 51 | 39 | 82 |

### EXAMPLE 14

Characterization of Formatted Anti-VEGFVHHs

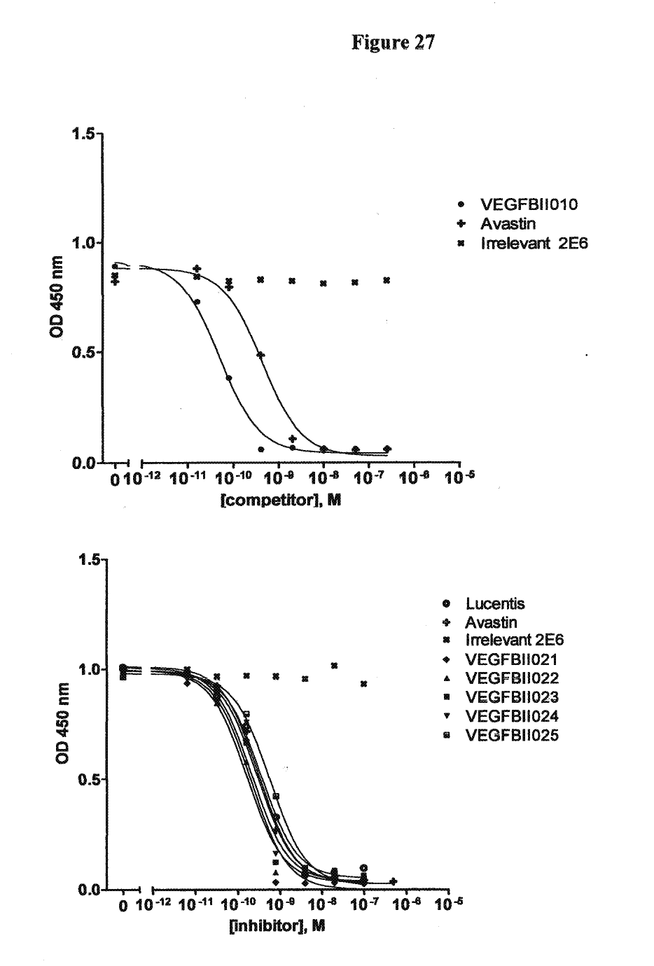

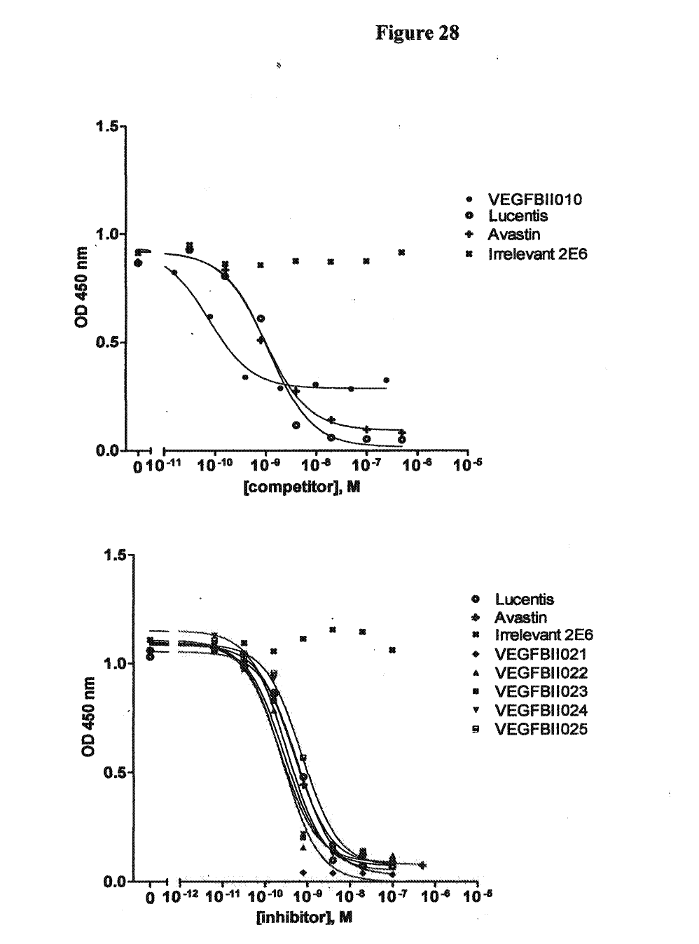

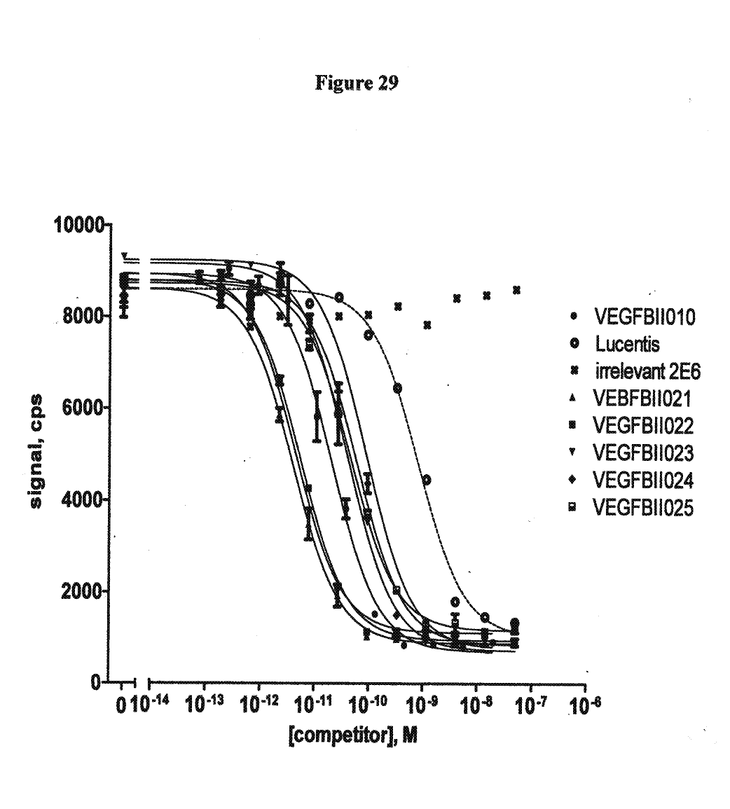

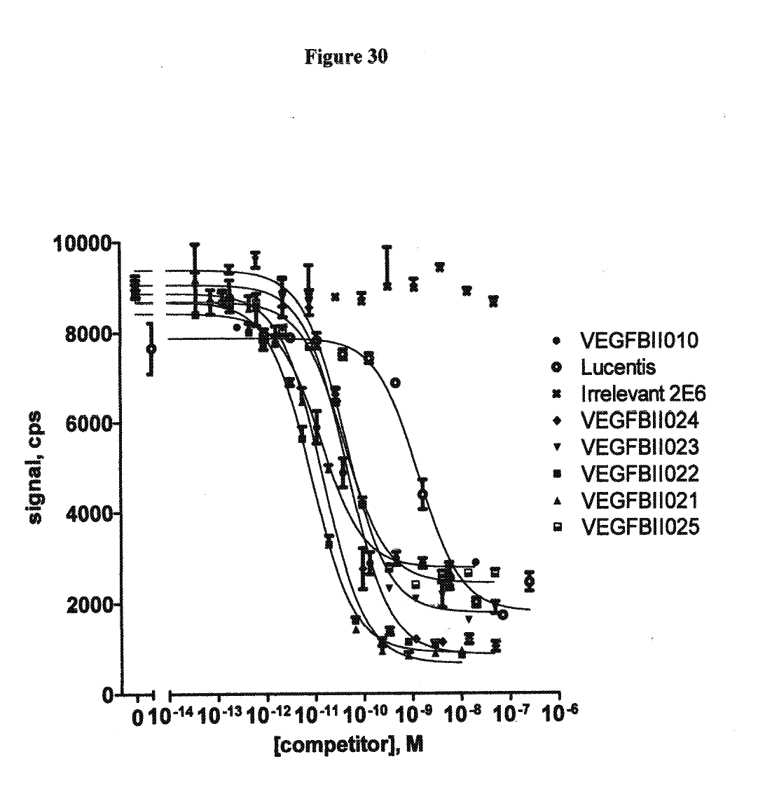

VHHs VEGFBII010, VEGFBII021, VEGFBII022, VEGFBII023, VEGFBII024 and VEGFBII025 are compared side-by side in the VEGFR2 and VEGFR1 blocking ELISA (FIGS. 27 and 28, Table 47 and Table 48 respectively) and AlphaScreen assay (FIGS. 29 and 30, Table 49 and 50) as described in Examples 12.1, 12.2, 12.3 and 12.4, respectively.

TABLE 47
IC₅₀ (pM) values and % inhibition for formatted VHHs in
hVEGF165/hVEGFR2-Fc competition ELISA

| | IC₅₀ | |
|---|---|---|
| VHH ID | (pM) | % inhibition |
| VEGFBII010 | 49 | 100 |
| VEGFBII021 | 204 | 100 |
| VEGFBII022 | 164 | 100 |
| VEGFBII023 | 213 | 100 |
| VEGFBII024 | 292 | 100 |
| VEGFBII025 | 577 | 100 |
| Bevacizumab | 315 | 100 |
| Ranibizumab | 349 | 100 |

TABLE 48
IC₅₀ (pM) values and % inhibition of formatted VHHs in
VEGF165/hVEGFR1-Fc competition ELISA

| | IC₅₀ | |
|---|---|---|
| VHH ID | (pM) | % inhibition |
| VEGFBII010 | 73.5 | 67 |
| VEGFBII021 | 254 | 97 |
| VEGFBII022 | 225 | 89 |
| VEGFBII023 | 279 | 91 |
| VEGFBII024 | 326 | 92 |
| VEGFBII025 | 735 | 91 |
| Bevacizumab | 484 | 91 |
| Ranibizumab | 594 | 96 |

TABLE 49
IC₅₀ (pM) values and % inhibition for formatted VHHs in
hVEGF165/hVEGFR2-Fc competition AlphaScreen

| VHH ID | IC₅₀ (pM) | % inhibition |
|---|---|---|
| VEGFBII010 | 16 | 100 |
| VEGFBII021 | 7 | 100 |
| VEGFBII022 | 7 | 100 |
| VEGFBII023 | 46 | 100 |
| VEGFBII024 | 50 | 100 |
| VEGFBII025 | 51 | 100 |
| Ranibizumab | 600 | 100 |

TABLE 50
IC₅₀ (pM) values and % inhibition of formatted VHHs in
VEGF165/hVEGFR1-Fc competition AlphaScreen

| VHH ID | IC₅₀ (pM) | % inhibition |
|---|---|---|
| VEGFBII010 | 21 | 70 |
| VEGFBII021 | 12 | 100 |
| VEGFBII022 | 9 | 98 |
| VEGFBII023 | 48 | 87 |
| VEGFBII024 | 69 | 98 |
| VEGFBII025 | 71 | 82 |
| Ranibizumab | 1300 | 87 |

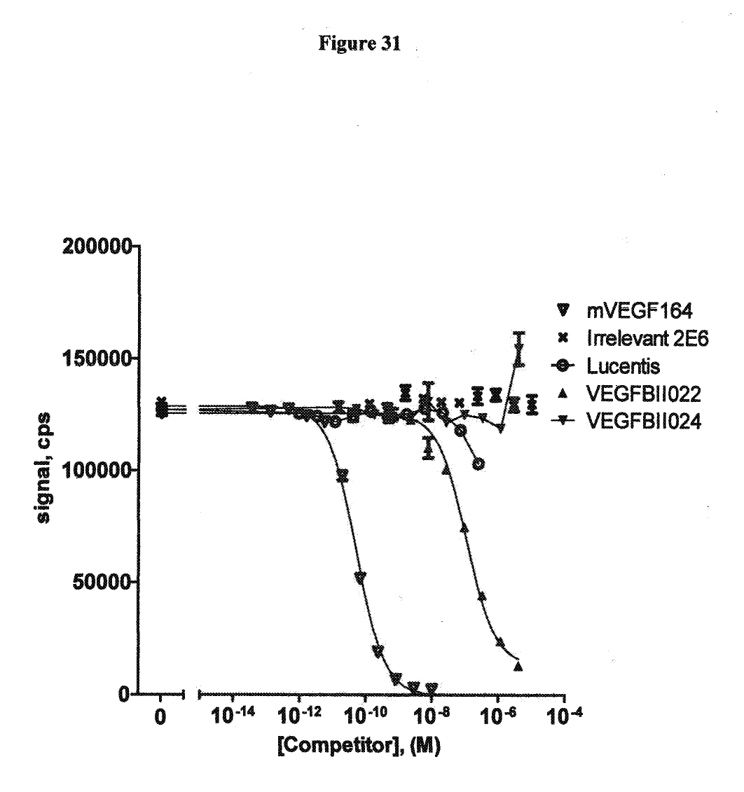

In addition, formatted VHHs are also tested for their capacity to block the mVEGF164/mVEGFR2-huFc interaction. In brief, serial dilutions of purified VHHs (concentration range: 4 μM-14.5 pM) in PBS buffer containing 0.03% Tween 20 (Sigma) are added to 0.1 nM biotinylated mVEGF164 and incubated for 15 min. Subsequently mouse VEGFR2-huFc (0.1 nM) and anti-huFc VHH-coated acceptor beads (20 μg/ml) are added and this mixture is incubated for 1 hour. Finally, streptavidin donor beads (20 μg/ml) are added and after 1 hour of incubation fluorescence is measured on the Envision microplate reader. Dose-response curves are shown in the FIG. 31. The IC₅₀ values for VHHs blocking the mouse VEGF164/VEGFR2-huFC interaction are summarized in Table 51.

TABLE 51
IC₅₀ (pM) values and % inhibition for formatted anti-VEGF VHHs in
mVEGF164/mVEGFR2-hFc competition AlphaScreen

| VHH ID | IC₅₀ (nM) | % inhibition |
|---|---|---|
| VEGFBII022 | 108 | 100 |
| VEGFBII024 | — | — |
| mVEGF164 | 0.05 | 100 |
| Ranibizumab | — | — |

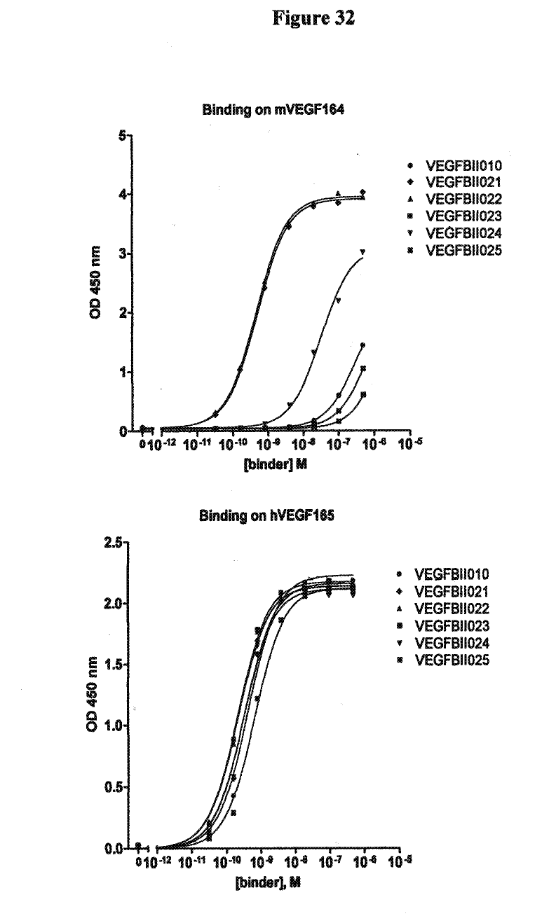

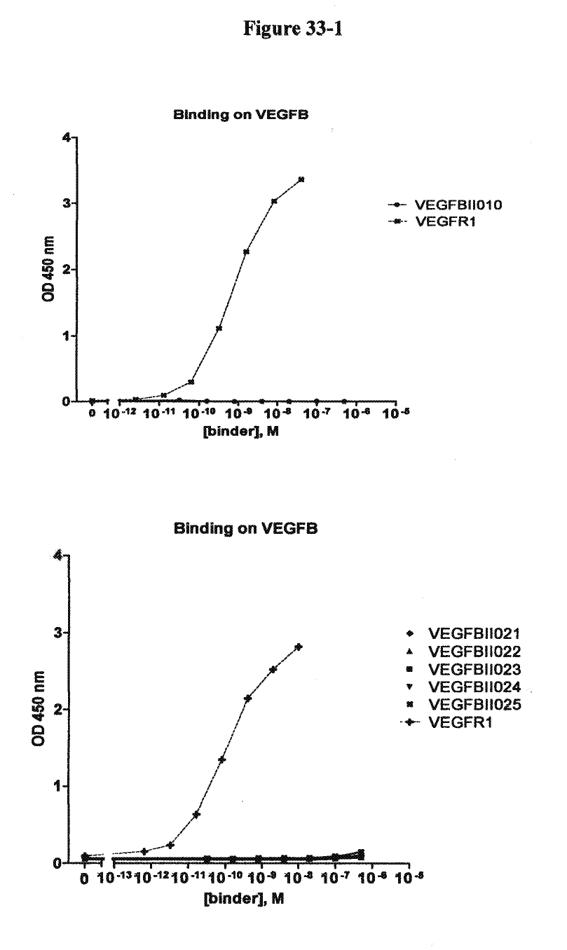

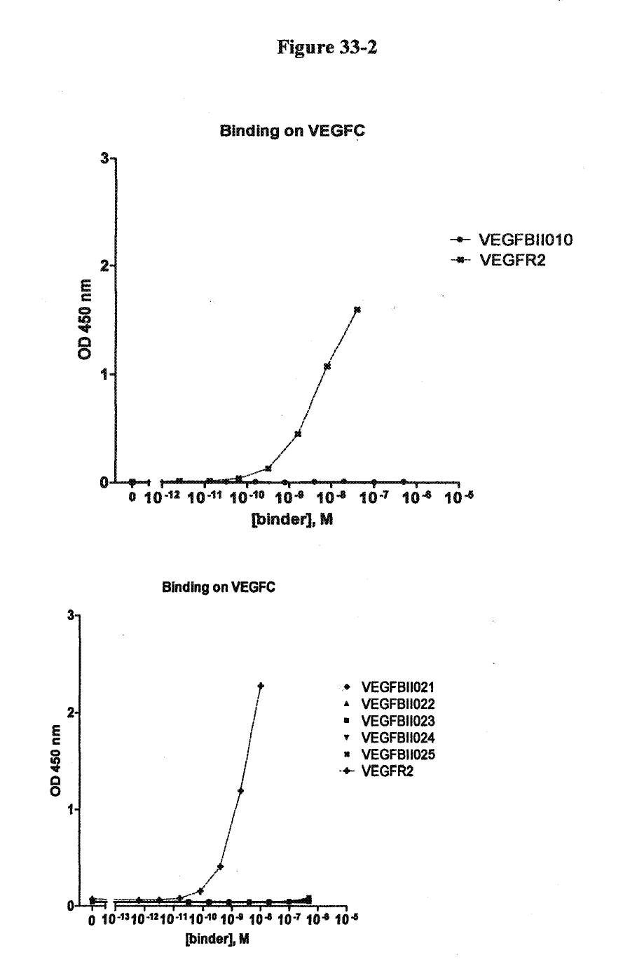

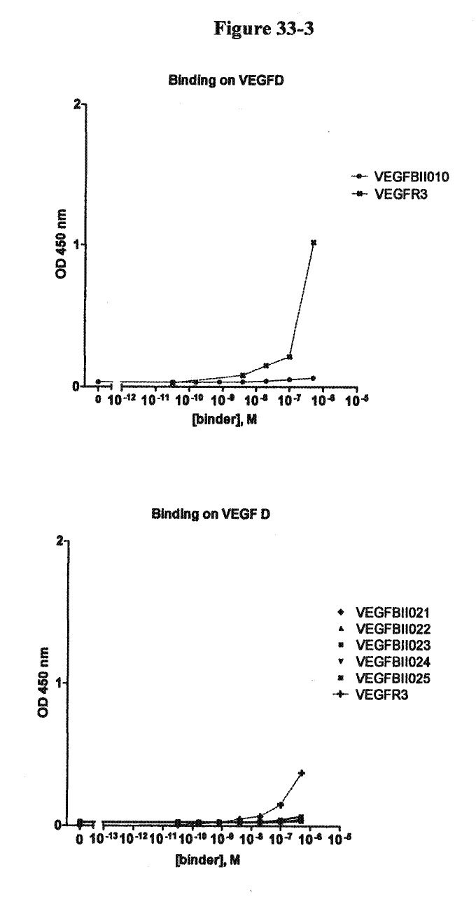

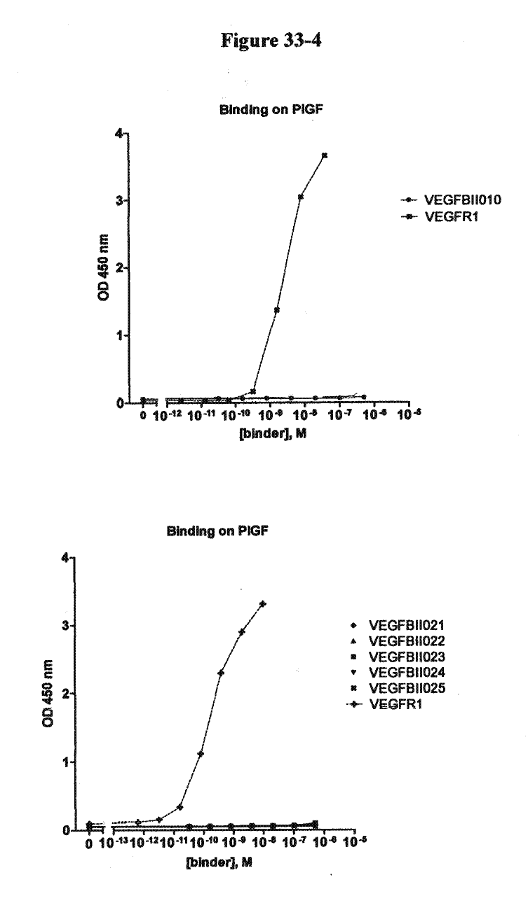

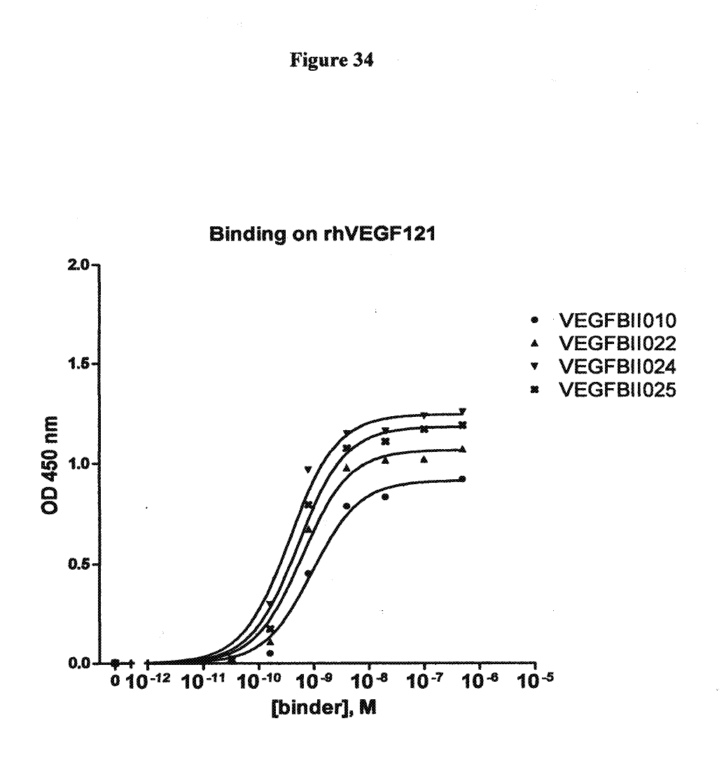

The formatted VHHs are also tested in ELISA for their ability to bind mVEGF164 and rhVEGF165 (Example 12.6; FIG. 32; Table 52), VEGF121 (Example 12.7; FIG. 34; Table 53) and the VEGF family members VEGFB, VEGFC, VEGFD and PIGF (Example 12.8; FIG. 33). Binding kinetics for human VEGF165 are analyzed as described in Example 12.5. The K_{D} values are listed in Table 54.

TABLE 52
EC₅₀ (pM) values for formatted VHHs in a recombinant human VEGF165
and mouse VEGF164 binding ELISA

| | rhVEGF165 | rmVEGF164 |
|---|---|---|
| VHH ID | EC₅₀ (pM) | EC₅₀ (pM) |
| VEGFBII010 | 428 | — |
| VEGFBII021 | 334 | 502 |
| VEGFBII022 | 224 | 464 |
| VEGFBII023 | 221 | — |
| VEGFBII024 | 320 | — |
| VEGFBII025 | 668 | — |

TABLE 53
EC₅₀ (pM) values for formatted VHHs in a recombinant human
VEGF121 binding ELISA

| | rhVEGF121 |
|---|---|
| VHH ID | EC₅₀ (pM) |
| VEGFBII010 | 920 |
| VEGFBII022 | 540 |
| VEGFBII024 | 325 |
| VEGFBII025 | 475 |

TABLE 54
Affinity K_{D} (nM) of purified formatted VHHs for recombinant human
VEGF165

| | | | | | K_{D} |
|---|---|---|---|---|---|
| VHH ID | kₐ₁ (1/Ms) | k_{d1} (1/s) | kₐ₂ (1/s) | k_{d2} (1/s) | (nM)^{(a)} |
| VEGFBII010^{(b)} | 4.5E+05 | 1.7E−02 | 2.9E−02 | 1.3E−04 | 0.16 |
| VEGFBII021^{(b)} | 1.2E+06 | 1.1E−02 | 2.3E−02 | 1.9E−04 | 0.07 |
| VEGFBII022^{(b)} | 1.2E+06 | 9.1E−03 | 1.4E−02 | 2.6E−04 | 0.14 |
| VEGFBII023^{(b)} | 3.0E+05 | 1.8E−02 | 2.4E−02 | 2.7E−04 | 0.69 |
| VEGFBII024^{(b)} | 3.0E+05 | 1.3E−02 | 2.6E−02 | 2.8E−04 | 0.47 |
| VEGFBII025^{(b)} | 3.3E+05 | 1.7E−02 | 1.8E−02 | 3.7E−04 | 1.1 |

^{(a)}K_{D} = k_{d1}/kₐ₁ * (k_{d2}/(k_{d2} + kₐ₂))
^{(b)}Curves are fitted using a Two State Reaction model by Biacore T100 Evaluation Software v2.0.1

VHHs VEGFBII010, VEGFBII022, VEGFBII024 and VEGFBII025 are also tested in the VEGF mediated HUVEC proliferation and Erk phosphorylation assay.

The potency of the selected formatted VHHs is evaluated in a proliferation assay. In brief, primary HUVEC cells (Technoclone) are supplement-starved over night and then 4000 cells/well are seeded in quadruplicate in 96-well tissue culture plates. Cells are stimulated in the absence or presence of VHHs with 33 ng/mL VEGF. The proliferation rates are measured by [³H] Thymidine incorporation on day 4. The results shown in Table 55 demonstrate that the formatted VHHs and Bevacizumab inhibit the VEGF induced HUVEC proliferation by more than 90%, with IC₅₀s<1 nM.

TABLE 55
IC₅₀ (nM) values and % inhibition of formatted VHHs in VEGF HUVEC
proliferation assay

| | IC₅₀ | |
|---|---|---|
| VHH ID | (nM) | % inhibition |
| VEGFBII010 | 0.22 | 95 |
| VEGFBII021 | 0.40 | 98 |
| VEGFBII022 | 0.34 | 100 |
| VEGFBII023 | 0.52 | 98 |
| VEGFBII024 | 0.38 | 96 |
| VEGFBII025 | 0.41 | 104 |
| Bevacizumab | 0.21 | 92 |

The potency of the selected formatted VHHs is also assessed in the HUVEC Erk phosphorylation assay. In brief, primary HUVEC cells are serum-starved over night and then stimulated in the absence or presence of VHHs with 10 ng/mL VEGF for 5 min. Cells are fixed with 4% Formaldehyde in PBS and ERK phosphorylation levels are measured by ELISA using phosphoERK-specific antibodies (anti-phosphoMAP Kinase pERK1 &2, M8159, Sigma) and polyclonal Rabbit Anti-Mouse-Immunoglobulin-HRP conjugate (PO161, Dako). As shown in Table 56, the formatted VHHs and Bevacizumab inhibit the VEGF induced Erk phosphorylation by more than 90%, with IC₅₀s<1 nM.

TABLE 56
IC₅₀ (nM) values and % inhibition of formatted VHHs in VEGF HUVEC
Erk phosphorylation assay

| | IC₅₀ | |
|---|---|---|
| VHH ID | (nM) | % inhibition |
| VEGFBII010 | 0.19 | 92 |
| VEGFBII021 | 0.21 | 103 |
| VEGFBII022 | 0.18 | 94 |
| VEGFBII023 | 0.25 | 100 |
| VEGFBII024 | 0.23 | 94 |
| VEGFBII025 | 0.23 | 99 |
| Bevacizumab | 0.63 | 98 |

### Example 15

Sequence Optimization

15.1 Sequence Optimization of VEGFBII23B04

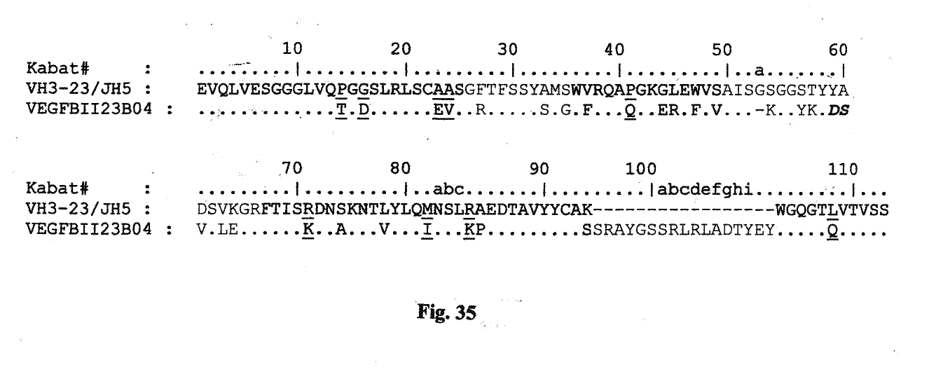

The amino acid sequence of VEGFBII23B04 is aligned to the human germline sequences VH3-23 (DP-47) and JHS, see FIG. 35 SEQ ID NO: 100. The alignment shows that VEGFBII23B04 contains 19 framework mutations relative to the reference germline sequence. Non-human residues at positions 14, 16, 23, 24, 41, 71, 82, 83 and 108 are selected for substitution with their human germline counterparts. A set of 8 VEGFBII23B04 variants is generated carrying different combinations of human residues on these positions (AA sequence are listed in Table 57). One additional variant is constructed in which the potential isomerization site at position D59S60 (CDR2 region, see FIG. 35 indicated as bold italic residues) is removed by introduction of a S60A mutation.

TABLE 57

AA sequences of sequence-optimized variants of VHH VEGFBII23B04
(FR, framework; CDR, complementary determining region)

| VHH ID/ | | | | | | | |
|---|---|---|---|---|---|---|---|
| SEQ ID | | | | | | | |
| NO: | FR1 | CDR1 | FR2 | CDR2 | FR3 | CDR3 | FR4 |
| VEGFBII111D05 | EVQLVESGGG | SYSM | WFRQAPGKERE | AISKGGYKY | RFTISRDNAKNTVYLQ | SRAYGSS | WGQGTLVTVS |
| 54 | LVQTGGSLRL | G | FVV | DSVSLEG | INSLRPEDTAVYYCAS | RLRLADT | S |
| | SCEASGRTFS | | | | | YEY | |
| VEGFBII111G06 | EVQLVESGGG | SYSM | WFRQAPGKERE | AISKGGYKY | RFTISRDNAKNTVYLQ | SRAYGSS | WGQGTLVTVS |
| 55 | LVQPGGSLRL | G | FVV | DSVSLEG | MNSLRPEDTAVYYCAS | RLRLADT | S |
| | SCAASGRTFS | | | | | YEY | |
| VEGFBII112D11 | EVQLVESGGG | SYSM | WFRQAPGKERE | AISKGGYKY | RFTISRDNAKNTVYLQ | SRAYGSS | WGQGTLVTVS |
| 56 | LVQPGGSLRL | G | FVV | DSVSLEG | INSLRPEDTAVYYCAS | RLRLADT | S |
| | SCEASGRTFS | | | | | YEY | |
| VEGFBII113A08 | EVQLVESGGG | SYSM | WFRQAPGKERE | AISKGGYKY | RFTISKDNAKNTVYLQ | SRAYGSS | WGQGTLVTVS |
| 57 | LVQTGGSLRL | G | FVV | DSVSLEG | INSLRPEDTAVYYCAS | RLRLADT | S |
| | SCEVSGRTFS | | | | | YEY | |
| VEGFBII113E03 | EVQLVESGGG | SYSM | WFRQAQGKERE | AISKGGYKY | RFTISKDNAKNTVYLQ | SRAYGSS | WGQGTLVTVS |
| 58 | LVQTGDSLRL | G | FVV | DSVSLEG | MNSLRPEDTAVYYCAS | RLRLADT | S |
| | SCEVSGRTFS | | | | | YEY | |
| VEGFBII114C09 | EVQLVESGGG | SYSM | WFRQAPGKERE | AISKGGYKY | RFTISKDNAKNTVYLQ | SRAYGSS | WGQGTLVTVS |
| 59 | LVQPGDSLRL | G | FVV | DSVSLEG | INSLRPEDTAVYYCAS | RLRLADT | S |
| | SCEVSGRTFS | | | | | YEY | |
| VEGFBII114D02 | EVQLVESGGG | SYSM | WFRQAPGKERE | AISKGGYKY | RFTISRDNAKNTVYLQ | SRAYGSS | WGQGTLVTVS |
| 60 | LVQTGGSLRL | G | FVV | DSVSLEG | INSLRPEDTAVYYCAS | RLRLADT | S |
| | SCEVSGRTFS | | | | | YEY | |
| VEGFBII114D03 | EVQLVESGGG | SYSM | WFRQAQGKERE | AISKGGYKY | RFTISKDNAKNTVYLQ | SRAYGSS | WGQGTLVTVS |
| 61 | LVQTGDSLRL | G | FVV | DSVSLEG | INSLRPEDTAVYYCAS | RLRLADT | S |
| | SCAVSGRTFS | | | | | YEY | |
| VEGFBII118E10 | EVQLVESGGG | SYSM | WFRQAQGKERE | AISKGGYKY | RFTISKDNAKNTVYLQ | SRAYGSS | WGQGTQVTVS |
| 62 | LVQTGDSLRL | G | FVV | DAVSLEG | INSLKPEDTAVYYCAS | RLRLADT | S |
| | SCEVSGRTFS | | | | | YEY | |

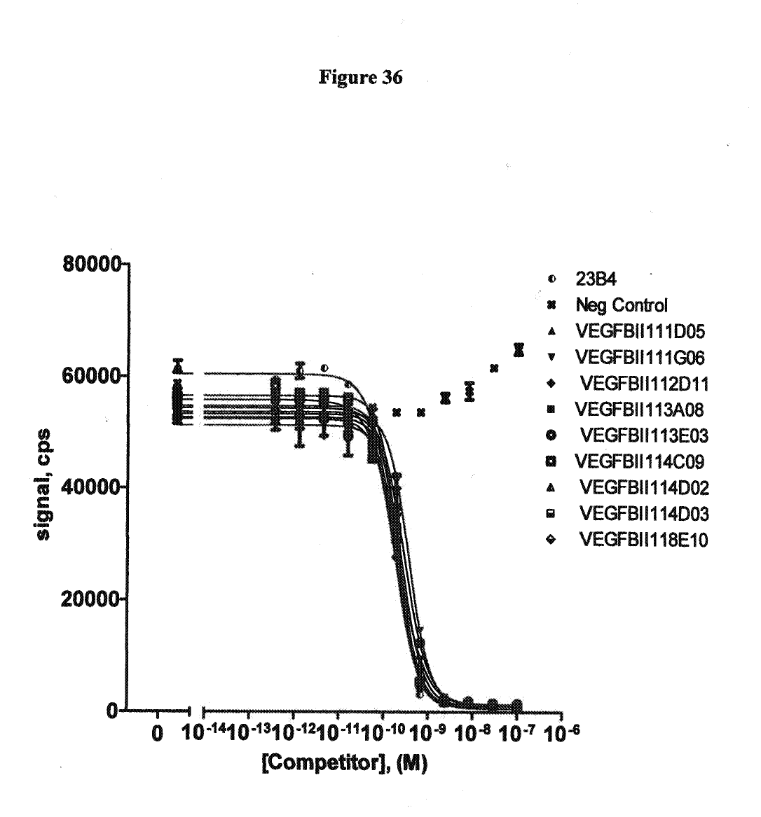

These variants are characterized as purified proteins in the VEGF165/VEGFR2 AlphaScreen (Example 12.3, FIG. 36). The melting temperature (Tₘ) of each clone is determined in a thermal shift assay, which is based on the increase in fluorescence signal upon incorporation of Sypro Orange (Invitrogen) (Ericsson et al, Anal. Biochem. 357 (2006), pp 289-298). All variants displayed comparable IC₅₀ when compared to VEGFBII23B04 and Tₘ values which are similar or higher when compared to the parental VEGFBII23B04. Table 58 summarizes the IC₅₀ values, % inhibition and Tₘ values at pH 7 for the 9 clones tested.

TABLE 58
IC₅₀ (pM) values, % inhibition and melting temperature (@pH 7) of
sequence-optimized variants of VEGFBII23B04

| | | % | Tₘ @ pH 7 |
|---|---|---|---|
| VHH ID | IC₅₀ (pM) | inhibition | (° C.) |
| VEGFBII23B04 | 169 | 100 | 63 |
| (wt) | | | |
| VEGFBII111D05 | 209 | 100 | 68 |
| VEGFBII111G06 | 366 | 100 | 71 |
| VEGFBII112D11 | 221 | 100 | 70 |
| VEGFBII113A08 | 253 | 100 | 69 |
| VEGFBII113E03 | 290 | 100 | 68 |
| VEGFBII114C09 | 215 | 100 | 71 |
| VEGFBII114D02 | 199 | 100 | 74 |
| VEGFBII114D03 | 227 | 100 | 64 |
| VEGFBII118E10 | 189 | 100 | 62 |

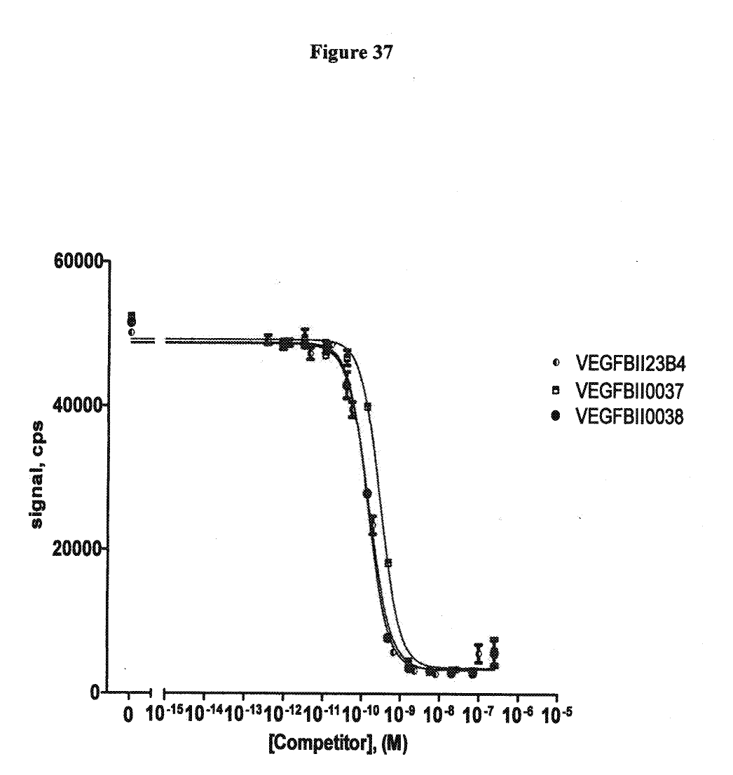

In a second cycle, tolerated mutations from the humanization effort (VEGFBII111G06) and mutations to avoid potential posttranslational modification at selected sites, (the D160, the S60A substitution and an E1D mutation) are combined resulting in a sequence-optimized clone derived from VEGFBII23B04: VEGFBII0037. One extra sequence-optimized variant (VEGFBII038) is anticipated which contains all substitutions as VEGFBII0037, with the exception of the I82M mutation, as this mutation may be associated with a minor drop in potency. The sequences of both sequence-optimized clones are listed in Table 59. VEGFBII0037 and VEGFBII0038 are characterized in the VEGF165/VEGFR2 blocking AlphaScreen (Example 13.3, FIG. 37), the melting temperature is determined in the thermal shift assay as described above and the affinity for binding on VEGF165 is determined in Biacore (Example 13.5). An overview of the characteristics of the 2 sequence-optimized VHHs is presented in Table 60.

TABLE 59

AA sequences of sequence-optimized variants of VHH
VEGFBII23B04

| VHH ID/ | | | | | | | |
|---|---|---|---|---|---|---|---|
| SEQ ID | | | | | | | |
| NO: | FR 1 | CDR 1 | FR2 | CDR 2 | FR3 | CDR 3 | FR 4 |
| VEGFBII037 | DVQLV | SYS | WFRQ | AISKGG | RFTISRDNAK | SRAYGSS | WGQGT |
| 63 | ESGGG | MG | APGK | YKYDAV | NTVYLQMNSL | RLRLADT | LVTVS |
| | LVQPG | | EREF | SLEG | RPEDTAVYYC | YEY | S |
| | GSLRL | | VV | | AS | | |
| | SCAAS | | | | | | |
| | GRTFS | | | | | | |
| VEGFBII038 | DVQLV | SYS | WFRQ | AISKGG | RFTISRDNAK | SRAYGSS | WGQGT |
| 64 | ESGGG | MG | APGK | YKYDAV | NTVYLQINSL | RLRLADT | LVTVS |
| | LVQPG | | EREF | SLEG | RPEDTAVYYC | YEY | S |
| | GSLRL | | VV | | AS | | |
| | SCAAS | | | | | | |
| | GRTFS | | | | | | |

TABLE 60
IC₅₀ (pM) values, % inhibition, melting temperature (@pH 7) and affinity
(pM) of sequence-optimized clones VEGFBII037 and VEGFBII038

| | IC₅₀ | % | Tₘ (° C.) | K_{D} |
|---|---|---|---|---|
| VHH ID | (pM) | inhibition | @ pH 7 | (pM) |
| VEGFBII23B04 | 152 | 100 | 63 | 560 |
| VEGFBII037 | 300 | 100 | 72 | 270 |
| VEGFBII038 | 143 | 100 | 71 | 360 |

15.2 Sequence Optimization of VEGFBII5B05

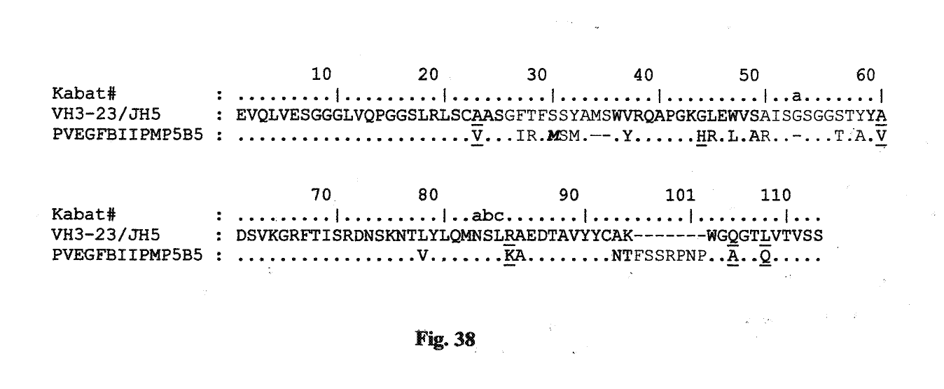

The amino acid sequence of VEGFBII5B05 is aligned to the human germline sequence VH3-23/JH5; see FIG. 38 and SEQ ID NO: 100. The alignment shows that VEGFBII5B05 contains 15 framework mutations relative to the reference germline sequence. Non-human residues at positions 23, 60, 83, 105, 108 are selected for substitution with their human germline counterparts while the histidine at position 44 is selected for substitution by glutamine. One humanization variant is constructed carrying the 6 described mutations (AA sequence is listed in Table 61).

TABLE 61

AA sequence of sequence-optimized variants of VHH VEGFBII5B05
(FR, framework; CDR, complementary determining region)

| VHH ID/ | | | | | | | |
|---|---|---|---|---|---|---|---|
| SEQ ID NO: | FR1 | CDR 1 | FR2 | CDR2 | FR3 | CDR3 | FR4 |
| VEGFBII119G11 | EVQLVESG | SMA | WYRQAPGK | RISSGG | RFTISRDNSKNT | FSSRP | WGQGTLV |
| 65 | GGLVQPGG | | QRELVA | TTAYAD | VYLQMNSLRAE | NP | TVSS |
| | SLRLSCAAS | | | SVKG | DTAVYYCNT | | |
| | GIRFM | | | | | | |
| VEGFBII120E10 | EVQLVESG | SMA | WYRQAPGK | RISSGG | RFTISRDNSKNT | FSSRP | WGAGTQV |
| 66 | GGLVQPGG | | HRELVA | TTAYVD | VYLQMNSLKAE | NP | TVSS |
| | SLRLSCVAS | | | SVKG | DTAVYYCNT | | |
| | GIRFI | | | | | | |

One additional variant is constructed in which the potential oxidation site at position M30 (CDR1 region, see FIG. 38 indicated as bold italic residue) is removed by introduction of a M30I mutation. Both variants are tested for their ability to bind hVEGF165 using the ProteOn. In brief, a GLC ProteOn Sensor chip is coated with human VEGF165. Periplasmic extracts of the variants are diluted 1/10 and injected across the chip coated with human VEGF165. Off-rates are calculated and compared to the off-rates of the parental VEGFBII5B05. Off-rates from the 2 variants are in the same range as the off-rates from the parental VEGFBII5B05 indicating that all mutations are tolerated (Table 62).

TABLE 62
Off-rates sequence-optimized variants VEGFBII5B05

| | binding | |
|---|---|---|
| | level | |
| VHH ID | (RU) | k_{d} (1/s) |
| VEGFBII5B05 | 242 | 6.15E−02 |
| VEGFBII119G11 | 234 | 7.75E−02 |
| VEGFBII120E10 | 257 | 4.68E−02 |

In a second cycle, mutations from the humanization effort and the M30I substitution are combined resulting in a sequence-optimized clone of VEGFBII5B05, designated VEGFBII032. The sequence is listed in Table 63. Affinity of VEGFBII032 is determined by Biacore (see Example 12.5) and the melting temperature is determined in the thermal shift assay as described above. An overview of the characteristics of the sequence-optimized VHH VEGFBII032 is presented in Table 64.

TABLE 63

AA sequence of sequence-optimized clone VEGFBII032
(FR, framework; CDR, complementary determining region)

| VHH ID/ | | | | | | | |
|---|---|---|---|---|---|---|---|
| SEQ ID NO: | FR1 | CDR 1 | FR2 | CDR2 | FR3 | CDR3 | FR4 |
| VEGFBII032 | EVQLVESG | SMA | WYRQAPGK | RISSGG | RFTISRDNSKNT | FSSRP | WGQGTLV |
| 67 | GGLVQPGG | | QRELVA | TTAYAD | VYLQMNSLRAE | NP | TVSS |
| | SLRLSCAAS | | | SVKG | DTAVYYCNT | | |
| | GIRFI | | | | | | |

TABLE 64
Melting temperature (@pH 7) and affinity (nM) of sequence-optimized
clone VEGFBII032

| | Tₘ (° C.) | |
|---|---|---|
| VHH ID | @ pH 7 | K_{D} (nM) |
| VEGFBII5B05(wt) | 69 | 32 |
| VEGFBII0032 | 71 | 44 |

The potency of the sequence-optimized clones VEGFBII037 and VEGFBII038 is evaluated in a proliferation assay. In brief, primary HUVEC cells (Technoclone) are supplement-starved over night and then 4000 cells/well are seeded in quadruplicate in 96-well tissue culture plates. Cells are stimulated in the absence or presence of VHHs with 33 ng/mL VEGF. The proliferation rates are measured by [³H] Thymidine incorporation on day 4. The results shown in Table 65 demonstrate that the activity (potency and degree of inhibition) of the parental VHH VEGFBII23B04 is conserved in the sequence-optimized clone VEGFBII038.

TABLE 65
IC₅₀ (nM) values and % inhibition of the sequence optimized clones
VEGFBII037 and VEGFBII038 in VEGF HUVEC proliferation assay

| VHH ID | IC₅₀ (nM) | % inhibition |
|---|---|---|
| VEGFBII23B04 | 0.68 | 92 |
| VEGFBII037 | 1.54 | 78 |
| VEGFBII038 | 0.60 | 92 |
| Bevacizumab | 0.29 | 94 |

### EXAMPLE 16

Construction and Characterization of Bispecific VHHs Targeting VEGF and DLL4 using PEGylation or Anti-Serum Albumin Binding as Half-Life Extension

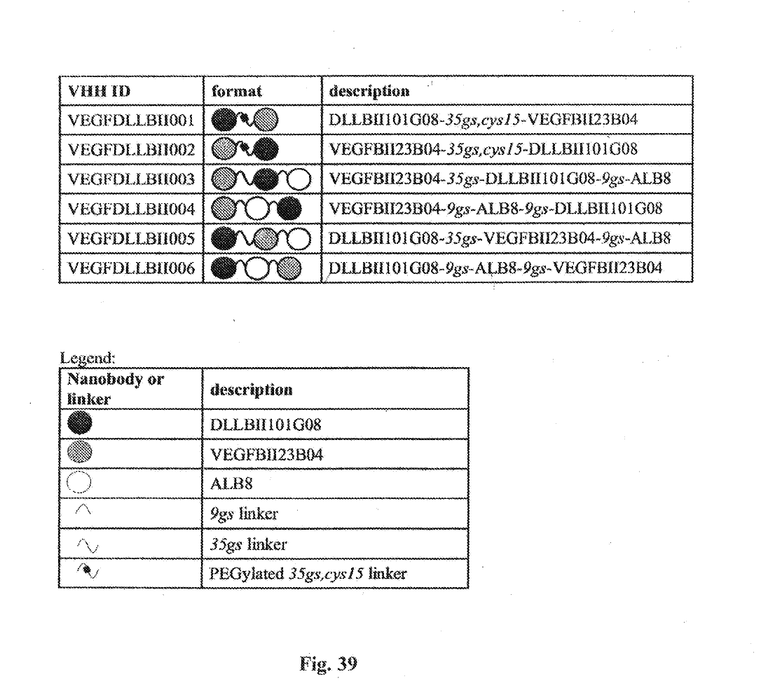

In a first cycle, VEGFBII23B04 and DLLBII101G08 are used as building blocks to generate bispecific VHHs VEGFDLLBII001-006. Two half-life extension methodologies are applied: i) PEGylation or ii) genetic fusion to a serum albumin binding VHH. Building blocks are linked via a 9 Gly-Ser, 35 Gly-Ser or 35 Gly-Ser (Cys at position 15) flexible linker. An overview of the format and sequence of all 6 bispecific VHHs is depicted in Table 66-A (linker sequences are underlined), SEQ ID Nos: 68-73 and in FIG. 39.

TABLE 66-A
Sequences of bispecific VHHs targeting VEGF and DLL4

| VHH ID/ | |
|---|---|
| SEQ ID NO: | AA sequence |
| VEGFDLLBII001 | EVQLVESGGGLVQAGGSLRLSCAASGRTFSSYAMAWFRQAPGKEREFVAAIRWSGGTAYYADSVQGRFTISRD |
| 68 | NAKNTVYLQMNSLKPEDTAVYYCANRAADTRLGPYEYDYWGQGTQVTVSSGGGGSGGGGSGGGGCGGGGSGGG |
| | GSGGGGSGGGGSEVQLVESGGGLVQTGDSLRLSCEVSGRTFSSYSMGWFRQAQGKEREFVVAISKGGYKYDSV |
| | SLEGRFTISKDNAKNTVYLQINSLKPEDTAVYYCASSRAYGSSRLRLADTYEYWGQGTQVTVSS |
| VEGFDLLBII002 | EVQLVESGGGLVQTGDSLRLSCEVSGRTFSSYSMGWFRQAQGKEREFVVAISKGGYKYDSVSLEGRFTISKDN |
| 69 | AKNTVYLQINSLKPEDTAVYYCASSRAYGSSRLRLADTYEYWGQGTQVTVSSGGGGSGGGGSGGGGCGGGGSG |
| | GGGSGGGGSGGGGSEVQLVESGGGLVQAGGSLRLSCAASGRTFSSYAMAWFRQAPGKEREFVAAIRWSGGTAY |
| | YADSVQGRFTISRDNAKNTVYLQMNSLKPEDTAVYYCANRAADTRLGPYEYDYWGQGTQVTVSS |
| VEGFDLLBII003 | EVQLVESGGGLVQTGDSLRLSCEVSGRTFSSYSMGWFRQAQGKEREFVVAISKGGYKYDSVSLEGRFTISKDN |
| 70 | AKNTVYLQINSLKPEDTAVYYCASSRAYGSSRLRLADTYEYWGQGTQVTVSSGGGGSGGGGSGGGGSGGGGSG |
| | GGGSGGGGSEVQLVESGGGLVQAGGSLRLSCAASGRTFSSYAMAWFRQAPGKEREFVAAIRWSGGTAYYADSV |
| | QGRFTISRDNAKNTVYLQMNSLKPEDTAVYYCANRAADTRLGPYEYDYWGQGTQVTVSSGGGGSGGGSEVQLV |
| | ESGGGLVQPGNSLRLSCAASGFTFSSFGMSWVRQAPGKGLEWVSSISGSGSDTLYADSVKGRFTISRDNAKTT |
| | LYLQMNSLRPEDTAVYYCTIGGSLSRSSQGTLVTVSS |
| VEGFDLLBII004 | EVQLVESGGGLVQTGDSLRLSCEVSGRTFSSYSMGWFRQAQGKEREFVVAISKGGYKYDSVSLEGRFTISKDN |
| 71 | AKNTVYLQINSLKPEDTAVYYCASSRAYGSSRLRLADTYEYWGQGTQVTVSSGGGGSGGGSEVQLVESGGGLV |
| | QPGNSLRLSCAASGFTFSSFGMSWVRQAPGKGLEWVSSISGSGSDTLYADSVKGRFTISRDNAKTTLYLQMNS |
| | LRPEDTAVYYCTIGGSLSRSSQGTLVTVSSGGGGSGGGSEVQLVESGGGLVQAGGSLRLSCAASGRTFSSYAM |
| | AWFRQAPGKEREFVAAIRWSGGTAYYADSVQGRFTISRDNAKNTVYLQMNSLKPEDTAVYYCANRAADTRLGP |
| | YEYDYWGQGTQVTVSS |
| VEGFDLLBII005 | EVQLVESGGGLVQAGGSLRLSCAASGRTFSSYAMAWFRQAPGKEREFVAAIRWSGGTAYYADSVQGRFTISRD |
| 72 | NAKNTVYLQMNSLKPEDTAVYYCANRAADTRLGPYEYDYWGQGTQVTVSSGGGGSGGGGSGGGGSGGGGSGGG |
| | GSGGGGSEVQLVESGGGLVQTGDSLRLSCEVSGRTFSSYSMGWFRQAQGKEREFVVAISKGGYKYDSVSLEGR |
| | FTISKDNAKNTVYLQINSLKPEDTAVYYCASSRAYGSSRLRLADTYEYWGQGTQVTVSSGGGGSGGGSEVQLV |
| | ESGGGLVQPGNSLRLSCAASGFTFSSFGMSWVRQAPGKGLEWVSSISGSGSDTLYADSVKGRFTISRDNAKTT |
| | LYLQMNSLRPEDTAVYYCTIGGSLSRSSQGTLVTVSS |
| VEGFDLLBII006 | EVQLVESGGGLVQAGGSLRLSCAASGRTFSSYAMAWFRQAPGKEREFVAAIRWSGGTAYYADSVQGRFTISRD |
| 73 | NAKNTVYLQMNSLKPEDTAVYYCANRAADTRLGPYEYDYWGQGTQVTVSSGGGGSGGGSEVQLVESGGGLVQP |
| | GNSLRLSCAASGFTFSSFGMSWVRQAPGKGLEWVSSISGSGSDTLYADSVKGRFTISRDNAKTTLYLQMNSLR |
| | PEDTAVYYCTIGGSLSRSSQGTLVTVSSGGGGSGGGSEVQLVESGGGLVQTGDSLRLSCEVSGRTFSSYSMGW |
| | FRQAQGKEREFVVAISKGGYKYDSVSLEGRFTISKDNAKNTVYLQINSLKPEDTAVYYCASSRAYGSSRLRLA |
| | DTYEYWGQGTQVTVSS |

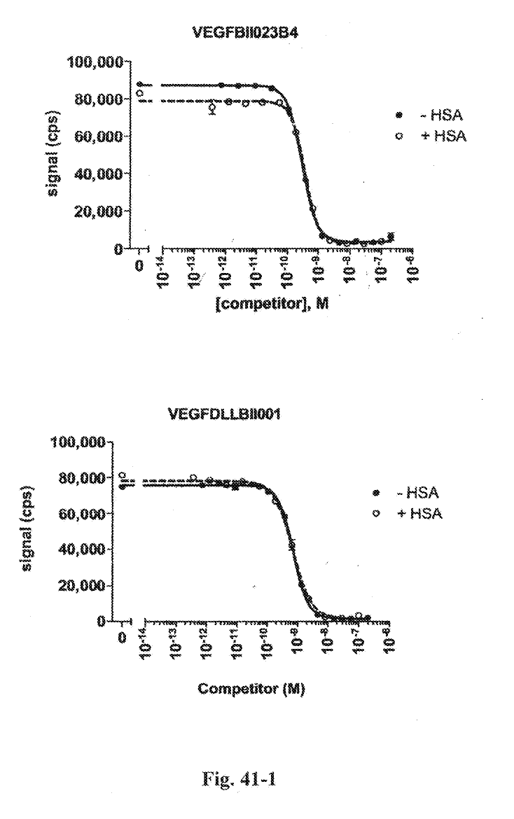

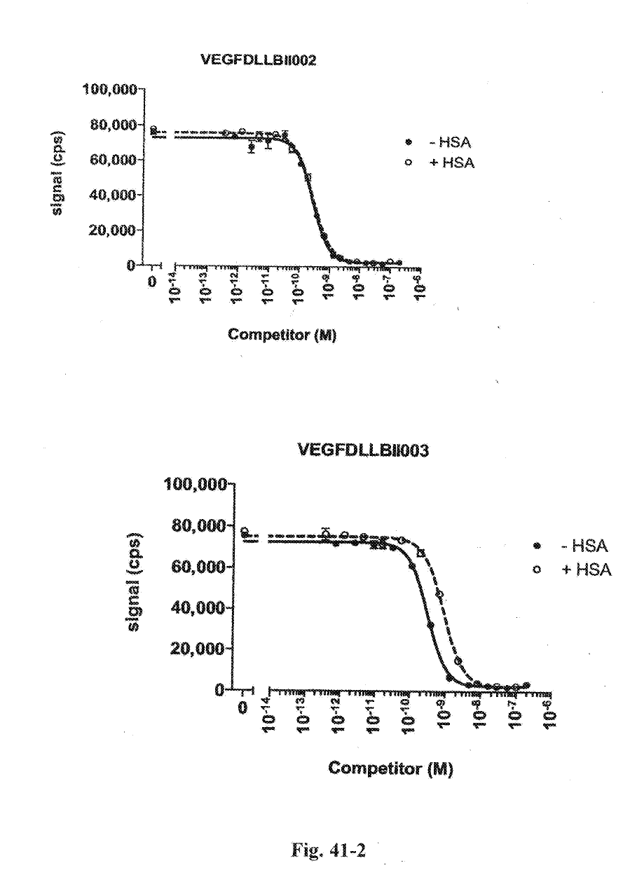

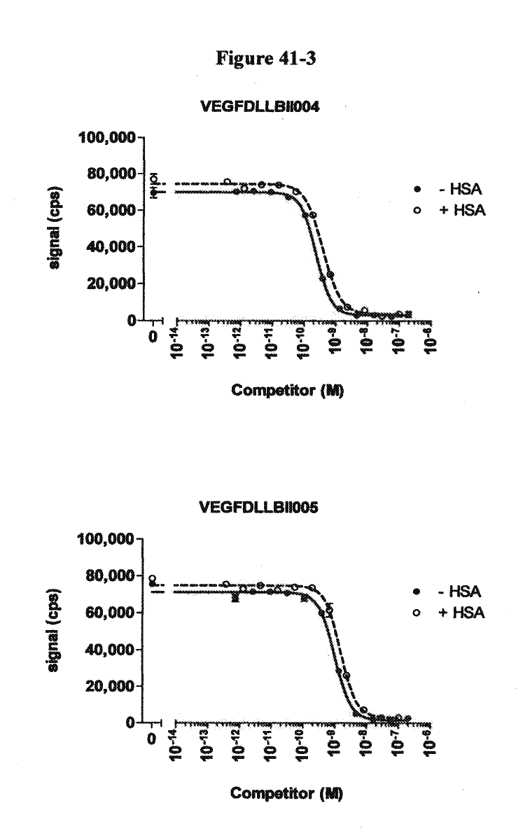

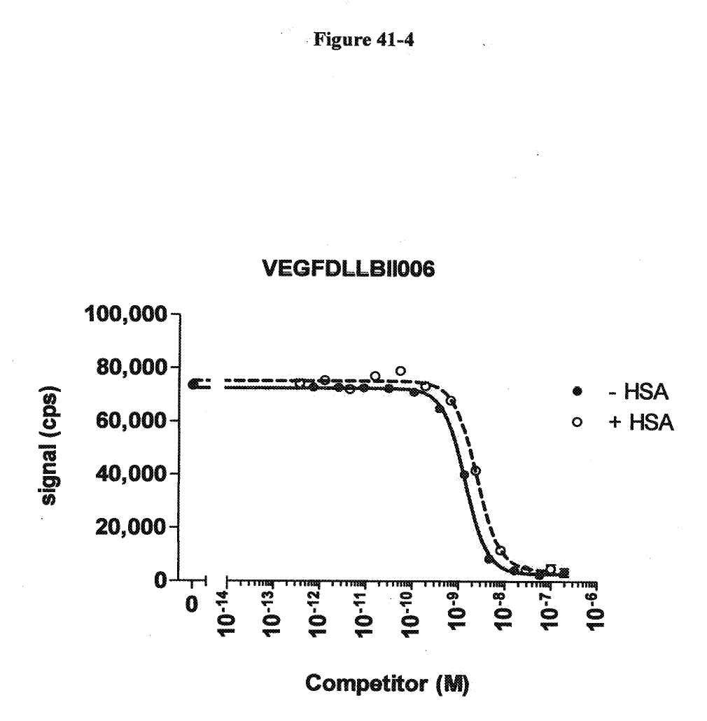

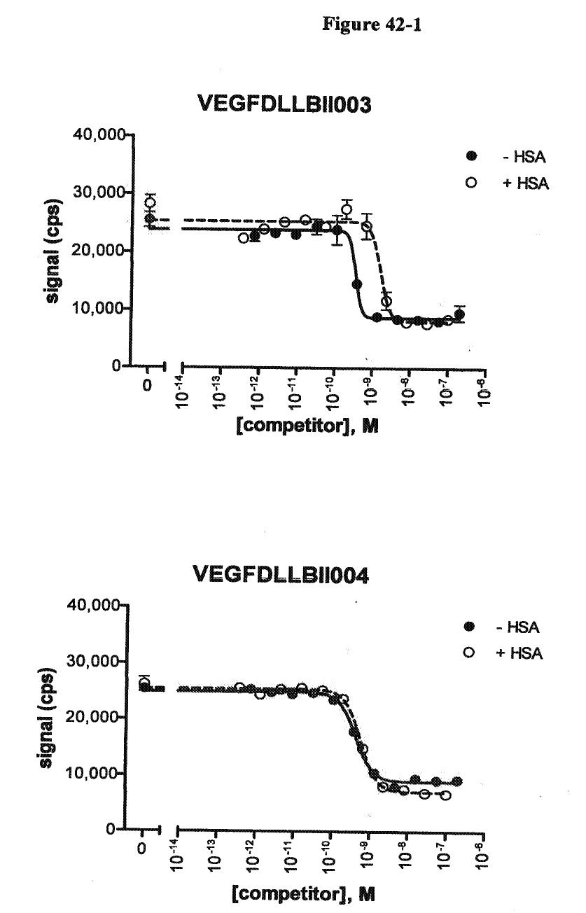

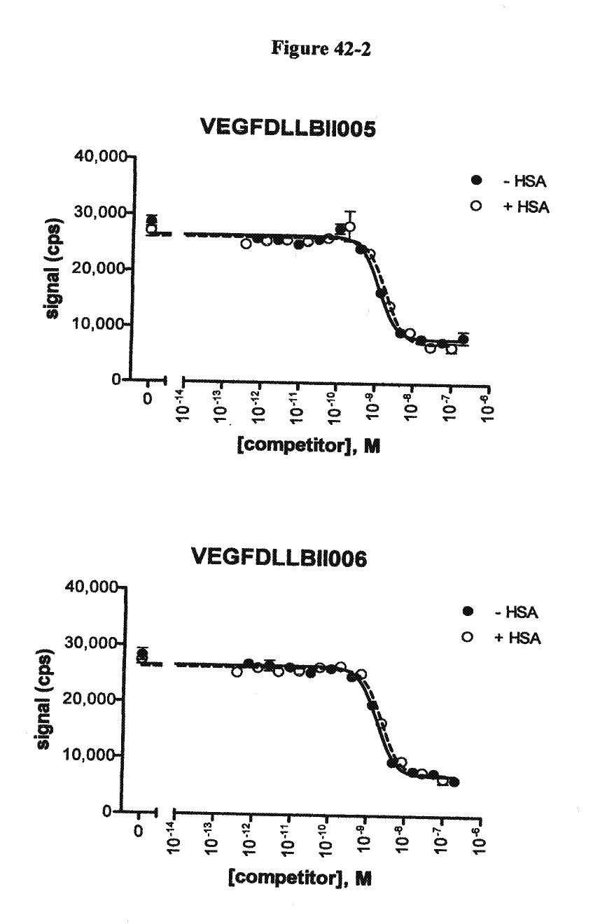

To explore the anti-VEGF blocking properties in comparison with the monovalent building block VEGFBII23B04, all six VHHs are analyzed in the VEGF/VEGFR2-Fc (Example 12.3; FIG. 41) and VEGF/VEGFR1-Fc (Example 12.4; FIG. 42) competition AlphaScreen. These 2 competition assays are also performed after preincubation of the VHHs with 5 μM human serum albumin. A summary of IC₅₀ values is shown in Table 66-B

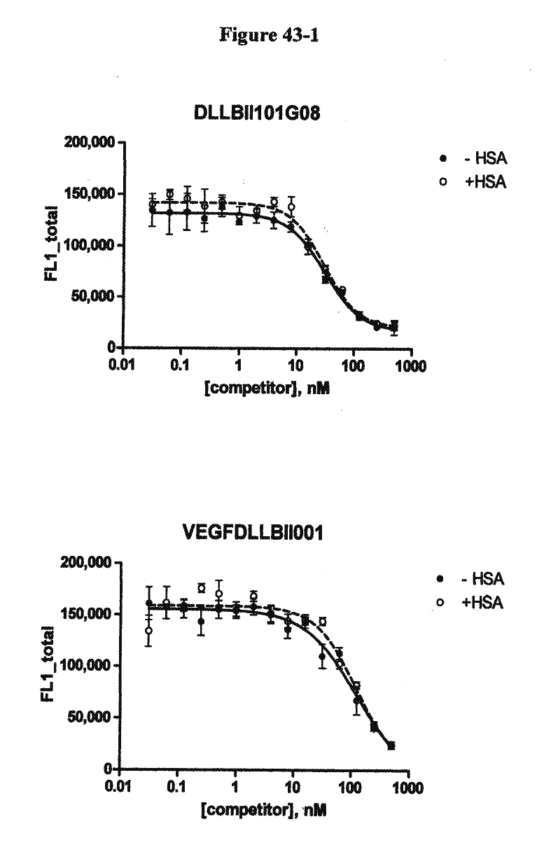

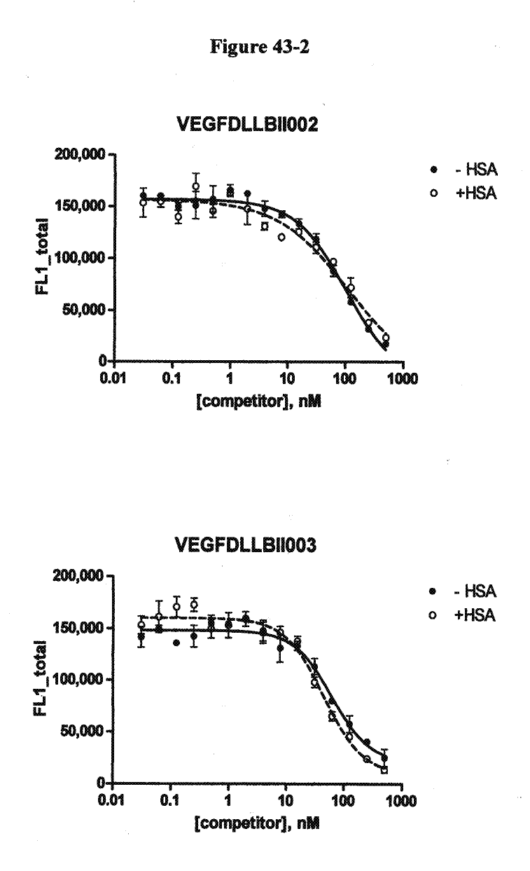

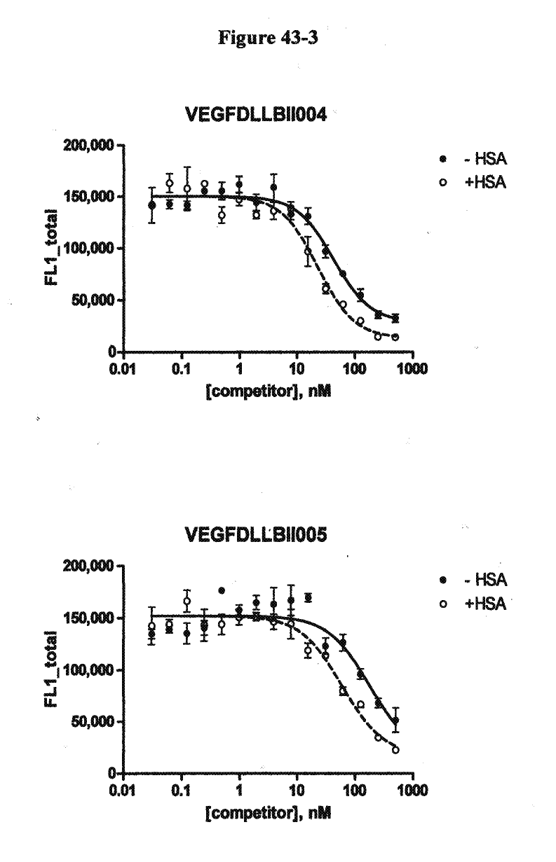

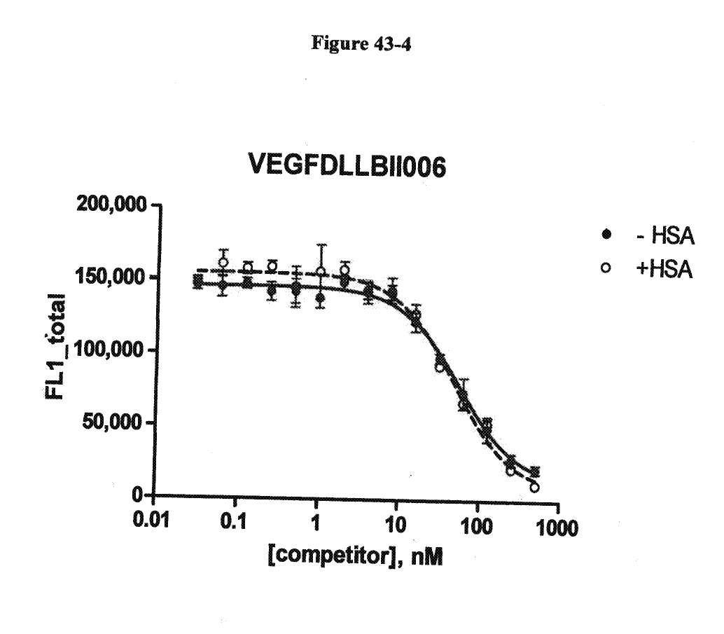

To explore the anti-DLL4 blocking properties in comparison with the monovalent building block DLLBII101 G08, all six VHHs are tested in the CHO-hDLL4/hNotch1-Fc competitive FMAT assay (Example 4; FIG. 43). This assay is also performed after preincubation of the VHHs with 25 μM human serum albumin. A summary of IC₅₀ values is shown in Table 67.

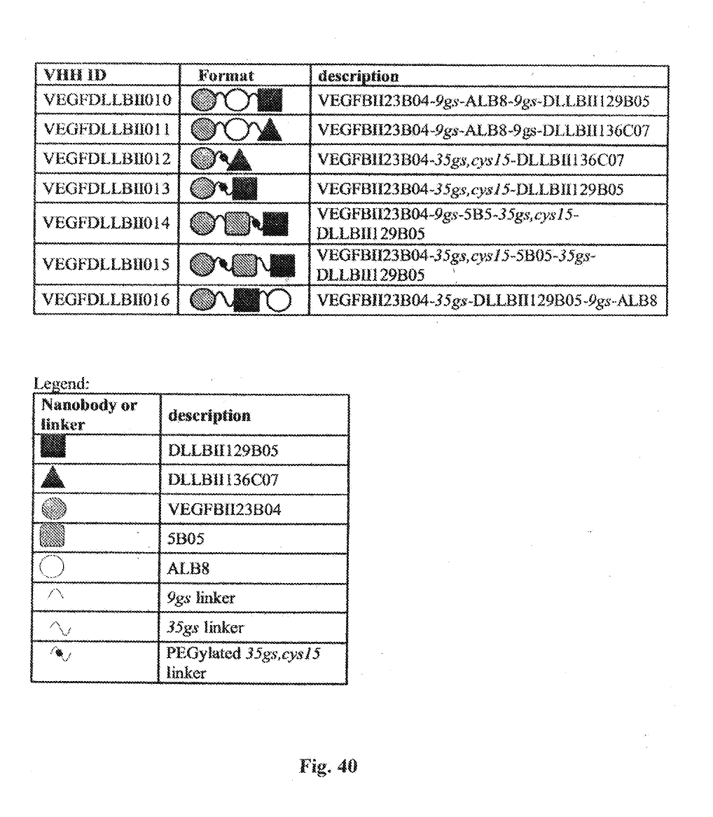

In a second cycle, seven bispecific VHHs targeting VEGF and DLL4 are constructed (VEGFDLLBII010, VEGFDLLBII011, VEGFDLLBII012, VEGFDLLBII013, VEGFDLLBII014, VEGFDLLBII015, VEGFDLLBII016). In these constructs, the DLLBII101G08 affinity-matured VHH DLLBII129B05 or the DLLBII115A05 affinity-matured VHH DLLBII136C07 are included. Additionally, in 2 constructs the bivalent anti-VEGF VHH comprising VEGFBII23B04 and VEGFBII5B05 is included. Two half-life extension methodologies are applied: i) PEGylation or ii) genetic fusion to a serum albumin binding VHH. Building blocks are linked via a 9 Gly-Ser, 35 Gly-Ser or 35 Gly-Ser (Cys at position 15) flexible linker. An overview of the format and sequence of all seven bispecific VHHs is depicted in Table 68-A (linker sequences are underlined), SEQ ID NOs: 74-80 and FIG. 40.

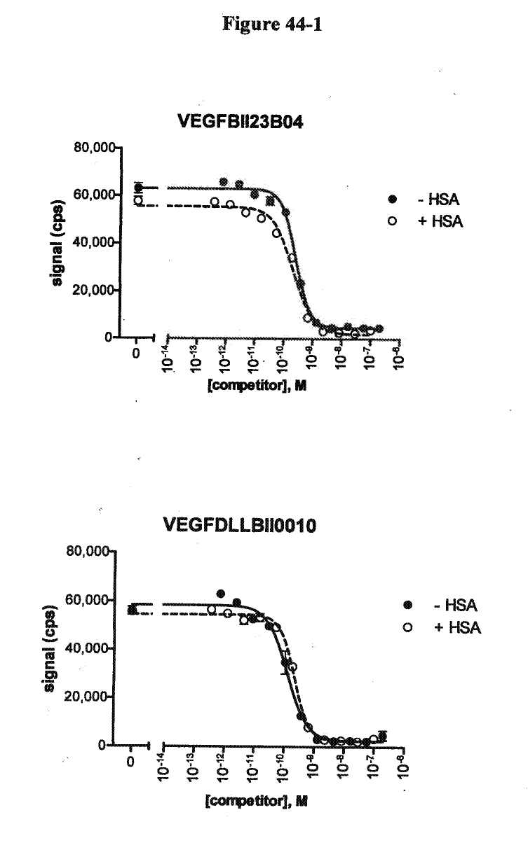

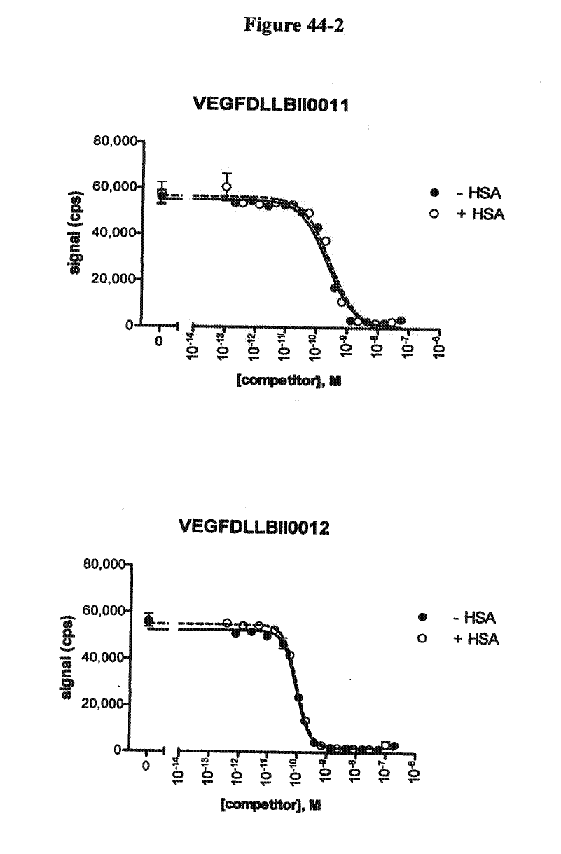

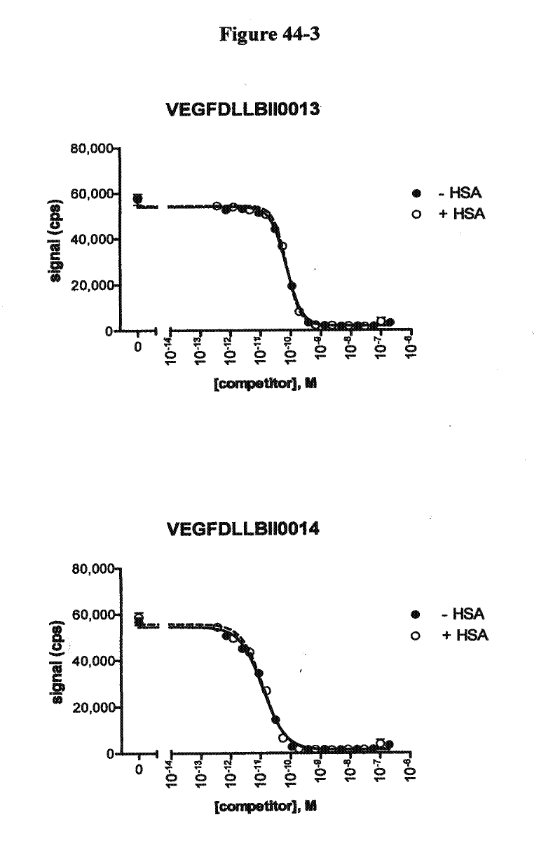

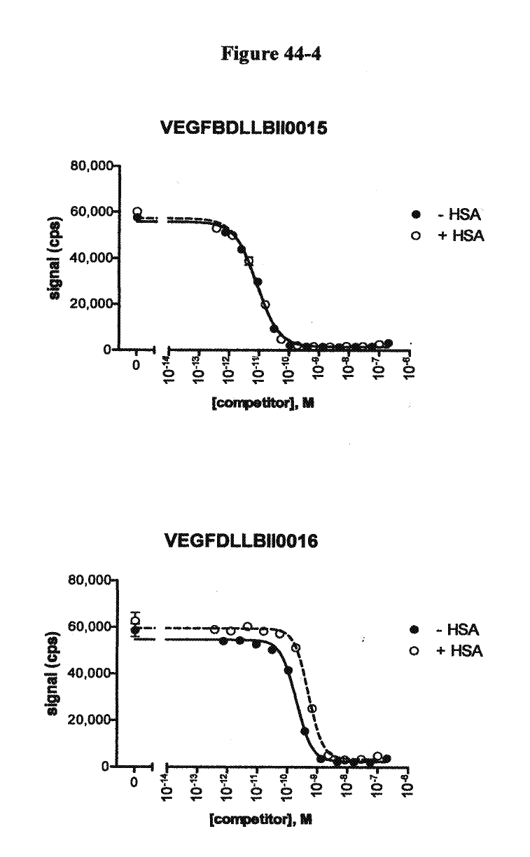

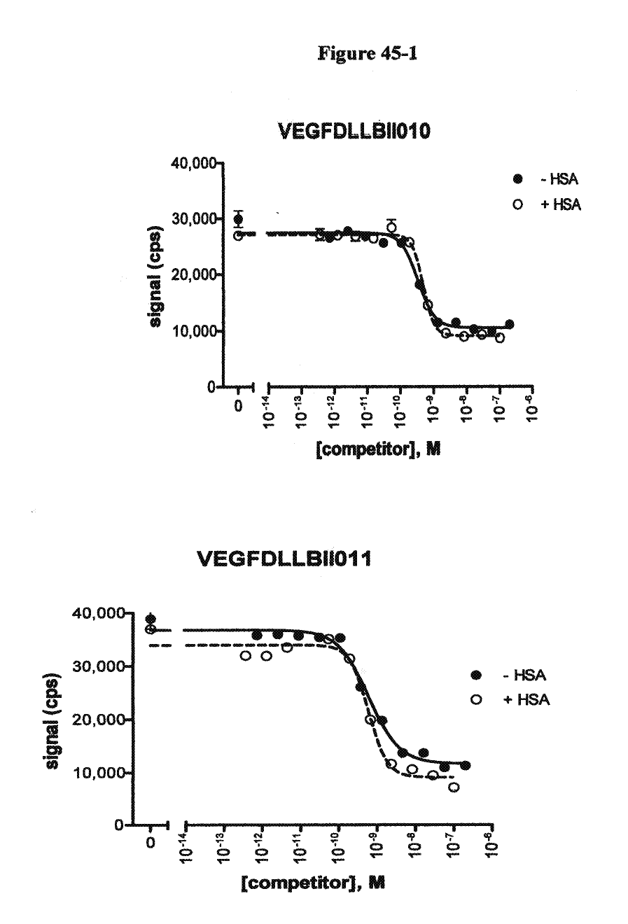

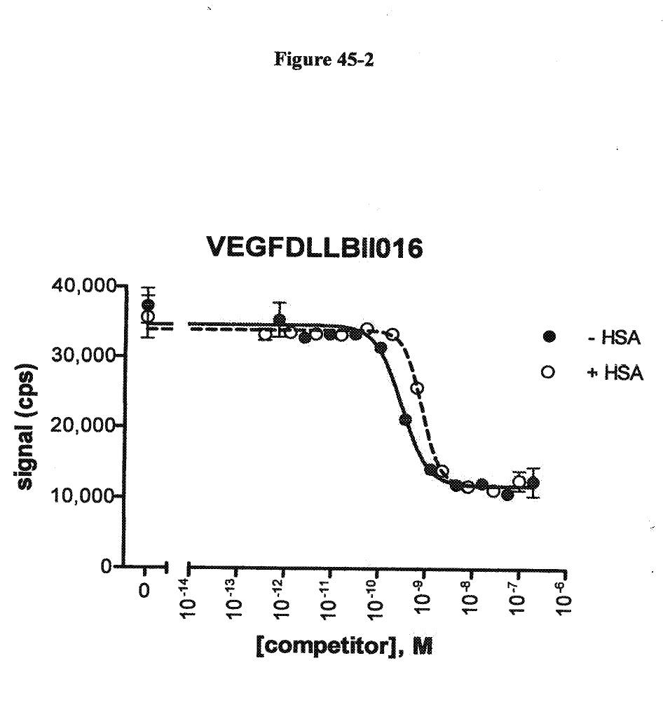

To explore the anti-VEGF blocking properties in comparison with the monovalent building block VEGFBII23B04, all seven VHHs are characterized in the VEGF/VEGFR2-Fc (Example 12.3; FIG. 44) and VEGF/VEGFR1-Fc (Example 12.4; FIG. 45) competition AlphaScreen. These 2 competition assays are also performed after preincubation of the VHHs with 5 μM human serum albumin. A summary of IC₅₀ values is shown in Table 68-B.

TABLE 68-A
Sequences of bispecific VHHs targeting VEGF and DLL4

| VHH ID/ | |
|---|---|
| SEQ ID NO: | AA sequence |
| VEGFDLLBII010 | EVQLVESGGGLVQTGDSLRLSCEVSGRTFSSYSMGWFRQAQGKEREFVVAISKGGYKYDSVSLEGRFTISKDN |
| 74 | AKNTVYLQINSLKPEDTAVYYCASSRAYGSSRLRLADTYEYWGQGTQVTVSSGGGGSGGGSEVQLVESGGGLV |
| | QPGNSLRLSCAASGFTFSSFGMSWVRQAPGKGLEWVSSISGSGSDTLYADSVKGRFTISRDNAKTTLYLQMNS |
| | LRPEDTAVYYCTIGGSLSRSSQGTLVTVSSGGGGSGGGSEVQLVESGGGLVQAGGSLRLSCAASGRTFSSYAM |
| | AWYRQAPGKEREYVAAIRWSGGTAYYADSVQGRFTISRDNAKNTVYLQMNSLKPEDTAVYYCANRAPDTRLAP |
| | YEYDHWGQGTQVTVSS |
| VEGFDLLBII011 | EVQLVESGGGLVQTGDSLRLSCEVSGRTFSSYSMGWFRQAQGKEREFVVAISKGGYKYDSVSLEGRFTISKDN |
| 75 | AKNTVYLQINSLKPEDTAVYYCASSRAYGSSRLRLADTYEYWGQGTQVTVSSGGGGSGGGSEVQLVESGGGLV |
| | QPGNSLRLSCAASGFTFSSFGMSWVRQAPGKGLEWVSSISGSGSDTLYADSVKGRFTISRDNAKTTLYLQMNS |
| | LRPEDTAVYYCTIGGSLSRSSQGTLVTVSSGGGGSGGGSEVQLVESGGGLVQPGGSLRLSCAASGFTFGSYDM |
| | SWVRRSPGKGPEWVSSINSGGGSTYYADYVKGRFTISRDNAKNTLYLQMNSLKPEDTAVYYCAADRYIWARQG |
| | EYWGAYEYDYWGQGTQVTVSS |
| VEGFDLLBII012 | EVQLVESGGGLVQTGDSLRLSCEVSGRTFSSYSMGWFRQAQGKEREFVVAISKGGYKYDSVSLEGRFTISKDN |
| 76 | AKNTVYLQINSLKPEDTAVYYCASSRAYGSSRLRLADTYEYWGQGTQVTVSSGGGGSGGGGSGGGGCGGGGSG |
| | GGGSGGGGSGGGGSEVQLVESGGGLVQPGGSLRLSCAASGFTFGSYDMSWVRRSPGKGPEWVSSINSGGGSTY |
| | YADYVKGRFTISRDNAKNTLYLQMNSLKPEDTAVYYCAADRYIWARQGEYWGAYEYDYWGQGTQVTVSS |
| VEGFDLLBII013 | EVQLVESGGGLVQTGDSLRLSCEVSGRTFSSYSMGWFRQAQGKEREFVVAISKGGYKYDSVSLEGRFTISKDN |
| 77 | AKNTVYLQINSLKPEDTAVYYCASSRAYGSSRLRLADTYEYWGQGTQVTVSSGGGGSGGGGSGGGGCGGGGSG |
| | GGGSGGGGSGGGGSEVQLVESGGGLVQAGGSLRLSCAASGRTFSSYAMAWYRQAPGKEREYVAAIRWSGGTAY |
| | YADSVQGRFTISRDNAKNTVYLQMNSLKPEDTAVYYCANRAPDTRLAPYEYDHWGQGTQVTVSS |
| VEGFDLLBII014 | EVQLVESGGGLVQTGDSLRLSCEVSGRTFSSYSMGWFRQAQGKEREFVVAISKGGYKYDSVSLEGRFTISKDN |
| 78 | AKNTVYLQINSLKPEDTAVYYCASSRAYGSSRLRLADTYEYWGQGTQVTVSSGGGGSGGGSEVQLVESGGGLV |
| | QPGGSLRLSCVASGIRFMSMAWYRQAPGKHRELVARISSGGTTAYVDSVKGRFTISRDNSKNTVYLQMNSLKA |
| | EDTAVYYCNTFSSRPNPWGAGTQVTVSSGGGGSGGGGSGGGGCGGGGSGGGGSGGGGSGGGGSEVQLVESGGG |
| | LVQAGGSLRLSCAASGRTFSSYAMAWYRQAPGKEREYVAAIRWSGGTAYYADSVQGRFTISRDNAKNTVYLQM |
| | NSLKPEDTAVYYCANRAPDTRLAPYEYDHWGQGTQVTVSS |
| VEGFDLLBII015 | EVQLVESGGGLVQTGDSLRLSCEVSGRTFSSYSMGWFRQAQGKEREFVVAISKGGYKYDSVSLEGRFTISKDN |
| 79 | AKNTVYLQINSLKPEDTAVYYCASSRAYGSSRLRLADTYEYWGQGTQVTVSSGGGGSGGGGSGGGGCGGGGSG |
| | GGGSGGGGSGGGGSEVQLVESGGGLVQPGGSLRLSCVASGIRFMSMAWYRQAPGKHRELVARISSGGTTAYVD |
| | SVKGRFTISRDNSKNTVYLQMNSLKAEDTAVYYCNTFSSRPNPWGAGTQVTVSSGGGGSGGGGSGGGGSGGGG |
| | SGGGGSGGGGSGGGGSEVQLVESGGGLVQAGGSLRLSCAASGRTFSSYAMAWYRQAPGKEREYVAAIRWSGGT |
| | AYYADSVQGRFTISRDNAKNTVYLQMNSLKPEDTAVYYCANRAPDTRLAPYEYDHWGQGTQVTVSS |
| VEGFDLLBII016 | EVQLVESGGGLVQTGDSLRLSCEVSGRTFSSYSMGWFRQAQGKEREFVVAISKGGYKYDSVSLEGRFTISKDN |
| 80 | AKNTVYLQINSLKPEDTAVYYCASSRAYGSSRLRLADTYEYWGQGTQVTVSSGGGGSGGGGSGGGGSGGGGSG |
| | GGGSGGGGSGGGGSEVQLVESGGGLVQAGGSLRLSCAASGRTFSSYAMAWYRQAPGKEREYVAAIRWSGGTAY |
| | YADSVQGRFTISRDNAKNTVYLQMNSLKPEDTAVYYCANRAPDTRLAPYEYDHWGQGTQVTVSSGGGGSGGGS |
| | EVQLVESGGGLVQPGNSLRLSCAASGFTFSSFGMSWVRQAPGKGLEWVSSISGSGSDTLYADSVKGRFTISRD |
| | NAKTTLYLQMNSLRPEDTAVYYCTIGGSLSRSSQGTLVTVSS |

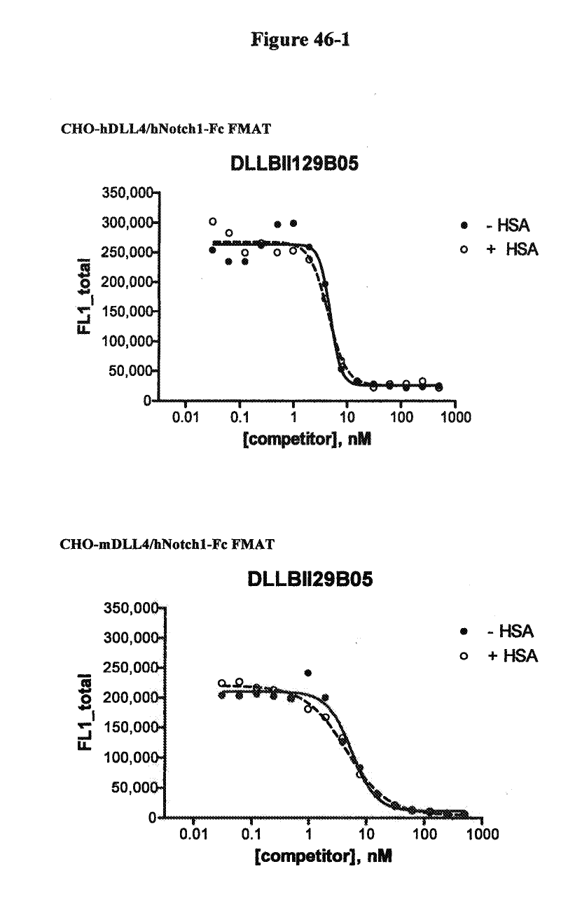

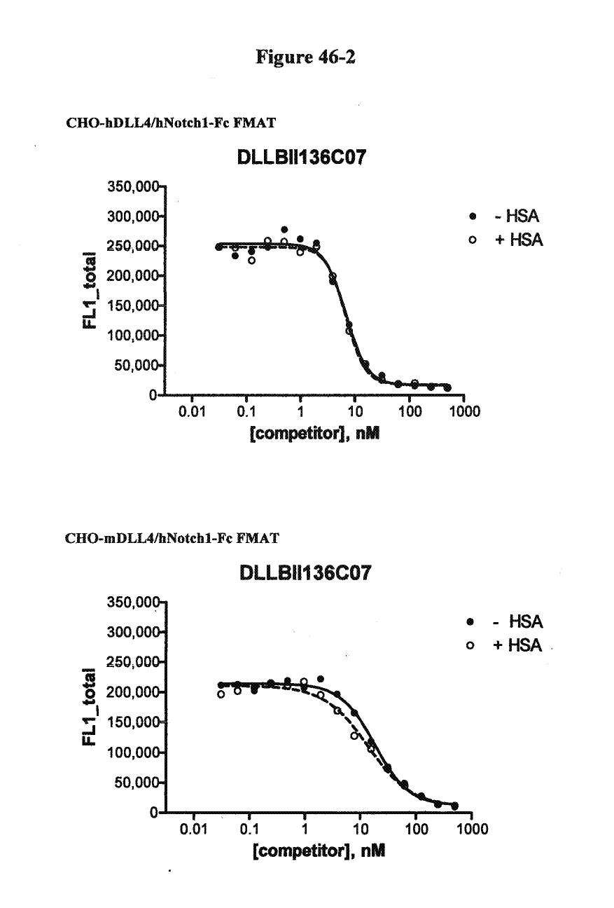

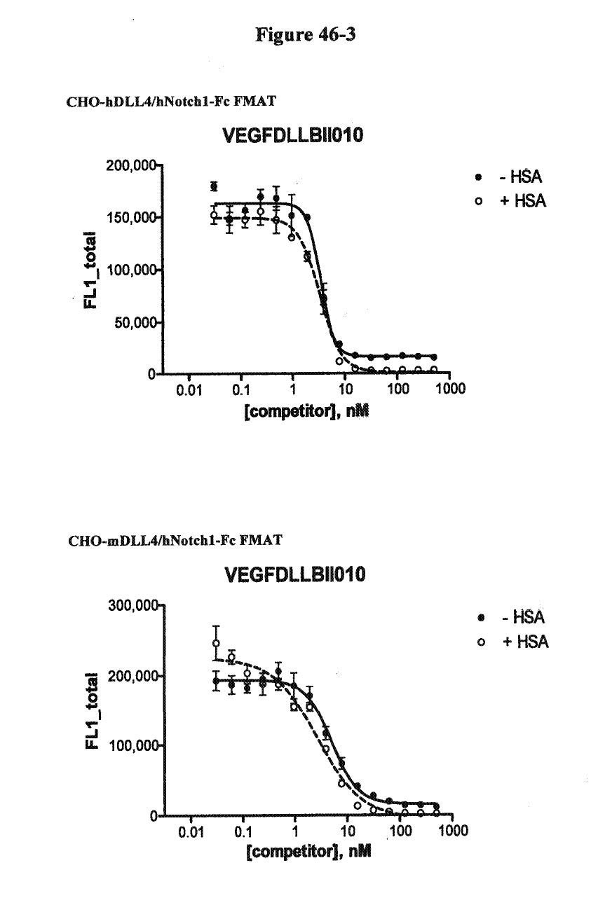

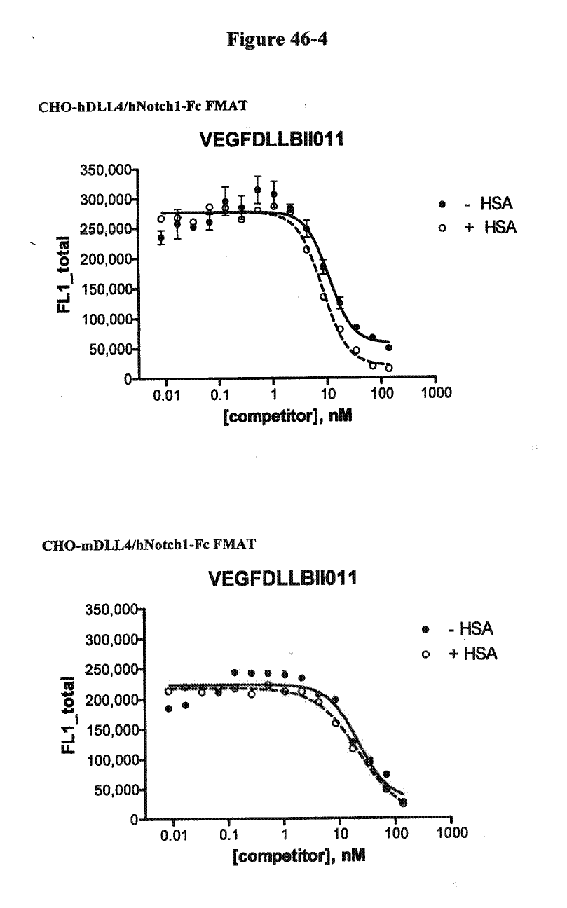

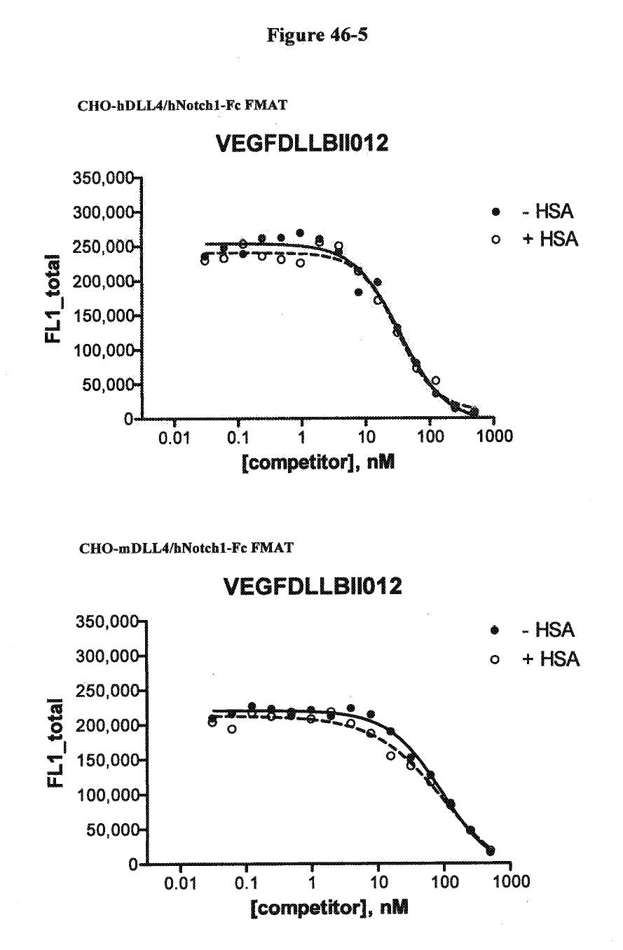

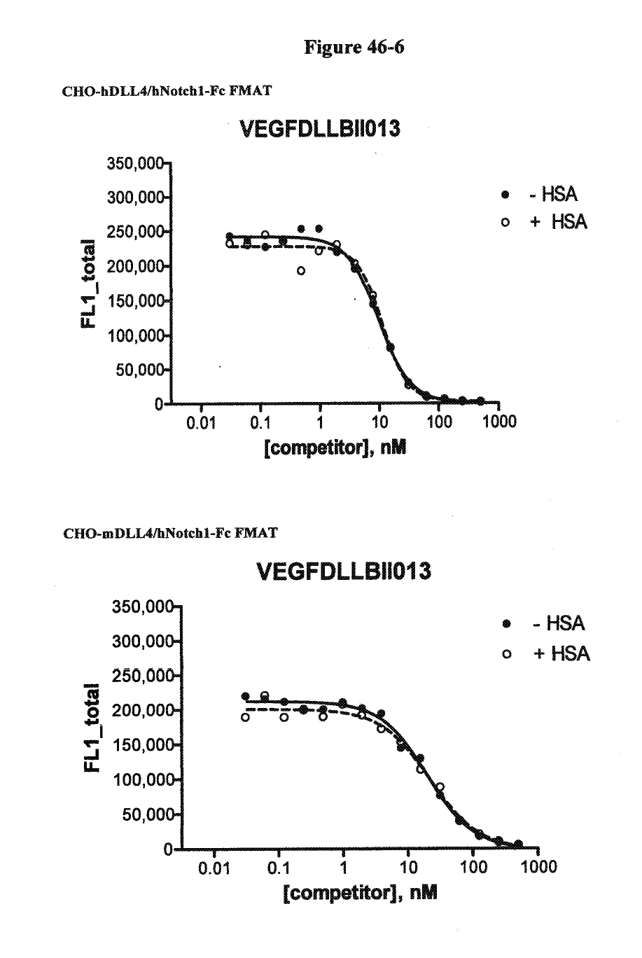

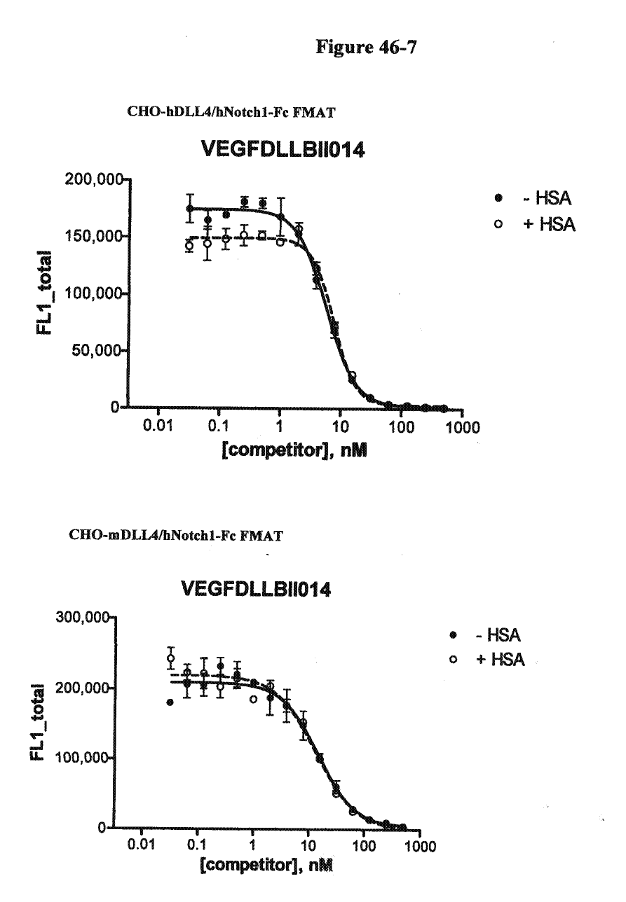

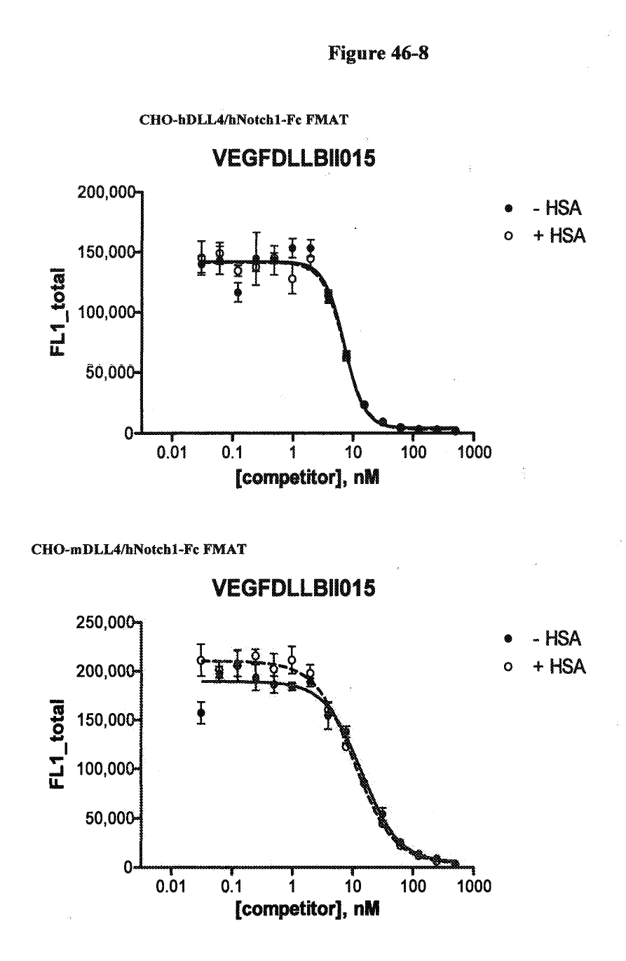

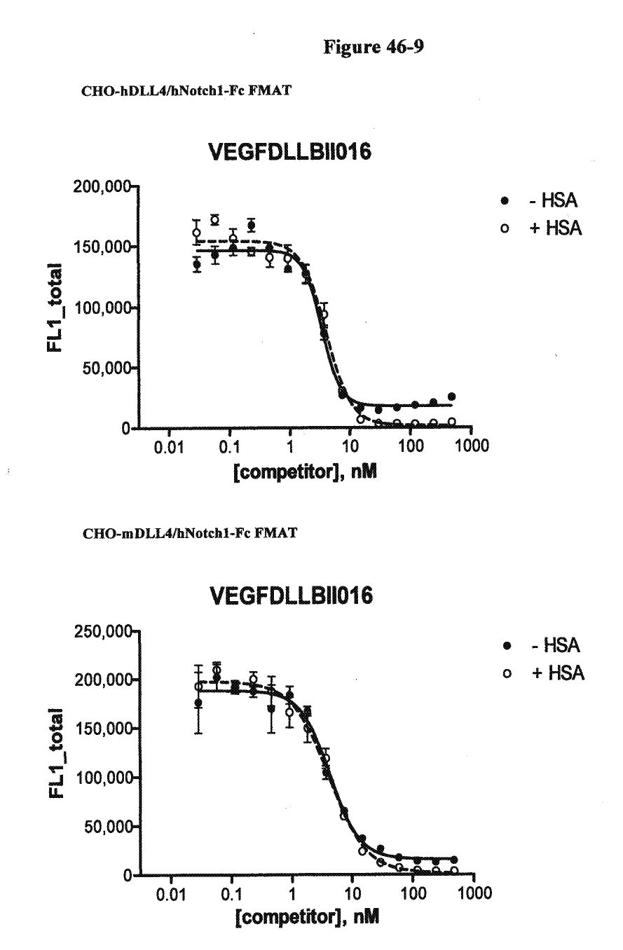

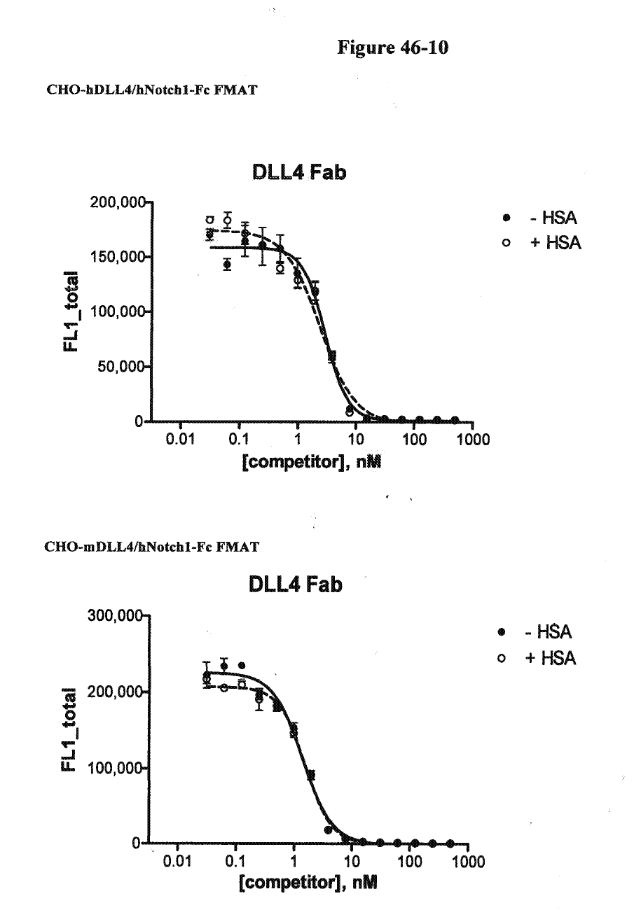

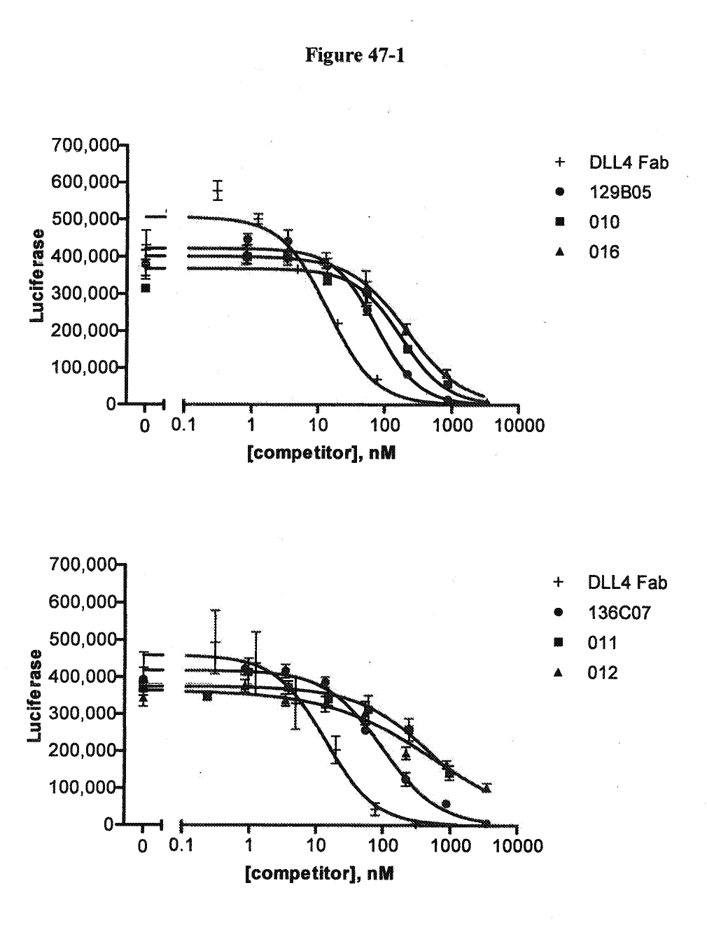

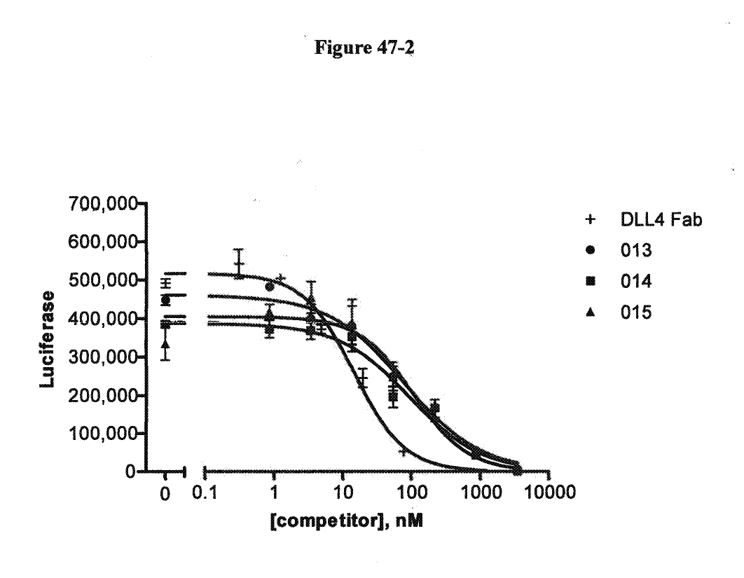

To explore the anti-DLL4 blocking properties in comparison with the monovalent affinity-matured building blocks DLLBII129B05 and DLLBII136C07, all seven VHHs are evaluated in the CHO-hDLL4/hNotch1-Fc and CHO-mDLL4/hNotch1-Fc competitive FMAT assay (Example 4; FIG. 46) and the DLL4-mediated reporter assay (Example 12.5; FIG. 47). These assays are also performed after preincubation of VHHs with 25 μM (FMAT assay) or 175 μM (reporter assay) human serum albumin. A summary of IC₅₀ values is shown in Table 69.

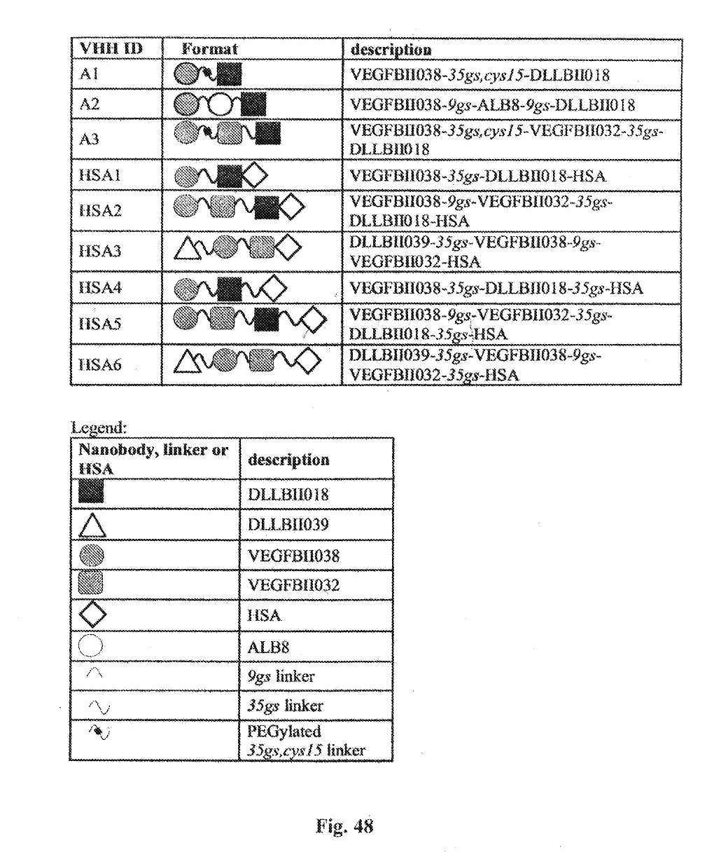

Finally, in a third cycle, the bispecific VHHs A1, A2, A3 and HSA1-6 are constructed. The following building blocks are used to generate these constructs: VEGFBII038 (sequence-optimized variant of VEGFBII23B04), VEGFBII032 (sequence-optimized variant of VEGFBII5B05), DLLBII018 (sequence-optimized variant of DLLBII129B05) and DLLBII039 (sequence-optimized variant of DLLBII136C7). Three half-life extension methodologies are applied: i) PEGylation, ii) genetic fusion to a serum albumin binding VHH and iii) genetic fusion to human serum albumin. Building blocks are linked via a 9 Gly-Ser, 35 Gly-Ser or 35 Gly-Ser (Cys at position 15) flexible linker. An overview of the format and sequence of all three bispecific VHHs is depicted in Table 70-A, SEQ ID Nos: 81-89 and in FIG. 48.

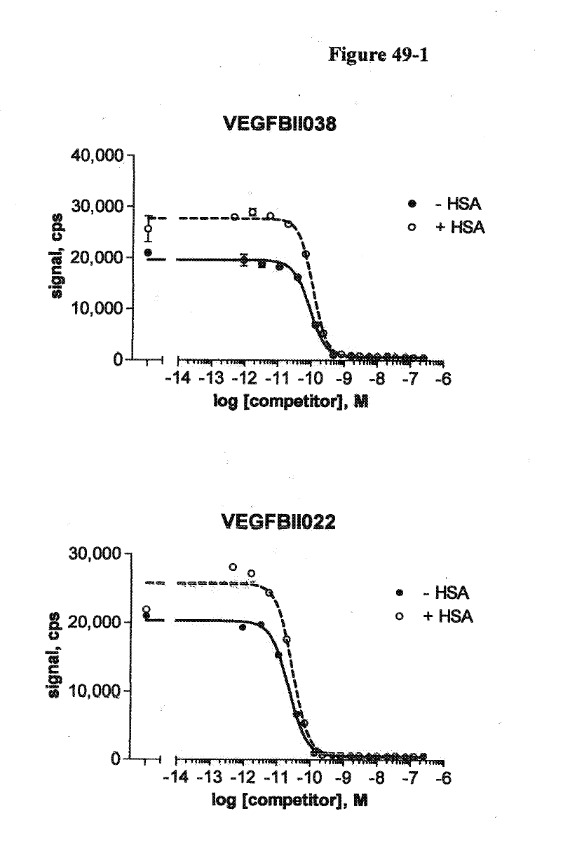

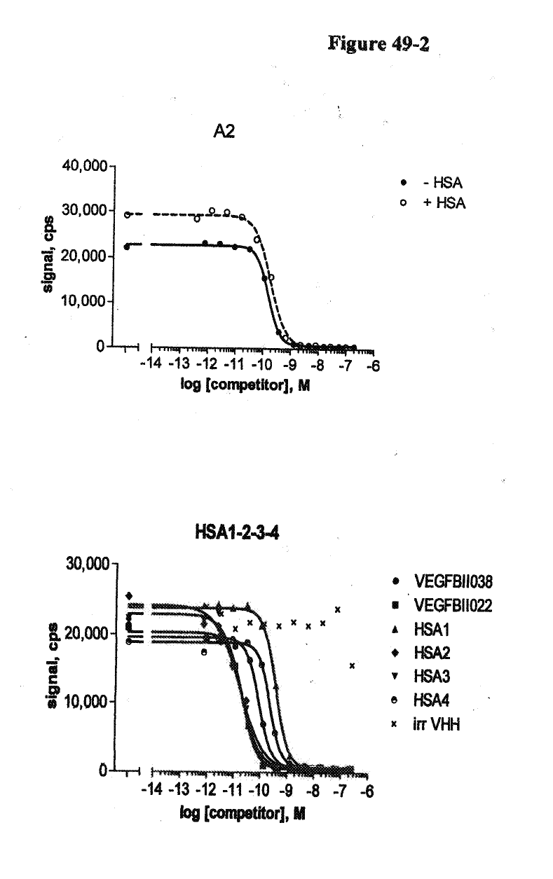

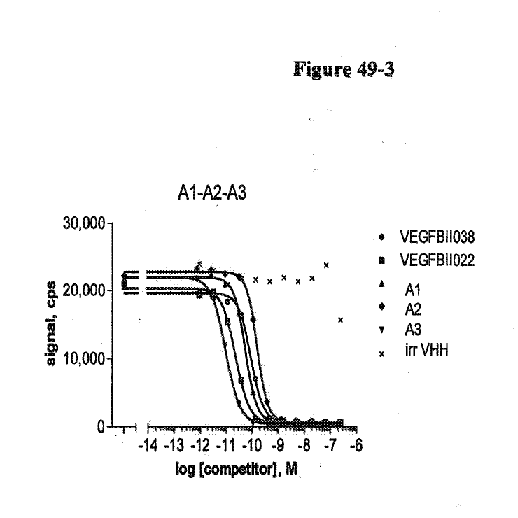

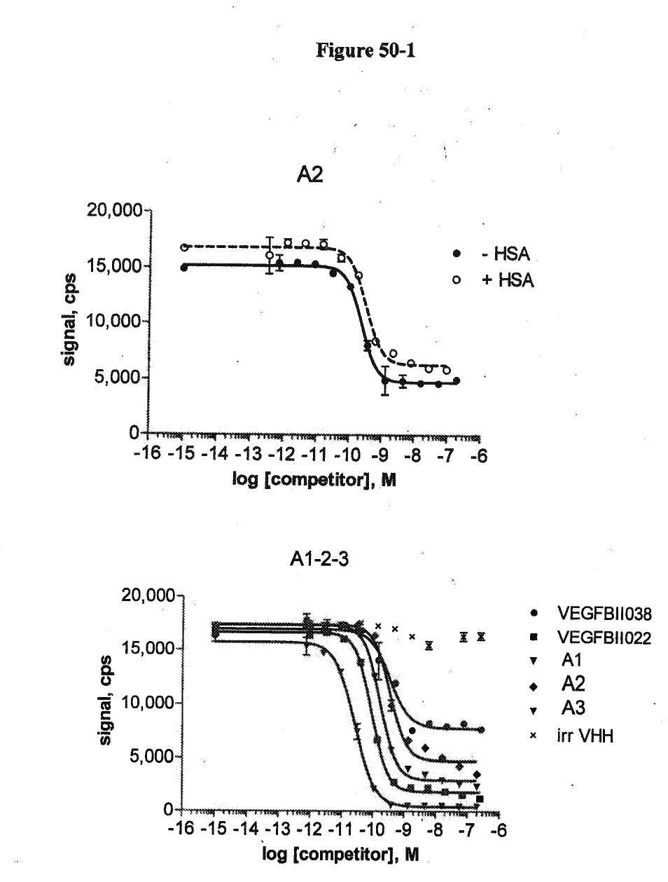

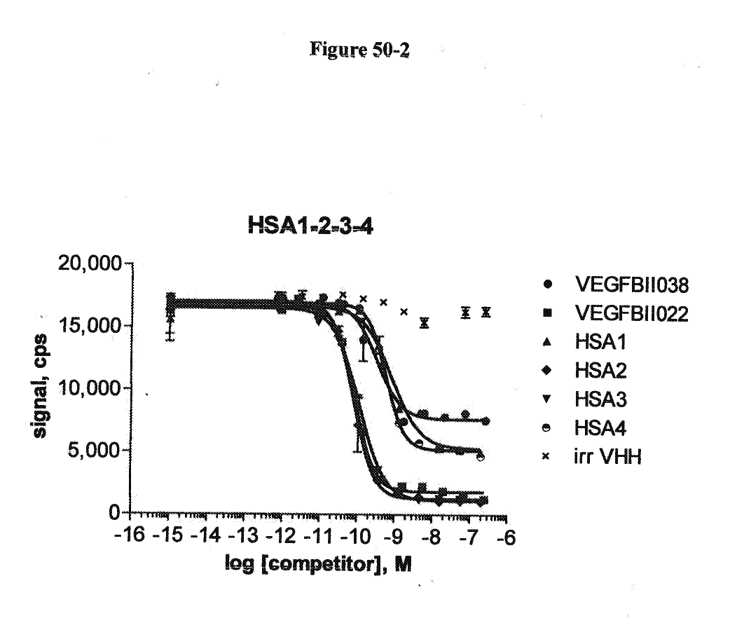

To explore the anti-VEGF blocking properties in comparison with the monovalent sequence optimized building block VEGFBII038 or biparatopic sequence optimized building block VEGFBII022, all seven VHHs are characterized in the VEGF/VEGFR2-Fc (Example 12.3; FIG. 49) and VEGF/VEGFR1-Fc (Example 12.4; FIG. 50) competition AlphaScreen. These 2 competition assays are also performed after preincubation of the VHHs with 5 μM human serum albumin. A summary of IC₅₀ values is shown in Table 70-B.

TABLE 70-A
Sequences of bispecific VHHs targeting VEGF and DLL4

| VHH ID/ | |
|---|---|
| SEQ ID NO: | AA sequence |
| A1/81 | VQLVESGGGLVQPGGSLRLSCAASGRTFSSYSMGWFRQAPGKEREFVVAISKGGYKYDAVSLE |
| | GRFTISRDNAKNTVYLQINSLRPEDTAVYYCASSRAYGSSRLRLADTYEYWGQGTLVTVSSGG |
| | GGSGGGGSGGGGCGGGGSGGGGSGGGGSGGGGSEVQLVESGGGLVQPGGSLRLSCAASGRTFS |
| | SYAMAWYRQAPGKEREYVAAIRWSGGTAYYADSVKGRFTISRDNAKNTVYLQMNSLRPEDTAV |
| | YYCANRAPDTRLAPYEYDHWGQGTLVTVSS |
| A2/82 | VQLVESGGGLVQPGGSLRLSCAASGRTFSSYSMGWFRQAPGKEREFVVAISKGGYKYDAVSLE |
| | GRFTISRDNAKNTVYLQINSLRPEDTAVYYCASSRAYGSSRLRLADTYEYWGQGTLVTVSSGG |
| | GGSGGGSEVQLVESGGGLVQPGNSLRLSCAASGFTFSSFGMSWVRQAPGKGLEWVSSISGSGS |
| | DTLYADSVKGRFTISRDNAKTTLYLQMNSLRPEDTAVYYCTIGGSLSRSSQGTLVTVSSGGGG |
| | SGGGSEVQLVESGGGLVQPGGSLRLSCAASGRTFSSYAMAWYRQAPGKEREYVAAIRWSGGTA |
| | YYADSVKGRFTISRDNAKNTVYLQMNSLRPEDTAVYYCANRAPDTRLAPYEYDHWGQGTLVTV |
| | SS |
| A3/83 | VQLVESGGGLVQPGGSLRLSCAASGRTFSSYSMGWFRQAPGKEREFVVAISKGGYKYDAVSLE |
| | GRFTISRDNAKNTVYLQINSLRPEDTAVYYCASSRAYGSSRLRLADTYEYWGQGTLVTVSSGG |
| | GGSGGGGSGGGGCGGGGSGGGGSGGGGSGGGGSEVQLVESGGGLVQPGGSLRLSCAASGIRFI |
| | SMAWYRQAPGKQRELVARISSGGTTAYADSVKGRFTISRDNSKNTVYLQMNSLRAEDTAVYYC |
| | NTFSSRPNPWGQGTLVTVSSGGGGSGGGGSGGGGSGGGGSGGGGSGGGGSGGGGSEVQLVESG |
| | GGLVQPGGSLRLSCAASGRTFSSYAMAWYRQAPGKEREYVAAIRWSGGTAYYADSVKGRFTIS |
| | RDNAKNTVYLQMNSLRPEDTAVYYCANRAPDTRLAPYEYDHWGQGTLVTVSS |
| HSA1/84 | DVQLVESGGGLVQPGGSLRLSCAASGRTFSSYSMGWFRQAPGKEREFVVAISKGGYKYDAVSL |
| | EGRFTISRDNAKNTVYLQINSLRPEDTAVYYCASSRAYGSSRLRLADTYEYWGQGTLVTVSSG |
| | GGGSGGGGSGGGGSGGGGSGGGGSGGGGSGGGGSEVQLVESGGGLVQPGGSLRLSCAASGRTF |
| | SSYAMAWYRQAPGKEREYVAAIRWSGGTAYYADSVKGRFTISRDNAKNTVYLQMNSLRPEDTA |
| | VYYCANRAPDTRLAPYEYDHWGQGTLVTVSSDAHKSEVAHRFKDLGEENFKALVLIAFAQYLQ |
| | QCPFEDHVKLVNEVTEFAKTCVADESAENCDKSLHTLFGDKLCTVATLRETYGEMADCCAKQE |
| | PERNECFLQHKDDNPNLPRLVRPEVDVMCTAFHDNEETFLKKYLYEIARRHPYFYAPELLFFA |
| | KRYKAAFTECCQAADKAACLLPKLDELRDEGKASSAKQRLKCASLQKFGERAFKAWAVARLSQ |
| | RFPKAEFAEVSKLVTDLTKVHTECCHGDLLECADDRADLAKYICENQDSISSKLKECCEKPLL |
| | EKSHCIAEVENDEMPADLPSLAADFVESKDVCKNYAEAKDVFLGMFLYEYARRHPDYSVVLLL |
| | RLAKTYETTLEKCCAAADPHECYAKVFDEFKPLVEEPQNLIKQNCELFEQLGEYKFQNALLVR |
| | YTKKVPQVSTPTLVEVSRNLGKVGSKCCKHPEAKRMPCAEDYLSVVLNQLCVLHEKTPVSDRV |
| | TKCCTESLVNRRPCFSALEVDETYVPKEFNAETFTFHADICTLSEKERQIKKQTALVELVKHK |
| | PKATKEQLKAVMDDFAAFVEKCCKADDKETCFAEEGKKLVAASQAALGL |
| HSA2/85 | DVQLVESGGGLVQPGGSLRLSCAASGRTFSSYSMGWFRQAPGKEREFVVAISKGGYKYDAVSL |
| | EGRFTISRDNAKNTVYLQINSLRPEDTAVYYCASSRAYGSSRLRLADTYEYWGQGTLVTVSSG |
| | GGGSGGGSEVQLVESGGGLVQPGGSLRLSCAASGIRFISMAWYRQAPGKQRELVARISSGGTT |
| | AYADSVKGRFTISRDNSKNTVYLQMNSLRAEDTAVYYCNTFSSRPNPWGQGTLVTVSSGGGGS |
| | GGGGSGGGGSGGGGSGGGGSGGGGSGGGGSEVQLVESGGGLVQPGGSLRLSCAASGRTFSSYA |
| | MAWYRQAPGKEREYVAAIRWSGGTAYYADSVKGRFTISRDNAKNTVYLQMNSLRPEDTAVYYC |
| | ANRAPDTRLAPYEYDHWGQGTLVTVSSDAHKSEVAHRFKDLGEENFKALVLIAFAQYLQQCPF |
| | EDHVKLVNEVTEFAKTCVADESAENCDKSLHTLFGDKLCTVATLRETYGEMADCCAKQEPERN |
| | ECFLQHKDDNPNLPRLVRPEVDVMCTAFHDNEETFLKKYLYEIARRHPYFYAPELLFFAKRYK |
| | AAFTECCQAADKAACLLPKLDELRDEGKASSAKQRLKCASLQKFGERAFKAWAVARLSQRFPK |
| | AEFAEVSKLVTDLTKVHTECCHGDLLECADDRADLAKYICENQDSISSKLKECCEKPLLEKSH |
| | CIAEVENDEMPADLPSLAADFVESKDVCKNYAEAKDVFLGMFLYEYARRHPDYSVVLLLRLAK |
| | TYETTLEKCCAAADPHECYAKVFDEFKPLVEEPQNLIKQNCELFEQLGEYKFQNALLVRYTKK |
| | VPQVSTPTLVEVSRNLGKVGSKCCKHPEAKRMPCAEDYLSVVLNQLCVLHEKTPVSDRVTKCC |
| | TESLVNRRPCFSALEVDETYVPKEFNAETFTFHADICTLSEKERQIKKQTALVELVKHKPKAT |
| | KEQLKAVMDDFAAFVEKCCKADDKETCFAEEGKKLVAASQAALGL |
| HSA3/86 | EVQLVESGGGLVQPGGSLRLSCAASGFTIGSYDMSWVRRAPGKGPEWVSSISSGGGSTYYADY |
| | VKGRFTISRDNAKNTLYLQMNSLRPEDTAVYYCAADRYIWARQGEYWGAYEYDYWGQGTLVTV |
| | SSGGGGSGGGGSGGGGSGGGGSGGGGSGGGGSGGGGSDVQLVESGGGLVQPGGSLRLSCAASG |
| | RTFSSYSMGWFRQAPGKEREFVVAISKGGYKYDAVSLEGRFTISRDNAKNTVYLQINSLRPED |
| | TAVYYCASSRAYGSSRLRLADTYEYWGQGTLVTVSSGGGGSGGGSEVQLVESGGGLVQPGGSL |
| | RLSCAASGIRFISMAWYRQAPGKQRELVARISSGGTTAYADSVKGRFTISRDNSKNTVYLQMN |
| | SLRAEDTAVYYCNTFSSRPNPWGQGTLVTVSSDAHKSEVAHRFKDLGEENFKALVLIAFAQYL |
| | QQCPFEDHVKLVNEVTEFAKTCVADESAENCDKSLHTLFGDKLCTVATLRETYGEMADCCAKQ |
| | EPERNECFLQHKDDNPNLPRLVRPEVDVMCTAFHDNEETFLKKYLYEIARRHPYFYAPELLFF |
| | AKRYKAAFTECCQAADKAACLLPKLDELRDEGKASSAKQRLKCASLQKFGERAFKAWAVARLS |
| | QRFPKAEFAEVSKLVTDLTKVHTECCHGDLLECADDRADLAKYICENQDSISSKLKECCEKPL |
| | LEKSHCIAEVENDEMPADLPSLAADFVESKDVCKNYAEAKDVFLGMFLYEYARRHPDYSVVLL |
| | LRLAKTYETTLEKCCAAADPHECYAKVFDEFKPLVEEPQNLIKQNCELFEQLGEYKFQNALLV |
| | RYTKKVPQVSTPTLVEVSRNLGKVGSKCCKHPEAKRMPCAEDYLSVVLNQLCVLHEKTPVSDR |
| | VTKCCTESLVNRRPCFSALEVDETYVPKEFNAETFTFHADICTLSEKERQIKKQTALVELVKH |
| | KPKATKEQLKAVMDDFAAFVEKCCKADDKETCFAEEGKKLVAASQAALGL |
| HSA4/87 | DVQLVESGGGLVQPGGSLRLSCAASGRTFSSYSMGWFRQAPGKEREFVVAISKGGYKYDAVSL |
| | EGRFTISRDNAKNTVYLQINSLRPEDTAVYYCASSRAYGSSRLRLADTYEYWGQGTLVTVSSG |
| | GGGSGGGGSGGGGSGGGGSGGGGSGGGGSGGGGSEVQLVESGGGLVQPGGSLRLSCAASGRTF |
| | SSYAMAWYRQAPGKEREYVAAIRWSGGTAYYADSVKGRFTISRDNAKNTVYLQMNSLRPEDTA |
| | VYYCANRAPDTRLAPYEYDHWGQGTLVTVSSGGGGSGGGGSGGGGSGGGGSGGGGSGGGGSGG |
| | GGSDAHKSEVAHRFKDLGEENFKALVLIAFAQYLQQCPFEDHVKLVNEVTEFAKTCVADESAE |
| | NCDKSLHTLFGDKLCTVATLRETYGEMADCCAKQEPERNECFLQHKDDNPNLPRLVRPEVDVM |
| | CTAFHDNEETFLKKYLYEIARRHPYFYAPELLFFAKRYKAAFTECCQAADKAACLLPKLDELR |
| | DEGKASSAKQRLKCASLQKFGERAFKAWAVARLSQRFPKAEFAEVSKLVTDLTKVHTECCHGD |
| | LLECADDRADLAKYICENQDSISSKLKECCEKPLLEKSHCIAEVENDEMPADLPSLAADFVES |
| | KDVCKNYAEAKDVFLGMFLYEYARRHPDYSVVLLLRLAKTYETTLEKCCAAADPHECYAKVFD |
| | EFKPLVEEPQNLIKQNCELFEQLGEYKFQNALLVRYTKKVPQVSTPTLVEVSRNLGKVGSKCC |
| | KHPEAKRMPCAEDYLSVVLNQLCVLHEKTPVSDRVTKCCTESLVNRRPCFSALEVDETYVPKE |
| | FNAETFTFHADICTLSEKERQIKKQTALVELVKHKPKATKEQLKAVMDDFAAFVEKCCKADDK |
| | ETCFAEEGKKLVAASQAALGL |
| HSA5/88 | DVQLVESGGGLVQPGGSLRLSCAASGRTFSSYSMGWFRQAPGKEREFVVAISKGGYKYDAVSL |
| | EGRFTISRDNAKNTVYLQINSLRPEDTAVYYCASSRAYGSSRLRLADTYEYWGQGTLVTVSSG |
| | GGGSGGGSEVQLVESGGGLVQPGGSLRLSCAASGIRFISMAWYRQAPGKQRELVARISSGGTT |
| | AYADSVKGRFTISRDNSKNTVYLQMNSLRAEDTAVYYCNTFSSRPNPWGQGTLVTVSSGGGGS |
| | GGGGSGGGGSGGGGSGGGGSGGGGSGGGGSEVQLVESGGGLVQPGGSLRLSCAASGRTFSSYA |
| | MAWYRQAPGKEREYVAAIRWSGGTAYYADSVKGRFTISRDNAKNTVYLQMNSLRPEDTAVYYC |
| | ANRAPDTRLAPYEYDHWGQGTLVTVSSGGGGSGGGGSGGGGSGGGGSGGGGSGGGGSGGGGSD |
| | AHKSEVAHRFKDLGEENFKALVLIAFAQYLQQCPFEDHVKLVNEVTEFAKTCVADESAENCDK |
| | SLHTLFGDKLCTVATLRETYGEMADCCAKQEPERNECFLQHKDDNPNLPRLVRPEVDVMCTAF |
| | HDNEETFLKKYLYEIARRHPYFYAPELLFFAKRYKAAFTECCQAADKAACLLPKLDELRDEGK |
| | ASSAKQRLKCASLQKFGERAFKAWAVARLSQRFPKAEFAEVSKLVTDLTKVHTECCHGDLLEC |
| | ADDRADLAKYICENQDSISSKLKECCEKPLLEKSHCIAEVENDEMPADLPSLAADFVESKDVC |
| | KNYAEAKDVFLGMFLYEYARRHPDYSVVLLLRLAKTYETTLEKCCAAADPHECYAKVFDEFKP |
| | LVEEPQNLIKQNCELFEQLGEYKFQNALLVRYTKKVPQVSTPTLVEVSRNLGKVGSKCCKHPE |
| | AKRMPCAEDYLSVVLNQLCVLHEKTPVSDRVTKCCTESLVNRRPCFSALEVDETYVPKEFNAE |
| | TFTFHADICTLSEKERQIKKQTALVELVKHKPKATKEQLKAVMDDFAAFVEKCCKADDKETCF |
| | AEEGKKLVAASQAALGL |
| HSA6/89 | EVQLVESGGGLVQPGGSLRLSCAASGFTIGSYDMSWVRRAPGKGPEWVSSISSGGGSTYYADY |
| | VKGRFTISRDNAKNTLYLQMNSLRPEDTAVYYCAADRYIWARQGEYWGAYEYDYWGQGTLVTV |
| | SSGGGGSGGGGSGGGGSGGGGSGGGGSGGGGSGGGGSDVQLVESGGGLVQPGGSLRLSCAASG |
| | RTFSSYSMGWFRQAPGKEREFVVAISKGGYKYDAVSLEGRFTISRDNAKNTVYLQINSLRPED |
| | TAVYYCASSRAYGSSRLRLADTYEYWGQGTLVTVSSGGGGSGGGSEVQLVESGGGLVQPGGSL |
| | RLSCAASGIRFISMAWYRQAPGKQRELVARISSGGTTAYADSVKGRFTISRDNSKNTVYLQMN |
| | SLRAEDTAVYYCNTFSSRPNPWGQGTLVTVSSGGGGSGGGGSGGGGSGGGGSGGGGSGGGGSG |
| | GGGSDAHKSEVAHRFKDLGEENFKALVLIAFAQYLQQCPFEDHVKLVNEVTEFAKTCVADESA |
| | ENCDKSLHTLFGDKLCTVATLRETYGEMADCCAKQEPERNECFLQHKDDNPNLPRLVRPEVDV |
| | MCTAFHDNEETFLKKYLYEIARRHPYFYAPELLFFAKRYKAAFTECCQAADKAACLLPKLDEL |
| | RDEGKASSAKQRLKCASLQKFGERAFKAWAVARLSQRFPKAEFAEVSKLVTDLTKVHTECCHG |
| | DLLECADDRADLAKYICENQDSISSKLKECCEKPLLEKSHCIAEVENDEMPADLPSLAADFVE |
| | SKDVCKNYAEAKDVFLGMFLYEYARRHPDYSVVLLLRLAKTYETTLEKCCAAADPHECYAKVF |
| | DEFKPLVEEPQNLIKQNCELFEQLGEYKFQNALLVRYTKKVPQVSTPTLVEVSRNLGKVGSKC |
| | CKHPEAKRMPCAEDYLSVVLNQLCVLHEKTPVSDRVTKCCTESLVNRRPCFSALEVDETYVPK |
| | EFNAETFTFHADICTLSEKERQIKKQTALVELVKHKPKATKEQLKAVMDDFAAFVEKCCKADD |
| | KETCFAEEGKKLVAASQAALGL |

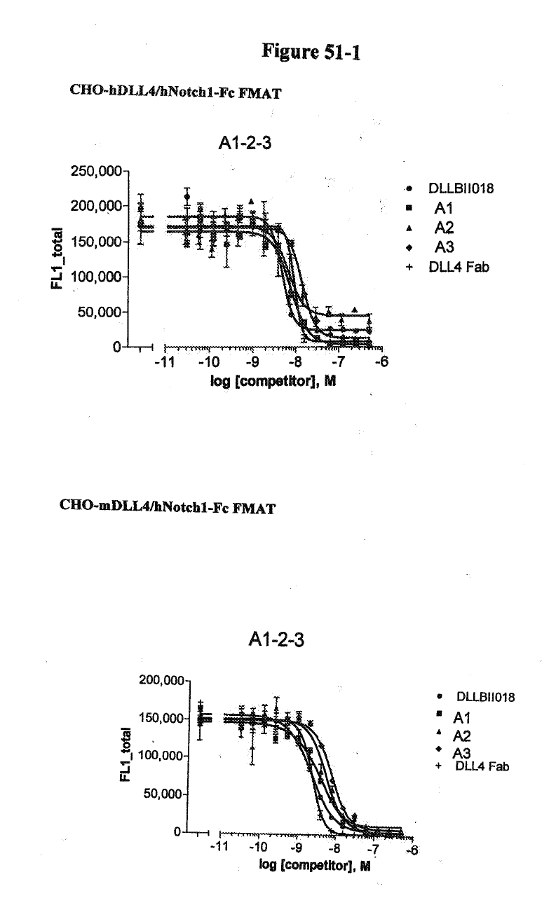

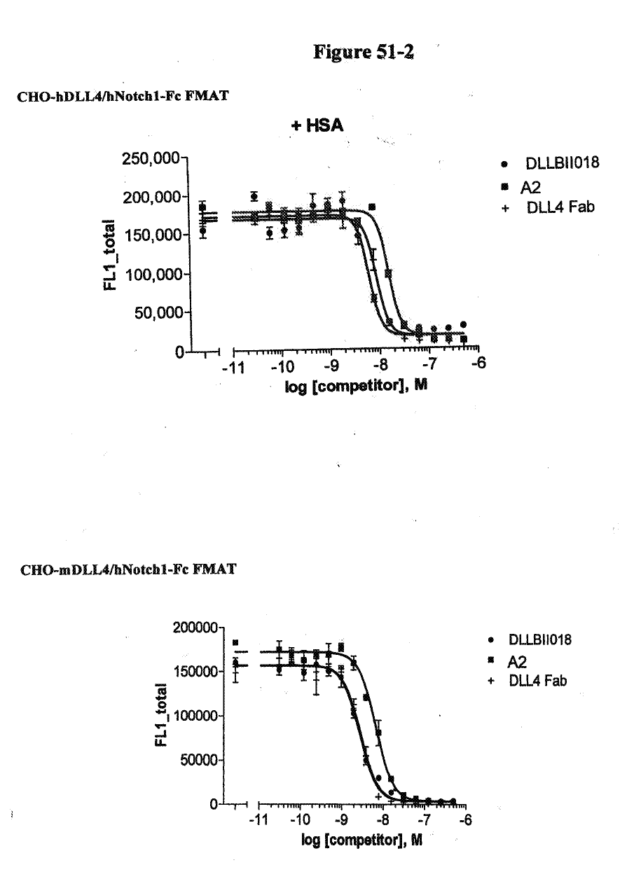

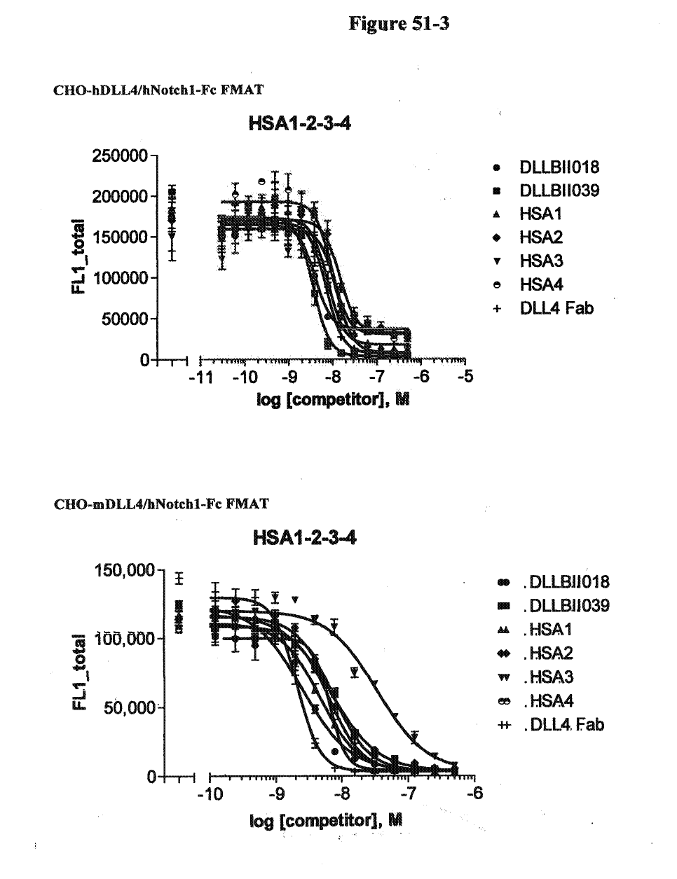

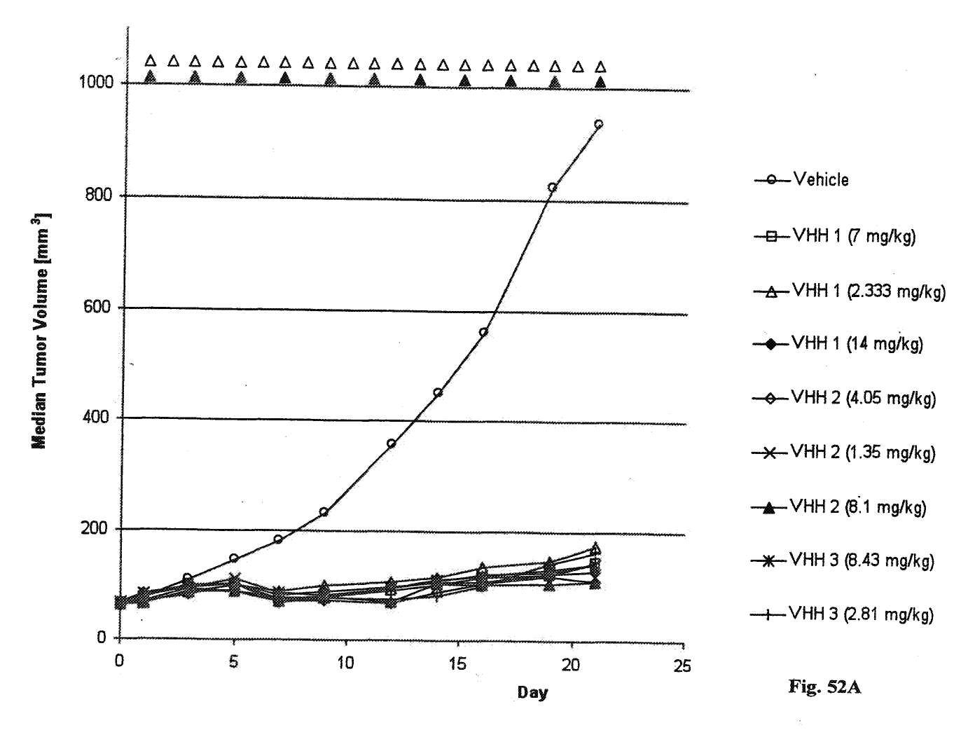

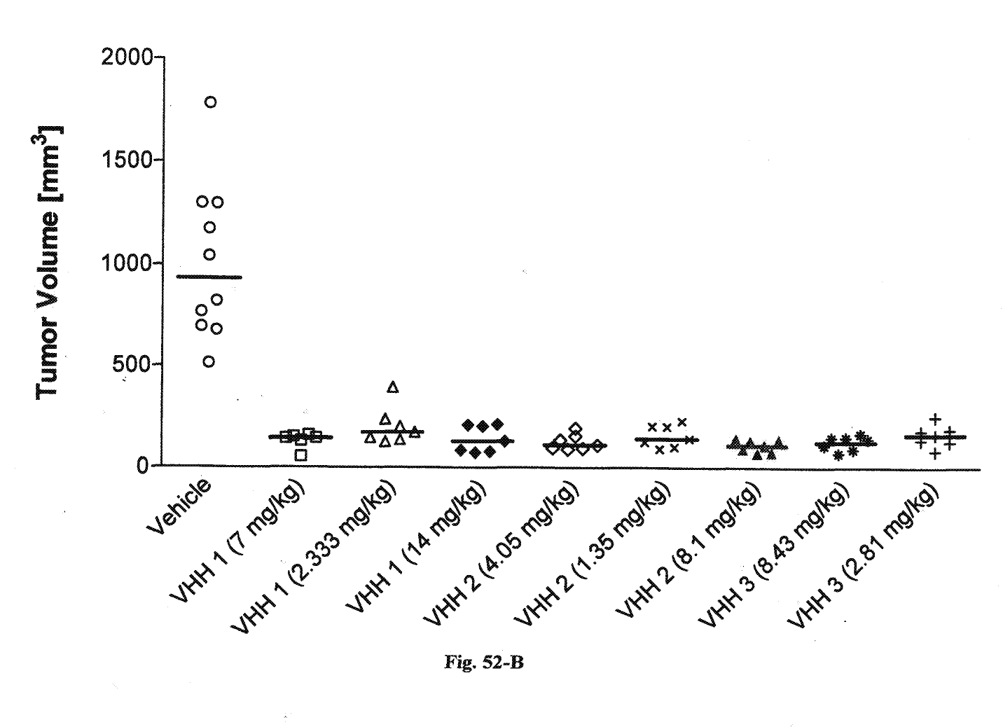

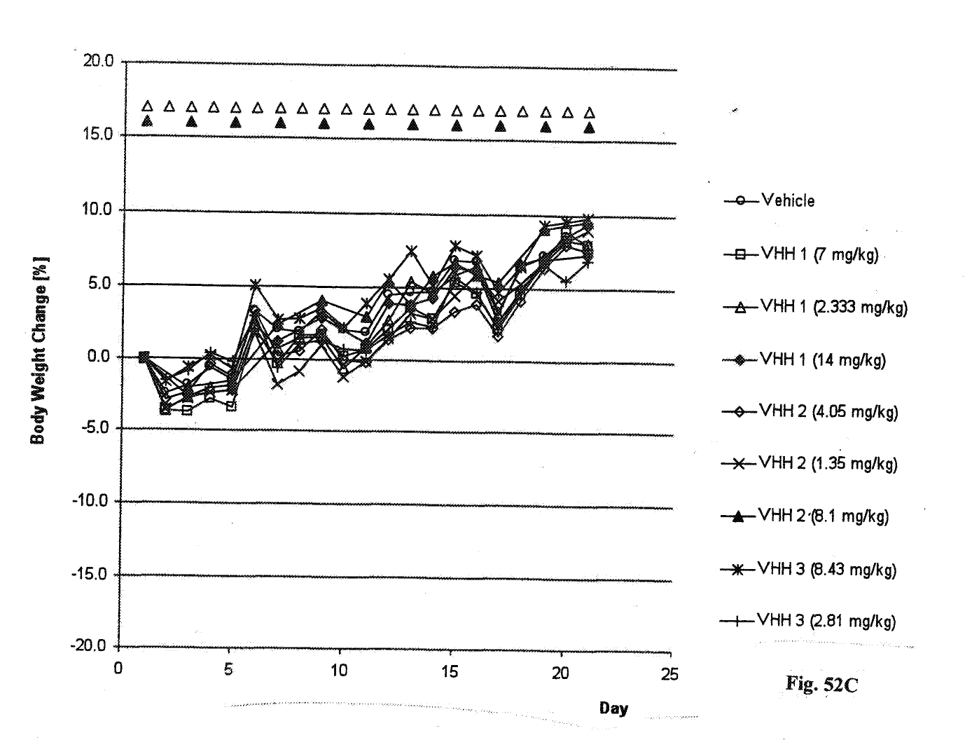

To explore the anti-DLL4 blocking properties in comparison with the monovalent sequence-optimized building blocks DLLBII018 and DLLBII039, all seven VHHs are evaluated in the CHO-hDLL4/hNotch1-Fc and CHO-mDLL4/hNotch1-Fc competitive FMAT assay (Example 4; FIG. 51). These assays are also performed after preincubation of VHHs with 25 μM (FMAT assay) human serum albumin. A summary of IC₅₀ values is shown in Table 71.

The potency of the bispecific VHHs is evaluated in the VEGF proliferation assay. In brief, primary HUVEC cells (Technoclone) are supplement-starved over night and then 4000 cells/well are seeded in quadruplicate in 96-well tissue culture plates. Cells are stimulated in the absence or presence of VHHs with 33 ng/mL VEGF. This assay is performed after preincubation of the VHHs with 520 nM human serum albumin, as indicated. The proliferation rates are measured by [³H] Thymidine incorporation on day 4. The results shown in Table 72 demonstrate that the bispecific VHHs and Bevacizumab inhibit the VEGF induced HUVEC proliferation by more than 90%, with IC₅₀s<1 nM.

The potency of the bispecific VHHs is assessed in the VEGF HUVEC Erk phosphorylation assay. In brief, primary HUVEC cells are serum-starved over night and then stimulated in the absence or presence of VHHs with 10 ng/mL VEGF for 5 min. This assay is performed after preincubation of the VHHs with 250 nM human serum albumin, as indicated. Cells are fixed with 4% Formaldehyde in PBS and ERK phosphorylation levels are measured by ELISA using phosphoERK-specific antibodies (anti-phosphoMAP Kinase pERK1&2, M8159, Sigma) and polyclonal Rabbit Anti-Mouse-Immunoglobulin-HRP conjugate (PO161, Dako). As shown in Table 73, the bispecific VHHs and Bevacizumab inhibit the VEGF induced Erk phosphorylation by more than 90%, with IC₅₀s<1 nM.

The potency of the bispecific VHHs is evaluated in the DII4 HUVEC proliferation assay, as described by Ridgway et al., Nature. 2006 Dec. 21; 444 (7122):1083-7, in modified form. In brief, 96-well tissue culture plates are coated with purified DII4-His (RnD Systems; C-terminal His-tagged human DII4, amino acid 27-524, 0.75 ml/well, 10 ng/ml) in coating buffer (PBS, 0.1% BSA). Wells are washed in PBS before 4000 HUVEC cells/well are seeded in quadruplicate. This assay is performed after preincubation of the VHHs with 50 μM human serum albumin, as indicated. Cell proliferation is measured by [³H]-Thymidine incorporation on day 4. The IC₅₀ values of the bispecific VHHs and the DLL4 Fab are summarized in Table 74.

### EXAMPLE 17

Efficacy of Selected Binding Molecules in a Mouse Model of Human Colon Cancer

The efficacy of the three selected VHHs VEGFDLLBII010, VEGFDLLBII013 and VEGFDLLBII015 is assessed in a mouse model of human colon cancer (cell line SW620) in nude mice.

SW620 cells are obtained from ATCC (CCL-227). Cells are cultured in T175 tissue culture flasks at 37° C. and 0% CO₂. The medium used is Leibovitz's L-15 Medium (Gibco Cat. 11415) and 10% fetal calf serum (JRH Cat. 12103-1000 ml). Cultures are split at subconfluency with a split ratio of 1:10 or 1:20. Mice are 7 week-old athymic female BomTac:NMRI-Foxn1^{nu}, purchased from Taconic, Denmark. To establish subcutaneous tumors, SW620 cells are trypsinized, washed, resuspended in PBS+5% FCS at 5×10⁷/ml. 100 μl cell suspension containing 5×10⁶ cells are then injected subcutaneously into the right flank of the mice (one site per mouse). When tumors are well established and have reached volumes of 47 to 93 mm³ (10 days after injecting the cells), mice are randomly distributed between the treatment and the vehicle control groups.

VHHs are diluted with PBS.

The doses are calculated to the Avastin (bevacizumab) equivalent doses of 7.5 mg/kg, 2.5 mg/kg and 15 mg/kg, respectively (Table 75). All doses are calculated according to the average body weight of all mice on day 0 (27.7 g) and administered in a volume of 100 μl per mouse. VHHs are administered daily or every second day intraperitoneally. Day 1 is the first, day 21 the last day of treatment.

Tumor diameters are measured three times a week (Monday, Wednesday and Friday) with a caliper. The volume of each tumor [in mm³] is calculated according to the formula “tumor volume=length*diameter²*π/6.” To monitor side effects of treatment, mice are inspected daily for abnormalities and body weight is determined three times a week (Monday, Wednesday and Friday). Animals are sacrificed when the control tumors reach a size of approximately 1000 mm³ on average.

The statistical evaluation is performed for the parameters tumor volume and body weight at the end of the experiment at day 21. For the tumor volume absolute values, and for the body weight the percentage of change refers to the initial weight of day 1 are used. Due to the observed variation, nonparametric methods are applied.

For descriptive considerations the number of observations, the median, the minimum and the maximum is calculated. For a quick overview of possible treatment effects, the median of the tumor volume of each treatment group T is referred to the median of the control C
relative tumor volume (T/C)

$$T / C = 100 * \frac{{T}_{d}}{{C}_{d}}$$

tumor growth inhibition (TGI) from day 1 until day d

$$TGI = 100 * \frac{( {C}_{d} - {C}_{1} ) - ( {T}_{d} - {T}_{1} )}{( {C}_{d} - {C}_{1} )}$$

where C₁, T₁=median tumor volumes in control and treatment group at start of the experiment at day 1,
C_{d}, T_{d}=median tumor volumes in control and treatment group at end of the experiment at day d

A one-sided decreasing Wilcoxon test is applied to compare the dosage groups of the three VHHs with the control, looking for a reduction in tumor volume as effect and a reduction in the body weight gain as adverse event.

The p values for the tumor volume (efficacy parameter) are adjusted for multiple comparisons according to Bonferroni-Holm, whereas the p values of the body weight (tolerability parameter) remain unadjusted in order not to overlook a possible adverse effect.

The level of significance is fixed at α=5%. An (adjusted) p-value of less than 0.05 is considered to show a difference between treatment groups; differences are seen as indicative whenever 0.05≦p-value<0.10.

The statistical evaluation is prepared using the software package SAS version 9.2 (SAS Institute Inc., Cary N.C., USA) and Proc StatXact (Cytel Software Corporation, Cambridge Mass., USA).

As shown in FIG. 52 and Table 75 and 76, VEGFDLLBII013, VEGFDLLBII010 and VEGFDLLBII015 show significant efficacy in the SW620 colon cancer model and are well tolerated.

FIG. 52A shows the SW620 tumor growth kinetics: SW620 tumor-bearing mice are treated daily (open symbols) with VEGFDLLBII013 (VHH 1), VEGFDLLBII010 (VHH 2) or VEGFDLLBII015 (VHH 3) or every second day (closed symbols) with VEGFDLLBII013 (VHH 1) or VEGFDLLBII010 (VHH 2). Median tumor volumes are plotted over time. Day 1 is the first day, day 21 the last day of the experiment. The triangles on the top of the graph indicate the treatment days.

FIG. 52B shows the absolute tumor volumes at the end of the study on day 21: SW620 tumor-bearing mice are treated daily (open symbols) with VEGFDLLBII013 (VHH 1), VEGFDLLBII010 (VHH 2) or VEGFDLLBII015 (VHH 3) or every second day (closed symbols) with VEGFDLLBII013 (VHH 1) or VEGFDLLBII010 (VHH 2). Individual absolute tumor volumes at day 21 are plotted. Each symbol represents an individual tumor. The horizontal lines represent the median tumor volumes.

FIG. 52C shows the change of body weight over time; SW620 tumor-bearing mice are treated daily (open symbols) with VEGFDLLBII013 (VHH 1), VEGFDLLBII010 (VHH 2) or VEGFDLLBII015 (VHH 3) or every second day (closed symbols) with VEGFDLLBII013 (VHH 1) or VEGFDLLBII010 (VHH 2). Day 1 is the first day, day 21 the last day of treatment. The triangles on the top of the graph indicate the treatment days.

TABLE 75
Tumor volume: treatment vs. control (results on day 21)

| | Dose | | TGI | |
|---|---|---|---|---|
| Compound | [mg/kg] | Schedule | [%] | p value |
| Vehicle | — | qdx21 | — | — |
| VEGFDLLBII013 | 7 | qdx21 | 92.2 | 0.0004 |
| | 2.33 | qdx21 | 87.6 | 0.0004 |
| | 14 | q2dx11 | 93.1 | 0.0004 |
| VEGFDLLBII010 | 4.05 | qdx21 | 97.1 | 0.0004 |
| | 1.35 | qdx21 | 93.4 | 0.0004 |
| | 8.1 | q2dx11 | 94.9 | 0.0004 |
| VEGFDLLBII015 | 8.43 | qdx21 | 91.6 | 0.0004 |
| | 2.81 | qdx21 | 90.1 | 0.0004 |

TABLE 76
Body weight: treatment vs. control (results on day 21)

| | | | Weight | |
|---|---|---|---|---|
| | Dose | | gain | |
| Compound | [mg/kg] | Schedule | [%] | p value |
| Vehicle | — | qdx21 | 8.85 | — |
| VEGFDLLBII013 | 7 | qdx21 | 7.97 | 0.3177 |
| | 2.33 | qdx21 | 8.63 | 0.3698 |
| | 14 | q2dx11 | 8.15 | 0.2681 |
| VEGFDLLBII010 | 4.05 | qdx21 | 6.61 | 0.3004 |
| | 1.35 | qdx21 | 9.05 | 0.4811 |
| | 8.1 | q2dx11 | 9.50 | 0.5937 |
| VEGFDLLBII015 | 8.43 | qdx21 | 10.9 | 0.6655 |
| | 2.81 | qdx21 | 6.56 | 0.6655 |

### EXAMPLE 18

Pharmacokinetics of Formatted VHHs in Mice

In order to determine the pharmacokinetics of selected VHHs in mice, a single dose of 33 nmol/kg in 0.1 mL is administered i.p. to six animals/group BomTac:NMRI-Foxn1nu female mice (6-7 weeks old). At different time points (3 mice per timepoint) approximately 50 μl blood is obtained by retroorbital bleeding under isoflurane anaesthesia. The samples are centrifuged after 30 min and the obtained 20 μL serum are stored at −20° C. until analysis. VHH concentrations are measured by a sandwich ELISA.

Microtiter plates (Medisorp Nunc) are coated with 100 μl per well of human VEGF (R&D Systems 293-VE/CF) diluted to 0.5 μg/ml in carbonate buffer pH 9.6 over night at +4° C. After washing with 300 μl deionized water, residual binding sites are blocked by addition of 200 μl blocking buffer (PBS/0.5% bovine serum albumin/0.05% Tween 20) for 0.5 hours.

After an additional washing step, 100 μl per well of dilutions of standards or samples in serum dilution medium (SDM, blocking buffer+2% mouse serum pool, PAA Labor GmbH) are added to the ELISA plates and incubated on a plate shaker for 1 hour at room temperature. For standard curve generation, VHHs are diluted to 100 (VEGFDLLBII013) or 10 (VEGFDLLBII010 and VEGFDLLBII015) ng/ml in serum dilution medium and added to the ELISA plates in 8 twofold dilutions in SDM in duplicates. Mouse serum samples are diluted a minimum of 1:50 in blocking buffer and further dilutions are made in SDM. Serum samples are added to the ELISA plates also in 8 twofold dilutions and duplicates.

Plates are washed once more and for detection of bound VHHs 100 μl per well of human DII4-HIS (R&D Systems 1506-D4/CF) diluted to 0.2 μg/ml in blocking buffer are added and incubated on the shaker for 1 hour as before. After washing the plates again 100 μl per well of anti-6×polyHistidine-HRPO (R&D Systems MAB050H) diluted 1:5000 in blocking buffer are added and plates incubated for 1 hour as before. After a threefold final wash with 300 μl de-ionized water each, bound VHHs are detected by addition of 100 μl per well of TMB staining solution (Bender MedSystems BMS406.1000) and color development stopped after about 10 minutes incubation at room temperature on the shaker by addition of 100 μl per well of 1 M phosphoric acid. Optical densities of the individual wells are quantified using a microtiter plate spectrophotometer (ThermoMax, Molecular Devices) and the ELISA Software SoftMax Pro (Molecular Devices). Sample results are derived from standard curves fitted using a four parameter logistic curve fit.

TABLE 77

| | VHH serum concentration (nM) | | |
|---|---|---|---|
| time (h) | VEGFDLLBII013 | VEGFDLLBII010 | VEGFDLLBII015 |
| 0.0833 | 2.49 | 4.85 | 0.88 |
| 0.5 | 69.8 | 60.5 | 40.7 |
| 1 | 230 | 264 | 113 |
| 4 | 376 | 335 | 308 |
| 24 | 151 | 184 | 160 |
| 72 | 15.2 | 48.7 | 26.8 |
| 168 | <0.86 | 0.88 | 1.71 |
| 240 | <0.78 | <0.14 | <0.21 |

Serum half lives of VHHs are determined to be 15 h (VEGFDLLBII013), 17 h (VEGFDLLBII010) and 24 h (VEGFDLLBII015), respectively. (Half life determination is done by fitting the last 3 data points from the mean plasma concentration curves with WinNonLin V6 to an exponential slope.)

1. A bispecific binding molecule comprising a DII4-binding component and a VEGF-binding component.

2. A bispecific binding molecule of claim 1, wherein said DII4-binding component and said VEGF-binding component comprise at least one DII4-binding immunoglobulin single variable domain and at least one VEGF-binding immunoglobulin single variable domain, respectively.

3. A bispecific binding molecule of claim 2, wherein said immunoglobulin single variable domains are VHHs.

4. A bispecific binding molecule of claim 2, wherein said VEGF-binding component is located N-terminally.

5. A bispecific binding molecule of claim 2, wherein said DII4-binding component and said VEGF-binding component comprise at least one VEGF-binding immunoglobulin single variable domain and at least one DII4-binding immunoglobulin single variable domain, respectively, wherein each of said immunoglobulin single variable domains has four framework regions and three complementarity determining regions CDR1, CDR2 and CDR3, respectively, wherein
a) a CDR3 of said at least one DII4-binding immunoglobulin single variable domain has an amino acid sequence selected from
i. Arg Ala Pro Asp Thr Arg Leu Xaa Pro Tyr Xaa Tyr Asp Xaa as shown in SEQ ID NO: 1, wherein Xaa at position 8 is Arg, Ala or Glu; Xaa at position 11 is Leu or Glu; and Xaa at position 14 is Tyr or His; and
ii. Asp Arg Tyr Ile Trp Ala Arg Gln Gly Glu Tyr Trp Gly Ala Tyr Xaa Asp Tyr as shown in SEQ ID NO: 2, wherein Xaa is Gln, Ala or Tyr; and wherein
b) a CDR3 of said at least one VEGF-binding immunoglobulin single variable domain has the amino acid sequence Ser Arg Ala Tyr Gly Ser Ser Arg Leu Arg Leu Ala Asp Thr Tyr Xaa Tyr, as shown in SEQ ID NO: 3, wherein Xaa is Asp or Glu, wherein said VEGF-binding immunoglobulin single variable domain is capable of blocking the interaction of human recombinant VEGF165 with the human recombinant VEGFR-2 with an inhibition rate of ≧60%.

6. A bispecific binding molecule of claim 5, wherein said immunoglobulin single variable domain is a VHH that has been obtained by sequence optimization, optionally after affinity maturation, of a parent immunoglobulin single variable domain VHH.

7. A bispecific binding molecule of claim 6, wherein said DII4-binding VHH has been obtained from a parent VHH with an amino acid sequence selected from sequences shown in SEQ ID NOs: 4-20 and in Table 5.

8. A bispecific binding molecule of claim 7, wherein said parent VHH has an amino acid sequence shown in SEQ ID NO: 10.

9. A bispecific binding molecule of claim 8, wherein said DII4-binding VHH has been obtained by sequence optimization of an affinity-matured VHH derived from the VHH with the sequence shown in SEQ ID NO: 10, wherein said affinity-matured VHH is selected from VHHs having amino acid sequences shown in SEQ ID NOs: 21-27 and in Table 16.

10. A bispecific binding molecule of claim 9, wherein said affinity-matured VHH has an amino acid sequence shown in SEQ ID NO:22 and wherein said sequence-optimized VHH has an amino acid sequence selected from sequences shown in SEQ ID NOs: 34 and 35 and in Table 23.

11. A bispecific binding molecule of claim 7, wherein said parent VHH has an amino acid sequence shown in SEQ ID NO: 12.

12. A bispecific binding molecule of claim 11, wherein said DII4-binding VHH has been obtained by sequence optimization of an affinity-matured VHH derived from the VHH with the sequence shown SEQ ID NO: 12, wherein said affinity-matured VHH is selected from VHHs having amino acid sequences shown in in SEQ ID NOs: 28-33 and in Table 17.

13. A bispecific binding molecule of claim 12, wherein said affinity-matured VHH has an amino acid sequence shown in SEQ ID NO: 32 and wherein said sequence-optimized VHH has an amino acid sequence selected from sequences shown in SEQ ID NOs: 40 and 41.

14. A bispecific binding molecule of claim 6, wherein said VEGF-binding is a VHH that is derived from a VHH having a sequence selected from sequences shown in SEQ ID NOs: 42-44 and Table 32.

15. A bispecific binding molecule of claim 14, wherein said VEGF-binding VHH has been obtained by sequence optimization of a VHH with an amino acid sequence shown in SEQ ID NO: 43.

16. A bispecific binding molecule of claim 15, wherein said sequence-optimized VHH has an amino acid sequence selected from sequences shown in SEQ ID NOs: 63 and 64 and Table 59.

17. A bispecific binding molecule of claim 3, wherein the VEGF-binding component is a biparatopic VHH, wherein the VHHs forming the building blocks of said biparatopic VHH bind to non-overlapping epitopes.

18. A bispecific binding molecule of claim 17, wherein at least one VHH is capable of blocking the interaction between recombinant human VEGF and the recombinant human VEGFR-2 with an inhibition rate of ≧60% and wherein at least one VHH is capable of blocking said interaction with an inhibition rate of ≦60%.

19. A bispecific binding molecule of claim 18, which said VHH with an inhibition rate of ≦60% is a sequence-optimized variant of a VHH with a sequence shown in SEQ ID NO: 45.

20. A bispecific binding molecule of claim 19, wherein said VHH has a sequences shown in SEQ ID Nos: 65 and 66 and in Table or a sequence shown in SEQ ID NO: 67 (Table 63).

21. A bispecific binding molecule of claim 5 comprising
a) as the DII4-binding component a VHH with a sequence selected from sequences in SEQ ID NO: 35 or 41, and
b) as the VEGF-binding component
i. a VHH with a sequence shown in SEQ ID NO: 64 or
ii. a biparatopic VHH comprising a VHH with a sequence shown in SEQ ID NO: 64 and a VHH with a sequence shown in SEQ ID NO: 67.

22. A bispecific binding molecule of claim 1, comprising one or more linker molecules and/or half-life-extending moieties.

23. A bispecific binding molecule of claim 22, wherein said half-life extending moiety is covalently linked to or fused to an immunoglobulin single variable domain and is selected from an Fc portion, an albumin, an albumin binding immunoglobulin single variable domain, or a polyoxyalkylene molecule.

24. A bispecific binding molecule of claim 21, which has an amino acid sequence shown in SEQ ID NO: 81.

25. A bispecific binding molecule of claim 21, which has an amino acid sequence shown in SEQ ID NO: 82.

26. A bispecific binding molecule of claim 21, which has an amino acid sequence shown in SEQ ID NO: 83.

27. A bispecific binding molecule of claim 21, which has an amino acid sequence shown in SEQ ID NO: 84.

28. A bispecific binding molecule of claim 21, which has an amino acid sequence shown in SEQ ID NO: 85.

29. A bispecific binding molecule of claim 21, which has an amino acid sequence shown in SEQ ID NO: 86.

30. A nucleic acid molecule encoding a bispecific binding molecule of claim 1 or a vector containing same.

31. A host cell containing a nucleic acid molecule of claim 30.

32. A pharmaceutical composition containing at least one VEGF-binding molecule of claim 1 as the active ingredient.

33. The pharmaceutical composition of claim 32 for the treatment of a disease that is associated with VEGF-mediated effects on angiogenesis.

34. The pharmaceutical composition of claim 32 for the treatment of cancer and cancerous diseases.

35. The pharmaceutical composition of claim 32 for the treatment of eye diseases.
